(19) United States
(12) Patent Application Publication
(10) Pub. No.: US 2005/0227243 A1
(43) Pub. Date: Oct. 13, 2005

(54) Cell cycle progression proteins

(75) Inventors: Peter Deak, Szeged (HU); Lisa Frenz, Dundee (GB); David Glover, Dundee (GB); Carol Midgley, Dundee (GB)

Correspondence Address: PALMER & DODGE, LLP;KATHLEEN M. WILLIAMS, 111 HUNTINGTON AVENUE, BOSTON, MA, 02199, US

(73) Assignee: Cyclacel Ltd.

(21) Appl. No.: 10/840,060

(22) Filed: May 5, 2004

Related U.S. Application Data
(63) Continuation of application No. PCT/GB02/04780, filed on Oct. 23, 2002.

(30) Foreign Application Priority Data
Nov. 5, 2001 (GB) GB 0126506.5
Nov. 27, 2001 (GB) GB 0128384.5
Feb. 11, 2002 (GB) GB 0203185.4

(51) Int. Cl. C12Q 1/68; G01N 33/574; A61K 39/395; A61K 48/00

(52) U.S. Cl. 435/6; 435/7.23; 514/44; 424/155.1

(57) ABSTRACT

Polynucleotides encoding a number of Drosophila gene products are provided. Polynucleotide probes derived from these nucleotide sequences, polypeptides encoded by the polynucleotides and antibodies that bind to the polypeptides are also provided.

The present invention relates to a number of genes implicated in the processes of cell cycle progression, including mitosis and meiosis.

We have now identified a number of genes in the X chromosome of Drosophila, mutations in which disrupt cell cycle progression, for example the processes of mitosis and/or meiosis. We have determined the phenotypes of these mutations and relate the mutations to the total genome sequence and so identify individual genes essential for cell cycle progression.

According to one aspect of the present invention, we provide a use of a polynucleotide as set out in Table 5, or a polypeptide encoded by the polypeptide, in a method of prevention, treatment or diagnosis of a disease in an individual.

Preferably, the polynucleotide comprises a human polypeptide as set out in column 3 of Table 5. In preferred embodiments, the polynucleotide or polypeptide is used to identify a substance capable of binding to the polypeptide, which method comprises incubating the polypeptide with a candidate substance under suitable conditions and determining whether the substance binds to the polypeptide.

Alternatively or in addition, the polynucleotide or polypeptide is used to identify a substance capable of modulating the function of the polypeptide, the method comprising the steps of: incubating the polypeptide with a candidate substance and determining whether activity of the polypeptide is thereby modulated.

The polynucleotide or polypeptide may be administered to an individual in need of such treatment. Alternatively, or in addition, the substance identified by the method is administered to an individual in need of such treatment.

The use may be for a method of diagnosis, in which the presence or absence of a polynucleotide is detected in a biological sample in a method comprising: (a) bringing the biological sample containing nucleic acid such as DNA or RNA into contact with a probe comprising a fragment of at least 15 nucleotides of the polynucleotide as set out in Table 5 under hybridising conditions; and (b) detecting any duplex formed between the probe and nucleic acid in the sample.

Alternatively, or in addition, the presence or absence of a polypeptide is detected in a biological sample in a method comprising: (a) providing an antibody capable of binding to the polypeptide; (b) incubating a biological sample with said antibody under conditions which allow for the formation of an antibody-antigen complex; and (c) determining whether antibody-antigen complex comprising said antibody is formed.

In highly preferred embodiments, the disease comprises a proliferative disease such as cancer.

In a further aspect of the invention, we provide a method of modulating, preferably down-regulating, the expression of a polynucleotide as set out in Table 5 in a cell, the method comprising introducing a double stranded RNA (dsRNA) corresponding to the polynucleotide, or an antisense RNA corresponding to the polynucleotide, or a fragment thereof, into the cell.

According to another aspect of the present invention, we provide a polynucleotide selected from: (a) polynucleotides comprising any one of the nucleotide sequences set out in Example 19, preferably Shp2 polynucleotide, or the complement thereof; (b) polynucleotides comprising a nucleotide sequence capable of hybridising to the nucleotide sequences set out in Example 19, preferably Shp2 polynucleotide, or a fragment thereof; (c) polynucleotides comprising a nucleotide sequence capable of hybridising to the complement of the nucleotide sequences set out in Example 19, preferably Shp2 polynucleotide, or a fragment thereof; (d) polynucleotides comprising a polynucleotide sequence which is degenerate as a result of the genetic code to the polynucleotides defined in (a), (b) or (c).

There is provided, according to a further aspect of the present invention, a polynucleotide selected from: (a) polynucleotides comprising any one of the nucleotide sequences set out in Example 28, preferably Dlg1 or Dlg2 polynucleotide, or the complement thereof; (b) polynucleotides comprising a nucleotide sequence capable of hybridising to the nucleotide sequences set out in Example 28, preferably Dlg1 or Dlg2 polynucleotide, or a fragment thereof; (c) polynucleotides comprising a nucleotide sequence capable of hybridising to the complement of the nucleotide sequences set out in Example 28, preferably Dlg1 or Dlg2 polynucleotide, or a fragment thereof, (d) polynucleotides comprising a polynucleotide sequence which is degenerate as a result of the genetic code to the polynucleotides defined in (a), (b) or (c).

We provide, according to another aspect of the present invention, a polynucleotide selected from: (a) polynucleotides comprising any one of the nucleotide sequences set out in Table 5 or the complement thereof, (b) polynucleotides comprising a nucleotide sequence capable of hybridising to the nucleotide sequences set out in Table 5, or a fragment thereof; (c) polynucleotides comprising a nucleotide sequence capable of hybridising to the complement of the nucleotide sequences set out in Table 5, or a fragment thereof; (d) polynucleotides comprising a polynucleotide sequence which is degenerate as a result of the genetic code to the polynucleotides defined in (a), (b) or (c).

As a further aspect of the present invention, there is provided a polynucleotide selected from: (a) polynucleotides comprising any one of the nucleotide sequences set out in Examples 1 to 18, 20 to 27 and 29 or the complement thereof, (b) polynucleotides comprising a nucleotide sequence capable of hybridising to the nucleotide sequences set out in Examples 1 to 18, 20 to 27 and 29, or a fragment thereof; (c) polynucleotides comprising a nucleotide sequence capable of hybridising to the complement of the nucleotide sequences set out in Examples 1 to 18, 20 to 27 and 29, or a fragment thereof; (d) polynucleotides comprising a polynucleotide sequence which is degenerate as a result of the genetic code to the polynucleotides defined in (a), (b) or (c).

We provide, according to a further aspect of the present invention, a polynucleotide selected from: (a) polynucleotides comprising any one of the nucleotide sequences set out in Examples 1, 2, 2A, 2B and 2C or the complement thereof; (b) polynucleotides comprising a nucleotide sequence capable of hybridising to the nucleotide sequences set out in Examples 1, 2, 2A, 2B and 2C, or a fragment thereof; (c) polynucleotides comprising a nucleotide sequence capable of hybridising to the complement of the nucleotide sequences set out in Examples 1, 2, 2A, 2B and 2C, or a fragment thereof; (d) polynucleotides comprising a polynucleotide sequence which is degenerate as a result of the genetic code to the polynucleotides defined in (a), (b) or (c).

The present invention, in another aspect, provides polynucleotide selected from: (a) polynucleotides comprising any one of the nucleotide sequences set out in Examples 3 to 9 and 9A or the complement thereof; (b) polynucleotides comprising a nucleotide sequence capable of hybridising to the nucleotide sequences set out in Examples 3 to 9 and 9A, or a fragment thereof; (c) polynucleotides comprising a nucleotide sequence capable of hybridising to the complement of the nucleotide sequences set out in Examples 3 to 9 and 9A, or a fragment thereof; (d) polynucleotides comprising a polynucleotide sequence which is degenerate as a result of the genetic code to the polynucleotides defined in (a), (b) or (c).

In a further aspect of the present invention, there is provided polynucleotide selected from: (a) polynucleotides comprising any one of the nucleotide sequences set out in Examples 10 to 29 or the complement thereof; (b) polynucleotides comprising a nucleotide sequence capable of hybridising to the nucleotide sequences set out in Examples 10 to 29, or a fragment thereof; (c) polynucleotides comprising a nucleotide sequence capable of hybridising to the complement of the nucleotide sequences set out in Examples 10 to 29, or a fragment thereof; (d) polynucleotides comprising a polynucleotide sequence which is degenerate as a result of the genetic code to the polynucleotides defined in (a), (b) or (c).

As a further aspect of the invention, we provide a polynucleotide probe which comprises a fragment of at least 15 nucleotides of a polynucleotide according to any of the above aspects of the invention.

The present invention also provides a polypeptide which comprises any one of the amino acid sequences set out in Examples 1 to 29 or in any of Examples 1 to 2, 2A, 2B and 2C, Examples 3 to 9 and 9A and Examples 10 to 29, or a homologue, variant, derivative or fragment thereof.

Preferably the polypeptide is encoded by a cDNA sequence obtainable from a eukaryotic cDNA library, preferably a metazoan cDNA library (such as insect or mammalian) said DNA sequence comprising a DNA sequence being selectively detectable with a nucleotide sequence, preferably a Drosophila nucleotide sequence, as shown in any one of Examples 1 to 29.

The term “selectively detectable” means that the cDNA used as a probe is used under conditions where a target cDNA is found to hybridize to the probe at a level significantly above background. The background hybridization may occur because of other cDNAs present in the cDNA library. In this event background implies a level of signal generated by interaction between the probe and a non-specific cDNA member of the library which is less than 10 fold, preferably less than 100 fold as intense as the specific interaction observed with the target cDNA. The intensity of interaction may be measured, for example, by radiolabelling the probe, e.g. with ³²P. Suitable conditions may be found by reference to the Examples, as well as in the detailed description below.

A polynucleotide encoding a polypeptide as described here is also provided.

We further provide a vector comprising a polynucleotide of the invention, for example an expression vector comprising a polynucleotide of the invention operably linked to a regulatory sequence capable of directing expression of said polynucleotide in a host cell.

Also provided is an antibody capable of binding such a polypeptide.

In a further aspect the present invention provides a method for detecting the presence or absence of a polynucleotide of the invention in a biological sample which method comprises: (a) bringing the biological sample containing DNA or RNA into contact with a probe comprising a nucleotide of the invention under hybridising conditions; and (b) detecting any duplex formed between the probe and nucleic acid in the sample.

In another aspect the invention provides a method for detecting a polypeptide of the invention present in a biological sample which comprises: (a) providing an antibody of the invention; (b) incubating a biological sample with said antibody under conditions which allow for the formation of an antibody-antigen complex; and (c) determining whether antibody-antigen complex comprising said antibody is formed.

Knowledge of the genes involved in cell cycle progression allows the development of therapeutic agents for the treatment of medical conditions associated with aberrant cell cycle progression. Accordingly, the present invention provides a polynucleotide of the invention for use in therapy. The present invention also provides a polypeptide of the invention for use in therapy. The present invention further provides an antibody of the invention for use in therapy.

In a specific embodiment, the present invention provides a method of treating a tumor or a patient suffering from a proliferative disease, comprising administering to a patient in need of treatment an effective amount of a polynucleotide, polypeptide and/or antibody of the invention.

The present invention also provides the use of a polypeptide of the invention in a method of identifying a substance capable of affecting the function of the corresponding gene. For example, in one embodiment the present invention provides the use of a polypeptide of the invention in an assay for identifying a substance capable of inhibiting cell cycle progression. The assay involves contacting the polypeptide with a candidate substance or molecule, and detecting modulation of activity of the polypeptide. In preferred embodiments, further steps of isolating or synthesising the substance so identified are carried out.

The substance may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. For example, possible functions of genes of the invention for which it may be desired to identify substances which affect such functions include chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In a further aspect the present invention provides a method for identifying a substance capable of binding to a polypeptide of the invention, which method comprises incubating the polypeptide with a candidate substance under suitable conditions and determining whether the substance binds to the polypeptide.

In an additional aspect, the invention provides kits comprising polynucleotides, polypeptides or antibodies of the invention and methods of using such kits in diagnosing the presence of absence of polynucleotides and polypeptides of the invention including deleterious mutant forms.

Also provided is a substance identified by the above methods of the invention. Such substances may be used in a method of therapy, such as in a method of affecting cell cycle progression, for example mitosis and/or meiosis.

The invention also provides a process comprising the steps of: (a) performing one of the above methods; and (b) preparing a quantity of those one or more substances identified as being capable of binding to a polypeptide of the invention.

Also provided is a process comprising the steps of: (a) performing one of the above methods; and (b) preparing a pharmaceutical composition comprising one or more substances identified as being capable of binding to a polypeptide of the invention.

We further provide a method for identifying a substance capable of modulating the function of a polypeptide of the invention or a polypeptide encoded by a polynucleotide of the invention, the method comprising the steps of: incubating the polypeptide with a candidate substance and determining whether activity of the polypeptide is thereby modulated.

A substance identified by a method or assay according to any of the above methods or processes is also provided, as is the use of such a substance in a method of inhibiting the function of a polypeptide. Use of such a substance in a method of regulating a cell division cycle function is also provided.

We further provide a method of identifying a human nucleic acid sequence, by: (a) selecting a Drosophila polypeptide identified in any of Examples 1 to 29; (b) identifying a corresponding human polypeptide; (c) identifying a nucleic acid encoding the polypeptide of (b).

Preferably, a human homologue of the Drosophila sequence, or a human sequence similar to the Drosophila sequence, is identified in step (b).

Preferably, the human polypeptide has at least one of the biological activities, preferably substantially all the biological activities of the Drosophila polypeptide.

We provide a human polypeptide identified by a method according to the previous aspect of the invention.

### BRIEF DESCRIPTION OF THE FIGURES

FIG. 1 shows mitotic index after RNAi knockdown of Corkscrew (CG3954) in Dme1-2 Drosophila cultured cells. Values are an average of triplicate samples. Positive controls are siRNA with the mitotic genes Polo kinase and Orbit, negative controls are siRNA with water and with an siRNA against non-endogenous gene GL3

FIG. 2 shows a BLASTP alignment of Drosophila Corkscrew (CG3954) (query sequence), identified in Example 19 as a cell cycle gene, and human Shp2 Protein-tyrosine phosphatase, non-receptor type 11 (genbank accession D13540 ) (subject sequence).

FIG. 3 shows a histogram of Facs analysis of cell cycle compartment as determined by DNA content in U20S cells after human Shp2 siRNA transfection for 48 hours. The negative control is transfection with siRNA against the non-endogenous gene GL3.

FIG. 4 shows fluorescence micrographs showing the effect of Shp2 siRNAi in U2OS cells. A) Irregular nuclear shape, B) Increase in apoptosis.

FIG. 5 shows Mitotic index after RNAi knockdown of Drosophila discs large 1 Dlg1 (CG1725) in Dme1-2 Drosophila cultured cells. Values are an average of triplicate samples. Positive controls are siRNA with the mitotic genes Polo kinase and Orbit, negative controls are siRNA with water and with an siRNA against non-endogenous gene GL3

FIG. 6A shows a BLASTP alignment of Drosophila discs large 1 Dlg1 (CG1725), identified in Example 28 as a cell cycle gene, and human discs, large (Drosophila) homolog 1 (genbank accession U13896).

FIG. 6B shows a ClustalW alignment of Drosophila discs large 1 Dlg1 (CG1725) and human discs, large (Drosophila) homolog 1 (genbank accession U13896).

FIG. 6C shows a BLASTP alignment of Drosophila discs large 1 Dlg1 (CG1725), and human discs, large (drosophila) homolog 2 (genbank accession U32376).

FIG. 6D shows a ClustalW alignment of Drosophila discs large 1 Dlg1 (CG1725) and human discs, large (drosophila) homolog 2 (genbank accession U32376).

FIG. 7 shows a ClustalW alignment Drosophila Dlg1 and 5 human Dlg genes (Dlg 1-5) so far described.

FIG. 8 shows a histogram of FACS analysis of cell cycle status after siRNA in U20S cells. Negative control is siRNA against the non-endogenous GL3 gene.

FIG. 9 fluorescence micrographs showing the dominant phenotype observed with Dlg1 COD1654 siRNAi in U20S cells. A) Multicentrosomal cells at prometaphase and anaphase. B) Cytokinesis defect

FIG. 10 fluorescence micrographs showing the dominant phenotype observed with Dlg2 COD1652 siRNAi in U20S cells. A) Multicentrosomal cell at telophase. B) Cytokinesis defects.

### DETAILED DESCRIPTION

We provide for polynucleotide sand polypeptides whose sequences are set out, or which are referred to, in any of Examples 1 to 29, including Drosophila and human sequences. In particular, we provide for the sequences, including human sequences, and their use in diagnosis and treatment of disease (including prevention and treatment of diseases, syndromes and symptoms) as described in further detail below. A particularly suitable disease for treatment or diagnosis is a proliferative disease such as cancer or any tumor. The polynucleotides and polypeptides disclosed here may be used in screening assays to identify compounds which are capable of binding to, or inhibiting an activity of, the polypeptide or polynucleotide.

Particularly preferred polypeptides include those set out in Example 19 and referred to as Shp2, as well as those set out in Example 28 and referred to as Dlg1 and Dlg2. Accordingly, we provide for Shp2 polypeptide and polynucleotide, as well as Dlg1 and Dlg2 polypeptide and polynucleotide, for the treatment and diagnosis of diseases such as cancer, as described in further detail below.

By the term “Shp2”, we mean a sequence as set out in Example 19 and having the accession number NM_{—}002834, together with its variants, homologues, derivatives, fragments and complements as described in further detail below. Preferably, the term “Shp2” should be taken to refer to the human sequence itself. Two transcript variants (variants 1 and 2 as set out in Example 19) are known, and both are encompassed in the term “Shp2”. Shp2 is also known as Homo sapiens protein tyrosine phosphatase, non-receptor type 11 (PTPN11). Furthermore, various sequences differing in length are known for Shp2, and each of these is intended to be included for the uses and compositions described here.

As used in this document, the terms “Dlg1” and “Dlg2” mean the sequences as set out in Example 28 and having the GENBANK accession numbers U13896 and U32376 respectively. Variants, homologues, derivatives, fragments and complements (as described in further detail below) of each of these sequences are also included within the meaning of these terms.

Dlg1 is also known as “human discs, large (Drosophila) homolog 1” while Dlg2 is also known as “human discs, large (Drosophila) homolog 2, chapsyn-110 channel-associated protein of synapses-110′”. Various sequences differing in length are known for Dlg1 and Dlg2, and each of these is intended to be included for the uses and compositions described here.

Preferably, the polypeptides and polynucleotides are such that they give rise to or are associated with defined phenotypes when mutated.

For example, mutations in the polypeptides and polynucleotides may be associated with female sterility; such polypeptides and polynucleotides are conveniently categorised as “Category 1”. Phenotypes associated with Category 1 polypeptides and polynucleotides include any one or more of the following, singly or in combination: Female semi-sterile, brown eggs laid; female sterile, few eggs laid, several fully matured eggs in ovarioles; female semi-sterile, lays eggs, but arrest before cortical migration; “Female sterile, no eggs laid. Fully mature eggs, but “retained eggs” phenotype. Also has a mitotic phenotype: higher mitotic index, uneven chromosome staining, tangled and badly defined chromosomes with frequent bridges”; Female sterile (semi-sterile), 2-3 fully matured eggs in each of the ovarioles.

Alternatively, mutations in the polypeptides and polynucleotides may be associated with male sterility; such polypeptides and polynucleotides are conveniently categorised as “Category 2”. Phenotypes associated with Category 2 polypeptides and polynucleotides include any one or more of the following, singly or in combination: Lethal phase pharate adult, cytokinesis defect—some onion stage cysts with large nebenkerns; reduced adult viability, cytokinesis defect—onion stage cysts have variable sized Nebenkerns—mitotic phenotype: tangled unevenly condensed chromosomes, anaphases with lagging chromosomes and bridges; semi-lethal male and female, cytokinesis defect—in some cysts, variable sized Nebenkerns; male sterile, cytokinesis defect, different meiotic stages within one cyst, variable sized nuclei, 2-4 nuclei, mitotic phenotype: semi-lethal, rod-like overcondensed chromosomes, high mitotic index, lagging chromosomes and bridges; male sterile, asynchronous meiotic divisions, cysts with large Nebenkern and 1-2 larger nuclei, testis from 2-3 old males become smaller, high mitotic index, colchicine type overcondensaton, many anaphases and telophases, no decondensation in telophase, mitotic phenotype: high mitotic index, colchicines-type overcondensed chromosomes, many ana- and relophases, no decondensation in telophase; cytokinesis defect, small testis, no meiosis observed, variable sized Nebenkerns with 2-4N nuclei; male sterile, cytokinesis defect, larger Nebenkerns with 2-4N nuclei; Male sterile, Cytokinesis defect: variable sized Nebenkerns with 4N nuclei, some nuclei detached from Nebenkern.

Mutations in the polypeptides and polynucleotides may be associated with a mitotic (neuroblast) phenotype (“Category 3”). Phenotypes associated with Category 3 polypeptides and polynucleotides include any one or more of the following, singly or in combination: lethal phase between pupil and pharate adult (P-pA), high mitotic index, rod-like overcondensed chromosomes, a few circular metaphases, many overcondensed anaphases and telophases, a few tetraploid cells; lethal phase pharate adult, high mitotic index, rod-like overcondensed chromosomes, lagging chromosomes and bridges in anaphase, highly condensed; lethal phase pupal—pharate adult, high mitotic index, colchicines-type overcondensation, high frequency of polyploids; lethal phase pupal—pharate adult, high mitotic index, colchicines-type overcondensed chromosomes, many strongly stained nuclei; lethal phase larval stage 3-pre-pupal-pupal, small optic lobes, missing or small imaginal discs, badly defined chromosomes; lethal phase pharate adult, Dot and rod-like overcondensed chromosomes, high mitotic index, overcondensed anaphases some with lagging chromosomes, a few tetraploid cells with overcondensed chromosomes, XYY males; lethal phase embryonic larval phase3-pre-pupal-pupal, high mitotic index, dot-like chromosomes, strong metaphase arrest; lethal phase larval phase 3D pre-pupal-pupal-pharate adult-adult, high mitotic index, dot and rod-like overcondensed chromosomes, high frequency of polyploids; lethal phase larval stage 3 (few pupae), high mitotic index, colchicine-type overcondensation of chromosomes, polyploid cells, mininuclei formation; lethal phase larval stage 1-2, low mitotic index, few cells in mitosis, metaphase with separated chromosomes; viable, high mitotic index, colchicines-type overcondensed chromosomes, a few polyploid cells; lethal phase pharate adult, high mitotic index, rod like overcondensed chromosomes, few anaphases with lagging chromosomes; lethal phase larval stage 3-pharate adult, small brain and optic lobes, high mitotic index, rod-like overcondensed chromosomes, fewer ana- and telophases, overcondensed chromosomes in ana- and telophase; lethal phase larval stage 3, small brain, few cells in mitosis, badly defined chromosomes, weak chromosome condensation, abnormal anaphases with broken chromosomes; lethal phase larval stage 3, small brain, high mitotic index, rod-like overcondensed chromosomes, fewer ana- and telophases; semilethal male and female, Low mitotic index, badly defined chromosomes, weak/uneven staining, fewer ana- and telophases; lethal phase pupal to pharate adult, lagging chromosomes and bridges in ana- and telophase; lethal phase, pupal, uneven chromosome condensation, lagging chromosomes in anaphase; lethal phase pupal, higher mitotic index, colchicine-like overcondensed chromosomes, many ana- and telophases, lagging chromosomes; lethal phase, prepupal—pupal, high mitotic index, colchicines-like chromosome condensation, metaphase arrest.

The polypeptides and polynucleotides described here may also be categorised according to their function, or their putative function.

For example, the polypeptides described here preferably comprise, and the polynucleotides described here are ones which preferably encode polypeptides comprising, any one or more of the following: CREB-binding proteins, transcription factors, casein kinases, serine threonine kinases, preferably involved in replication and cell cycle, protein phosphatases, membrane associated proteins, preferably involved in priming synaptic vesicles, dynein light chains, microtubule motor proteins, protein phosphatases, protein phosphatases with p53 dependent expression, proteins capable of inhibiting cell division, ribosomal proteins, motor proteins, cytoskeletal binding proteins linking to plama membrane, proteins involved in cytokinesis and cell shape, phosphatidylinositol 3-kinases, C-myc oncogenes, transcription factors, dehydrogenases, thioredoxin reductases, cell cycle regulators preferably involved in cyclin degradation; centrosome components, protein tyrosine phosphatases, Wnt oncogenes, ubiquitin ligases, ubiquitin conjugating enzymes, vesicle trafficking proteins, protein kinases (including protein kinases which regulate the G1/S phase transition and/or DNA replication in mammalian cells), serine/threonine kinases, including serine/threonine kinases involved in winglwess signaling pathway, components of cell junctions, including components of cell junctions having a role in proliferation and Ras associated effector proteins; hydroxymethyltransferase; glycosylation/membrane protein; hydrogen transporting ATP synthase; role in cell cycle progression.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Polypeptides

It will be understood that polypeptides as described here are not limited to polypeptides having the amino acid sequence set out in Examples 1 to 29 or fragments thereof but also include homologous sequences obtained from any source, for example related viral/bacterial proteins, cellular homologues and synthetic peptides, as well as variants or derivatives thereof.

Thus polypeptides also include those encoding homologues from other species including animals such as mammals (e.g. mice, rats or rabbits), especially primates, more especially humans. More specifically, such homologues include human homologues.

Thus, we describe variants, homologues or derivatives of the amino acid sequence set out in Examples 1 to 29, as well as variants, homologues or derivatives of the nucleotide sequence coding for the amino acid sequences as described here.

In the context of this document, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200, 300, 400 or 500 amino acids with any one of the polypeptide sequences shown in the Examples. In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i.e. amino acid residues having similar chemical properties/functions), in the context of this document, it is preferred to express homology in terms of sequence identity.

Homology comparisons can be conducted by eye, or more usually, with the aid of readily available sequence comparison programs. These publicly and commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et aL, 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences includes any substitution of, variation of, modification of, replacement of, deletion of or addition of one (or more) amino acids from or to the sequence providing the resultant amino acid sequence retains substantially the same activity as the unmodified sequence, preferably having at least the same activity as the polypeptides presented in the sequence listings in the Examples.

Polypeptides having the amino acid sequence shown in the Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides also include fragments of the full length sequences mentioned above. Preferably said fragments comprise at least one epitope. Methods of identifying epitopes are well known in the art. Fragments will typically comprise at least 6 amino acids, more preferably at least 10, 20, 30, 50 or 100 amino acids.

Proteins as described here are typically made by recombinant means, for example as described below. However they may also be made by synthetic means using techniques well known to skilled persons such as solid phase synthesis. Proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins as described here may also be obtained by purification of cell extracts from animal cells.

The proteins may be in a substantially isolated form. It will be understood that the protein may be mixed with carriers or diluents which will not interfere with the intended purpose of the protein and still be regarded as substantially isolated. A protein may also be in a substantially purified form, in which case it will generally comprise the protein in a preparation in which more than 90%, e.g. 95%, 98% or 99% of the protein in the preparation is a protein as described in this document.

A polypeptide may be labeled with a revealing label. The revealing label may be any suitable label which allows the polypeptide to be detected. Suitable labels include radioisotopes, e.g. ¹²⁵I, enzymes, antibodies, polynucleotides and linkers such as biotin. Labeled polypeptides as described here may be used in diagnostic procedures such as immunoassays to determine the amount of a polypeptide in a sample. Polypeptides or labeled polypeptides may also be used in serological or cell-mediated immune assays for the detection of immune reactivity to said polypeptides in animals and humans using standard protocols.

A polypeptide or labeled polypeptide or fragment thereof may also be fixed to a solid phase, for example the surface of an immunoassay well or dipstick. Such labeled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The polypeptides described here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which such polypeptides are expressed may be used in assay systems to identify candidate substances which interfere with or enhance the functions of the polypeptides described here in the cell.

Polynucleotides

We demonstrate here that mutations in genes encoding the polypeptides disclosed in the Examples demonstrate a cell cycle defect, and that. accordingly these genes and the proteins encoded by them are responsible for cell cycle function.

Polynucleotides as described in this document include polynucleotides that comprise any one or more of the nucleic acid sequences encoding the polypeptides set out in Examples 1 to 29 and fragments thereof. Such polynucleotides also include polynucleotides encoding the polypeptides described here. It is straightforward to identify a nucleic acid sequence which encodes such a polypeptide, by reference to the genetic code. Furthermore, computer programs are available which translate a nucleic acid sequence to a polypeptide sequence, and/or vice versa. Each and all of sequences which are capable of encoding the polypeptides disclosed in the Examples is considered disclosed in this document, and the disclosure of a polypeptide sequence includes a disclosure of all nucleic acids (and their sequences) which encodes that polypeptide sequence.

It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

In preferred embodiments, the polynucleotides comprise those polypeptides, such as cDNA, MRNA, and genomic DNA of the relevant organism, which encode the polypeptides disclosed in the Examples. Such polynucleotides may typically comprise Drosophila cDNA, MRNA, and genomic DNA, Homo sapiens cDNA, MRNA, and genomic DNA, etc. Accession numbers are provided in the Examples for the polypeptide sequences, and it is straightforward to derive the encoding nucleic acid sequences by use of such accession numbers in a relevant database, such as a Drosophila sequence database, a human sequence database, including a Human Genome Sequence database, GadFly, FlyBase, etc. in particular, the annotated Drosophila sequence database of the Berkeley Drosophila Genome Project (GadFly: Genome Annotation Database of Drosophil at http://www.fruitfly.org/annot/) may be used to identify such Drosophila and human polynucleotide sequences. Relevant sequences may also be obtained by searching sequence databases such as BLAST with the polypeptide sequences. In particular, a search using TBLASTN may be employed.

Furthermore, we provide a method of identifying a human nucleic acid sequence, by: (a) selecting a Drosophila polypeptide identified in any of Examples 1 to 29; (b) identifying a corresponding human polypeptide; (c) identifying a nucleic acid encoding the polypeptide of (b). Step (b) may in particular involve identifying a human homologue of the Drosophila sequence, or a human sequence similar to the Drosophila sequence. Preferably, such a polypeptide has at least one of the biological activities, preferably substantially all the biological activities (such as identified in the Examples) of the Drosophila polypeptide. Preferably, the human polypeptide is involved in an aspect of cell cycle control. A human polypeptide identified as above, as well as a sequence of the human polypeptide and a sequence of the human nucleic acid are also provided.

Polynucleotides as described here may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides described herein may be modified by any method available in the art. Such modifications may be carried out in order to enhance the in vivo activity or life span of polynucleotides.

The terms “variant”, “homologue” or “derivative” in relation to a nucleotide sequence include any substitution of, variation of, modification of, replacement of, deletion of or addition of one (or more) nucleic acid from or to the sequence. Preferably said variant, homologues or derivatives code for a polypeptide having biological activity.

As indicated above, with respect to sequence homology, preferably there is at least 50 or 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

This document also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides which capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined 37 stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, we describe nucleotide sequences that can hybridise to the nucleotide sequence as described here under stringent conditions (e.g. 65° C. and 0.1×SSC {1×SSC=0.15 M NaCl, 0.015 M Na₃ Citrate pH 7.0).

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

Polynucleotides which are not 100% homologous to the sequences of described here but are encompassed can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to sequences which encode the polypeptides shown in the Examples. Such sequences may be obtained by probing cDNA libraries made from or genomic DNA libraries from other animal species, and probing such libraries with probes comprising all or part of any on of the sequences under conditions of medium to high stringency. The nucleotide sequences of or which encode the human homologues described in the Examples, may preferably be used to identify other primate/mammalian homologues since nucleotide homology between human sequences and mammalian sequences is likely to be higher than is the case for the Drosophila sequences identified herein.

Similar considerations apply to obtaining species homologues and allelic variants of the polypeptide or nucleotide sequences described here.

Variants and strain/species homologues may also be obtained using degenerate PCR which will use primers designed to target sequences within the variants and homologues encoding conserved amino acid sequences within the sequences described here. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labeled fragments.

In addition, homologous sequences may be identified by searching nucleotide and/or protein databases using search algorithms such as the BLAST suite of programs. This approach is described below and in the Examples.

Alternatively, such polynucleotides may be obtained by site directed mutagenesis of characterised sequences, such as the sequences encoding polypeptides disclosed in the Examples. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. For example, further changes may be desirable to represent particular coding changes found in the sequences coding polypeptides disclosed in the Examples which give rise to mutant genes which have lost their regulatory function. Probes based on such changes can be used as diagnostic probes to detect such mutants.

The polynucleotides described here may be used to produce a primer, e.g. a PCR primer, a primer for an alternative amplification reaction, a probe e.g. labeled with a revealing label by conventional means using radioactive or non-radioactive labels, or the polynucleotides may be cloned into vectors. Such primers, probes and other fragments will be at least 8, 9, 10, or 15, preferably at least 20, for example at least 25, 30 or 40 nucleotides in length, and are also encompassed by the term “polynucleotides” as used herein.

Polynucleotides such as a DNA polynucleotides and probes as described here may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

The polynucleotides or primers may carry a revealing label. Suitable labels include radioisotopes such as ³²P or ³⁵S, enzyme labels, or other protein labels such as biotin. Such labels may be added to the polynucleotides or primers and may be detected using by techniques known per se.

Polynucleotides or primers or fragments thereof labeled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer as described here under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides include bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or TIU residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing nucleotides in a biological sample may be used to determine particular sequences within cells in individuals who have, or are suspected to have, an altered gene sequence, for example within cancer cells including leukaemia cells and solid tumours such as breast, ovary, lung, colon, pancreas, testes, liver, brain, muscle and bone tumours. Cells from patients suffering from a proliferative disease may also be tested in the same way.

In addition, the identification of the genes described in the Examples will allow the role of these genes in hereditary diseases to be investigated. In general, this will involve establishing the status of the gene (e.g. using PCR sequence analysis), in cells derived from animals or humans with, for example, neurological disorders or neoplasms.

The probes as described here may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like.

Homology Searching

Sequence homology (or identity) may be determined using any suitable homology algorithm, using for example default parameters.

Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

The five BLAST programs available at http://www.ncbi.nlm.nih.gov perform the following tasks:
blastp compares an amino acid query sequence against a protein sequence database;
blastn compares a nucleotide query sequence against a nucleotide sequence database;
blastx compares the six-frame conceptual translation products of a nucleotide query sequence (both strands) against a protein sequence database;
tblastn compares a protein query sequence against a nucleotide sequence database dynamically translated in all six reading frames (both strands).
tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191 -201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX” ).

Filtering is only applied to the query sequence (or its translation products), not to database sequences. Default filtering is DUST for BLASTN; SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Nucleic Acid Vectors

Polynucleotides as described in this document can be incorporated into a recombinant replicable vector. The vector may be used to replicate the nucleic acid in a compatible host cell. Thus in a further embodiment, we provide a method of making polynucleotides by introducing a polynucleotide as described here into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors as described here may be transformed or transfected into a suitable host cell as described below to provide for expression of a protein. This process may comprise culturing a host cell transformed with an expression vector as described above under conditions to provide for expression by the vector of a coding sequence encoding the protein, and optionally recovering the expressed protein. Vectors will be chosen that are compatible with the host cell used.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding a polypeptide described here include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells, such as insect cells, may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

The polynucleotides may also be inserted into the vectors described above in an antisense orientation to provide for the production of antisense RNA. Antisense RNA or other antisense polynucleotides may also be produced by synthetic means. Such antisense polynucleotides may be used in a method of controlling the levels of RNAs transcribed from genes comprising any one of the polynucleotides as described.

Host Cells

The vectors and polynucleotides may be introduced into host cells for the purpose of replicating the vectors/polynucleotides and/or expressing the polypeptides encoded by the polynucleotides described here. Although such polypeptides may be produced using prokaryotic cells as host cells, it is preferred to use eukaryotic cells, for example yeast, insect or mammalian cells, in particular mammalian cells.

Vectors/polynucleotides as described here may be introduced into suitable host cells using a variety of techniques known in the art, such as transfection, transformation and electroporation. Where vectors/polynucleotides are to be administered to animals, several techniques are known in the art, for example infection with recombinant viral vectors such as retroviruses, herpes simplex viruses and adenoviruses, direct injection of nucleic acids and biolistic transformation.

Protein Expression and Purfication

Host cells comprising polynucleotides as described here may be used to express polypeptides. Host cells may be cultured under suitable conditions which allow expression of the proteins. Expression of the polypeptides as described may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Polypeptides can be extracted from host cells by a variety of techniques known in the art, including enzymatic, chemical and/or osmotic lysis and physical disruption.

The polypeptides may also be produced recombinantly in an in vitro cell-free system, such as the TnT™ (Promega) rabbit reticulocyte system.

Antibodies

We also provide monoclonal or polyclonal antibodies to polypeptides as described here, or fragments thereof. Thus, we further provide a process for the production of monoclonal or polyclonal antibodies to polypeptides.

If polyclonal antibodies are desired, a selected mammal (e.g., mouse, rabbit, goat, horse, etc.) is immunised with an immunogenic polypeptide bearing an epitope(s) from a polypeptide as described here. Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order that such antibodies may be made, we also provide polypeptides as described here, or fragments thereof, haptenised to another polypeptide for use as immunogens in animals or humans.

Monoclonal antibodies directed against epitopes in the polypeptides described here can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against epitopes from polypeptides described here are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this document, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in method of detecting polypeptides as described in this document present in biological samples by a method which comprises: (a) providing an antibody as described here; (b) incubating a biological sample with said antibody under conditions which allow for the formation of an antibody-antigen complex; and (c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues.

Such antibodies may be bound to a solid support and/or packaged into kits in a suitable container along with suitable reagents, controls, instructions and the like.

Assays

We also provide assays that are suitable for identifying substances which bind to polypeptides as described here and which affect, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, cytokinesis functions, chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In addition, assays suitable for identifying substances that interfere with binding of polypeptides as described here, where appropriate, to components of cell division cycle machinery. This includes not only components such as microtubules but also signalling components and regulatory components as indicated above. Such assays are typically in vitro. Assays are also provided that test the effects of candidate substances identified in preliminary in vitro assays on intact cells in whole cell assays. The assays described below, or any suitable assay as known in the art, may be used to identify these substances.

In particular, we provide for the use of a polynucleotide as set out in Table 5, or a polypeptide encoded by the polypeptide, in a method of identifying a substance capable of binding to the polypeptide, which method comprises incubating the polypeptide with a candidate substance under suitable conditions and determining whether the substance binds to the polypeptide.

We further provide for use of a polynucleotide as set out in Table 5, or a polypeptide encoded by the polypeptide, in a method of identifying a substance capable of modulating the function of the polypeptide, the method comprising the steps of: incubating the polypeptide with a candidate substance and determining whether activity of the polypeptide is thereby modulated.

The substance identified may be isolated or synthesised, and used for prevention, treatment or diagnosis of a disease in an individual. The substance may be adminstered to an individual in need of such treatment. Alternatively or in addition, the substance identified by the assay is administered to an individual in need of such treatment. Preferably, the polynucleotide comprises a human polypeptide as set out in column 3 of Table 5.

Therefore, we provide one or more substances identified by any of the assays described below, viz, mitosis assays, meiotic assays, polypeptide binding assays, microtubule binding/polymerisation assays, microtubule purification and binding assays, microtubule organising centre (MTOC) nucleation activity assays, motor protein assay, assay for spindle assembly and function, assays for dna replication, chromosome condensation assays, kinase assays, kinase inhibitor assays, and whole cell assays, each as described in further detail below.

Candidate Substances

A substance that inhibits cell cycle progression as a result of an interaction with a polypeptide as described here may do so in several ways. For example, if the substance inhibits cell division, mitosis and/or meiosis, it may directly disrupt the binding of a polypeptide as described here to a component of the spindle apparatus by, for example, binding to the polypeptide and masking or altering the site of interaction with the other component. A substance which inhibits DNA replication may do so by inhibiting the phosphorylation or de-phosphorylation of proteins involved in replication. For example, it is known that the kinase inhibitor 6-DMAP (6-dimethylaminopurine) prevents the initiation of replication (Blow, J J, 1993, J Cell Bioll 22,993-1002). Candidate substances of this type may conveniently be preliminarily screened by in vitro binding assays as, for example, described below and then tested, for example in a whole cell assay as described below. Examples of candidate substances include antibodies which recognise a polypeptide as described in this document.

A substance which can bind directly to such a polypeptide may also inhibit its function in cell cycle progression by altering its subcellular localisation and hence its ability to interact with its normal substrate. The substance may alter the subcellular localisation of the polypeptide by directly binding to it, or by indirectly disrupting the interaction of the polypeptide with another component. For example, it is known that interaction between the p68 and p180 subunits of DNA polymerase alpha-primase enzyme is necessary in order for p180 to translocate into the nucleus (Mizuno et al (1998) Mol Cell Biol 18,3552-62), and accordingly, a substance which disrupts the interaction between p68 and p180 will affect nuclear translocation and hence activity of the primase. A substance which affects mitosis may do so by preventing the polypeptide and components of the mitotic apparatus from coming into contact within the cell.

These substances may be tested using, for example the whole cells assays described below. Non-functional homologues of a polypeptide as described here may also be tested for inhibition of cell cycle progression since they may compete with the wild type protein for binding to components of the cell division cycle machinery whilst being incapable of the normal functions of the protein or block the function of the protein bound to the cell division cycle machinery. Such non-functional homologues may include naturally occurring mutants and modified sequences or fragments thereof.

Alternatively, instead of preventing the association of the components directly, the substance may suppress the biologically available amount of a polypeptide as described here. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

Suitable candidate substances include peptides, especially of from about 5 to 30 or 10 to 25 amino acids in size, based on the sequence of the polypeptides described in the Examples, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for a polypeptide as described here. Furthermore, combinatorial libraries, peptide and peptide mimetics, defined chemical entities, oligonucleotides, and natural product libraries may be screened for activity as inhibitors of binding of a polypeptide as described here to the cell division cycle machinery, for example mitotic/meiotic apparatus (such as microtubules). The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of any of the stages of the cell cycle.

Polypeptide Binding Assays

One type of assay for identifying substances that bind to a polypeptide as described here involves contacting a polypeptide as described here, which is immobilised on a solid support, with a non-immobilised candidate substance determining whether and/or to what extent the polypeptide as described here and candidate substance bind to each other. Alternatively, the candidate substance may be immobilised and the polypeptide non-immobilised.

In a preferred assay method, the polypeptide is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads (Smith and Johnson, 1988). As a control, binding of the candidate substance, which is not a GST-fusion protein, to the immobilised polypeptide is determined in the absence of the polypeptide as described here. The binding of the candidate substance to the immobilised polypeptide is then determined. This type of assay is known in the art as a GST pulldown assay. Again, the candidate substance may be immobilised and the polypeptide non-immobilised.

It is also possible to perform this type of assay using different affinity purification systems for immobilising one of the components, for example Ni-NTA agarose and histidine-tagged components.

Binding of the polypeptide as described here to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Microtubule Binding/Polymerisation Assays

In the case of polypeptides as described here that bind to microtubules, another type of in vitro assay involves determining whether a candidate substance modulates binding of such a polypeptide to microtubules. Such an assay typically comprises contacting a polypeptide as described here with microtubules in the presence or absence of the candidate substance and determining if the candidate substance has an affect on the binding of the polypeptide as described here to the microtubules. This assay can also be used in the absence of candidate substances to confirm that a polypeptide as described here does indeed bind to microtubules. Microtubules may be prepared and assays conducted as follows:

Microtubule Purification and Binding Assays

Microtubules are purified from 0-3 h-old Drosophila embryos essentially as described previously (Saunders, et al., 1997). About 3 ml of embryos are homogenized with a Dounce homogenizer in 2 volumes of ice-cold lysis buffer (0.1 M Pipes/NaOH, pH6.6, 5 mM EGTA, 1 mM MgSO4, 0.9 M glycerol, 1 mM DTT, 1 mM PMSF, 1 μg/ml aprotinin, 1 μg/ml leupeptin and 1 μg/ml pepstatin). The microtubules are depolymerized by incubation on ice for 15 min, and the extract is then centrifuged at 16,000 g for 30 min at 4° C. The supernatant is recentrifuged at 135,000 g for 90 min at 4° C. Microtubules in this later supernatant are polymerized by addition of GTP to 1 mM and taxol to 20 μM and incubation at room temperature for 30 min. A 3 ml aliquot of the extract is layered on top of 3 ml 15% sucrose cushion prepared in lysis buffer. After centrifuging at 54,000g for 30 min at 20° C. using a swing out rotor, the microtubule pellet is resuspended in lysis buffer.

Microtubule overlay assays are performed as previously described (Saunders et al., 1997). 500 ng per lane of recombinant Asp, recombinant polypeptide, and bovine serum albumin (BSA, Sigma) are fractionated by 10% SDS-PAGE and blotted onto PVDF membranes (Millipore). The membranes are preincubated in TBST (50 mM Tris pH 7.5, 150 mM NaCl, 0.05% Tween 20) containing 5% low fat powdered milk (LFPM) for 1 h and then washed 3 times for 15 min in lysis buffer. The filters are then incubated for 30 minutes in lysis buffer containing either 1 mM GDP, 1 mM GTP, or 1 mM GTP-γ-S. MAP-free bovine brain tubulin (Molecular Probes) is polymerised at a concentration of 2 μg/ml in lysis buffer by addition of GTP to a final concentration of 1 mM and incubated at 37° C. for 30 min. The nucleotide solutions are removed and the buffer containing polymerised microtubules added to the membanes for incubation for 1 h at 37° C. with addition of taxol at a final concentration of 10 μM for the final 30 min. The blots are then washed 3 times with TBST and the bound tubulin detected using standard Western blot procedures using anti-β-tubulin antibodies (Boehringer Manheim) at 2.5 μg/ml and the Super Signal detection system (Pierce).

It may be desirable in one embodiment of this type of assay to deplete the polypeptide as described here from cell extracts used to produce polymerise microtubules. This may, for example, be achieved by the use of suitable antibodies.

A simple extension to this type of assay would be to test the effects of purified polypeptide as described here upon the ability of tubulin to polymerise in vitro (for example, as used by Andersen and Karsenti, 1997) in the presence or absence of a candidate substance (typically added at the concentrations described above). Xenopus cell-free extracts may conveniently be used, for example as a source of tubulin.

Microtubule Organising Centre (MTOC) Nucleation Activity Assays

Candidate substances, for example those identified using the binding assays described above, may be screening using a microtubule organising centre nucleation activity assay to determine if they are capable of disrupting MTOCs as measured by, for example, aster formation. This assay in its simplest form comprises adding the candidate substance to a cellular extract which in the absence of the candidate substance has microtubule organising centre nucleation activity resulting in formation of asters.

In a preferred embodiment, the assay system comprises (i) a polypeptide as described here and (ii) components required for microtubule organising centre nucleation activity except for functional polypeptide as described here, which is typically removed by immunodepletion (or by the use of extracts from mutant cells). The components themselves are typically in two parts such that microtubule nucleation does not occur until the two parts are mixed. The polypeptide as described here may be present in one of the two parts initially or added subsequently prior to mixing of the two parts.

Subsequently, the polypeptide as described here and candidate substance are added to the component mix and microtubule nucleation from centrosomes measured, for example by immunostaining for the polypeptide and visualising aster formation by immuno-fluorescence microscopy. The polypeptide may be preincubated with the candidate substance before addition to the component mix. Alternatively, both the polypeptide as described here and the candidate substance may be added directly to the component mix, simultaneously or sequentially in either order.

The components required for microtubule organising centre formation typically include salt-stripped centrosomes prepared as described in Moritz et al., 1998. Stripping centrosome preparations with 2 M KI removes the centrosome proteins CP60, CP 190, CNN and γ-tubulin. Of these, neither CP60 nor CP190 appear to be required for microtubule nucleation. The other minimal components are typically provided as a depleted cellular extract, or conveniently, as a cellular extract from cells with a non-functional variant of a polypeptide as described here. Typically, labeled tubulin (usually β-tubulin) is also added to assist in visualising aster formation.

Alternatively, partially purified centrosomes that have not been salt-stripped may be used as part of the components. In this case, only tubulin, preferably labeled tubulin is required to complete the component mix.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

The degree of inhibition of aster formation by the candidate substance may be determined by measuring the number of normal asters per unit area for control untreated cell preparation and measuring the number of normal asters per unit area for cells treated with the candidate substance and comparing the result. Typically, a candidate substance is considered to be capable of disrupting MTOC integrity if the treated cell preparations have less than 50%, preferably less than 40, 30, 20 or 10% of the number of asters found in untreated cells preparations. It may also be desirable to stain cells for γ-tubulin to determine the maximum number of possible MTOCs present to allow normalisation between samples.

Motor Protein Assay

The polypeptides may interact with motor proteins such as the Eg5-like motor protein in vitro. The effects of candidate substances on such a process may be determined using assays wherein the motor protein is immobilised on coverslips. Rhodamine labeled microtubules are then added and their translocation can be followed by fluorescent microscopy. The effect of candidate substances may thus be determined by comparing the extent and/or rate of translocation in the presence and absence of the candidate substance. Generally, candidate substances known to bind to a polypeptide as described here, would be tested in this assay. Alternatively, a high throughput assay may be used to identify modulators of motor proteins and the resulting identified substances tested for affects on a polypeptide as described above.

Typically this assay uses microtubules stabilised by taxol (e.g. Howard and Hyman 1993; Chandra and Endow, 1993—both chapters in “Motility Assays for Motor Proteins” Ed Jon Scholey, pub Academic Press). If however, a polypeptide as described here were to promote stable polymerisation of microtubules (see above) then these microtubules could be used directly in motility assays.

Simple protein-protein binding assays as described above, using a motor protein and a polypeptide as described here may also be used to confirm that the polypeptide binds to the motor protein, typically prior to testing the effect of candidate substances on that interaction.

Assay for Spindle Assembly and Function

A further assay to investigate the function of polypeptide as described here and the effect of candidate substances on those functions is an assay which measures spindle assembly and function. Typically, such assays are performed using Xenopus cell free systems, where two types of spindle assembly are possible. In the “half spindle” assembly pathway, a cytoplasmic extract of CSF arrested oocytes is mixed with sperm chromatin. The half spindles that form subsequently fuse together. A more physiological method is to induce CSF arrested extracts to enter interphase by addition of calcium, whereupon the DNA replicates and kinetochores form. Addition of fresh CSF arrested extract then induces mitosis with centrosome duplication and spindle formation (for discussion of these systems see Tournebize and Heald, 1996).

Again, generally, candidate substances known to bind to a polypeptide as described here, or non-functional polypeptide variants, would be tested in this assay. Alternatively, a high throughput assay may be used to identify modulators of spindle formation and function and the resulting identified substances tested for affects binding of the polypeptide as described above.

Assays for DNA Replication

Another assay to investigate the function of polypeptide as described here and the effect of candidate substances on those functions is as assay for replication of DNA. A number of cell free systems have been developed to assay DNA replication. These can be used to assay the ability of a substance to prevent or inhibit DNA replication, by conducting the assay in the presence of the substance. Suitable cell-free assay systems include, for example the SV-40 assay (Li and Kelly, 1984, Proc. Natl. Acad. Sci USA 81, 6973-6977; Waga and Stillman, 1994, Nature 369, 207-212.). A Drosophila cell free replication system, for example as described by Crevel and Cotteril (1991), EMBO J 10, 4361-4369, may also be used. A preferred assay is a cell free assay derived from Xenopus egg low speed supernatant extracts described in Blow and Laskey (1986, Cell 47,577-587) and Sheehan et al. (1988, J Cell Biol. 106, 1-12), which measures the incorporation of nucleotides into a substrate consisting of Xenopus sperm DNA or HeLa nuclei. The nucleotides may be radiolabelled and incorporation assayed by scintillation counting. Alternatively and preferably, bromo-deoxy-uridine (BrdU) is used as a nucleotide substitute and replication activity measured by density substitution. The latter assay is able to distinguish genuine replication initiation events from incorporation as a result of DNA repair. The human cell-free replication assay reported by Krude, et al (1997), Cell 88, 109-19 may also be used to assay the effects of substances on the polypeptides.

Other In Vitro Assays

Other assays for identifying substances that bind to a polypeptide as described here are also provided. For example, substances which affect chromosome condensation may be assayed using the in vitro cell free system derived from Xenopus eggs, as known in the art.

Substances which affect kinase activity or proteolysis activity are of interest. It is known, for example, that temporal control of ubiquitin-proteasome mediated protein degradation is critical for normal G1 and S phase progression (reviewed in Krek 1998, Curr Opin Genet Dev 8, 36-42). A number of E3 ubiquitin protein ligases, designated SCFs (Skp1-cullin-F-box protein ligase complexes), confer substrate specificity on ubiquitination reactions, while protein kinases phosphorylate substrates destined for destruction and convert them into preferred targets for ubiquitin modification catalyzed by SCFs. Furthermore, ubiquitin-mediated proteolysis due to the anaphase-promoting complex/cyclosome (APC/C) is essential for separation of sister chromatids during mitosis, and exit from mitosis (Listovsky et al., 2000, Exp Cell Res 255, 184-191).

Substances which inhibit or affect kinase activity may be identified by means of a kinase assay as known in the art, for example, by measuring incorporation of ³²P into a suitable peptide or other substrate in the presence of the candidate substance. Similarly, substances which inhibit or affect proteolytic activity may be assayed by detecting increased or decreased cleavage of suitable polypeptide substrates.

Assays for these and other protein or polypeptide activities are known to those skilled in the art, and may suitably be used to identify substances which bind to a polypeptide and affect its activity.

Whole Cell Assays

Candidate substances may also be tested on whole cells for their effect on cell cycle progression, including mitosis and/or meiosis. Preferably the candidate substances have been identified by the above-described in vitro methods. Alternatively, rapid throughput screens for substances capable of inhibiting cell division, typically mitosis, may be used as a preliminary screen and then used in the in vitro assay described above to confirm that the affect is on a particular polypeptide.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance identified by the method as described here on a particular stage of the cell division cycle comprises administering the candidate substance to a cell and determining whether the substance inhibits that stage of the cell division cycle. Techniques for measuring progress through the cell cycle in a cell population are well known in the art. The extent of progress through the cell cycle in treated cells is compared with the extent of progress through the cell cycle in an untreated control cell population to determine the degree of inhibition, if any. For example, an inhibitor of mitosis or meiosis may be assayed by measuring the proportion of cells in a population which are unable to undergo mitosis/meiosis and comparing this to the proportion of cells in an untreated population.

The concentration of candidate substances used will typically be such that the final concentration in the cells is similar to that described above for the in vitro assays.

A candidate substance is typically considered to be an inhibitor of a particular stage in the cell division cycle (for example, mitosis) if the proportion of cells undergoing that particular stage (i.e., mitosis) is reduced to below 50%, preferably below 40, 30, 20 or 10% of that observed in untreated control cell populations.

Therapeutic Uses

Many tumours are associated with defects in cell cycle progression, for example loss of normal cell cycle control. Tumor cells may therefore exhibit rapid and often aberrant mitosis. One therapeutic approach to treating cancer may therefore be to inhibit mitosis in rapidly dividing cells. Such an approach may also be used for therapy of any proliferative disease in general. Thus, since the polypeptides described here appear to be required for normal cell cycle progression, they represent targets for inhibition of their functions, particularly in tumor cells and other proliferative cells.

The term proliferative disorder is used herein in a broad sense to include any disorder that requires control of the cell cycle, for example, cardiovascular disorders such as restenosis and cardiomyopathy, auto-immune disorders such as glomerulonephritis and rheumatoid arthritis, dermatological disorders such as psoriasis, anti-inflammatory, anti-fingal, antiparasitic disorders such as malaria, emphysema and alopecia.

One possible approach is to express anti-sense constructs directed against polynucleotides described in this document, preferably selectively in tumor cells, to inhibit gene function and prevent the tumor cell from progressing through the cell cycle. Anti-sense constructs may also be used to inhibit gene function to prevent cell cycle progression in a proliferative cell. Such anti-sense constructs may comprise anti-sense molecules corresponding to any of the polynucleotides, in particular, those identified in Table 5.

Alternatively, or in addition, RNAi may be used to modulate expression of the polynucleotide in a cell. Double stranded RNA may be made as described in the Examples, e.g., by transcribing both strands of a polynucleotide sequence in a suitable vector (e.g., from T7 or other promoters on either side of the cloned sequence), denatured and annealed. The double stranded RNA (ds RNA) may then be introduced into a relevant cell to inhibit the transcription or expression of the relevant polynucleotide or polypeptide.

We therefore describe a method of modulating, preferably down-regulating, the expression of a polynucleotide as described here, preferably a polynucleotide as set out in Table 5 in a cell, the method comprising introducing a double stranded RNA (dsRNA) corresponding to the polynucleotide, or an antisense RNA corresponding to the polynucleotide, or a fragment thereof, into the cell.

Another approach is to use non-functional variants of the polypeptides that compete with the endogenous gene product for cellular components of cell cycle machinery, resulting in inhibition of function. Alternatively, compounds identified by the assays described above as binding to a polypeptide may be administered to tumor or proliferative cells to prevent the function of that polypeptide. This may be performed, for example, by means of gene therapy or by direct administration of the compounds. Suitable antibodies may also be used as therapeutic agents.

Alternatively, double-stranded (ds) RNA is a powerful way of interfering with gene expression in a range of organisms that has recently been shown to be successful in mammals (Wianny and Zernicka-Goetz, 2000, Nat Cell Biol 2000, 2, 70-75). Double stranded RNA corresponding to the sequence of a polynucleotide can be introduced into or expressed in oocytes and cells of a candidate organism to interfere with cell division cycle progression.

In addition, a number of the mutations described herein exhibit aberrant meiotic phenotypes. Aberrant meiosis is an important factor in infertility since mutations that affect only meiosis and not mitosis will lead to a viable organism but one that is unable to produce viable gametes and hence reproduce. Consequently, the elucidation of genes involved in meiosis is an important step in diagnosing and preventing/treating fertility problems. Thus the polypeptides identified in mutant Drosophila having meiotic defects (as is clearly indicated in the Examples) may be used in methods of identifying substances that affect meiosis. In addition, these polypeptides, and corresponding polynucleotides, may be used to study meiosis and identify possible mutations that are indicative of infertility. This will be of use in diagnosing infertility problems.

Administration

Substances identified or identifiable by the assay methods described here may preferably be combined with various components to produce compositions. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition as described here may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components (or antisense constructs) for use in inhibiting cell cycle progression, for example, inhibiting mitosis or meiosis, may be administered directly as a naked nucleic acid construct. They may further comprise flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. It is particularly preferred to use polynucleotides/vectors that target specifically tumor or proliferative cells, for example by virtue of suitable regulatory constructs or by the use of targeted viral vectors.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide, polypeptide, compound or vector described here may be conjugated, joined, linked, fused, or otherwise associated with a membrane translocation sequence.

Preferably, the polynucleotide, polypeptide, compound or vector, etc described here may be delivered into cells by being conjugated with, joined to, linked to, fused to, or otherwise associated with a protein capable of crossing the plasma membrane and/or the nuclear membrane (i.e., a membrane translocation sequence). Preferably, the substance of interest is fused or conjugated to a domain or sequence from such a protein responsible for the translocational activity. Translocation domains and sequences for example include domains and sequences from the HIV-1-trans-activating protein (Tat), Drosophila Antennapedia homeodomain protein and the herpes simplex-1 virus VP22 protein. In a highly preferred embodiment, the substance of interest is conjugated with penetratin protein or a fragment of this. Penetratin comprises the sequence RQIKIWFQNRRMKWKK (SEQ ID NO:1) and is described in Derossi, et al., (1994), J. Biol. Chem. 269, 10444-50; use of penetratin-drug conjugates for intracellular delivery is described in WO/00/01417. Truncated and modified forms of penetratin may also be used, as described in WO/00/29427.

Preferably the polynucleotide, polypeptide, compound or vector is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

Further Aspects

Further aspects of the invention are set out in the following numbered paragraphs; it is to be understood that the invention includes these aspects.

Paragraph 1. A polynucleotide selected from: (a) polynucleotides encoding any one of the polypeptide sequences set out in Examples 1 to 30 or the complement thereof; (b) polynucleotides comprising a nucleotide sequence capable of hybridising to the polynucleotides defined in (a) above, or a fragment thereof; (c) polynucleotides comprising a nucleotide sequence capable of hybridising to the complement of the polynucleotides defined in (a) above, or a fragment thereof; (d) polynucleotides comprising a polynucleotide sequence which is degenerate as a result of the genetic code to the polynucleotides defined in (a), (b) or (c).

Paragraph 2. A polynucleotide selected from: (a) polynucleotides encoding any one of the polypeptide sequences set out in Examples 1, 2, 2A, 2B and 2C or the complement thereof; (b) polynucleotides comprising a nucleotide sequence capable of hybridising to the polynucleotides defined in (a) above, or a fragment thereof; (c) polynucleotides comprising a nucleotide sequence capable of hybridising to the complement of the polynucleotides defined in (a) above, or a fragment thereof; (d) polynucleotides comprising a polynucleotide sequence which is degenerate as a result of the genetic code to the polynucleotides defined in (a), (b) or (c).

Paragraph 3. A polynucleotide selected from: (a) polynucleotides encoding any one of the polypeptide sequences set out in Examples 3 to 9 and 9A or the complement thereof; (b) polynucleotides comprising a nucleotide sequence capable of hybridising to the polynucleotides defined in (a) above, or a fragment thereof; (c) polynucleotides comprising a nucleotide sequence capable of hybridising to the complement of the polynucleotides defined in (a) above, or a fragment thereof;(d) polynucleotides comprising a polynucleotide sequence which is degenerate as a result of the genetic code to the polynucleotides defined in (a), (b) or (c).

Paragraph 4. A polynucleotide selected from: (a) polynucleotides encoding any one of the polypeptide sequences set out in Examples 10 to 29 or the complement thereof; (b) polynucleotides comprising a nucleotide sequence capable of hybridising to the polynucleotides defined in (a) above, or a fragment thereof; (c) polynucleotides comprising a nucleotide sequence capable of hybridising to the complement of the polynucleotides defined in (a) above, or a fragment thereof; (d) polynucleotides comprising a polynucleotide sequence which is degenerate as a result of the genetic code to the polynucleotides defined in (a), (b) or (c).

Paragraph 5. A polynucleotide probe which comprises a fragment of at least 15 nucleotides of a polynucleotide according to any of Paragraph s 1 to 4.

Paragraph 6. A polypeptide which comprises any one of the amino acid sequences set out in Examples 1 to 30 or in any of Examples 1 to 2, 2A, 2B and 2C, Examples 3 to 9 and 9A and Examples 10 to 29 or a homologue, variant, derivative or fragment thereof.

Paragraph 7. A polynucleotide encoding a polypeptide according to Paragraph 6.

Paragraph 8. A vector comprising a polynucleotide according to any of Paragraph s 1 to 5 and 7.

Paragraph 9. An expression vector comprising a polynucleotide according to any of Paragraph s 1 to 5 and 7 operably linked to a regulatory sequence capable of directing expression of said polynucleotide in a host cell.

Paragraph 10. An antibody capable of binding a polypeptide according to Paragraph 6.

Paragraph 11. A method for detecting the presence or absence of a polynucleotide according to any of Paragraph s 1 to 5 and 7 in a biological sample which comprises: (a) bringing the biological sample containing DNA or RNA into contact with a probe according to Paragraph under hybridising conditions; and (b) detecting any duplex formed between the probe and nucleic acid in the sample.

Paragraph 12. A method for detecting a polypeptide according to Paragraph 6 present in a biological sample which comprises: (a) providing an antibody according to Paragraph 10; (b) incubating a biological sample with said antibody under conditions which allow for the formation of an antibody-antigen complex; and (c) determining whether antibody-antigen complex comprising said antibody is formed.

Paragraph 13. A polynucleotide according to according to any of Paragraph s 1 to 5 and 7 for use in therapy.

Paragraph 14. A polypeptide according to Paragraph 6 for use in therapy.

Paragraph 15. An antibody according to Paragraph 10 for use in therapy.

Paragraph 16. A method of treating a tumor or a patient suffering from a proliferative disease comprising administering to a patient in need of treatment an effective amount of a polynucleotide according to any of Paragraph s 1 to 5 and 7.

Paragraph 17. A method of treating a tumor or a patient suffering from a proliferative disease, comprising administering to a patient in need of treatment an effective amount of a polypeptide according to Paragraph 6.

Paragraph 18. A method of treating a tumor or a patient suffering from a proliferative disease, comprising administering to a patient in need of treatment an effective amount of an antibody according to Paragraph 10 to a patient.

Paragraph 19. Use of a polypeptide according to Paragraph 6 in a method of identifying a substance capable of affecting the function of the corresponding gene.

Paragraph 20. Use of a polypeptide according to Paragraph 6 in an assay for identifying a substance capable of inhibiting the cell division cycle.

Paragraph 21. Use as Paragraph ed in Paragraph 20, in which the substance is capable of inhibiting mitosis and/or meiosis.

Paragraph 22. A method for identifying a substance capable of binding to a polypeptide according to Paragraph 6, which method comprises incubating the polypeptide with a candidate substance under suitable conditions and determining whether the substance binds to the polypeptide.

Paragraph 23. A method for identifying a substance capable of modulating the function of a polypeptide according to Paragraph 6 or a polypeptide encoded by a polynucleotide according to any of Paragraph s 1 to 5 and 7, the method comprising the steps of: incubating the polypeptide with a candidate substance and determining whether activity of the polypeptide is thereby modulated.

Paragraph 24. A substance identified by a method or assay according to any of Paragraph s 19 to23.

Paragraph 25. Use of a substance according to Paragraph 24 in a method of inhibiting the function of a polypeptide.

Paragraph 26. Use of a substance according to Paragraph 24 in a method of regulating a cell division cycle function.

Paragraph 27. A method of identifying a human nucleic acid sequence, by: (a) selecting a Drosophila polypeptide identified in any of Examples 1 to 30; (b) identifying a corresponding human polypeptide; (c) identifying a nucleic acid encoding the polypeptide of (b).

Paragraph 28. A method according to Paragraph 27, in which a human homologue of the Drosophila sequence, or a human sequence similar to the Drosophila sequence, is identified in step (b).

Paragraph 29. A method according to Paragraph 27 or 28, in which the human polypeptide has at least one of the biological activities, preferably substantially all the biological activities of the Drosophila polypeptide.

Paragraph 30. A human polypeptide identified by a method according to Paragraph 27, 28 or 29.

The invention will now be further described by way of Examples, which are meant to serve to assist one of ordinary skill in the art in carrying out the invention and are not intended in any way to limit the scope of the invention.

### EXAMPLE

### Example Section A

### Identification of Human Cell Cycle Genes

Introduction

In order to identify new cell cycle regulatory genes in Drosophila and their human counterparts, we investigated 33 fly lines obtained by P-element mutagenesis carried out on the X chromosome. All those fly lines are screened directly for mitotic phenotypes at developmental stages where division is crucial (i.e. the syncytial embryo, larval brains, and male and female meiosis). In each case, the P-element insertion site is identified leading to the selection of 62 genes flanking the insertion site.

In order to clarify the identity of the mutated “mitotic genes”, we use an RNAi-based knockdown approach in cultured Drosophila cells followed by FACS analysis, mitotic index evaluation (Cellomics Arrayscan) and immunofluorescence observations of mitotic phenotypes for all 63 genes.

The microscope phenotyping approach led to the identification of 30 gene candidates that are required for cell cycle progression, some of which are also detected as presenting some changes in the FACS profile and/or in the mitotic index (see Table 5 for a full summary). Data relating to these genes is presented in Examples Section B, Examples 1 to 29 below.

These genes encode a variety of novel proteins: 6 protein kinases; 2 protein phosphatases, 2 proteins of the ubiquitin-mediated protein degradation pathway, a cytosketal protein, a microtubule-binding protein, a homologue of a suspected kinesin-like protein, a RNA polymerase 2 associated cyclin, a ribosomal protein; a protein involved in retrograde (Golgi to ER) transport, a member of the family of thioredoxin reductases, a hydroxymethyltransferase, a Cdk associated protein, an RNA binding protein, an O-acetyl transferase and 9 other novel proteins with no particularly characteristic identifying features.

Human counterparts of the selected genes are identified and tested as described below. A short list of Drosophila and human genes and proteins useful for screening for anti-proliferative molecules is presented as Table 5.

TABLE 5
Short list of potentially new interesting gene candidates

| Drosophila Gene | | Human Homologue Accession |
|---|---|---|
| Name | Human Homologue Gene Name | Number |
| CG2028 | Casein kinase I | P48729 |
| CG3011 | Serine hydroxymethyl transferase | AAA63258 |
| CG15309 | DiGeorge syndrome related protein | AAL09354 |
| | FKSG4 | |
| CG15305 | Human homologue of CG15305 | None |
| CG2222 | Hypothetical protein FLJ13912 | NP_073607 |
| CG2938 | CAS1 O-acetyltransferase | NP_075051 |
| CG1524 | Ribosomal protein S14 | A25220 |
| CG10778 | Hypothetical protein FLJ13102 | NP_079163 |
| | (kinesin like) | |
| CG18292 | Cdk associated protein 1 (deleted in | BAA22937 |
| | oral cancer) | |
| CG10701 | Moesin | A41289 |
| CG10648 | Mak16-like RNA binding protien | NP_115898 |
| CG2854 | CAD38627 hypothetical protein | CAD38627 |
| CG2845 | B-raf | AAA35609 |
| CG1486 | BAA19780 novel protein | BAA19780 |
| CG10964 | 11-cis retinal dehydrogenase | AAC50725 |
| CG2151 | Thioredoxin reductase beta | XP_033135 |
| CG10988 | Gamma tubulin ring complex 3 | AAC39727 |
| CG1558 | Human homologue of CG1558 | NONE |
| CG11697 | Novel protein | BAB14444 unamed protein - similar |
| | | to a hypothetical protein in the |
| | | region deleted in human familial |
| CG3954 | Protein tyrosine phosphatase non- | AAH08692 |
| | receptor type 11 (Shp2) | |
| CG16903 | Cyclin L ania-6a | AAD53184 |
| CG16983 | Skp1 ubiquitin ligase | XP_054159 |
| CG13363 | CGI-85 | NP_057112 |
| CG18319 | Ubc13 ubiquitin conjugating enzyme | BAA11675 |
| CG14813 | archain | CAA57071 |
| CG8655 | Cdc7 | AAB97512 |
| CG2621 | GSK 3 beta | NP_002084 |
| CG1725 | Dlg1/Dlg2 | XP_012060 |
| CG1594 | JAK-2 Janus kinase 2 | NP_004963 |
| CG2096 | Protein phosphatase 1 | NP_002700 |

Results

Table 6 shows all significant cell cycle phenotypes observed after RNAi with the Drosophila genes flanking P-element insertion sites identified in Examples 1 to 29. The PCR primers used to create the double stranded RNA (see Materials and Methods above) are shown in each case together with the RNA ID number. Results derived from Facs analysis of cell cycle compartment, mitotic index as determined by the Cellomics mitotic index assay, and cellular phenotypes determined by microscopy are shown.

FACS Analysis of Cell Cycle

FACS analysis is used to assess the effects of Drosophila gene specific RNAi on the cell cycle. Through the determination of the DNA content by propidium iodide quantitation, any changes in the cell cycle distribution in sub-G1 (apoptotic), G1, G2/M can be observed. 24 genes in the Facs assessment present some changes in cell cycle distribution. (Table 6).

Mitotic Index Evaluation with Cellomics Arrayscan

An evaluation of mitotic index is performed using the Cellomics arrayscan and the Cellomics proprietary mitotic index HitKit procedure (see Materials and Methods above).

The basic principle of this method is that cells in mitosis are decorated by an antibody directed against a specific mitotic marker. Their proportion relatively to the total number of cells is determined, giving a proportion of cells in mitosis. This automated method presents the advantage of being more rapid than the microscope observations, however it only measures one feature of the cycling cells. Some mitotic genes that do not significantly affect the overall proportion of cells in mitosis will therefore not be detected. The reverse is also true as the knockdown of some gene products might affect the mitotic index without displaying any obvious increase in chromosomal or spindle defects. Table 6 presents data only where there was a statistically significant variation in the mitotic index (determined by a Ttest value of <0.1) as compared to the RFP RNAi control.

An increase in mitotic index can indicate that the knockdown of a gene essential for completion of mitosis has blocked more cells in mitosis, however many of the gene knockdowns listed in Table 6 result in a decrease in the mitotic index, suggesting that the population of cells overall are spending less time in mitosis. Possible interpretations of this, are that defects in the centrosome duplication cycle block some cells in G1/S and they are unable to enter mitosis, or that defects in cytokinesis block cells on the exit from mitosis at a point after the assay specific marker is lost. The loss of checkpoints at mitosis may also allow cells to move faster through mitosis. The increase in mitotic defects observed for most of these genes might then be the result of this lack of checkpoint control.

13 genes in the phenotype assessment present some changes in the mitotic index (Table 6).

Microscope Observation and Cellular Phenotyping

The primary goal of the cell phenotype assessment is to find abnormalities in the following: chromosome number in prometaphase (ploidy), chromosome behaviour in metaphase or anaphase, spindle morphology, number of centrosomes, and cell viability. The secondary goal of the assessment is to evaluate and quantify these abnormalities, this is an essential step as control cells also present some defects.

The wild-type Drosophila DMEL2 cells present a large range and a significant proportion of chromosomal defects (between 30-40%). Therefore, between 300 and 500 mitotic cells were counted for each experiment in order to obtain a statistically significant evaluation of any change in the proportion of defects. The cells categorized as presenting chromosomal defects in the study encompass aneuploid and polyploid prometaphase cells, cells that apparently fail to align their chromosomes at metaphase and the cells with lagging or stretched chromosomes in anaphase. Spindle defects are also noted, but not quantified in the same group. Some candidates are also noted as presenting a significant decrease in the number of mitotic cells (mitotic index) or as affecting the viability of the cells (decrease in cell confluency or presence of apoptotic cells).

A noteworthy observation is that it is difficult to find a unique representative phenotype for most of the genes tested. Rather than one gene=one phenotype, an overall increase in the different categories of chromosomal defects is observed. However, one can often see a more significant increase in one particular subcategory of defects as for example in the proportion of lagging chromatids or the number of centrosomes.

Table 6 describes the data obtained from these studies for genes where a significant phenotype is observed. 30 of the candidate genes show a significant phenotype, 26 of which show an increase in chromosomal defects. This increase in mitotic chromosome behaviour abnormalities is sometimes associated with an increase in mitotic spindle defects. Of the remaining 4 with no increase in chromosomal defects, CG1725 (RNA528/529) shows a clear increase in spindle defects, with CG1524 (RNA 482/483) there are not enough mitotic cells to do a proper quantification (as the gene product is a ribosomal protein, it is highly probable that its inactivation results in a net increase in the proportion of cell death explaining the drop in cell confluency also observed) and for CG14813 (RNA 586/587), a large proportion of cells are dying and there is an obvious decrease in the number of mitotic cells, this might affect the relative proportion of normal and abnormal mitotic cells. Finally CG10648 (RNA 488/489) had a lower proportion of chromosomal defects but a high proportion of monopolar and small spindles. The proportion of prometaphase cells and apoptotic cells was also high.

Conclusion

From a collection of Drosophila P-element insertion lines which display phenotypes consistent with an effect on mitosis we derived a series of novel Drosophila and human genes which represent targets for the development of anti-proloiferative therapies. We used three different approaches to validate the role of each gene in the cell cycle and to gather phenotype information following an RNAi-based gene knockdown approach.

Table 5 shows a short list of 30 new interesting human genes demonstrated to play a role in mitosis. This short list is mainly based on the results of the detailed microscope phenotype evaluation (see Table 6), although all of the 42 genes listed in Table 6 show a cell cycle related phenotype in one or more of the 3 assays.

Materials and Methods

Generation and Identification of Lethal, Semi-Lethal and Sterile X Chromosome Mutants Having Defects in Mitosis and/or Meiosis

P-Element Mutagenesis

Transposable elements are widely used for mutagenesis in Drosophila melanogaster as they couple the advantages of providing effective genetic lesions with ease of detecting disrupted genes for the purpose of molecular cloning. To achieve near saturation of the genome with mutations resulting from mobilisation of the P-lacW transposon (a P-element marked with a mini-white gene, bearing the E.coli lacZ gene as an enhancer trap, and an E. coli replicon and ampicillin resistance gene to facilitate ‘plasmid rescue’ of sequences at the site of the P-insertion), Drosophila females that are homozygous for P-lacW(inserted on the second chromosome) are crossed with males carrying the transposase source P(A2-3) (Deak et al., 1997). Random transpositions of the mutator element are then ‘captured’ in lines lacking transposase activity. Stable, or balanced, stocks bearing single lethal P-lacW insertions are made to give a collection of 501 lines (Peter et al., submitted) and a further 73 lines that are either sterile or carry a mutation giving a visible morphological phenotype.

Screening for Mitotic and Meiotic Defects

About half of the mutants in the collection are embryonic lethals.

Screens for mutants affecting spermatogenesis within this collection of 501 recessive lethal, semi-lethal and sterile mutants were carried out.

We have carried out cytological screens of the lines that comprise late larval lethals, pupal lethals, pharate and adult semi-lethals and steriles for defective mitosis in the developing larval CNS. This has identified 20 complementation groups that affect all stages of the mitotic cycle. The cytological screens involve examining orcein-stained squashed preparations of the larval CNS to detect abnormal mitotic cells. In lines where defects are identified, the larval CNS is subjected to immunostaining to identify centromeres, spindle microtubules and DNA for further examination. This leads to clarification of the mitotic defect.

As a set of common functions are essential to both mitosis and meiosis, we then identify mutations resulting in sterility and failed progression through male meiosis. This involves examining squashed preparations larval, pupal or adult testes by phase contrast microscopy. We examine “onion stage” spermatids in the 24 pupal and pharate lethal lines and adult “semi-lethal” and viable lines for variations in size and number of nuclei which provides an indication of whether there have been defects in either chromosome segregation or cytokinesis, respectively. A total of 8 lines show such defects.

Further phenotype information for each mutant described in the results section, as observed by phase contrast microscopy of dividing meiocytes, is provided in the “Phenotype” field.

We then examined the ovaries and eggs of females that when homozygous are either sterile or produce embryos that fail to develop. Dissected ovaries are examined by microscopy for defects in the mitotic divisions that lead to the formation of the 16 cell egg chambers, for defects in the endoreduplication of 15 nurse cell nucleic; for cytoskeletal defects in the development of the egg chamber; for defects in meiosis; and for mitotic defects in embryos derived from mutant mothers.

We examined 24 lines that show female sterility or maternal effect lethality when homozygous and identify 5 that display defects of the type described above. In the Examples 1 to 29 below, lines exhibiting mitotic and meiotic phenotypes are categorised generally into three categories:
Category 1: Female Sterile
Category 2: Male Sterile
Category 3: Mitotic (Neuroblast) Phenotypes

Category 1 phenotypes are exhibited by mutations in Examples 1, 2, 2A, 2B and 2C; while Category 2 phenotypes are exhibited by mutations in Examples 3 to 9 and 9A. Category 3 phenotypes are exhibited by mutations in Examples 10 to 29.

Plasmid Rescue of P-Elements from Mutant Drosophila Lines

Genomic DNA was isolated from adult flies by the method of Jowett et al., 1986. Inverse PCR is used to identify flanking chromosomal sequences. The position of the inserted P-element is indicated in the Examples.

Sequence Analysis of P Element Insertion Lines

The open reading frame(s) (ORF(s)) immediately adjacent to the insertion site are identified from the annotated total genome sequence of Drosophila with reference to the ‘GADFLY’ section of the ‘FLYBASE’ Drosophila genome database (database of the Berkeley Drosophila Genome Project). The site of P element insertion and the GenBank accession number of the genomic file which contains the insertion site are included in the results section.

Where the insertion site was within a gene or close to the 5′ end of a gene, disruption of this gene is likely to be responsible for the phenotype, and it is included in the results section under the field heading “Annotated Drosophila Genome Complete Genome Candidate”, as both an accession number and an amino acid sequence. Where the insertion site indicates that the P-element may be affecting expression of two diverging genes (on opposite strands of the DNA) both are included in the results section.

The Drosophila gene sequence is then used to identify a human homologue. Data on homologues is derived from the Blink (“BLAST Link”) facility provided by the NCBI (National Center for Biotechnology Information) database. Where homologues are not apparent, further searches are made against the NCBI database using BLASTX (which compares the nucleotide query sequence virtually translated in all 6 frames against an amino acid database) or TBLASTN (amino acid query sequence against a nucleotide database virtually translated in all 6 frames) or TBLASTX (nucleotide query sequence against nucleotide database, both virtually translated in all 6 frames). Human homologues are included in the results section under the heading “Human Homologue of Complete Genome Candidate”, as both an accession number and an amino acid.

Additional Sequence Analysis using the Annotated D. melanogaster Sequence (GadFly)

As indicated above, rescue sequences are also used to search the fully annotated version of the Drosophila genome (GadFly; Adams, et al., 2000, Science 287, 2185-2195), using GlyBLAST at the Berkeley Drosophila Genome Projects web site (http://www.fruitfly.org/annot/) to identify the genome segment (usually approximately 200-250 kb) containing the P-element insertion site. The graphic representation of the genomic fragment available at GadFly allows the identification of all real and theoretical genes which flank the site of insertion. Candidate genes where the P-element is either inserted within the gene or close to the 5! end of the gene are identified. In GadFly, the Drosophila genes are given the designation CG (Complete gene) and usually details of human homologues are also given. Such human sequences may also be obtained using the fly sequences to screen databases using the BLAST series of programs. They may also be found by nucleic acid hybridisation techniques. In both cases homologies are defined using the parameters taught earlier in this patent. In most cases, this data confirms the data derived from the sequence analysis procedure described above, and in some cases new data is obtained. Where available both sets of data are included in the individual Examples described below.

Confirmation of Cell Cycle Involvement of Candidate Genes Using Double Stranded RNA Interference (RNAi)

P-elements usually insert into the region 5′ to a Drosophila gene. This means that there is sometimes more than one candidate gene affected, as the P-element can insert into the 5′ regions of two diverging genes (one on each DNA strand). In order to confirm which of the candidate genes is responsible for the cell cycle phenotype observed in the fly line, we use the technique of double stranded RNA interference to specifically knock out gene expression in Drosophila cells in tissue culture (Clemens, et al., 2000, Proc. Natl. Acad. Sci. USA, 6499-6503). The overall strategy is to prepare double stranded RNA (dsRNA) specific to each gene of interest and to transfect this into Schneider's Drosophila line 2 (Dme1-2) to inhibit the expression of the particular gene. The dsRNA is prepared from a double stranded, gene specific PCR product with a T7 RNA polymerase binding site at each end. The PCR primers consist of 25-30 bases of gene specific sequence fused to a T7 polymerase binding site (TAATACGACTCACTATAGGGACA) (SEQ ID NO:2), and are designed to amplify a DNA fragment of around 500 bp. Although this is the optimal size, the sequences in fact range from 450 bp to 650 bp. Where possible, PCR amplification is performed using genomic DNA purified from Schneider's Drosophila line 2 (Dme1-2) as a template. This is only feasible where the gene has an exon of 450 bp or more. In instances where the gene possesses only short exons of less than 450 bp, primers are designed in different exons and PCR amplification is performed using cDNA derived from Schneider's Drosophila line 2 (Dme1-2) as a template.

A sample of PCR product is analysed by horizontal gel electrophoresis and the DNA purified using a Qiagen QiaQuick PCR purification kit. 1 μg of DNA is used as the template in the preparation of gene specific single stranded RNA using the Ambion T7 Megascript kit. Single stranded RNA is produced from both strands of the template and is purified and immediately annealed by heating to 90 degrees C. for 15 mins followed by gradual cooling to room temperature overnight. A sample of the dsRNA is analysed by horizontal gel electrophoresis.

3 μg of dsRNA is transfected into Schneider's Drosophila line 2 (Dme1-2) using the transfection agent, Transfect (Gibco) and the cells incubated for 72 hours prior to fixation. The DNA content of the cells is analysed by staining with propidium iodide and standard FACS analysis for DNA content. The cells in G1 and G2/S phases of the cell cycle are visualised as two separate population peaks in normal cycling S2 cells. In each experiment, Red Fluorescent Protein dsRNA is used as a negative control.

Preparation of dsRNA

RNA is prepared using an Ambion T7 Megascript kit in the following reaction: μl 10×T7 reaction buffer, 2 μl 75 mM ATP, 2 μl 75 mM GTP, 2 μl 75 mM UTP, 2 μl 75 mM CTP, 2 μl T7 RNA polymerase enzyme mix, 8 μl purified PCR product

Incubate at 37° C. for 6 hours. For convenience this can be done overnight in a PCR machine, such that the reaction is due to finish the next day e.g. 10 hrs 4° C., 6 hrs 37° C., 4° C. œ (prog. LISA6)

To degrade the DNA, add 1 ml DNase I (2 U/ml) and incubate at 37° C. for 15 mins.

Add 115 μl DEPC-treated water and 15 μl ammonium acetate stop solution (5M ammonium acetate, 100 mM EDTA)

Extract with an equal volume of phenol/chloroform, an equal volume of chloroform and then precipitate the RNA by adding 1 volume of isopropanol. Chill at −20° C. for 15-30 mins, then spin at top speed in a microfuge at 4° C. Remove the supernatant avoiding the RNA pellet, which appears as a clear, jelly-like pellet at the base of the tube. Dry briefly then dissolve the RNA in 20-100 μl DEPC-treated water, depending on the size of the pellet.

At this stage there are 2 complimentary single stranded RNAs. To anneal these, incubate the tube at 90° C. for 10 mins, then cool slowly, by transferring to a hot block at 37° C. and then setting the thermostat to room temperature.

Once the hot block has reduced to room temperature, spin down the liquid to the bottom of the tube and run 1 μl on a 1% agarose TBE horizontal gel to check the RNA yield and size.

Transfection of Schneider Line 2 (Dme1-2) Cells with dsRNA (Adherent Protocol)

Transfect 3 μg dsRNA into Schneider line 2 (Dme1-2) cells using Promega Transfast transfectjon reagent.

Schneider line 2 (Dme1-2) cells are grown in Schneider's medium 30 10% FCS+penicillin/Streptomycin, at 25° C. For the purpose of transfection with dsRNA, 25 ml of a healthy growing culture should be sufficient for 24-30 transfections. Knock off cells adhering to the bottom of the flask by banging it sharply against the side of the bench, then aliquot 1 ml into each well of 5 six-well plates. Add an additional 2 ml Schneider's medium+10% FCS+penicillin/Streptomycin to each well and incubate the plates overnight in a humid chamber at 25° C.

Vortex the Transfast, then add 9 μl to a sterile eppendorf containing the 3 μg dsRNA. Add 1 ml Schneider's medium (no additives), vortex immediately and incubate at room temperature for 15 mins. In the mean time, carefully remove the Schneider's medium from the six-well plates and replace with Schneider's medium (no additives); ˜1 ml/well.

Once the dsRNA+Transfast has finished its 15 min incubation, remove the medium from the cells in the six-well plates, replace with the 1 ml dsRNA/Transfast/Schneider's medium and incubate at 25° C. for 1 hr in a humid chamber.

Add 2 ml Schneider's medium containing 10%FCS+pen/strep and return to humid chamber in 25° C. incubator for 24-72 hrs.

Initially, observations of the affects of dsRNA transfection on the Schneider line 2 cell cycle are made after 72 hrs incubation, but where a significant phenotype is observed, additional transfections are performed and observations made at earlier time points.

For each experiment, transfection with RFP dsRNA is used as a negative control. Cells which have been treated with transfast, but which have not been transfected with dsRNA are also included as a control. Transfection with polo or orbit dsRNA, shown in preliminary studies to have an observable affect on Schneider line 2 cell cycle, is used as a positive control in each experiment.

Immunostaining of DMEL-2 Cells for Microscopic Analysis

For microscopic analysis of DMEL-2 insect cell line, ˜4×106 cells (0.5×10⁶ cells for 3 day incubations) are grown on coverslips in the bottom of the wells of six-well plates

Following any required treatments, the media is carefully removed and replaced with 1 ml PHEMgSO₄ fixation buffer (60 mM PIPES, 25 mM Hepes, 10 mM EGTA, 4 mM MgSO₄, pH to 6.8 with KOH)+3.7% formaldehyde. Until the cells are fixed they do not adhere strongly to the coverslip, so it is important to pipette gently at this stage.
The cells are left to fix for 20 mins, then the buffer replaced with PBS+0.1% Triton X-100 for 2 mins to permeablise the cells.
Cells are then blocked using PBS+0.1% Triton X-100+1% BSA (freshly prepared) and incubated for 1 hr at RT.
Next cells are incubated with the primary rat α-tubulin antibody YL1/2 (1:300 dil.) (+ any other primary antibodies to be used, ex: gamma-tub at 1/500) in PBS+0.1% Triton X-100+1% BSA 2-3 hrs at RT or alternatively overnight at 4° C.
Wash the cells 3 times for 5 mins in PBS +0.1% Triton X-100 and then incubate with the secondary antibody, TRITC-donkey anti-rat (1:500 dil.) (+ any other secondary antibodies to be used) in PBS+0.1% Triton X-100+1% BSA, at room temperature for 1 hr.
Wash the cells 3 times for 5 mins in PBS+0.1% Triton X-100 and once in PBS alone, then mount on a slide on a drop of N-propyl gallate mounting medium containing DAPI to stain the DNA and seal with nail varnish
View using fluorescent microscopy.

Primary antibodies: anti α-tub, 1:300 (rat YL1/2; SEROTEC); anti γ-tub, 1:500 (mouse; Sigma GTU-88)

Secondary antibodies: TRITC donkey anti-rat IgG at 1:300 (Jackson Immunoresearch, 712-026-150); AlexaFluor 488 goat anti-mouse, 1:300 (Molecular Probes; A-11001)

Transfections of S2 cells were carried out in 6 well tissue culture plates using 3 μg ds RNA per gene. The cells were harvested following three days for immunostaining.

Microscope Observations and Cellular Phenotyping

All studies were performed using a standard operating procedure. For every gene, each phenotypic test was performed following a 48 hours period of RNAi induction in duplicate and in two independent sets of experiments. The observations were carried out using a Zeiss Axioskop 2 motorized microscope with a 63×/1.4 plan-apochromat Zeiss objective.

Cells were fixed and stained with DAPI, alpha-tubulin and gamma-tubulin to visualise the nucleus/DNA, the microtubule network/spindle and the centrosomes respectively (see immunostaining section).

For each experiment, the number of normal looking mitotic cells in prophase/prometaphase, metaphase, anaphase and telophase is quantified as well as the abnormal looking ones in those various stages. These comprise abnormal chromosome number in prometaphase, misaligned chromosomes and lagging chromosomes in metaphase and anaphase respectively. Also, the abnormalities in the spindle morphology and the number of centrosomes are carefully noted. To get a more complete characterisation of the phenotype, the cell viability (cell confluency and number of apoptotic cells) is also assessed as well as the number of multinucleated interphase cells and the nucleus and cell morphology if different from control. If a phenotype appears to be more representative some images were stored for presentation of data.

FACS Analysis of Transfected Schneider Line 2 Cells

Following transfection and incubation for the desired length of time, then transfer the cells to a 15 ml centrifuge tube and pellet by spinning at 2000 rpm for 5 mins. Remove the supernatant, resuspend the cell pellet in 1 ml PBS and pellet a second time by spinning at 2000 rpm for 5 mins. Remove 900 μl of the PBS, resuspend the cells in the remaining PBS and then add 900 μl ethanol drop-wise while vortexing the tube. Transfer the cells to an eppendorf tube and store at −20° C.

On the day of analysis, pellet the cells by spinning in a microfuge for 5 mins at 2000 rpm, remove the supernatant, resuspend the cells in the residual ethanol and add 500 μl PBS. To remove clumps take the cells up through a 25 gauge needle and transfer to FACS tube. Add 3 μl 6 mg/ml Rnase A (Pharmacia) and 2.5 μl 25 mg/ml propidium iodide and incubate at 37° C. for 30 mins, then store on ice.

Analyse DNA content of the Schneider line 2 cells using FACSCalibur at Babraham Institute. Mutant phenotypes are determined by comparing profiles relative to cells transfected with RFP dsRNA.

Cellomics Mitotic Index HitKit Procedure

To Packard Viewplates containing pre-aliquoted dsRNA samples (1000 ng/well) add 35 μl of logarithmically growing D.Mel-2 cells diluted to 2.3×10⁵ cells/ml in fresh Drosophila-SFM/glutamine/Pen-Strep pre-warmed to 28° C.

Incubate the cells with the dsRNA (60 nM) in a humid chamber at 28° C. for 1 hr.

Add 100 μl Drosophila-SFM/glutamine/Pen-Strep pre-warmed to 28° C. and return the cells containing the dsRNA to the humid chamber at 28° C. for 72 hrs.

Gently remove the medium and slowly add 100 μl Fixation Solution (3.7% formaldehyde, 1.33 mM CaCl₂, 2.69 mM KCl, 1.47 mM KH₂PO₄, 0.52 mM MgCl₂-6H₂O, 137 nM NaCl, 8.50 mM Na₂HPO₄-7H₂O ) pre-warmed to 28° C. Incubate in the fume hood for 15 minutes. It is imperative to use care when manipulating cells before and during fixation.

Remove the Fixation Solution and wash with 100 μl Wash Buffer (1.33 mM CaCl₂, 2.69 mM KCI, 1.47 mM KH₂PO₄, 0.52 mM MgCl₂-6H₂O, 137 mM NaCl, 8.50 mM Na₂HPO₄-7H₂O).

Remove the Wash buffer, add 100 μl Permeabilisation Buffer (30.8 mM NaCl, 0.31 mM KH₂PO₄, 0.57 mM Na₂HPO₄-7H₂O, 0.02% Triton X-100), and incubate for 15 minutes.

Remove the Permeabilisation Buffer and wash with 100 μl Wash Buffer.

remove the Wash Buffer and add 50 μl of Staining Solution (1 μg/ml Hoechst 33258, 1.33 mM CaCl₂, 2.69 mM KCl, 1.47 mM KH₂PO₄, 0.52 mM MgCl₂-6H₂O, 137 mM NaCl, 8.50 Na₂HPO₄-7H₂O ) per well. Incubate for 1 hour protected from the light.

Remove the Staining Solution and wash twice with 100 μl Wash Buffer.

Remove the Wash Buffer and replace with 200 μL Wash Buffer containing 0.02% sodium azide.

Seal the plates and analyse the transfection efficiency using the ArrayScan HCS System, running the Application protocol Percent_Transfection_{—}200602_{—}10×_p2.0 with the 10× objective and the QuadBGRFR filter set.

TABLE 6
Results of Facs, Mitotic Index, and Cell phenotype assays after siRNA gene knockdown in Dmel-2 cells

| | | | |
|---|---|---|---|
| Example | Fly | Drosophila | RNA |

| | | | | |
|---|---|---|---|---|
| number | Line | gene | ID | RNAi primers |

| | | | | |
|---|---|---|---|---|
| 1 | 464 | CG15319 | 452 | TAATACGACTCACTATAGGGAGAACGGCACTTCTTTTTCTTGTCACCT (SEQ ID NO:3) |
| | | | 453 | TAATACGACTCACTATAGGGAGAATGATGAGCAGCTCCAGCAGTCTCT (SEQ ID NO:4) |
| 2 | 492 | CG2028 | 458 | TAATACGACTCACTATAGGGAGAGAAGCGGATCGTTTGGCGACATTTA (SEQ ID NO:5) |
| | | | 459 | TAATACGACTCACTATAGGGAGAAGATGGGCATTGATCGAGGCATAGC (SEQ ID NO:6) |
| 2A | ccr-a2 | CG3011 | 598 | TAATACGACTCACTATAGGGAGATGGCAACGAGTACATCGACCGCATA (SEQ ID NO:7) |
| | | | 599 | TAATACGACTCACTATAGGGAGATACCTTGTCTCCATTGGCCTTGGTG (SEQ ID NO:8) |
| 2B | ewv-b | CG2446 | 602 | TAATACGACTCACTATAGGGACACCCCAAGGCGATAGATACCACGATA (SEQ ID NO:9) |
| | | | 603 | TAATACGACTCACTATAGGGAGAATCTCTGGTATGGCCATCAGGCACT (SEQ ID NO:10) |
| 2C | Fs(1)06 | CG15309 | 608 | TAATACGACTCACTATAGGGAGAGGTGAAGACGTTTCAGGCCTATCTA (SEQ ID NO:11) |
| | | | 609 | TAATACGACTCACTATAGGGAGATCCCAGCCGTTCTCCTTGATCATGT (SEQ ID NO:12) |
| 3 | 167 | CG15305 | 462 | TAATACGACTCACTATAGGGAGATATGTGCATCCATTCGAAAGACTTT (SEQ ID NO:13) |
| | | | 463 | TAATACGACTCACTATAGCGAGAATAGGGGAGGTTGTTCTTAGATTGA (SEQ ID NO:14) |
| 4 | 224 | CG2096 | 468 | TAATACGACTCACTATAGGGAGATGAAACCATCCGAGAAGAAGGCCAA (SEQ ID NO:15) |
| | | | 469 | TAATACGACTCACTATAGGGAGACAGATAATCATCAAATGCAGGAATC (SEQ ID NO:16) |
| | | CG2222 | 464 | TAATACGACTCACTATAGGGAGAACGGAATGAACTATTTTCCGAACTATTACT (SEQ ID NO:17) |
| | | | 465 | TAATACGACTCACTATAGGAGAGATGTACTGACTGTTGGTGCGCACT (SEQ ID NO:18) |
| 5 | 231 | CG2941 | 470 | TAATACGACTCACTATAGGGAGAATCTGTAGACAGACGGCAGAATTGC (SEQ ID NO:19) |
| | | | 471 | TAATACGACTCACTATAGGGAGACGCAATAGCAGTACTTCCATCTTGT (SEQ ID NO:20) |
| | | CG2938 | 474 | TAATACGACTCACTATAGGGAGAATTGGATTGCGAATCGCTCAGGATC (SEQ ID NO:21) |
| | | | 475 | TAATACGACTCACTATAGGGAGATTTTCGCGAAGGACATCAATATCAG (SEQ ID NO:22) |
| 6 | 248 | CG6998 | 476 | TAATACGACTCACTATAGCGAGAGGCCTACATCAAGAAGGAGTTCGAC (SEQ ID NO:23) |
| | | | 477 | TAATACGACTCACTATAGGGAGATGGTTAGTTGTATTTGCGAATCTTC (SEQ ID NO:24) |
| 8 | ms(1)04 | CG1524 | 482 | TAATACGACTCACTATAGGGAGAGTTGCTGATCGACAAACAAACCCAG (SEQ ID NO:25) |
| | | | 483 | TAATACGACTCACTATAGGGAGACTTTCCAGATACTGCCATCTACAGA (SEQ ID NO:26) |
| | | CG10778 | 484 | TAATACGACTCACTATAGGGAGAGAGTGTCGCGTGTAGAGGCATTCTT (SEQ ID NO:27) |
| | | | 485 | TAATACGACTCACTATAGGGAGAAAGTACACATGGACGGAGCGGATAG (SEQ ID NO:28) |
| 9 | thb-a | CG1453 | 556 | TAATACGACTCACTATAGGGAGAGGCTGCCGTTTTTCCTTTTGTTATCC (SEQ ID NO:29) |
| | | | 557 | TAATACGACTCACTATAGGGAGATGATCCTTCCTCTTTGACTCCACCT GTT (SEQ ID NO:30) |
| | | CG18292 | 558 | TAATACGACTCACTATAGGGAGACGCTAAAAACTAGTAGTTTTGTGTGCCAGG (SEQ ID NO:31) |
| | | | 559 | TAATACGACTCACTATACGCAGAACCACCATTGCTGGAGCACATGTTG (SEQ ID NO:32) |
| 9A | ms(1)13 | CG5941 | 610 | TAATACGACTCACTATAGGGAGAGGATTAGCACCGTCGACCACGAAAA (SEQ ID NO:33) |
| | | | 611 | TAATACCACTCACTATAGGGAGAAATTTCCTGTGTGGATAACGTGAGGAGTCC (SEQ ID NO:34) |
| 10 | 187 | CG10701 | 490 | TAATACGACTCACTATAGGGAGACGTTCCTGCTGTTTGGCATTCTTCT (SEQ ID NO:35) |
| | | | 491 | TAATACGACTCACTATAGGGAGAACCACAATAAGACCACCCACACAGC (SEQ ID NO:36) |
| | | CG10648 | 488 | TAATACGACTCACTATACGCAGACACCTTCTGCCGCCATGAGTACAAT (SEQ ID NO:37) |
| | | | 489 | TAATACGACTCACTATAGGGAGATTCCGCCTCCAGAGCCTTGTTGAAA (SEQ ID NO:38) |
| 11 | 226 | CG2865 | 492 | TAATACGACTCACTATACCGAGATCAAGGCGTCCATGATCACCTCGAAAT (SEQ ID NO:39) |
| | | | 493 | TAATACGACTCACTATAGGGAGAACCTGTCCAGCTGCAACTTGGTCAA (SEQ ID NO:40) |
| | | CG2854 | 494 | TAATACGACTCACTATAGGGAGAGGAGATGGAAAAGGAGCTCGGAAAA (SEQ ID NO:41) |
| | | | 495 | TAATACCACTCACTATAGGCAGATCTCAATCCGTATGCCAAGGAGCAC (SEQ ID NO:42) |
| | | CG2845 | 496 | TAATACGACTCACTATAGGGAGAAGTTGACCTCCAAGCTCCACGAACT (SEQ ID NO:43) |
| | | | 497 | TAATACGACTCACTATAGGGAGACTGGTGCTTGATGTGTGTCCTAATG (SEQ ID NO:44) |
| 12 | 269 | CG1696 | 500 | TAATACGACTCACTATAGGGAGACACTTGGCGATTGAACATGAAACAA (SEQ ID NO:45) |
| | | | 501 | TAATACGACTCACTATAGGGAGAATATAAAAAGCCCCCAAAAGAATTG (SEQ ID NO:46) |
| | | CG1486 | 502 | TAATACGACTCACTATAGGGAGAATTGCACTTTGATTGCAGTCGATTGCG (SEQ ID NO:47) |
| | | | 503 | TAATACGACTCACTATAGCGAGAGATGTGGAATGGTGTGACCGTAGTG (SEQ ID NO:48) |
| 13 | 291 | CG10798 | 504 | TAATACGACTCACTATAGGGAGAGACAGGCATATAACTCAGGAACTTA (SEQ ID NO:49) |
| | | | 505 | TAATACGACTCACTATAGGGAGACTTGATGATCACCGGCATGTTCTCG (SEQ ID NO:50) |
| 15 | 379 | CG10964 | 552 | TAATACGACTCACTATAGGGAGACGGAGTGCCGTCGTAGTTGACAAAA (SEQ ID NO:51) |
| | | | 553 | TAATACGACTCACTATAGGGAGATGACCAAGGACCAAGGCCTCAATGT (SEQ ID NO:52) |
| | | CG2151 | 554 | TAATACGACTCACTATAGGGAGAAGCCCACTGTGATGGTGCGTTCTAT (SEQ ID NO:53) |
| | | | 555 | TAATACGACTCACTATAGGGAGAATCTCATCGGCTCCGAACTGCTTGA (SEQ ID NO:54) |
| 17 | 121 | CG10988 | 560 | TAATACGACTCACTATAGUCAGACATTTAAGCAAAATGATTGCCGCCAATAGT (SEQ ID NO:55) |
| | | | 561 | TAATACCACTCACTATAGGGAGATCTCAATCCGATGCTGGACTGTGTG (SEQ ID NO:56) |
| 18 | 237 | CG1558 | 562 | TAATACGACTCACTATAGGGAGAGCCCAGAAGGAGCAGCAAAAGTTCT (SEQ ID NO:57) |
| | | | 563 | TAATACGACTCACTATAGGGAGATAAGTTACCTGCATCGAGGCATTGT (SEQ ID NO:58) |
| | | CG11697 | 564 | TAATACGACTCACTATAGGGAGAATGATTTATGCGATCGTGATACACA (SEQ ID NO:59) |
| | | | 565 | TAATACGACTCACTATAGGGAGACCGCTTCTCTTCCAACTGCCTTTTG (SEQ ID NO:60) |
| 19 | 171 | CG3954 | 566 | TAATACGACTCACTATAGGGAGAGGAGCCGAGTACATCAATGCCAACT (SEQ ID NO:61) |
| | | | 567 | TAATACGACTCACTATACCUAGAATGTAGGTCTTAAACATCTCGCGCT (SEQ ID NO:62) |
| | | CG16903 | 568 | TAATACGACTCACTATAGGGAGAGGAAATCTCGCCCATGGTGCTAGAT (SEQ ID NO:63) |
| | | | 569 | TAATACGACTCACTATAGGAGATGTTCCGATCCACGGTGATTACAGC (SEQ ID NO:64) |
| 20 | 500 | CG4399 | 570 | TAATACGACTCACTATAGGGAGATGCCCCCCTGGATGATAATGCCAAT (SEQ ID NO:65) |
| | | | 571 | TAATACGACTCACTATAGGCAGAACTTGCAGCTCGTGACTCTGATGCT (SEQ ID NO:66) |
| | | CG4406 | 572 | TAATACGACTCACTATAGGGAGAATGCTTGTTAAATTTGTTGTCATCTTTGCC (SEQ ID NO:67) |
| | | | 573 | TAATACGACTCACTATAGGCAGAATCTCCTCCGAGTCCTGGAACTTGA (SEQ ID NO:68) |
| 23 | 37 | CG16983 | 580 | TAATACGACTCACTATAGGGAGAATGCCCAGCATCAAGTTGCAATCTT (SEQ ID NO:69) |
| | | | 581 | TAATACGACTCACTATAGGGAGACGAAATGCCGCGCTTTACTTCTCCT (SEQ ID NO:70) |
| | | CG13363 | 582 | TAATACGACTCACTATAGGGAGATCCGATACCTGCGCGTCTTTGACAA (SEQ ID NO:71) |
| | | | 583 | TAATACGACTCACTATAGGGAGAGCCATTATTACCAGGTCCACTGCTG (SEQ ID NO:72) |
| 24 | 186 | CG18319 | 584 | TAATACGACTCACTATAGGGAGACTCAACGAGAAGGTCCAGACTCAAC (SEQ ID NO:73) |
| | | | 585 | TAATACGACTCACTATAGGGAGATCGACGGCATATTTCTGGGTCCACT (SEQ ID NO:74) |
| 25 | 301 | CG14813 | 586 | TAATACGACTCACTATAGGGAGAAATGTGCAGCCTTCGGTGGCGGAGTACGAC (SEQ ID NO:75) |
| | | | 587 | TAATACGACTCACTATAGGCAGACAATTACTCGCTCTGAGAAGCTGTC (SEQ ID NO:76) |
| 26 | 148 | CG8655 | 590 | TAATACGACTCACTATAGGUAGAATGCCCTTCATGGCACATGACCGAT (SEQ ID NO:77) |
| | | | 591 | TAATACGACTCACTATAGGGAGATTGCTGCTCTTGCTGCACTAGCTGT (SEQ ID NO:78) |
| 27 | 335 | CG2621 | 594 | TAATACGACTCACTATAGGGAGAAATAATAATAACAACGTTATAAGCCAGCCG (SEQ ID NO:79) |
| | | | 595 | TAATACGACTCACTATAGGGAGATAATGCGGCTGCGCAAGATGCTGTT (SEQ ID NO:80) |
| 28 | 342 | CG1725 | 528 | TAATACGACTCACTATAGOCAGAGCCACGTTGAAATCGATCACCGACA (SEQ ID NO:81) |
| | | CT4934 | 529 | TAATACGACTCACTATAGGGAGAATAGAAGGAGTTGGCGGGTGGAGAT (SEQ ID NO:82) |
| | | CT41310 | 530 | TAATACGACTCACTATAGGGAGATCTCTTTCGATTTCTTCTCTTCTGT (SEQ ID NO:83) |
| | | | 531 | TAATACGACTCACTATAGGGAGATTGATGAACACGGCGACGGGATACA (SEQ ID NO:84) |
| | | CG1594 | 532 | TAATACGACTCACTATAGOGAGAAGGGAATCGTGTGGAAAGACTCGCA (SEQ ID NO:85) |
| | | | 533 | TAATACGACTCACTATAGGGAGAACAAGGACAAATCAACGGGACTGGC (SEQ ID NO:86) |
| 29 | 419 | CG12638 | 596 | TAATACGACTCACTATAGUGAGATGTTTGCCATATCATTGCAGCTGCT (SEQ ID NO:87) |
| | | | 597 | TAATACGACTCACTATAGCGAGAGATGTCATATTGGCCAGGTCACTGG (SEQ ID NO:88) |

RNAi phenotype

Mitotic
Index
(% of

| | | |
|---|---|---|
| Example | RFP | Human |

| | | | | |
|---|---|---|---|---|
| number | Facs | control) | Microscopy | homologue |

| | | | | |
|---|---|---|---|---|
| 1 | Fewer G1 | wt | wt | AAC51331- |
| | cells, with | | | CREB-binding |
| | correspond- | | | protein |
| | ing increase | | | |
| | in G2/M | | | |
| 2 | Fewer cells | | 20% increase in | P48729 Casein |
| | in G2/M, | | chromosomal defects. | kinase I, alpha |
| | with a | | Some bright spots scattered | isoform |
| | correspond- | | in the cytoplasm in the | |
| | ing increase | | DAPI channel, most of the | |
| | in sub-G1 | | nuclei are irregularly | |
| | events | | shaped, MI decreases, and | |
| | | | DNA appears | |
| | | | hypocondensed Shape of | |
| | | | the cells is also very | |
| | | | affected. | |
| 2A | wt | 91% | 12% increase in | AAA63258- |
| | | | chromosomal defects | serine |
| | | | Multipolar and tripolar | hydroxymethyl- |
| | | | spindles | transferase |
| 2B | wt | 74% | wt | none |
| 2C | wt | 111% | 20% increase in | AAL09354 |
| | | | chromosomal defects | DiGeorge |
| | | | spindle defects, | syndrome-related |
| | | | some bipolar spindle | protein FKSG4 |
| 3 | Very slightly | wt | 20% increase in | None |
| | fewer | | chromosomal defects | |
| | cycling cells | | Difficult to see a normal | |
| | & a corre- | | spindle | |
| | sponding | | | |
| | increase in | | | |
| | sub-G1 cells | | | |
| 4 | wt | wt | 20% increase in | NP_002700 |
| | | | chromosomal defects, no | protein |
| | | | defects in centrosomes or | phosphatase I |
| | | | spindle | |
| | wt | Not done | 40% increase in | NP_073607 |
| | | | chromosomal defects | hypothetical |
| | | | Multipolar and monopolar | protein |
| | | | spindles | FLJ13912 |
| | | | Many polyploid cells | |
| | | | Some hyper-condensed | |
| | | | chromosomes | |
| 5 | Fewer cells | wt | wt | None |
| | in G2/M, | | | |
| | with a | | | |
| | correspond- | | | |
| | ing increase | | | |
| | in sub-G1 | | | |
| | events | | | |
| | wt | wt | 10% increase in | NP_075051 Cas1 |
| | | | chromosomal defects | O- |
| | | | Fewer cells indicating cell | acetyltransferase |
| | | | death | |
| | | | Multipolar spindles | |
| 6 | Very slightly | wt | wt | AAH10744 |
| | fewer cells in | | | Similar to |
| | G2/M & a | | | RIKEN cDNA |
| | correspond- | | | 6720463E02 |
| | ing increase | | | gene |
| | in sub-G1 | | | |
| | cells | | | |
| 8 | Fewer G2/M | 63% | Only 38 mitotic cells | A25220 |
| | events, with | | remained on the | ribosomal |
| | a corre- | | slide, cells are | protein S14 |
| | sponding | | very scattered and some | |
| | increase in | | are dying. Nuclei are | |
| | sub-G1 | | degraded. | |
| | events and a | | | |
| | different G1 | | | |
| | profile | | | |
| | wt | 78% | 20% increase in | hypothetical |
| | | | chromosomal defects | protein |
| | | | High number of multipolar | FLJ13102 |
| | | | spindles | (54%) Similarity |
| | | | | to Mouse |
| | | | | kinesin-like |
| | | | | protein KIF4 |
| 9 | Slight | wt | wt | (CG1453)- |
| | increase in | | | CAA69621- |
| | G1 and sub- | | | kinesin-2 |
| | G1 cells, but | | | |
| | no obvious | | | |
| | correspond- | | | |
| | ing decrease | | | |
| | in S or G2/M | | | |
| | cells | | | |
| | wt | 91% | 20% increase in | BAA22937- |
| | | | chromosomal defects | cdk2- |
| | | | Possible decrease in mitotic | associated |
| | | | index | protein 1; |
| | | | Some multipolar spindles, | cdk2ap1, |
| | | | few normal looking spindles | deleted in oral |
| | | | | cancer 1 |
| 9A | Very slight | wt | wt | MCT-1 (multiple |
| | decrease in | | | copies in a T-cell |
| | G1 peak, but | | | malignancies) |
| | no other | | | (BAA86055), |
| | obvious | | | |
| | variation | | | |
| | from wt | | | |
| | profile | | | |
| 10 | Fewer G2/M | wt | 20% increase in | A41289 human |
| | events with a | | chromosomal defects, | moesin |
| | correspond- | | misaligned chromosome | |
| | ing increase | | (40%), spindle with free | |
| | in sub-G1 | | extracentrosome, cells with | |
| | events | | more than one spindle. | |
| | wt | wt | Proportion of mitotic | NP_115898 |
| | | | chromosomal defects a bit | Mak16-like RNA |
| | | | lower than normal, high | binding protein |
| | | | proportion of monopolar | |
| | | | spindles and small spindles. | |
| | | | Very high proportion of | |
| | | | prometaphase cells | |
| | | | Cell death | |
| 11 | Fewer cells | wt | wt | none |
| | in G2/M and | | | |
| | also S. | | | |
| | Increased | | | |
| | percentage | | | |
| | of cells in | | | |
| | sub-G1 and | | | |
| | G1 | | | |
| | wt | wt | 17% increase in | CAD38627 |
| | | | chromosomal defects | hypothetical |
| | | | Higher level of polyploid, | protein |
| | | | prometaphase cells and | |
| | | | misaligned chromosomes, | |
| | | | anaphase normal | |
| | wt | wt | More than 20% increase in | AAA35609 B- |
| | | | chromosomal defects | raf protein |
| | | | More multipolar spindles | |
| 12 | Fewer cells | wt | wt | NP_056158 |
| | in G2/M and | | | hypothetical |
| | also S. | | | protein |
| | Increased | | | |
| | percentage | | | |
| | of cells in | | | |
| | sub-G1 and | | | |
| | G1 | | | |
| | wt | wt | 10% increase in | BAA19780 |
| | | | chromosomal defects | Similar to a |
| | | | More prometaphase cells | C. elegans protein |
| | | | | in cosmid |
| | | | | C14H10 |
| 13 | Fewer cells | wt | wt | CAA23831 c- |
| | in G2/M. | | | myc oncogene |
| | Increased | | | |
| | percentage | | | |
| | of cells in | | | |
| | sub-G1 and | | | |
| | G1 | | | |
| 15 | wt | wt | 15% increase in | AAC50725 11- |
| | | | chromosomal defects | cis retinol |
| | | | high number of disorganised | dehydrogenase |
| | | | spindles | |
| | wt | 81% | 20% increase in | XP_033135 |
| | | | chromosomal defects | thioredoxin |
| | | | High proportion of | reductase beta |
| | | | polyploid cells | |
| 17 | wt | wt | 22% increase of | AAC39727- |
| | | | chromosomal defects | spindle pole |
| | | | Main feature is a high | body protein |
| | | | proportion of metaphase | spc98 homolog |
| | | | figures with misaligned | GCP3 |
| | | | chromosomes (75% vs 20% | |
| | | | in normal cells) Some cells | |
| | | | without any centrosomes | |
| 18 | wt | 117% | 18% increase in | none |
| | | | chromosomal defects | |
| | | | Abnormal spindle structures | |
| | | | (increased number of | |
| | | | centrosomes) | |
| | Fewer G2/M | wt | 18% increase in | BAB14444 |
| | events, with | | chromosomal defects | unamed protein- |
| | a corre- | | More polyploid cells | similar to a |
| | sponding | | | hypothetical |
| | increase in | | | protein in the |
| | sub-G1 | | | region deleted in |
| | events. Also | | | human familial |
| | a different | | | adenomatous |
| | G1 profile | | | polyposis 1 |
| | from wt. | | | |
| 19 | Very slight | 45% | 20% increase in | AAH08692- |
| | increase in | | chromosomal defects | protein tyrosine |
| | G1 and sub- | | Spindle and centrosome | phosphatase, |
| | G1 cells, but | | seem normal. | non-receptor |
| | no obvious | | Higher level of aneuploidy | type 11 |
| | correspond- | | and polyploidy | |
| | ing decrease | | | |
| | in S or G2/M | | | |
| | cells | | | |
| | wt | wt | 20% increase in | AAD53184- |
| | | | chromosomal defects | cyclin L ania-6a |
| | | | Clear decrease in mitotic | |
| | | | index | |
| | | | A lot of spindles seem to be | |
| | | | affected in their structure, | |
| | | | poles not well defined and | |
| | | | microtubule array irregular | |
| | | | Many cells with fused | |
| | | | interphase or decondensed | |
| | | | nuclei | |
| 20 | Fewer cells | 88% | wt | AAF13722- |
| | in G2/M, | | | neurofilament |
| | with a | | | protein |
| | correspond- | | | |
| | ing increase in | | | |
| | sub-G1 | | | |
| | events. Also | | | |
| | a different | | | |
| | G1 profile | | | |
| | from wt. | | | |
| | Slight | wt | wt | XP_131206 |
| | decrease in | | | similar to GPI- |
| | G2/M and | | | anchor |
| | correspond- | | | transamidase |
| | ing slight | | | |
| | increase in | | | |
| | sub-G1 cells. | | | |
| 23 | Significant | wt | 30% increase in | XP_054159- |
| | decrease in | | chromosomal defects | hypothetical |
| | sub-G1 & | | All types of spindle and | protein |
| | G1 peaks, | | chromosomal defects are | |
| | with a | | visible but no obvious main | |
| | correspond- | | one | |
| | ing increase | | Higher proportion of | |
| | in the G2/M | | aneuploid and polyploid | |
| | peak, indica- | | cells | |
| | ting mitotic | | Possible decrease in mitotic | |
| | arrest. | | index | |
| | | | Cells with excess | |
| | | | centrosomes | |
| | wt | wt | 40% increase in | NP_057112 |
| | | | chromosomal defects | CGI-85 protein |
| | | | A lot of polyploid cells, | |
| | | | multicentrosome but some | |
| | | | normal spindle also | |
| 24 | Significant | 91% | 30% increase in | BAA11675- |
| | decrease in | | chromosomal defects | ubiquitin- |
| | sub-G1 & | | Various chromosomal | conjugating |
| | G1 peaks, | | defects ranging from | enzyme E2 |
| | but no | | number of centrosomes, | UbcH-ben |
| | correspond- | | spindle structure and | |
| | ing increase | | stretched/lagging chromatids | |
| | in the G2/M | | High number of abnormal | |
| | peak. | | anaphases 75% of anaphases | |
| | Probably | | (compared to 10-15% in | |
| | indicates | | normal cells) | |
| | mitotic | | | |
| | arrest. | | | |
| 25 | Fewer G1 | 81% | Cell death | CAA57071- |
| | events, with | | Lower proportion of | archain |
| | an increased | | chromosomal defects | |
| | number of | | | |
| | cells in | | | |
| | G2/M | | | |
| | indicating | | | |
| | mitotic arrest | | | |
| 26 | very slight | wt | 40% increase in | AAB97512- |
| | decrease in | | chromosomal defects | HsCdc7 |
| | G1 and | | Some chromosomal defects | |
| | G2/M peaks, | | in spindle structure but no | |
| | but no | | clear single phenotype | |
| | significant | | | |
| | increase in | | | |
| | sub-G1 cells | | | |
| | or polypoid | | | |
| | cells. | | | |
| 27 | wt | wt | 20% increase in | NP_002084- |
| | | | chromosomal defects | glycogen |
| | | | Many obvious mitotic | synthase kinase 3 |
| | | | chromosomal defects and | beta |
| | | | too many centrosomes per | |
| | | | cell | |
| | | | Very difficult to find a | |
| | | | normal looking mitotic | |
| | | | spindle | |
| | | | Most of the anaphases are | |
| | | | abnormal with lagging | |
| | | | chromosomes | |
| 28 | Essentially | | No increase in chromosomal | XP_012060- |
| | wt profile. | | defects but many with more | discs, large |
| | Very slight | | than two centrosomes | (Drosophila) |
| | reduction in | | | homolog 2 |
| | G1 peak, but | | | |
| | no obvious | | | |
| | correspond- | | | |
| | ing increase | | | |
| | in other | | | |
| | peaks | | | |
| | Very slight | wt | 20% increase in | NP_004963 |
| | reduction in | | chromosomal defects | JAK-2 kinase |
| | G1 peak, | | Polyploid cells | (Janus kinase 2), |
| | with a | | Abnormal number of | involved in |
| | correspond- | | centrosomes in many cells | cytokine receptor |
| | ing increase | | but some normal bipolar | signaling |
| | in sub-G1 | | spindles | |
| | cells. | | | |
| 29 | Decrease in | 94% | wt | B38637-Ras |
| | the number | | | inhibitor (clone |
| | of cells in | | | JC265)-human |
| | G2/M, with | | | (fragment) |
| | an increase | | | |
| | in the sub- | | | |
| | G1 popula- | | | |
| | tion. The G1 | | | |
| | peak differs | | | |
| | in profile | | | |
| | from wt. | | | |

### Example Section B

### P-Element Screening Results

The layout of a typical entry in the results section is shown below. Not all fields present in the actual results section contain information for each individual Drosophila line described.

Results Layout (Examples 1 to 29)

Line ID ‘(Drosophila line designation)

Phenotype
(Description of Drosophila phenotype)

Annotated Drosophila genome genomic segment containing P element insertion site (and map position)
(Accession number, map position according to the Bridges map, Lefevre, 1976)

P element Insertion site
(Base pair position within genomic segment)

Annotated Drosophila Genome Complete Genome candidate
(Derived from GADFLY Berkley Drosophila Genome Project database, accession number, MRNA sequence (complete CDS) and Peptide sequence)

Human homologue of Complete Genome candidate
(Derived from Blink and BLAST searches, accession number, mRNA sequence (complete CDS) and peptide sequence)

Putative function
(Derived from homologies or Drosophila experimental data)
   A specific example is as follows (Example 5, Category 2):

Line ID—231

Phenotype—Semi-lethal male and female, cytokinesis defect. In some cysts, variable sized Nebenkerns

Annotated Drosophila genome genomic segment containing P element insertion site (and map position)—AE003429 (3F)

P element insertion site—153,730

Annotated Drosophila genome Complete Genome candidate

CG5014—vap-33-1 vesicle associated membrane protein

(SEQ ID NO:124)

CACATGACTAGCTGACAGAATATATGGCTTTTTTACATTTTGCGTTTTCA
ACTGAAGTTTGCGAAGAAAGCGAAGCGTGGTAAACGAGTGAAATCGAAAA
TATCGACAGAAAAGCGACCTAAAGTCGGTGAAGAAGTGGCACGTTGATCG
TTGTGTTTTTTTCCCGAAATTTTCTGCAAAAAGCCCGTGCGTGCGTGAGT
TTCTCTGGCTCTTGCTTTTTTTTTGTCCATGCGTGTGTGTGTGGTCGCAT
AAATTTACCGATATTTCGGGTGTGAGAGCGAAACGAACGAAAAACGAAAG
AAAAAAAGAGAGACGAGTAAAGTAAAACGAAACAGGCATAAAAACAGCAG
CAGTTTTCTTGATATATTTGGCTAAAAAAGGCAAACCAAACAGCCAGCAA
GAACAACAAATAGCTGGGCAAAAACAGGACGCAGAAAAAATAAAATTAAA
ACGATAAGAGGCGAAAAGCGGAGAGAGTGAAATTCTCGGCAGCAACAACG
ACAAGAACAAGACCAGGAGCAGCAGCAACAACAACAACAAAAGCCAGCCG
CCACAATGAGCAAATCACTCTTTGATCTTCCGTTGACCATFGAACCAGAA
CATGAGTTGCGTTTTGTGGGTCCCTTCACCCGACCCGTTGTCACAATCAT
GACTCTGCGCAACAACTCGGCTCTGCCTCTGGTCTTCAAGATCAAGACAA
CCGCCCCGAAACGCTACTGCGTACGTCCAAACATCGGCAAGATAATTCCC
TTTCGATCAACCCAGGTGGAGATCTGCCTTCAGCCATTCGTCTACGATCA
GCAGGAGAAGAACAAGCACAAGTTCATGGTGCAGAGCGTCCTGGCACCCA
TGGATGGTGATCTAAGCGATTTAAATAAATTGTGGAAGGATCTGGAGCCC
GAGCAGCTGATGGACGCCAAACTGAAGTGCGTTTTCGAGATGCCCACCGG
TGAGGCAAATGCTGAGAACACCAGCGGTGGTGGTGCCGTTGGCGGCGGAA
CCGGAGCTGGCGGAGGCGGAAGCGCGGGTGCCAATACTAGCTGAGCCAGC
GCTGAGGCGCTCGAGAGCAAGCCGAAGCTCTCCAGCGAGGATAAGTTTAA
GCGATCCAATTTGCTCGAAACGTCTGAGAGTCTGGACTTGCTGTCCGGAG
AGATCAAAGCGCTGCGTGAATGCAACATTGAATTGCGAAGAGAGAATCTT
CAC1TGAAGGATCAAATCACACGTTTCCGGAGCTCGCCGGCCGTCAAACA
GGTGAATGAGCCCTATGCCCCAGTCCTGGCTGAGAAGCAGATTCCGGTCT
TTTACATTGCAGTTGCCATTGCTGCGGCCATCGTTAGCGTGCTGCTGGGC
AAATTCTITCTCTGA

(SEQ ID NO:125)

MSKSLFDLPLTIEPEHELRFVGPFTRPVVTIMTLRINNSALPLVFKIKTTA
PKRYCVRPNIGKIIPFRSTQVEICLQPFVYDQQEKNKHKFMVQSVLAPMD
ADLSDLNKLWKDLEPEQLMDAKLKCVFEMPTAEANAENTSGGGAVGGGTG
AAGGGSAGANTSSASAEALESKPKLSSEDKFKPSNLLETSESLDLLSGEI
KALRECNIELRRENLHLKDQITRFRSSPAVKQVNEPYAPVLAEKQIPVFY
IAVAIAAAIVSLLLGKFFL

Human homologue of Complete Genome candidate

AAD13577 VAMP-associated protein B

| | | |
|---|---|---|
| 1 | gcgcgcccac ccggtagagg acccccgccc gtgccccgac cggtccccgc ctttttgtaa | (SEQ ID NO:126) |
| 61 | aacttaaagc gggcgcagca ttaacgcttc ccgccccggt gacctctcag gggtctcccc | |
| 121 | gccaaaggtg ctccgccgct aaggaacatg gcgaaggtgg agcaggtcct gagcctcgag | |
| 181 | ccgcagcacg agctcaaatt ccgaggtccc ttcaccgatg ttgtcaccac caacctaaag | |
| 241 | cttggcaacc cgacagaccg aaatgtgtgt tttaaggtga agactacagc accacgtagg | |
| 301 | tactgtgtga ggcccaacag cggaatcatc gatgcagggg cctcaattaa tgtatctgtg | |
| 361 | atgttacagc ctttcgatta tgatcccaat gagaaaagta aacacaagtt tatggttcag | |
| 421 | tctatgtttg ctccaactga cacttcagat atggaagcag tatggaagga ggcaaaaccg | |
| 481 | gaagacctta tggattcaaa acttagatgt gtgtttgaat tgccagcaga gaatgataaa | |
| 541 | ccacatgatg tagaaataaa taaaattata tccacaactg catcaaagac agaaacacca | |
| 601 | atagtgtcta agtctctgag ttcttctttg gatgacaccg aagttaagaa ggttatggaa | |
| 661 | gaatgtaaga ggctgcaagg tgaagttcag aggctacggg aggagaacaa gcagttcaag | |
| 721 | gaagaagatg gactgcggat gaggaagaca gtgcagagca acagccccat ttcagcatta | |
| 781 | gccccaactg ggaaggaaga aggccttagc acccggctct tggctctggt ggttttgttc | |
| 841 | tttatcgttg gtgtaattat tgggaagatt gccttgtaga ggtagcatgc acaggatggt | |
| 901 | aaattggatt ggtggatcca ccatatcatg ggatttaaat ttatcataac catgtgtaaa | |
| 961 | aagaaattaa tgtatgatga catctcacag gtcttgcctt taaattaccc ctccctgcac | |
| 1021 | acacatacac agatacacac acacaaatat aatgtaacga tcttttagaa agttaaaaat | |
| 1081 | gtatagtaac tgattgaggg ggaaaagaat gatctttatt aatgacaagg gaaaccatga | |
| 1141 | gtaatgccac aatggcatat tgtaaatgtc attttaaaca ttggtaggcc ttggtacatg | |
| 1201 | atgctggatt acctctctta aaatgacacc cttcctcgcc tgttggtgct ggcccttggg | |
| 1261 | gagctggagc ccagcatgct ggggagtgcg gtcagctcca cacagtagtc cccacgtggc | |
| 1321 | ccactcccgg cccaggctgc tttccgtgtc ttcagttctg tccaagccat cagctccttg | |
| 1381 | ggactgatga acagagtcag aagcccaaag gaattgcact gtggcagcat cagacgtact | |
| 1441 | cgtcataagt gagaggcgtg tgttgactga ttgacccagc gctttggaaa taaatggcag | |
| 1501 | tgctttgttc acttaaaggg accaagctaa atttgtattg gttcatgtag tgaagtcaaa | |
| 1561 | ctgttattca gagatgttta atgcatattt aacttattta atgtatttca tctcatgttt | |
| 1621 | tcttattgtc acaagagtac agttaatgct gcgtgctgct gaactctgtt gggtgaactg | |
| 1681 | gtattgctgc tggagggctg tgggctcctc tgtctctgga gagtctggtc atgtggaggt | |
| 1741 | ggggtttatt gggatgctgg agaagagctg ccaggaagtg ttttttctgg gtcagtaaat | |
| 1801 | aacaactgtc ataggcaggg aaattctcag tagtgacagt caactctagg ttaccttttt | |
| 1861 | taatgaagag tagtcagtct tctagattgt tcttatacca cctctcaacc attactcaca | |
| 1921 | cttccagcgc ccaggtccaa gtttgagcct gacctcccct tggggaccta gcctggagtc | |
| 1981 | aggacaaatg gatcgggctg caaagggtta gaagcgaggg caccagcagt tgtgggtggg | |
| 2041 | gagcaaggga agagagaaac tcttcagcga atccttctag tactagttga gagtttgact | |
| 2101 | gtgaattaat tttatgccat aaaagaccaa cccagttctg tttgactatg tagcatcttg | |
| 2161 | aaaagaaaaa ttataataaa gccccaaaat taaga | |
| 1 | makveqvlsl epqhelkfrg pftdvvttnl klgnptdrnv cfkvkttapr rycvrpnsgi | (SEQ ID NO:127) |
| 61 | idagasinvs vmlqpfdydp nekskhkfmv qsmfaptdts dmeavwkeak pedlmdsklr | |
| 121 | cvfelpaend kphdveinki isttasktet pivskslsss lddtevkkvm eeckrlqgev | |
| 181 | qrlreenkqf keedglrmrk tvqsnspisa laptgkeegl strllalvvl ffivgviigk | |
| 241 | ial | |

Putative function
Membrane associated protein which may be involved in priming synaptic vesicles

Results Layout for Examples 2A, 2B, 2C and 9A

The results layout for Examples 2A, 2B, 2C and 9A includes, in place of the fourth field “P Element Insertion Site”, a field “P Element Insertion Site Sequence”. This field shows the actual sequence of the insertion site which is determined experimentally, as opposed to the base pair position within genomic segment present in the other Examples.

### Category 1—Female Sterile

### Example 1

### Category 1

Line ID—464

Phenotype—Female semi-sterile, brown eggs laid

Annotated Drosophila genome genomic segment containing P element insertion site (and map position)—AE003448 (8F)

Pelement Insertion site—44,575

Annotated Drosophila genome Complete Genome candidate

CG1 5319—nejire (CREB binding protein, p300/CBP)

(SEQ ID NO:89)

CTTAACCAAACAAACAACCTGTGCAACAATTGTCAAAGTGCTAGGCGACA
AATAATTTCTGAAAGAAGATTTGACAAGTTCCAATAACGAAAATATCAGA
ACACACTCGAACTCCAACATAGACGGATCATTGGAGAGTTAGTGAAAAAA
AAAAGCGAAAAATCAGAAAAACTTTATAAACTAATAGAAACAATACTACT
CAGATTTTTCGAACGTTTTTCGTCTGCGTTTCTGTTTTTTTCCGAATCGA
AAGAATCAAACTAACTCTATATGATGGCCGATCACTTAGACGAACCGCCC
CAAAAGCGGGTTAAAATGGATCCAACGGATATCTCTTACTTTCTGGAGGA
GAACCTGCCCGATGAGCTGGTGTCCTCGAATAGTGGCTGGTCGGATCAGC
TGACCGGCGGAGCAGGCGGTGGCAATGGAGGTGGCGGCGCCTCCGGTGTA
ACCACAAATCCCACATCCGGCCCAAATCCCGGTGGCGGACCCAACAAGCC
GGCAGCCCAAGGACCCGGCTCTGGCACAGGCGGAGTCGGTGTTGGAGTGA
ATGTGGGTGTCGGCGGTGTTGTTGGCGTCGGCGTTGTGCCTTCCCAGATG
AACGGAGCCGGCGGCGGCAACGGATCCGGAACGGGTGGCGACGACGGCAG
TGGCAACGGCTCAGGAGCGGGCAACAGAATCAGTCAAATGCAACACCAGC
AACTGCAGCACCTACTCCAGCAGCAGCAGCAGGGCCAGAAGGGCGCCATG
GTGGTGCCCGGCATGCAGCAGCTGGGCAGCAAGTCGCCCAACCTGCAGTC
ACCCAACCAGGGCGGCATGCAGCAGGTGGTGGGCACTCAGATGGGTATGG
TCAACTCAATGCCCATGTCAATATCGAATAATGGCAACAATGGCATGAAC
GCCATACCAGGCATGAACACCATTGCGCAGGGCAATCTGGGAAACATGGT
GCTGACCAACAGCGTTGGCGGCGGCATGGGCGGCATGGTTAATCATCTTA
AGCAGCAGCCTGGCGGCGGCGGCGGTGGGATGATCAATTCCGTTTCAGTA
CCCGGAGGACCTGGAGCAGGAGCTGGTGGCGTTGGAGCTGGCGGCGGAGG
AGCCGTTGCCGCAAACCAAGGCATGCATATGCAGAACGGCCCAATGATGG
GACGCATGGTGGGGCAACAGCATATGCTTCGTGGCCCGCATCTCATGGGT
GCCTCTGGAGGAGCTGGTGGGCCAGGAAACGGGCCTGGTGGCGGAGGACC
ACGCATGCAGAATCCGAACATGCAAATGACTCAACTCAACAGTCTGCCCT
ACGGAGTGGGTCAGTATGGTGGCCCAGGCGGTGGTAACAATCCTCAGCAA
CAGCAGCAGCAACAGCAGCAACAACTTCTCGCCCAGCAGATGGCCCAAAG
AGGTGGCGTCGTACCGGGCATGCCGCAGGGTAATCGGCCCGTTGGCACAG
TGGTGCCCATGTCCACACTCGGCGGCGATGGATCAGGGCCCGCGGGGCAG
CTGGTAAGCGGGAATCCTCAGCAGCAGCAGATGCTGGCGCAGCAGCAAAC
CGGAGCCATGGGCCCGCGTCCTCCGCAACCAAACCAGCTGCTCGGTCATC
CCGGCCAGCAGCAGCAGCAGCAACAGCAGCCTGGCACCTCGCAGCAGCAG
CAACAGCAGCAGGGAGTCGGAATCGGAGGAGCAGGCGTTGTGGCCAATGC
AGGAACCGTGGCTGGCGTGCCGGCAGTGGCAGGCGGCGGAGCCGGTGGTG
CCGTACAATCTAGCGGCCCTGGTGGCGCCAATCGCGATGTGCCCGACGAC
CGTAAGCGACAGATCCAGCAGCAACTGATGCTGCTCCTCCATGCACACAA
ATGCAATCGCAGGGAGAACCTGAATCCGAACAGGGAAGTGTGCAACGTTA
ACTACTGCAAGGCGATGAAATCCGTGCTGGCCCACATGGGCACTTGCAAA
CAGAGCAAGGACTGCACCATGCAGCATTGTGCCTCTTCGCGCCAAATTCT
GTTGCATTATAAAACGTGCCAGAACAGTGGCTGCGTCATTTGCTATCCCT
TCCGGCAGAATCATTCGGTTTTTCAAAATGCGAATGTGCCGCCAGGAGGC
GGACCGGCAGGAATTGGAGGTGCGCCACCAGGTGGCGGCGGAGCGGGTGG
TGGAGCGGCTGGAGCAGGCGGTAATCTTCAGCAGCAACAGCAGCAGCAAC
AACAGCAGCAGCAGAACCAGCAGCCCAATCTGACGGGTCTGGTAGTGGAT
GGCAAGCAAGGACAGCAGGTTGCACCGGGAGGTGGCCAAAATACTGCCAT
AGTTCTTCCCCAGCAACAGGGAGCGGGCGGTGCACCGGGTGCGCCGAAAA
CGCCTGCGGATATGGTGCAACAATTGACCCAACAGCAGCAGCAGCAGCAA
CAGCAGGTTCACCAGCAACAGGTTCAGCAACAGGAACTCCGTCGATTCGA
TGGCATGAGCCAGCAAGTCGTAGCAGGTGGTATGCAACAGCAGCAGCAGC
AGGGTTTGCCTCCTGTGATTCGCATTCAAGGCGCTCAGCCGGCCGTCAGG
GTACTGGGACCAGGTGGTCCCGGCGGCCCAAGTGGACCAAATGTTCTGCC
GAACGATGTTAACAGCCTGCATCAACAACAGCAACAAATGCTGCAACAGC
AGCAGCAACAGGGCCAGAATCGACGACGCGGTGGCCTGGCCACCATGGTG
GAGCAACAACAGCAGCATCAGCAACAACAGCAGCAACCCAATCCCGCCCA
GCTGGGTGGCAACATTCCAGCACCACTCTCTGTCAACGTCGGTGGCTTTG
GCAATACCAATTTCGGTGGTGCAGCTGCCGGCGGAGCCGTGGGAGCCAAC
GATAAGCAGCAACTGAAGGTGGCCCAAGTGCATCCGCAGAGCCATGGCGT
AGGAGCGGGCGGTGCATCAGCGGGCGCCGGGGCGAGTGGTGGTCAAGTGG
CAGCCGGTTCCAGTGTCCTGATGCCAGCCGATACCACGGGCAGTGGTAAT
GCGGGCAATCCCAACCAGAATGCAGGCGGTGTAGCTGGAGGTGCCGGCGG
TGGCAATGGCGGAAACACTGGACCTCCGGGCGACAACGAGAAAGACTGGC
GGGAATCGGTGACCGCCGATCTGCGCAACCACCTCGTCCACAAACTGGTG
CAGGCCATCTTCCCCACCTCGGATCCTACGACCATGCAGGACAAACGGAT
GCATAATCTCGTTTCATACGCGGAAAAGGTCGAGAAGGACATGTACGAAA
TGGCCAAGTCCAGATCGGAGTACTATCACCTGCTGGCCGAGAAGATCTAC
AAGATTCAAAAGGAGCTGGAGGAGAAGCGACTGAAGCGTAAGGAGCAGCA
TCAGCAGATGCTGATGCAGCAACAGGGCGTTGCGAATCCAGTGGCTGGAG
GAGCGGCTGGCGGAGCAGGCAGTGCAGCTGGTGTAGCGGGCGGTGTAGTC
TTGCCCCAGCAGCAACAGCAGCAGCAACAACAACAGCAGCAGCAGGGTCA
GCAGCCTCTGCAGAGCTGTATCCATCCAAGCATCAGTCCAATGGGCGGTG
TGATGCCGCCGCAGCAGCTGCGTCCACAGGGACCACCTGGAATACTGGGC
CAACAGACGGCAGCAGGCCTGGGCGTCGGCGTGGGAGTGACCAACAATAT
GGTTACCATGCGCAGTCATTCGCCCGGTGGCAACATGCTCGCCTTGCAGC
AACAACAGCGCATGCAGTTCCCGCAACAACAGCAGCAACAACCGCCAGGG
TCTGGAGCCGGCAAAATGCTGGTCGGTCCACCAGGACCCAGTCCCGGTGG
CATGGTGGTCAATCCCGCGCTCTCGCCTTACCAGACGACCAATGTGCTCA
CCAGTCCGGTGCCAGGACAGCAGCAACAGCAGCAGTTCATTAATGCGAAC
GGCGGCACTGGCGCCAATCCTCAACTGAGCGAAATCATGAAGCAGCGTCA
CATTCACCAGCAGCAGCAGCAACAACAACAGCAGCAGCAGCAGGGAATGT
TGTTGCCGCAGTCGCCATTTAGCAATTCAACACCTCTACAACAACAACAG
CAGCAGCAGCAGCAACAACAGCAGCAGCAGGCGACTAGCAACAGTTTTAG
CTCACCAATGCAGCAACAGCAGCAAGGTCAGCAACAGCAACAACAGAAGC
CCGGCAGTGTGCTGAATAATATGCCGCCCACGCCCACGAGTCTGGAAGCC
CTGAATGCGGGGGCCGGAGCGCCGGGAACTGGAGGATCCGCCTCCAATGT
AACGGTTTCAGCTCCGAGCCCATCGCCTGGCTTCTTGTCCAACGGCCCGT
CGATTGGCACGCCCTCCAACAATAATAATAATAGTAGTGCTAACAACAAC
CCGCCCTCGGTGAGCAGTCTAATGCAACAGCCGCTGAGCAATCGGCCGGG
TACGCCTCCTTACATACCCGCTTCCCCAGTGCCGGCGACAAGTGCCTCCG
GATTAGCGGCGAGCAGTACGCCCGCATCAGCAGCAGCCACCTGTGCGAGT
AGTGGCAGTGGCAGCAATAGCAGCAGCGGAGCAACTGCAGCGGGTGCAAG
TTCCACGTCATCATCTTCCTCGGCGGGCTCGGGTACACCACTCAGCTCGG
TATCGACTCCTACATCGGCCACGATGGCCACCAGCAGCGGTGGTGGTGGT
GGTGGTGGGGGCAATGCAGGAGGCGGATCATCCACTACGCCCGCTAGCAA
TCCACTGCTCCTCATGTCTGGAGGAACGGCAGGAGGCGGAACGGGAGCAA
CGACCACCACATCGACATCCTCGAGCAGTCGCATGATGAGCAGCTCCAGC
AGTCTCTCCTCACAGATGGCTGCCCTGGAGGCTGCGGCGCGAGACAACGA
CGATGAGACGCCCTCGCCATCCGGCGAGAATACGAACGGCAGTGGTGGCA
GTGGAAATGCCGGCGGTATGGCCTCCAAGGGCAAACTGGACTCCATTAAG
CAAGATGATGATATCAAGAAGGAGTTTATGGATGACAGCTGTGGCGGAAA
TAACGATAGCTCGCAGATGGATTGCTCGACGGGTGGTGGCAAGGGCAAGA
ATGTGAACAACGACGGAACAAGCATGATCAAAATGGAGATCAAGACGGAG
GATGGACTCGATGGCGAGGTAAAGATCAAAACGGAGGCCATGGATGTGGA
CGAGGCTGGAGGATCGACAGCCGGAGAGCATCATGGCGAAGGTGGCGGCG
GCAGTGGTGTTGGCGGCGGTAAGGATAACATAAATGGTGCGCACGATGGC
GGAGCGACAGGCGGTGCTGTGGACATAAAACCCAAGACGGAGACGAAACC
ACTCGTACCGGAGCCACTGGCACCCAATGCAGGTGACAAGAAAAAGAAGT
GCCAATTCAATCCCGAGGAACTGCGCACCGCTCTCCTGCCAACGCTAGAG
AAGCTCTACAGGCAGGAGCCCGAATCCGTGCCCTTTCGCTACCCAGTTGA
TCCCCAGGCGCTGGGCATACCTGATTACTTTGAAATCGTTAAGAAGCCCA
TGGACCTGGGCACTATACGCACCAACATCCAGAATGGAAAGTACAGTGAT
CCCTGGGAATATGTGGACGACGTTTGGCTGATGTTCGACAATGCCTGGCT
GTATAATCGCAAAACATCGCGGGTCTATCGCTATTGCACAAAGCTTTCCG
AAGTCTTTGAGGCGGAGATTGATCCTGTGATGCAGGCACTGGGATATTGC
TGCGGCAGGAAGTACACATTCAATCCACAGGTGCTATGCTGCTACGGCAA
GCAGCTCTGCACGATTCCGCGGGATGCCAAGTACTACAGCTACCAGAACA
GTCTAAAGGAATACGGTGTCGCCTCAAATAGATACACCTACTGCCAAAAG
TGCTTTAACGACATCCAGGGCGATACGGTCACACTGGGCGACGATCCACT
GCAATCGCAAACCCAAATCAAAAAGGATCAGTTCAAGGAGATGAAGAACG
ATCACCTCGAACTGGAGCCGTTTGTCAATTGCCAGGAGTGCGGACGCAAA
CAGCACCAAATCTGCGTACTCTGGCTGGATTCTATCTGGCCCGGTGGCTT
CGTGTGCGATAACTGCCTGAAAAAGAAGAACTCAAAGCGGAAGGAGAACA
AGTTCAATGCGAAACGCCTGCCCACCACCAAGCTGGGCGTGTACATAGAG
ACGCGGGTGAATAATTTCCTCAAGAAGAAGGAGGCTGGTGCCGGCGAGGT
GCACATTCGTGTGGTCAGCTCATCGGACAAGTGTGTAGAGGTGAAGCCCG
GCATGCGTCGACGATTCGTCGAGCAGGGCGAGATGATGAACGAGTTCCCA
TACCGAGCCAAAGCGCTCTTTGCCTTCGAGGAGGTGGATGGCATCGATGT
GTGCTTCTTTGGCATGCACGTTCAGGAGTATGGATCCGAGTGCCCGGCGC
CGAATACGCGGCGTGTGTATATTGCCTATTTGGATTCCGTTCATTTCTTC
CGGCCAAGACAGTACCGTACAGCGGTATATCACGAAATCCTGCTCGGCTA
TATGGACTACGTGAAACAGCTGGGCTACACAATGGCCCATATCTGGGCCT
GTCCGCCATCCGAGGGCGATGACTACATCTTTCACTGCCATCCCACGGAC
CAGAAGATACCCAAGCCCAAGCGCCTGCAGGAGTGGTACAAAAAGATGCT
TGACAAGGGAATGATCGAGCGCATCATACAGGACTACAAGGATATCCTGA
AGCAGGCGATGGAGGACAAACTGGGCTCTGCCGCAGAGCTGCCCTACTTT
GAGGGCGACTTCTGGCCCAATGTGCTGGAGGAGAGCATCAAGGAACTGGA
CCAGGAGGAGGAAGAGAAGCGCAAACAGGCCGAGGCCGCGGAAGCAGCAG
CTGCGGCAAATCTTTTCTCTATCGAGGAAAATGAAGTAAGCGGCGATGGC
AAAAAGAAGGGCCAGAAGAAGGCCAAAAAGTCGAACAAATCGAAAGCGGC
GCAGCGTAAGAACAGCAAAAAGTCCAACGAACATCAGTCGGGCAATGATC
TCTCCACAAAGATATATGCGACCATGGAGAAGCACAAGGAGGTCTTCTTC
GTTATCCGTCTGCATTCGGCGCAGTCGGCAGCTAGTTTAGCGCCCATCCA
GGATCCCGATCCGCTGCTCACATGCGATCTGATGGATGGACGCGATGCCT
TCCTCACGCTCGCCCGCGACAAGCACTTTGAGTTCTCGTCGCTGCGGCGC
GCACAATTCTCCACTCTGTCCATGTTGTATGAGCTGCATAACCAGGGTCA
GGACAAGTTTGTTTACACCTGCAACCACTGCAAGACGGCCGTGGAGACGC
GCTACCACTGTACTGTTTGTGATGACTTCGATCTGTGTATCGTGTGCAAG
GAGAAGGTTGGCCATCAGCACAAGATGGAGAAGCTCGGCTTCGACATCGA
CGACGGCTCTGCGCTGGCGGATCACAAGCAGGCTAATCCACAGGAGGCCC
GCAAGCAATCCATCCAGCGTTGCATCCAATCGCTGGCGCACGCCTGCCAG
TGTCGCGATGCCAACTGCCGCCTGCCATCGTGCCAGAAGATGAAGCTCGT
TGTCCAGCATACGAAGAACTGCAAGCGCAAGCCCAACGGAGGATGCCCCA
TTTGCAAGCAGCTTATCGCACTCTGTTGCTATCACGCGAAGAACTGTGAG
GAGCAGAAGTGCCCCGTGCCGTTCTGTCCCAACATCAAGCACAAGCTCAA
GCAGCAGCAGTCACAGCAGAAATTCCAGCAGCAGCAGTTGCTGCGTCGCC
GTGTGGCGCTCATGTCGCGTACAGCAGCTCCAGCGGCTCTGCAAGGCCCA
GCTGCAGTAAGCGGTCCGACCGTCGTCTCTGGAGGAGTGCCCGTGGTGGG
CATGTCCGGTGTGGCAGTTAGCCAACAGGTGATCCCCGGCCAGGCGGGTA
TACTGCCTCCAGGGGCGGGTGGCATGTCGCCATCTACCGTGGCAGTTCCA
TCGCCTGTTTCAGGAGGAGCGGGAGCCGGTGGAATGGGTGGAATGACATC
ACCACATCCGCATCAACCAGGTATAGGTATGAAACCTGGTGGCGGTCACT
CGCCGTCTCCAAATGTCCTACAAGTGGTGAAGCAGGTCCAGGAAGAGGCA
GCTCGTCAGCAGGTATCGCATGGCGGTGGCTTCGGCAAGGGCGTACCCAT
GGCGCCGCCCGTAATGAATCGACCAATGGGCGGCGCTGGGCCCAACCAAA
ATGTTGTTAATCAACTTGGTGGCATGGGCGTTGGAGTTGAAGGTGTCGGT
GGTGTTGGCGTCGGAGGCGTTGGTGGAGTGGGTGTTAATCAACTGAATTC
GGGTGGTGGCAATACACCCGGTGCACCCATTTCCGGTCCCGGAATGAATG
TCAATCATCTAATGTCCATGGATCAGTGGGGCGGTGGCGGAGCCGGCGGC
GGAGGTGCCAATCCCGGCGGTGGCAATCCACAAGCCCGCTATGCCAACAA
TACCGGCGGCATGCGCCAACCCACCCATGTGATGCAAACGAATCTGATAC
CGCCGCAGCAACAGCAACAGATGATGGGCGGACTGGGCGGACCCAACCAA
CTGGGAGGTGGCCAAATGCCAGTCGGCGGACAGCATGGAGGAATGGGAAT
GGGCATGGGAGCACCACCAATGGCCGGAACTGTTGGCGGAGTGCGTCCAT
CTCCCGGAGCAGGAGGTGGAGGTGGAAGTGCGACTGGGGGCGGTCTAAAT
ACGCAACAACTCGCCCTGATTATGCAAAAGATTAAGAACAATCCCACCAA
CGAGAGCAACCAGCACATCCTTGCCATACTAAAACAGAATCCGCAGATCA
TGGCGGCGATCATCAAGCAGCGCCAGCAGTCGCAGAACAATGCGGCAGCG
GGCGGAGGAGCACCTGGCCCAGGTGGAGCCCTACAGCAGCAGCAGGCCGG
TAACGGACCGCAAAATCCTCAACAGCAGCAGCAGCAGCAGCAACAGCAAC
AGGTGATGCAGCAACAGCAGATGCAGCACATGATGAACCAGCAGCAGGGC
GGCGGCGGTCCACAGCAGATGAATCCCAACCAGCAGCAGCAACAGCAGCA
GGTTAATCTCATGCAGCAGCAGCAACAAGGTGGACCCGGAGGACCAGGTT
CTGGACTTCCCACGCGCATGCCCAATATGCCCAATGCCTTGGGTATGCTG
CAGAGTCTTCCGCCCAACATGTCGCCAGGCGTTTCTACTCAGGGAGGAAT
GGTGCCCAACCAAAACTGGAACAAGATGCGTTACATGCAAATGAGCCAGT
ACCCGCCACCGTATCCGCAGCGCCAGCGTGGCCCGCACATGGGCGGAGCG
GGACCTGGTCCCGGCCAGCAACAGTTCCCCGGTGGCGGAGGTGGAGCGGG
CAACTTTAATGCGGGTGGTGCTGGTGGTGCAGGCGGCGTTGTCGGTGTGG
GCGGAGTGCCCGGAGGTGCCGGCACGGTGCCCGGTGGCGATCAATACTCG
ATGGCGAATGCCGCGGCTGCCTCCAATATGCTGCAACAGCAGCAGGGCCA
GGTGGGCGTCGGAGTGGGCGTGGGCGTGAAACCAGGACCCGGCCAACAGC
AACAGCAGATGGGCGTTGGCATGCCGCCGGGTATGCAGCAGCAACAGCAG
CAACAGCAACCGCTGCAGCAGCAGCAGATGATGCAGGTAGCAATGCCAAA
TGCGAATGCCCAGAATCCGTCGGCGGTGGTTGGCGGACCCAATGCTCAGG
TGATGGGTCCGCCGACGCCGCACTCTCTGCAGCAGCAGCTGATGCAATCG
GCCCGCTCGTCGCCGCCTATTCGCTCCCCGCAGCCAACGCCATCGCCACG
TTCGGCTCCATCGCCACGTGCTGCTCCATCCGCCTCGCCTAGGGCACAGC
CCTCGCCGCACCATGTGATGAGCAGTCACTCGCCAGCGCCGCAGGGACCA
CCGCATGACGGCATGCACAATCATGGCATGCATCATCAGTCGCCACTGCC
AGGAGTGCCGCAGGATGTTGGCGTCGGAGTCGGTGTCGGCGTTGGCGTTG
GCGTTAACGTTAACGTCGGCAACGTGGGCGTCGGCAATGCCGGAGGAGCC
CTGCCCGACGCCTCCGACCAGCTGACCAAGTTTGTGGAGCGACTCTAGTG
CAGCAACAGCAGCAGCACCAGCACCAGCACCACCACCAGCTACAATGGTT
GGTAGGCGATGTGGCTAGAGGGCTAGGGCTAGACTGAATGAATGAATGAG
TGTCCAGTAGCCGCAGACGGGATGACGACGAAGACCAACCGGCAGGGATA
ACCAGTGTGTGTTAAGCGAATTAACAACTATTACTAACTTAAATCTTTTT
TTTTTTTTTAAACGGCACCACAAATAATTGTATATTGTTATAATTAAATC
AACAAATATCGCGCCTAATGTGTACTGTAGATTAAGATGACCCACCATTA
CAACCACTAACAAATACCTTATTATTTAAGTTTAAGACGAAAGTTGGACA
GAGCATTATGATTCGATTTCCATTTTATGTCCGCGATTTAGCAAATATAT
AATATCATATATTTCATATGCCCCCAAAACACACACACACCATGTATTAA
TTAATGCGATTCCTTCGTTTCCACTAAGCAGATATAGAAAAAAAAAAA

(SEQ ID NO:90)

MMADHLDEPPQKRVKMDPTDISYFLEENLPDELVSSNSGWSDQLTGGAGG
GNGGGGASGVTTNPTSGPNPGGGPNKPAAQGPGSGTGGVGVGVNVGVGGV
VGVGVVPSQMNGAGGGNGSGTGGDDGSGNGSGAGNRISQMQHQQLQHLLQ
QQQQGQKGAMVVPGMQQLGSKSPNLQSPNQGGMQQVVGTQMGMVNSMPMS
ISNNGNNGMNAIPGMNTIAQGNLGNMVLTNSVGGGMGGMVNHLKQQPGGG
GGGMINSVSVPGGPGAGAGGVGAGGGGAVAANQGMHMQNGPMMGRMVGQQ
HMLRGPHLMGASGGAGGPGNGPGGGGPRMQNPNMQMTQLNSLPYGVGQYG
GPGGGNNPQQQQQQQQQQLLAQQMAQRGGVVPGMPQGNRPVGTVVPMSTL
GGDGSGPAGQLVSGNPQQQQMLAQQQTGAMGPRPPQPNQLLGHPGQQQQQ
QQQPGTSQQQQQQQGVGIGGAGVVANAGTVAGVPAVAGGGAGGAVQSSGP
GGANRDVPDDRKRQIQQQLMLLLHAHKCNRRENLNPNREVCNVNYCKAMK
SVLAHMGTCKQSKDCTMQHCASSRQILLHYKTCQNSGCVICYPFRQNHSV
FQNANVPPGGGPAGIGGAPPGGGGAGGGAAGAGGNLQQQQQQQQQQQQNQ
QPNLTGLVVDGKQGQQVAPGGGQNTAIVLPQQQGAGGAPGAPKTPADMVQ
QLTQQQQQQQQQVHQQQVQQQELRRFDGMSQQVVAGGMQQQQQQGLPPVI
RIQGAQPAVRVLGPGGPGGPSGPNVLPNDVNSLHQQQQQMLQQQQQQGQN
RRRGGLATMVEQQQQHQQQQQQPNPAQLGGNIPAPLSVNVGGFGNTNFGG
AAAGGAVGANDKQQLKVAQVHPQSHGVGAGGASAGAGASGGQVAAGSSVL
MPADTTGSGNAGNPNQNAGGVAGGAGGGNGGNTGPPGDNEKDWRESVTAD
LRNHLVHKLVQAIFPTSDPTTMQDKRMHNLVSYAEKVEKDMYEMAKSRSE
YYHLLAEKIYKIQKELEEKRLKRKEQHQQMLMQQQGVANPVAGGAAGGAG
SAAGVAGGVVLPQQQQQQQQQQQQQGQQPLQSCIHPSISPMGGVMPPQQL
RPQGPPGILGQQTAAGLGVGVGVTNNMVTMRSHSPGGNMLALQQQQRMQF
PQQQQQQPPGSGAGKMLVGPPGPSPGGMVVNPALSPYQTTNVLTSPVPGQ
QQQQQFINANGGTGANPQLSEIMKQRHIHQQQQQQQQQQQQGMLLPQSPF
SNSTPLQQQQQQQQQQQQQQATSNSFSSPMQQQQQGQQQQQQKPGSVLNN
MPPTPTSLEALNAGAGAPGTGGSASNVTVSAPSPSPGFLSNGPSIGTPSN
NNNNSSANNNPPSVSSLMQQPLSNRPGTPPYIPASPVPATSASGLAASST
PASAAATCASSGSGSNSSSGATAAGASSTSSSSSAGSGTPLSSVSTPTSA
TMATSSGGGGGGGGNAGGGSSTTPASNPLLLMSGGTAGGGTGATTTTSTS
SSSRMMSSSSSLSSQMAALEAAARDNDDETPSPSGENTNGSGGSGNAGGM
ASKGKLDSIKQDDDIKKEFMDDSCGGNNDSSQMDCSTGGGKGKNVNNDGT
SMIKMEIKTEDGLDGEVKIKTEAMDVDEAGGSTAGEHHGEGGGGSGVGGG
KDNINGAHDGGATGGAVDIKPKTETKPLVPEPLAPNAGDKKKKCQFNPEE
LRTALLPTLEKLYRQEPESVPFRYPVDPQALGIPDYFEIVKKPMDLGTIR
TNIQNGKYSDPWEYVDDVWLMFDNAWLYNRKTSRVYRYCTKLSEVFEAEI
DPVMQALGYCCGRKYTFNPQVLCCYGKQLCTIPRDAKYYSYQNSLKEYGV
ASNRYTYCQKCFNDIQGDTVTLGDDPLQSQTQIKKDQFKEMKNDHLELEP
FVNCQECGRKQHQICVLWLDSIWPGGFVCDNCLKKKNSKRKENKFNAKRL
PTTKLGVYIETRVNNFLKKKEAGAGEVHIRVVSSSDKCVEVKPGMRRRFV
EQGEMMNEFPYRAKALFAFEEVDGIDVCFFGMHVQEYGSECPAPNTRRVY
IAYLDSVHFFRPRQYRTAVYHEILLGYMDYVKQLGYTMAHIWACPPSEGD
DYIFHCHPTDQKIPKPKRLQEWYKKMLDKGMIERIIQDYKDILKQAMEDK
LGSAAELPYFEGDFWPNVLEESIKELDQEEEEKRKQAEAAEAAAAANLFS
IEENEVSGDGKKKGQKKAKKSNKSKAAQRKNSKKSNEHQSGNDLSTKIYA
TMEKHKEVFFVIRLHSAQSAASLAPIQDPDPLLTCDLMDGRDAFLTLARD
KHFEFSSLRRAQFSTLSMLYELHNIQGQDKFVYTCNHCKTAVETRYHCTVC
DDFDLCIVCKEKVGHQHKMEKLGFDIDDGSALADHKQANPQEARKQSIQR
CIQSLAHACQCRDANCRLPSCQKMKLVVQHTKNCKRKPNGGCPICKQLIA
LCCYHAKNCEEQKCPVPFCPNIKHKLKQQQSQQKFQQQQLLRRRVALMSR
TAAPAALQGPAAVSGPTVVSGGVPVVGMSGVAVSQQVIPGQAGILPPGAG
GMSPSTVAVPSPVSGGAGAGGMGGMTSPHPHQPGIGMKPGGGHSPSPNVL
QVVKQVQEEAARQQVSHGGGFGKGVPMAPPVMNRPMGGAGPNQNVVNQLG
GMGVGVEGVGGVGVGGVGGVGVNQLNSGGGNTPGAPISGPGMNVNHLMSM
DQWGGGGAGGGGANPGGGNPQARYANNTGGMRQPTHVMQTNLIPPQQQQQ
MMGGLGGPNQLGGGQMPVGGQHGGMGMGMGAPPMAGTVGGVRPSPGAGGG
GGSATGGGLNTQQLALIMQKIKNNPTNESNQHILAILKQNPQIMAAIIKQ
RQQSQNNAAAGGGAPGPGGALQQQQAGNGPQNPQQQQQQQQQQQVMQQQQ
MQHMMNQQQGGGGPQQMNPNQQQQQQQVNLMQQQQQGGPGGPGSGLPTRM
PNMPNALGMLQSLPPNMSPGVSTQGGMVPNQNWNKMRYMQMSQYPPPYPQ
RQRGPHMGGAGPGPGQQQFPGGGGGAGNFNAGGAGGAGGVVGVGGVPGGA
GTVPGGDQYSMANAAAASNMLQQQQGQVGVGVGVGVKPGPGQQQQQMGVG
MPPGMQQQQQQQQPLQQQQMMQVAMPNANAQNPSAVVGGPNAQVMGPPTP
HSLQQQLMQSARSSPPIRSPQPTPSPRSAPSPRAAPSASPRAQPSPHHVM
SSHSPAPQGPPHDGMHNHGMHHQSPLPGVPQDVGVGVGVGVGVGVNVNVG
NVGVGNAGGALPDASDQLTKFVERL

Human homologue of Complete Genome candidate

AAC51331—CREB-binding protein

| | | |
|---|---|---|
| 1 | tccgaattcc ttttttttaa ttgaggaatc aacagccgcc atcttgtcgc ggacccgacc | (SEQ ID NO:91) |
| 61 | ggggcttcga gcgcgatcta ctcggccccg ccggtcccgg gccccacaac cgcccgcgca | |
| 121 | ccccgctccg cccggccggc ccgctccgcc cggccctcgg cgcccgcccc ggcggccccg | |
| 181 | ctcgcctctc ggctcggcct cccggagccc ggcggcggcg gcggcggcag cggcggcggc | |
| 241 | ggcggcggaa cggggggtgg gggggccgcg gcggcggcgg cgaccccgct cggcgcattg | |
| 301 | tttttcctca cggcggcggc ggcggcgggc cgcgggccgg gagcggagcc cggagccccc | |
| 361 | tcgtcgtcgg gccgcgagcg aattcattaa gtggggcgcg gggggggagc gaggcggcgg | |
| 421 | cggcggcggc accatgttct cggggactgc ctgagccgcc cggccgggcg ccgtcgctgc | |
| 481 | cagccgggcc cgggggggcg gccgggccgc cggggcgccc ccaccgcgga gtgtcgcgct | |
| 541 | cgggaggcgg gcaggggatg agggggccgc ggccggcggc ggcggcggcg gccgggggcg | |
| 601 | ggcggtgagc gctgcggggc gctgttgctg tggctgagat ttggccgccg cctcccccac | |
| 661 | ccggcctgcg ccctccctct ccctcggcgc ccgcccgcgc cgctcgcggc gcccgcgctc | |
| 721 | gctcctctcc ctcgcagccg gcagggcccc cgacccccgt ccgggccctc gccggcccgg | |
| 781 | ccgcccgtgc ccggggctgt tttcgcgagc aggtgaaaat ggctgagaac ttgctggacg | |
| 841 | gaccgcccaa ccccaaaaga gccaaactca gctcgcccgg tttctcggcg aatgacagca | |
| 901 | cagattttgg atcattgttt gacttggaaa atgatcttcc tgatgagctg atacccaatg | |
| 961 | gaggagaatt aggcctttta aacagtggga accttgttcc agatgctgct tccaaacata | |
| 1021 | aacaactgtc ggagcttcta cgaggaggca gcggctctag tatcaaccca ggaataggaa | |
| 1081 | atgtgagcgc cagcagcccc gtgcagcagg gcctgggtgg ccaggctcaa gggcagccga | |
| 1141 | acagtgctaa catggccagc ctcagtgcca tgggcaagag ccctctgagc cagggagatt | |
| 1201 | cttcagcccc cagcctgcct aaacaggcag ccagcacctc tgggcccacc cccgctgcct | |
| 1261 | cccaagcact gaatccgcaa gcacaaaagc aagtggggct ggcgactagc agccctgcca | |
| 1321 | cgtcacagac tggacctggt atctgcatga atgctaactt taaccagacc cacccaggcc | |
| 1381 | tcctcaatag taactctggc catagcttaa ttaatcaggc ttcacaaggg caggcgcaag | |
| 1441 | tcatgaatgg atctcttggg gctgctggca gaggaagggg agctggaatg ccgtacccta | |
| 1501 | ctccagccat gcagggcgcc tcgagcagcg tgctggctga gaccctaacg caggtttccc | |
| 1561 | cgcaaatgac tggtcacgcg ggactgaaca ccgcacaggc aggaggcatg gccaagatgg | |
| 1621 | gaataactgg gaacacaagt ccatttggac agccctttag tcaagctgga gggcagccaa | |
| 1681 | tgggagccac tggagtgaac ccccagttag ccagcaaaca gagcatggtc aacagtttgc | |
| 1741 | ccaccttccc tacagatatc aagaatactt cagtcaccaa cgtgccaaat atgtctcaga | |
| 1801 | tgcaaacatc agtgggaatt gtacccacac aagcaattgc aacaggcccc actgcagatc | |
| 1861 | ctgaaaaacg caaactgata cagcagcagc tggttctact gcttcatgct cataagtgtc | |
| 1921 | agagacgaga gcaagcaaac ggagaggttc gggcctgctc gctcccgcat tgtcgaacca | |
| 1981 | tgaaaaacgt tttgaatcac atgacgcatt gtcaggctgg gaaagcctgc caagttgccc | |
| 2041 | attgtgcatc ttcacgacaa atcatctctc attggaagaa ctgcacacga catgactgtc | |
| 2101 | ctgtttgcct ccctttgaaa aatgccagtg acaagcgaaa ccaacaaacc atcctggggt | |
| 2161 | ctccagctag tggaattcaa aacacaattg gttctgttgg cacagggcaa cagaatgcca | |
| 2221 | cttctttaag taacccaaat cccatagacc ccagctccat gcagcgagcc tatgctgctc | |
| 2281 | tcggactccc ctacatgaac cagccccaga cgcagctgca gcctcaggtt cctggccagc | |
| 2341 | aaccagcaca gcctcaaacc caccagcaga tgaggactct caaccccctg ggaaataatc | |
| 2401 | caatgaacat tccagcagga ggaataacaa cagatcagca gcccccaaac ttgatttcag | |
| 2461 | aatcagctct tccgacttcc ctgggggcca caaacccact gatgaacgat ggctccaact | |
| 2521 | ctggtaacat tggaaccctc agcactatac caacagcagc tcctccttct agcaccggtg | |
| 2581 | taaggaaagg ctggcacgaa catgtcactc aggacctgcg gagccatcta gtgcataaac | |
| 2641 | tcgtccaagc catcttccca acacctgatc ccgcagctct aaaggatcgc cgcatggaaa | |
| 2701 | acctggtagc ctatgctaag aaagtggaag gggacatgta cgagtctgcc aacagcaggg | |
| 2761 | atgaatatta tcacttatta gcagagaaaa tctacaagat acaaaaagaa ctagaagaaa | |
| 2821 | aacggaggtc gcgtttacat aaacaaggca tcttggggaa ccagccagcc ttaccagccc | |
| 2881 | cgggggctca gccccctgtg attccacagg cacaacctgt gagacctcca aatggacccc | |
| 2941 | tgtccctgcc agtgaatcgc atgcaagttt ctcaagggat gaattcattt aaccccatgt | |
| 3001 | ccttggggaa cgtccagttg ccacaagcac ccatgggacc tcgtgcagcc tccccaatga | |
| 3061 | accactctgt ccagatgaac agcatgggct cagtgccagg gatggccatt tctccttccc | |
| 3121 | gaatgcctca gcctccgaac atgatgggtg cacacaccaa caacatgatg gcccaggcgc | |
| 3181 | ccgctcagag ccagtttctg ccacagaacc agttcccgtc atccagcggg gcgatgagtg | |
| 3241 | tgggcatggg gcagccgcca gcccaaacag gcgtgtcaca gggacaggtg cctggtgctg | |
| 3301 | ctcttcctaa ccctctcaac atgctggggc ctcaggccag ccagctacct tgccctccag | |
| 3361 | tgacacagtc accactgcac ccaacaccgc ctcctgcttc cacggctgct ggcatgccat | |
| 3421 | ctctccagca cacgacacca cctgggatga ctcctcccca gccagcagct cccactcagc | |
| 3481 | catcaactcc tgtgtcgtct tccgggcaga ctcccacccc gactcctggc tcagtgccca | |
| 3541 | gtgctaccca aacccagagc acccctacag tccaggcagc agcccaggcc caggtgaccc | |
| 3601 | cgcagcctca aaccccagtt cagcccccgt ctgtggctac ccctcagtca tcgcagcaac | |
| 3661 | agccgacgcc tgtgcacgcc cagcctcctg gcacaccgct ttcccaggca gcagccagca | |
| 3721 | ttgataacag agtccctacc ccctcctcgg tggccagcgc agaaaccaat tcccagcagc | |
| 3781 | caggacctga cgtacctgtg ctggaaatga agacggagac ccaagcagag gacactgagc | |
| 3841 | ccgatcctgg tgaatccaaa ggggagccca ggtctgagat gatggaggag gatttgcaag | |
| 3901 | gagcttccca agttaaagaa gaaacagaca tagcagagca gaaatcagaa ccaatggaag | |
| 3961 | tggatgaaaa gaaacctgaa gtgaaagtag aagttaaaga ggaagaagag agtagcagta | |
| 4021 | acggcacagc ctctcagtca acatctcctt cgcagccgcg caaaaaaatc tttaaaccag | |
| 4081 | aggagttacg ccaggccctc atgccaaccc tagaagcact gtatcgacag gacccagagt | |
| 4141 | cattaccttt ccggcagcct gtagatcccc agctcctcgg aattccagac tattttgaca | |
| 4201 | tcgtaaagaa tcccatggac ctctccacca tcaagcggaa gctggacaca gggcaatacc | |
| 4261 | aagagccctg gcagtacgtg gacgacgtct ggctcatgtt caacaatgcc tggctctata | |
| 4321 | atcgcaagac atcccgagtc tataagtttt gcagtaagct tgcagaggtc tttgagcagg | |
| 4381 | aaattgaccc tgtcatgcag tcccttggat attgctgtgg acgcaagtat gagttttccc | |
| 4441 | cacagacttt gtgctgctat gggaagcagc tgtgtaccat tcctcgcgat gctgcctact | |
| 4501 | acagctatca gaataggtat catttctgtg agaagtgttt cacagagatc cagggcgaga | |
| 4561 | atgtgaccct gggtgacgac ccttcacagc cccagacgac aatttcaaag gatcagtttg | |
| 4621 | aaaagaagaa aaatgatacc ttagaccccg aacctttcgt tgattgcaag gagtgtggcc | |
| 4681 | ggaagatgca tcagatttgc gttctgcact atgacatcat ttggccttca ggttttgtgt | |
| 4741 | gcgacaactg cttgaagaaa actggcagac ctcgaaaaga aaacaaattc agtgctaaga | |
| 4801 | ggctgcagac cacaagactg ggaaaccact tggaagaccg agtgaacaaa tttttgcggc | |
| 4861 | gccagaatca ccctgaagcc ggggaggttt ttgtccgagt ggtggccagc tcagacaaga | |
| 4921 | cggtggaggt caagcccggg atgaagtcac ggtttgtgga ttctggggaa atgtctgaat | |
| 4981 | ctttcccata tcgaaccaaa gctctgtttg cttttgagga aattgacggc gtggatgtct | |
| 5041 | gcttttttgg aatgcacgtc caagaatacg gctctgattg cccccctcca aacacgaggc | |
| 5101 | gtgtgtacat ttcttatctg gatagtattc atttcttccg gccacgttgc ctccgcacag | |
| 5161 | ccgtttacca tgagatcctt attggatatt tagagtatgt gaagaaatta gggtatgtga | |
| 5221 | cagggcacat ctgggcctgt cctccaagtg aaggagatga ttacatcttc cattgccacc | |
| 5281 | cacctgatca aaaaataccc aagccaaaac gactgcagga gtggtacaaa aagatgctgg | |
| 5341 | acaaggcgtt tgcagagcgg atcatccatg actacaagga tattttcaaa caagcaactg | |
| 5401 | aagacaggct caccagtgcc aaggaactgc cctattttga aggtgatttc tggcccaatg | |
| 5461 | tgttagaaga gagcattaag gaactagaac aagaagaaga ggagaggaaa aaggaagaga | |
| 5521 | gcactgcagc cagtgaaacc actgagggca gtcagggcga cagcaagaat gccaagaaga | |
| 5581 | agaacaacaa gaaaaccaac aagaacaaaa gcagcatcag ccgcgccaac aagaagaagc | |
| 5641 | ccagcatgcc caacgtgtcc aatgacctgt cccagaagct gtatgccacc atggagaagc | |
| 5701 | acaaggaggt cttcttcgtg atccacctgc acgctgggcc tgtcatcaac accctgcccc | |
| 5761 | ccatcgtcga ccccgacccc ctgctcagct gtgacctcat ggatgggcgc gacgccttcc | |
| 5821 | tcaccctcgc cagagacaag cactgggagt tctcctcctt gcgccgctcc aagtggtcca | |
| 5881 | cgctctgcat gctggtggag ctgcacaccc agggccagga ccgctttgtc tacacctgca | |
| 5941 | acgagtgcaa gcaccacgtg gagacgcgct ggcactgcac tgtgtgcgag gactacgacc | |
| 6001 | tctgcatcaa ctgctataac acgaagagcc atgcccataa gatggtgaag tgggggctgg | |
| 6061 | gcctggatga cgagggcagc agccagggcg agccacagtc aaagagcccc caggagtcac | |
| 6121 | gccggctgag catccagcgc tgcatccagt cgctggtgca cgcgtgccag tgccgcaacg | |
| 6181 | ccaactgctc gctgccatcc tgccagaaga tgaagcgggt ggtgcagcac accaagggct | |
| 6241 | gcaaacgcaa gaccaacggg ggctgcccgg tgtgcaagca gctcatcgcc ctctgctgct | |
| 6301 | accacgccaa gcactgccaa gaaaacaaat gccccgtgcc cttctgcctc aacatcaaac | |
| 6361 | acaagctccg ccagcagcag atccagcacc gcctgcagca ggcccagctc atgcgccggc | |
| 6421 | ggatggccac catgaacacc cgcaacgtgc ctcagcagag tctgccttct cctacctcag | |
| 6481 | caccgcccgg gacccccaca cagcagccca gcacacccca gacgccgcag ccccctgccc | |
| 6541 | agccccaacc ctcacccgtg agcatgtcac cagctggctt ccccagcgtg gcccggactc | |
| 6601 | agccccccac cacggtgtcc acagggaagc ctaccagcca ggtgccggcc cccccacccc | |
| 6661 | cggcccagcc ccctcctgca gcggtggaag cggctcggca gatcgagcgt gaggcccagc | |
| 6721 | agcagcagca cctgtaccgg gtgaacatca acaacagcat gcccccagga cgcacgggca | |
| 6781 | tggggacccc ggggagccag atggcccccg tgagcctgaa tgtgccccga cccaaccagg | |
| 6841 | tgagcgggcc cgtcatgccc agcatgcctc ccgggcagtg gcagcaggcg ccccttcccc | |
| 6901 | agcagcagcc catgccaggc ttgcccaggc ctgtgatatc catgcaggcc caggcggccg | |
| 6961 | tggctgggcc ccggatgccc agcgtgcagc cacccaggag catctcaccc agcgctctgc | |
| 7021 | aagacctgct gcggaccctg aagtcgccca gctcccctca gcagcaacag caggtgctga | |
| 7081 | acattctcaa atcaaacccg cagctaatgg cagctttcat caaacagcgc acagccaagt | |
| 7141 | acgtggccaa tcagcccggc atgcagcccc agcctggcct ccagtcccag cccggcatgc | |
| 7201 | aaccccagcc tggcatgcac cagcagccca gcctgcagaa cctgaatgcc atgcaggctg | |
| 7261 | gcgtgccgcg gcccggtgtg cctccacagc agcaggcgat gggaggcctg aacccccagg | |
| 7321 | gccaggcctt gaacatcatg aacccaggac acaaccccaa catggcgagt atgaatccac | |
| 7381 | agtaccgaga aatgttacgg aggcagctgc tgcagcagca gcagcaacag cagcagcaac | |
| 7441 | aacagcagca acagcagcag cagcaaggga gtgccggcat ggctgggggc atggcggggc | |
| 7501 | acggccagtt ccagcagcct caaggacccg gaggctaccc accggccatg cagcagcagc | |
| 7561 | agcgcatgca gcagcatctc cccctccagg gcagctccat gggccagatg gcggctcaga | |
| 7621 | tgggacagct tggccagatg gggcagccgg ggctgggggc agacagcacc cccaacatcc | |
| 7681 | agcaagccct gcagcagcgg attctgcagc aacagcagat gaagcagcag attgggtccc | |
| 7741 | caggccagcc gaaccccatg agcccccagc aacacatgct ctcaggacag ccacaggcct | |
| 7801 | cgcatctccc tggccagcag atcgccacgt cccttagtaa ccaggtgcgg tctccagccc | |
| 7861 | ctgtccagtc tccacggccc cagtcccagc ctccacattc cagcccgtca ccacggatac | |
| 7921 | agccccagcc ttcgccacac cacgtctcac cccagactgg ttccccccac cccggactcg | |
| 7981 | cagtcaccat ggccagctcc atagatcagg gacacttggg gaaccccgaa cagagtgcaa | |
| 8041 | tgctccccca gctgaacacc cccagcagga gtgcgctgtc cagcgaactg tccctggtcg | |
| 8101 | gggacaccac gggggacacg ctagagaagt ttgtggaggg cttgtag | |
| 1 | maenlldgpp npkraklssp gfsandstdf gslfdlendl pdelipngge lgllnsgnlv | (SEQ ID NO:92) |
| 61 | pdaaskhkql sellrggsgs sinpgignvs asspvqqglg gqaqgqpnsa nmaslsamgk | |
| 121 | splsqgdssa pslpkqaast sgptpaasqa lnpqaqkqvg latsspatsq tgpgicmnan | |
| 181 | fnqthpglln snsghslinq asqgqaqvmn gslgaagrgr gagmpyptpa mqgasssvla | |
| 241 | etltqvspqm tghaglntaq aggmakmgit gntspfgqpf sqaggqpmga tgvnpqlask | |
| 301 | qsmvnslptf ptdikntsvt nvpnmsqmqt svgivptqai atgptadpek rkliqqqlvl | |
| 361 | llhahkcqrr eqangevrac slphcrtmkn vlnhmthcqa gkacqvahca ssrqiishwk | |
| 421 | nctrhdcpvc lplknasdkr nqqtilgspa sgiqntigsv gtgqqnatsl snpnpidpss | |
| 481 | mqrayaalgl pymnqpqtql qpqvpgqqpa qpqthqqmrt lnplgnnpmn ipaggittdq | |
| 541 | qppnlisesa lptslgatnp lmndgsnsgn igtlstipta appsstgvrk gwhehvtqdl | |
| 601 | rshlvhklvq aifptpdpaa lkdrrmenlv ayakkvegdm yesansrdey yhllaekiyk | |
| 661 | iqkeleekrr srlhkqgilg nqpalpapga qppvipqaqp vrppngplsl pvnrmqvsqg | |
| 721 | mnsfnpmslg nvqlpqapmg praaspmnhs vqmnsmgsvp gmaispsrmp qppnmmgaht | |
| 781 | nnmmaqapaq sqflpqnqfp sssgamsvgm gqppaqtgvs qgqvpgaalp nplnmlgpqa | |
| 841 | sqlpcppvtq splhptpppa staagmpslq httppgmtpp qpaaptqpst pvsssgqtpt | |
| 901 | ptpgsvpsat qtqstptvqa aaqaqvtpqp qtpvqppsva tpqssqqqpt pvhaqppgtp | |
| 961 | lsqaaasidn rvptpssvas aetnsqqpgp dvpvlemkte tqaedtepdp geskgeprse | |
| 1021 | mmeedlqgas qvkeetdiae qksepmevde kkpevkvevk eeeesssngt asqstspsqp | |
| 1081 | rkkifkpeel rqalmptlea lyrqdpeslp frqpvdpqll gipdyfdivk npmdlstikr | |
| 1141 | kldtgqyqep wqyvddvwlm fnnawlynrk tsrvykfcsk laevfeqeid pvmqslgycc | |
| 1201 | grkyefspqt lccygkqlct iprdaayysy qnryhfcekc fteiqgenvt lgddpsqpqt | |
| 1261 | tiskdqfekk kndtldpepf vdckecgrkm hqicvlhydi iwpsgfvcdn clkktgrprk | |
| 1321 | enkfsakrlq ttrlgnhled rvnkflrrqn hpeagevfvr vvassdktve vkpgmksrfv | |
| 1381 | dsgemsesfp yrtkalfafe eidgvdvcff gmhvqeygsd cpppntrrvy isyldsihff | |
| 1441 | rprclrtavy heiligyley vkklgyvtgh iwacppsegd dyifhchppd qkipkpkrlq | |
| 1501 | ewykkmldka faeriihdyk difkqatedr ltsakelpyf egdfwpnvle esikeleqee | |
| 1561 | eerkkeesta asettegsqg dsknakkknn kktnknkssi srankkkpsm pnvsndlsqk | |
| 1621 | lyatmekhke vffvihlhag pvintlppiv dpdpllscdl mdgrdafltl ardkhwefss | |
| 1681 | lrrskwstlc mlvelhtqgq drfvytcnec khhvetrwhc tvcedydlci ncyntkshah | |
| 1741 | kmvkwglgld degssqgepq skspqesrrl siqrciqslv hacqcrnanc slpscqkmkr | |
| 1801 | vvqhtkgckr ktnggcpvck qliaiccyha khcqenkcpv pfclnikhkl rqqqiqhrlq | |
| 1861 | qaqlmrrrma tmntrnvpqq slpsptsapp gtptqqpstp qtpqppaqpq pspvsmspag | |
| 1921 | fpsvartqpp ttvstgkpts qvpappppaq pppaaveaar qiereaqqqq hlyrvninns | |
| 1981 | mppgrtgmgt pgsqmapvsl nvprpnqvsg pvmpsmppgq wqqaplpqqq pmpglprpvi | |
| 2041 | smqaqaavag prmpsvqppr sispsalqdl lrtlkspssp qqqqqvlnil ksnpqlmaaf | |
| 2101 | ikqrtakyva nqpgmqpqpg lqsqpgmqpq pgmhqqpslq nlnamqagvp rpgvppqqqa | |
| 2161 | mgglnpqgqa lnimnpghnp nmasmnpqyr emlrrqllqq qqqqqqqqqq qqqqqqgsag | |
| 2221 | maggmaghgq fqqpqgpggy ppamqqqqrm qqhlplqgss mgqmaaqmgq lgqmgqpglg | |
| 2281 | adstpniqqa lqqrilqqqq mkqqigspgq pnpmspqqhm lsgqpqashl pgqqiatsls | |
| 2341 | nqvrspapvq sprpqsqpph sspspriqpq psphhvspqt gsphpglavt massidqghl | |
| 2401 | gnpeqsamlp qlntpsrsal sselslvgdt tgdtlekfve gl | |

Putative function
CREB-binding protein, transcription factor

### Example 2

### Category 1

Line ID—492

Phenotype—Female sterile, few eggs laid, several fully matured eggs in ovarioles

Annotated Drosophila genome genomic segment containing P element insertion site (and map position)—AE003490 (11B4-14)

P element insertion site—30,773

Annotated Drosophila genome Complete Genome candidate

CG2028—CK1 alpha (2 splice variants)

(SEQ ID NO:93)

TAAAGTGCAAGCTGGAAAAGAAAAGCAAAACAAATTCCGGAGAGCAGAAA
GAGAGTTTTTCAAGTGAACGCGTCCAACTGTTTTTGAAGCGAAGCGCTTA
GGCGGAGGAGCAGCTAGCCAGGATGGACAAGATGCGGATATTGAAGGAAA
GTCGCCCCGAGATAATCGTCGGTGGCAAATATCGGGTGATCAGGAAGATT
GGAAGCGGATCGTTTGGCGACATTTACCTGGGCATGAGCATCCAGAGCGG
CGAAGAAGTGGCCATCAAGATGGAGAGCGCCCACGCCCGCCATCCGCAGC
TGTTGTACGAGGCCAAGCTGTACCGCATTCTGAGCGGCGGCGTTGGATTC
CCTCGTATACGTCACCATGGCAAGGAAAAGAACTTCAACACCCTGGTCAT
GGACCTGCTGGGACCCTCGCTGGAGGATCTGTTCAATTTCTGTACGCGCC
ATTTCACAATCAAAACGGTTCTGATGCTCGTCGACCAGATGATCGGACGC
TTGGAGTACATCCATCTCAAGTGCTTCATCCATCGCGACATCAAGCCGGA
TAACTTCCTAATGGGCATTGGTCGGCACTGCAATAAGCTGTTCCTGATCG
ATTTCGGTCTGGCCAAGAAGTTCCGCGATCCGCACACGCGCCATCACATC
GTTTACCGCGAGGACAAGAACCTCACCGGCACTGCCCGCTATGCCTCGAT
CAATGCCCATCTGGGCATCGAGCAGTCGCGGCGTGACGACATGGAATCGC
TTGGATACGTGATGATGTACTTCAATCGCGGCGTACTGCCATGGCAAGGC
ATGAAGGCCAACACCAAGCAGCAGAAATACGAGAAGATCTCCGAAAAGAA
GATGTCCACGCCCATCGAGGTCCTCTGCAAGGGCTCGCCGGCCGAGTTCT
CCATGTATCTGAACTATTGTCGTAGCCTGCGCTTCGAGGAGCAGCCAGAT
TACATGTACCTACGTCAATTGTTCCGCATACTGTTCAGAACGCTGAACCA
TCAGTATGACTACATCTACGACTGGACAATGCTGAAGCAGAAGACCCATC
AGGGTCAACCCAATCCAGCTATACTCTTGGAGCAATTGGACAAGGACAAG
GAGAAGCAGAACGGCAAGCCCCTGATCGCGGACTAAGAGCTGCAGCGCAT
TCAGACGAATGGGGGGAGTGCATCAGAGAAGGAGAACGTGGATGCGTGGA
TGTAAATGACGTTGATGTGGGCGAAAGGCCCGGCAAGGAGCGGAGCAAAT
ATGAAACAGACGCAACCGTAAAATTGAGTAACACCAGCGGTCGTCCGAAT
GTTTCTTAATATTAATTTAAATTCAATACTAAACAAATAAGGAACCACAA
ACAAGCAAGCAAC

(SEQ ID NO:94)

MDKMRILKESRPEIIVGGKYRVIRKIGSGSFGDIYLGMSIQSGEEVAIKM
ESAHARHPQLLYEAKLYRILSGGVGFPRIRHHGKEKNFNTLVMDLLGPSL
EDLFNFCTRHFTIKTVLMLVDQMIGRLEYIHLKCFIHRDIKPDNFLMGIG
RHCNKLFLIDFGLAKKFRDPHTRHHIVYREDKNLTGTARYASINAHLGIE
QSRRDDMESLGYVMMYFNRGVLPWQGMKANTKQQKYEKISEKKMSTPIEV
LCKGSPAEFSMYLNYCRSLRFEEQPDYMYLRQLFRILFRTLNHQYDYIYD
WTMLKQKTHQGQPNPAILLEQLDKDKEKQNGKPLIAD

(SEQ ID NO:95)

TTTGGTTGAACCTATCGGGCCCTATCGATATAAGCAAAAGCATTTTTGCT
GGATCTACCATTTTATTTTAGTTAATAAAATACATATATTTCCTCTCTTT
TTGTTCCGTTTGTGCGCGTACAAAACTAGCTGCGAACTCGTGCAATATTT
CATAAACTGAATGGGAAAACAACGATAACGACGAAAGAAAACGAAAACGG
ATCTGCGACGAAATTTTCCCCGTTCCGTTTTTTTTTCTCCACCAGCAGCA
GAAGCAGCAGAGCAAAAGCAGCGAATATATTTGTAAAAGAGAGCCCCAAC
CTTGAGAAAAAACAACCAGCAGGGCAATAATTAGTTGAATTTATCGTCTG
CTGTTTTTCAAGTGAACGCGTCCAACTGTTTTTGAAGCGAAGCGCTTAGG
CGGAGGAGCAGCTAGCCAGGATGGACAAGATGCGGATATTGAAGGAAAGT
CGCCCCGAGATAATCGTCGGTGGCAAATATCGGGTGATCAGGAAGATTGG
AAGCGGATCGTTTGGCGACATTTACCTGGGCATGAGCATCCAGAGCGGCG
AAGAAGTGGCCATCAAGATGGAGAGCGCCCACGCCCGCCATCCGCAGCTG
TTGTACGAGGCCAAGCTGTACCGCATTCTGAGCGGCGGCGTTGGATTCCC
TCGTATACGTCACCATGGCAAGGAAAAGAACTTCAACACCCTGGTCATGG
ACCTGCTGGGACCCTCGCTGGAGGATCTGTTCAATTTCTGTACGCGCCAT
TTCACAATCAAAACGGTTCTGATGCTCGTCGACCAGATGATCGGACGCTT
GGAGTACATCCATCTCAAGTGCTTCATCCATCGCGACATCAAGCCGGATA
ACTTCCTAATGGGCATTGGTCGGCACTGCAATAAGCTGTTCCTGATCGAT
TTCGGTCTGGCCAAGAAGTTCCGCGATCCGCACACGCGCCATCACATCGT
TTACCGCGAGGACAAGAACCTCACCGGCACTGCCCGCTATGCCTCGATCA
ATGCCCATCTGGGCATCGAGCAGTCGCGGCGTGACGACATGGAATCGCTT
GGATACGTGATGATGTACTTCAATCGCGGCGTACTGCCATGGCAAGGCAT
GAAGGCCAACACCAAGCAGCAGAAATACGAGAAGATCTCCGAAAAGAAGA
TGTCCACGCCCATCGAGGTCCTCTGCAAGGGCTCGCCGGCCGAGTTCTCC
ATGTATCTGAACTATTGTCGTAGCCTGCGCTTCGAGGAGCAGCCAGATTA
CATGTACCTACGTCAATTGTTCCGCATACTGTTCAGAACGCTGAACCATC
AGTATGACTACATCTACGACTGGACAATGCTGAAGCAGAAGACCCATCAG
GGTCAACCCAATCCAGCTATACTCTTGGAGCAATTGGACAAGGACAAGGA
GAAGCAGAACGGCAAGCCCCTGATCGCGGACTAAGAGCTGCAGCGCATTC
AGACGAATGGGGGGAGTGCATCAGAGAAGGAGAACGTGGATGCGTGGATG
TAAATGACGTTGATGTGGGCGAAAGGCCCGGCAAGGAGCGGAGCAAATAT
GAAACAGACGCAACCGTAAAATTGAGTAACACCAGCGGTCGTCCGAATGT
TTCTTAATATTAATTTAAATTCAATACTAAACAAATAAGGAACCACAAAC
AAGCAAGCAAC

(SEQ ID NO:96)

MDKMRILKESRPEIIVGGKYRVIRKIGSGSFGDIYLGMSIQSGEEVAIKM
ESAHARHPQLLYEAKLYRILSGGVGFPRIRHHGKEKNFNTLVMDLLGPSL
EDLFNFCTRHFTIKTVLMLVDQMIGRLEYIHLKCFIHRDIKPDNFLMGIG
RHCNKLFLIDFGLAKKFRDPHTRHHIVYREDKNLTGTARYASINAHLGIE
QSRRDDMESLGYVMMYFNRGVLPWQGMKANTKQQKYEKISEKKMSTPIEV
LCKGSPAEFSMYLNYCRSLRFEEQPDYMYLRQLFRILFRTLNHQYDYIYD
WTMLKQKTHQGQPNPAILLEQLDKDKEKQNGKPLIAD

Human homologue of Complete Genome candidate

P48729 Casein kinase I, alpha isoform (cki-alpha) (ck1)

| | | |
|---|---|---|
| 1 | ccgcctccgt gttccgtttc ctgccgccct cctctcgtag ccttgcctag tgtggagccc | (SEQ ID NO:97) |
| 61 | caggcctccg tcctcttccc agaggtgtcg aggcttggcc ccagcctcca tcttcgtctc | |
| 121 | tcaggatggc gagtagcagc ggctccaagg ctgaattcat tgtcggtggg aaatataaac | |
| 181 | tggtacggaa gatcgggtct ggctccttcg gggacatcta tttggcgatc aacatcacca | |
| 241 | acggcgagga agtggcactg aagctagaat ctcagaaggc caggcatccc cagttgctgt | |
| 301 | acgagagcaa gctctataag attcttcaag gtggggttgg catcccccac atacggtggt | |
| 361 | atggtcagga aaaagactac aatgtactag tcatggatct tctgggacct agcctcgaag | |
| 421 | acctcttcaa tttctgttca agaaggttca caatgaaaac tgtacttatg ttagctgacc | |
| 481 | agatgatcag tagaattgaa tatgtgcata caaagaattt tatacacaga gacattaaac | |
| 541 | cagataactt cctaatgggt attgggcgtc actgtaataa gttattcctt attgattttg | |
| 601 | gtttggccaa aaagtacaga gacaacagga caaggcaaca cataccatac agagaagata | |
| 661 | aaaacctcac tggcactgcc cgatatgcta gcatcaatgc acatcttggt attgagcaga | |
| 721 | gtcgccgaga tgacatggaa tcattaggat atgttttgat gtattttaat agaaccagcc | |
| 781 | tgccatggca agggctaaag gctgcaacaa agaaacaaaa atatgaaaag attagtgaaa | |
| 841 | agaagatgtc cacgcctgtt gaagttttat gtaaggggtt tcctgcagaa tttgcgatgt | |
| 901 | acttaaacta ttgtcgtggg ctacgctttg aggaagcccc agattacatg tatctgaggc | |
| 961 | agctattccg cattcttttc aggaccctga accatcaata tgactacaca tttgattgga | |
| 1021 | caatgttaaa gcagaaagca gcacagcagg cagcctcttc aagtgggcag ggtcagcagg | |
| 1081 | cccaaacccc cacaggcaag caaactgaca aatccaagag taacatgaaa ggtttctaat | |
| 1141 | ttctaagcat gaattgagga acagaagaag cagacgagat gatcggagca gcatttgttt | |
| 1201 | ctccccaaat ctagaaattt tagttcatat gtacactagc cagtggttgt ggacaacca | |
| 1 | masssgskae fivggkyklv rkigsgsfgd iylainitng eevalklesq karhpqllye | (SEQ ID NO:98) |
| 61 | sklykilqgg vgiphirwyg qekdynvlvm dllgpsledl fnfcsrrftm ktvlmladqm | |
| 121 | isrieyvhtk nfihrdikpd nflmgigrhc nklflidfgl akkyrdnrtr qhipyredkn | |
| 181 | ltgtaryasi nahlgieqsr rddmeslgyv lmyfnrtslp wqglkaatkk qkyekisekk | |
| 241 | mstpvevlck gfpaefamyl nycrglrfee apdymylrql frilfrtlnh qydytfdwtm | |
| 301 | lkqkaaqqaa sssgqgqqaq tptgkqtdks ksnmkgf | |

Putative function
Casein kinase

### Example 2A

### Category 1

Line ID—ccr-a2

Phenotype—Female semi-sterile, Lays eggs, but arrest before cortical migration

Annotated Drosophila genome genomic segment containing P element insertion site (and map position)—AE003435 (5C6)

P element insertion site sequence

(SEQ ID NO:99)
GATCAGACGATATTCGGACTCCAAGCAGAGCACTTTGAAGGTGAGTTCGCCGGAAA
CCAGGCAAAGCGCCATTCGCCATTCAGGCTGCGCAACTGTTGGGAAGGGCGATCGG
TGCGGGCCTCTTCGCTATTACGCCAGCTGGCGAAAGGGGGATGTGCTGCAAGGCGA
TTAAGTTGGGTAACGCCAGGGTTTTCCCAGTCACGACGTTGTAAAACGACGGCCAGT
GCCAAGCTCTGCTGCTCTAAACGACGCATTTCGTACTCCAAAGTACGAATTTTTTCCC
TCAAGCTCTTATTTTCATTAAACAATGAACAGGACCTAACGCCACAGTA

Annotated Drosophila genome Complete Genome candidate

CG3011—glycine hydroxymethyltransferase

(SEQ ID NO:100)

GTAAATGTTGTTTACCAACGTAACGCGTGTTTTCGCTTCGTTGTATTTTC
GGTGTCGAATATTTTGGATGCTGGCCAAGAGATAGCGCAGCGATCGGGTC
GGAACTCTTGGGCGGACTTATCACTGGGTCGGTCAGGGGTCACGGGTTAT
CGTTATCGCTTATCAGCCAGCGGCGGCGTCATCTCAGCGCCGGCGACTCT
TCTCACTTTGCGGCAGTTCCGATTCGAACGCAGCCGTTTACAAAGACATG
CAGCGGGCGCGCTCTACACTGACACAAAAGCTTCGGTTTTGCCTTAGTCG
GGACCTGAACACCAAAGTTGGCAACCCGGTTAACTTCGAGACTGGAAAGC
TTAGCGGAGCTTTAACTCGCATCGCCGCCAAAAAACAACCATCACCAACG
CCATTCTTACCGGCGATCAGACGATATTCGGACTCCAAGCAGAGCACTTT
GAAGAATATGGCCGATCAGAAACTGCTGCAAACCCCGCTGGCACAGGGCG
ATCCGGAGCTGGCCGAGCTGATCAAGAAGGAGAAGGAGCGCCAGCGCGAA
GGACTCGAGATGATCGCCAGTGAGAACTTCACCTCGGTGGCGGTTCTCGA
GAGCCTGAGCTCCTGCCTGACCAACAAGTACTCCGAGGGATATCCCGGCA
AGAGGTACTACGGTGGCAACGAGTACATCGACCGCATAGAGCTGCTCGCC
CAGCAACGCGGACGCGAGCTGTTCAACCTGGACGATGAGAAGTGGGGCGT
TAATGTGCAGCCTTATTCCGGATCCCCGGCCAATCTGGCTGTCTACACGG
GCGTCTGCCGGCCCCACGATCGCATCATGGGCCTGGATCTGCCCGATGGC
GGTCACTTGACGCACGGTTTCTTCACGCCCACCAAGAAGATATCGGCCAC
ATCGATCTTCTTCGAGAGCATGCCGTACAAAGTGAACCCGGAGACGGGCA
TCATCGATTACGATAAGTTGGCGGAGGCGGCGAAGAATTTCCGGCCGCAG
ATCATCATTGCTGGCATATCGTGCTACTCCCGTCTGCTGGACTATGCGCG
TTTCCGACAGATTTGCGATGATGTGGGCGCCTACCTGATGGCCGACATGG
CCCATGTGGCGGGCATTGTGGCCGCGGGATTGATACCATCGCCGTTCGAA
TGGGCCGACATTGTGACCACCACCACGCACAAGACACTGCGAGGTCCGCG
CGCCGGCGTGATCTTCTTCCGCAAGGGCGTGCGCAGCACCAAGGCCAATG
GAGACAAGGTACTCTACGATCTGGAGGAGCGCATCAACCAGGCGGTGTTT
CCATCACTCCAGGGTGGTCCGCACAACAACGCCGTGGCTGGCATTGCCAC
CGCCTTCAAGCAGGCCAAGAGTCCCGAATTCAAGGCCTACCAGACGCAGG
TGCTCAAGAATGCCAAGGCCCTGTGCGATGGCCTCATTTCGCGAGGCTAT
CAGGTGGCCACCGGCGGCACCGACGTCCATTTGGTGCTGGTCGATGTGCG
TAAGGCTGGCCTGACCGGCGCCAAGGCCGAGTACATCCTCGAGGAGGTGG
GCATCGCGTGCAACAAGAACACTGTGCCCGGCGACAAGTCCGCCATGAAT
CCCTCCGGCATCCGGCTGGGCACACCGGCCCTGACCACTCGCGGCCTTGC
CGAGCAGGACATCGAGCAGGTGGTGGCCTTCATCGATGCTGCCCTAAAGG
TTGGCGTCCAGGCAGCCAAGCTGGCCGGCAGTCCCAAGATAACCGATTAC
CACAAGACGCTGGCCGAGAATGTGGAGCTCAAGGCCCAGGTGGACGAGAT
CCGCAAGAATGTGGCCCAGTTCAGCAGGAAATTCCCGCTGCCCGGCCTGG
AGACCCTGTAG

(SEQ ID NO:101)

MQRARSTLTQKLRFCLSRDLNTKVGNPVNFETGKLSGALTRIAAKKQPSP
TPFLPAIRRYSDSKQSTLKNMADQKLLQTPLAQGDPELAELIKKEKERQR
EGLEMIASENFTSVAVLESLSSCLTNKYSEGYPGKRYYGGNEYIDRIELL
AQQRGRELFNLDDEKWGVNVQPYSGSPANLAVYTGVCRPHDRIMGLDLPD
GGHLTHGFFTPTKKISATSIFFESMPYKVNPETGIIDYDKLAEAAKNFRP
QIIIAGISCYSRLLDYARFRQICDDVGAYLMADMAHVAGIVAAGLIPSPF
EWADIVTTTTHKTLRGPRAGVIFFRKGVRSTKANGDKVLYDLEERINQAV
FPSLQGGPHNNAVAGIATAFKQAKSPEFKAYQTQVLKNAKALCDGLISRG
YQVATGGTDVHLVLVDVRKAGLTGAKAEYILEEVGIACNKNTVPGDKSAM
NPSGIRLGTPALTTRGLAEQDIEQVVAFIDAALKVGVQAAKLAGSPKITD
YHKTLAENVELKAQVDEIRKNVAQFSRKFPLPGLETL

Human homologue of Complete Genome candidate

AAA63258—serine hydroxymethyltransferase

| | | |
|---|---|---|
| 1 | ggcacgaggc ctgcgacttc cgagttgcga tgctgtactt ctctttgttt tgggcggctc | (SEQ ID NO:102) |
| 61 | ggcctctgca gagatgtggg cagctggtca ggatggccat tcgggctcag cacagcaacg | |
| 121 | cagcccagac tcagactggg gaagcaaaca ggggctggac aggccaggag agcctgtcgg | |
| 181 | acagtgatcc tgagatgtgg gagttgctgc agagggagaa ggacaggcag tgtcgtggcc | |
| 241 | tggagctcat tgcctcagag aacttctgca gccgagctgc gctggaggcc ctggggtcct | |
| 301 | gtctgaacaa caagtactcg gagggttatc ctggcaagag atactatggg ggagcagagg | |
| 361 | tggtggatga aattgagctg ctgtgccagc gccgggcctt ggaagccttt gacctggatc | |
| 421 | ctgcacagtg gggagtcaat gtccagccct actccgggtc cccagccaac ctggccgtct | |
| 481 | acacagccct tctgcaacct cacgaccgga tcatggggct ggacctgccc gatgggggcc | |
| 541 | agtgatctca cccacggcta catgtctgac gtcaagcgga tatcagccac gtccatcttc | |
| 601 | ttcgagtcta tgccctataa gctcaacccc aaaactggcc tcattgacta caaccagctg | |
| 661 | gcactgactg ctcgactttt ccggccacgg ctcatcatag ctggcaccag cgcctatgct | |
| 721 | cgcctcattg actacgcccg catgagagag gtgtgtgatg aagtcaaagc acacctgctg | |
| 781 | gcagacatgg cccacatcag tggcctggtg gctgccaagg tgattccctc gcctttcaag | |
| 841 | cacgcggaca tcgtcaccac cactactcac aagactcttc gaggggccag gtcagggctc | |
| 901 | atcttctacc ggaaaggggt gaaggctgtg gaccccaaga ctggccggga gatcccttac | |
| 961 | acatttgagg accgaatcaa ctttgccgtg ttcccatccc tgcagggggg cccccacaat | |
| 1021 | catgccattg ctgcagtagc tgtggcccta aagcaggcct gcacccccat gttccgggag | |
| 1081 | tactccctgc aggttctgaa gaatgctcgg gccatggcag atgccctgct agagcgaggc | |
| 1141 | tactcactgg tatcaggtgg tactgacaac cacctggtgc tggtggacct gcggcccaag | |
| 1201 | ggcctggatg gagctcgggc tgagcgggtg ctagagcttg tatccatcac tgccaacaag | |
| 1261 | aacacctgtc ctggagaccg aagtgccatc acaccgggcg gcctgcggct tggggcccca | |
| 1321 | gccttaactt ctcgacagtt ccgtgaggat gacttccgga gagttgtgga ctttatagat | |
| 1381 | gaaggggtca acattggctt agaggtgaag agcaagactg ccaagctcca ggatttcaaa | |
| 1441 | tccttcctgc ttaaggactc agaaacaagt cagcgtctgg ccaacctcag gcaacgggtg | |
| 1501 | gagcagtttg ccagggcctt ccccatgcct ggttttgatg agcattgaag gcacctggga | |
| 1561 | aatgaggccc acagactcaa agttactctc cttcccccta cctgggccag tgaaatagaa | |
| 1621 | agcctttcta ttttttggtg cgggagggaa gacctctcac ttagggcaag agccaggtat | |
| 1681 | agtctccctt cccagaattt gtaactgaga agatcttttc tttttccttt ttttggtaac | |
| 1741 | aagacttaga aggagggccc aggcactttc tgtttgaacc cctgtcatga tcacagtgtc | |
| 1801 | agagacgcgt cctctttctt ggggaagttg aggagtgccc ttcagagcca gtagcaggca | |
| 1861 | ggggtgggta ggcaccctcc ttcctgtttt tatctaataa aatgctaacc tgcaaaaaaa | |
| 1921 | aaaaaaaaaa a | |
| 1 | aaqtqtgean rgwtgqesls dsdpemwell qrekdrqcrg leliasenfc sraalealgs | (SEQ ID NO:103) |
| 61 | clnnkysegy pgkryyggae vvdeiellcq rraleafdld paqwgvnvqp ysgspanlav | |
| 121 | ytallqphdr imgldlpdgg hlthgymsdv krisatsiff esmpyklnpk tglidynqla | |
| 181 | ltarlfrprl iiagtsayar lidyarmrev cdevkahlla dmahisglva akvipspfkh | |
| 241 | adivtttthk tlrgarsgli fyrkgvkavd pktgreilyt fedrinfavf pslqggphnh | |
| 301 | aiaavavalk qactpmfrey slqvlknara madallergy slvsggtdnh lvlvdlrpkg | |
| 361 | ldgaraervl elvsitankn tcpgdrsait pgglrlgapa ltsrqfredd frrvvdfide | |
| 421 | gvniglevks ktaklqdfks fllkdsetsq rlanlrqrve qfarafpmpg fdeh | |

Putative function
hydroxymethyltransferase

### Example 2B

### Category 1

Line ID—ewv-b

Phenotype—Female sterile, No eggs laid. Fully mature eggs, but “retained eggs” phenotype. Also has a mitotic phenotype: higher mitotic index, uneven chromosome staining, tangled and badly defined chromosomes with frequent bridges

Annotated Drosophila genome genomic segment containing P element insertion site (and map position)—AE003486 (10D4-6)

P element insertion site sequence

| (SEQ ID NO:104) | |
|---|---|
| | GACAGGAGCAGCTCGGAACGGACAGGAAAAGCAGGAGACTAAACAGTAAGCAATA |
| | AATTGATTTGGCGTATAGTAGCTTACACCAAAGTACATATATTGCCGCATATATAGC |
| | CAGCCGGTCACTTGCGGATCAGCCAACGTCCTGGGCCCCAAGGCGATAGATACCAC |
| | GATAAGGAGATACAGCGATACCACCAATCATTAGCAGGCGACAACGACACATCCGC |
| | ATCCGCAGAAGATGTCCAACGGCAAGGCGACGGTCTCGTTCTTCGAGACCGGGAGC |
| | ACCAAACAGTTCGAGTACTGCTACCAGCTCTATCCCCAGGTTCTTAAGCTAAAGGCC |
| | GAGAAGCGCAACTGTTGGGAAGGGCGATCGGTGCGGGCCTCTTCGCTATTACGCCA |
| | GCTGGCGAAAGGGGGATGTGCTGCAAGGCGATTAAGTTGGGTAACGCCAGGGTTTT |
| | CCCAGNCACGACGNTGNAAAACGACGGNCANNGCCANNCTNTGNTGNTNTAAACN |
| | ACNCATT |

Annotated Drosophila genome Complete Genome candidate

CG2446 (2 transcripts)—encodes a novel protein which may be a glycosylation/membrane protein

(SEQ ID NO:105)

AGATAGAACGACAACTCCTGTTCCCGGTTCGTCGTCGTTCGTCATTCCCA
TATTCGCTTCTCGTATTCCCTCCCATTCCCATTCGCAATCCCAATTCCCA
ATTCCCGTCACACGAGTTAGCAGCACATCGCACAGCTGCATCGCTCCGCT
CCGATCCTTTTTAATTTTTTGTTGTGCCTTCGGTGGCGTGCTCATTTCGA
GAACAGAGTAACCCCTTTTTATTTGTCAGTTGTCAACGGCGCCCCTGCAG
GCAGAAAGCAGAAACTGAAACAGCAGAGGAAGAAGAAGAAGCAGCACAGC
ACGGGCACAGCACGAAGCACGCAGCACAGCACAAGCACAGAGGCGAAGCG
AAGCAAAGCAAAGCAGAGGCAACACAGAAAAACAGCAAAGCATTGGAGTA
GTTGTTTGGATGTGGACGGAAAGGAAGACTGGCGGCGACTAACTAAAAGC
AGTACGTTGACAGGAGCAGCTCGGAACGGACAGGAAAAGCAGGAGACTAA
ACACCAGCCGGTCACTTGCGGATCAGCCAACGTCCTGGGCCCCAAGGCGA
TAGATACCACGATAAGGAGATACAGCGATACCACCAATCATTAGCAGGCG
ACAACGACACATCCGCATCCGCAGAAGATGTCCAACGGCAAGGCGACGGT
CTCGTTCTTCGAGACCGGGAGCACCAAACAGTTCGAGTACTGCTACCAGC
TCTATCCCCAGGTTCTTAAGCTAAAGGCCGAGAAGCGCTGCAAGAAGCCG
CAAGAGCTGATCCGCCTGGATCAGTGGTATCAGAATGAACTGCCCAAATT
GATTAAGGCACGCGGCAAGGACGCGCATATGGTATACGATGAGCTCGTCC
AGTCGATGAAGTGGAAGCAGTCGCGCGGCAAATTCTATCCGCAGCTATCC
TACCTGGTCAAGGTCAACACACCGCGCGCCGTCATCCAGGAGACAAAGAA
GGCCTTCCGCAAGCTGCCCAATCTGGAGCAGGCGATCACAGCTTTATCGA
ACCTCAAGGGCGTTGGCACCACAATGGCCAGTGCACTGCTGGCAGCCGCA
GCTCCCGATTCGGCACCATTCATGGCCGACGAGTGCCTGATGGCCATACC
AGAGATCGAGGGCATCGATTACACCACCAAGGAGTACCTCAACTTCGTCA
ATCACATTCAGGCCACCGTGGAGCGCCTCAATGCGGAGGTGGGCGGGGAT
ACGCCGCACTGGTCGCCTCATCGCGTGGAGCTGGCCCTCTGGTCACACTA
TGTGGCCAATGATCTCAGTCCCGAGATGCTCGACGATATGCCGCCGCCTG
GATCCGGCGCCTCCACTGGCACCGGTTCACTCAGCACAAACGGCAACAGC
AGCAAGGTGCTCGATGGCGACGATACCAACGATGGTGTGGGTGTTGATTT
GGACGACGAAAGCCAAGGAGCAGGCGGTCGCAACACTGCTACAGAATCGG
AGACAGAGAATGAGAACACCAACCCGGCTGCTCTGACGCCTCTACAGTCG
GGCGAGGCCAAGAACAACGCAGCTGCCGTTGGCGCCGCCCTGCAGGACGG
TGACTCCAACTTTGTTTCGAACGATTCCACCTCCCAGGAGCCGATCATCG
ATGACAACGATGGCACCACACAGACAACGGCCACCACTTCCACAGAGGAC
GGTGAGCCCATCGCCCTAGACATTGGCATTGGCATCGGTTCGAGTGGAAC
ACCGCTCGCCTCGGACTCTGAAAGCAATCAGGAGGCGCCGCCCAAGACCA
ACAGCCTGCCCATCCTGACTCCCACACAGCACTCGAGCCAGAATCAGAAT
CAAAAGCAGTCGCCGAGCCAGCCCCACAAAACTAACAATTCGATCACCAA
CAACGGTCAGCCTGCTCCTTTGGCAGAAGAGGAAGCGGTTACAGCAGCAC
CACAGCCAGCCAGCAAAGCGACTGCAGCACCAGCCAATGGAAATGGTAAC
GGGAACGGCGTCCTGGGCGACGAGGATGAGGATGAGGCGGAGGACGAGGA
GGAAGATGAGCTGGACGAGGAGGAGGATAATGAGGCGGAGCTAGAGGCTG
ACGAGAGCAATAGCAGCAACGGCATTGTGAGGGACAGTAAACTGCAGCAG
CTGGCGGCGAACAAGGCGGTGGATGCGGTTTCACCGGTAGCAGCGGGTGC
AGACTCGGCACCAGCCATTGGACAGAAGCGTACTGCCCTGCACTGCGATA
TGGAGCTGAAGAACGCCGGCGGAGTGGGTGTGGGCGTGGGGGAGAAGTCA
CCGGATCTAAAGAAACTGCGCAGCGAATGA

(SEQ ID NO:106)

MSNGKATVSFFETGSTKQFEYCYQLYPQVLKLKAEKRCKKPQELIRLDQW
YQNELPKLIKARGKDAHMVYDELVQSMKWKQSRGKFYPQLSYLVKVNTPR
AVIQETKKAFRKLPNLEQAITALSNLKGVGTTMASALLAAAAPDSAPFMA
DECLMAIPEIEGIDYTTKEYLNFVNHIQATVERLNAEVGGDTPHWSPHRV
ELALWSHYVANDLSPEMLDDMPPPGSGASTGTGSLSTNGNSSKVLDGDDT
NDGVGVDLDDESQGAGGRNTATESETENENTNPAALTPLQSGEAKNNAAA
VGAALQDGDSNFVSNDSTSQEPIIDDNDGTTQTTATTSTEDGEPIALDIG
IGIGSSGTPLASDSESNQEAPPKTNSLPILTPTQHSSQNQNQKQSPSQPH
KTNNSITNNGQPAPLAEEEAVTAAPQPASKATAAPANGNGNGNGVLGDED
EDEAEDEEEDELDEEEDNEAELEADESNSSNGIVRDSKLQQLAANKAVDA
VSPVAAGADSAPAIGQKRTALHCDMELKNAGGVGVGVGEKSPDLKKLRSE

(SEQ ID NO:107)

GCCTGTCAGTTTGACTGTGTGAGTGCATGGCGGACTAAAAAGAACCCGAC
GACAGCACTGTAAAAATTCGATTTGTGTGCTGTGCAAACGGCGGCGGAAG
CGAGCAGATTTTTGGCAAATAGTGAGCGATTATCGGATTGAGTAAATACA
ACAAACAACAGAGACACGGCCGCAGCAGCAGCAGCATTAACACAGTACGT
TGACAGGAGCAGCTCGGAACGGACAGGAAAAGCAGGAGACTAAACACCAG
CCGGTCACTTGCGGATCAGCCAACGTCCTGGGCCCCAAGGCGATAGATAC
CACGATAAGGAGATACAGCGATACCACCAATCATTAGCAGGCGACAACGA
CACATCCGCATCCGCAGAAGATGTCCAACGGCAAGGCGACGGTCTCGTTC
TTCGAGACCGGGAGCACCAAACAGTTCGAGTACTGCTACCAGCTCTATCC
CCAGGTTCTTAAGCTAAAGGCCGAGAAGCGCTGCAAGAAGCCGCAAGAGC
TGATCCGCCTGGATCAGTGGTATCAGAATGAACTGCCCAAATTGATTAAG
GCACGCGGCAAGGACGCGCATATGGTATACGATGAGCTCGTCCAGTCGAT
GAAGTGGAAGCAGTCGCGCGGCAAATTCTATCCGCAGCTATCCTACCTGG
TCAAGGTCAACACACCGCGCGCCGTCATCCAGGAGACAAAGAAGGCCTTC
CGCAAGCTGCCCAATCTGGAGCAGGCGATCACAGCTTTATCGAACCTCAA
GGGCGTTGGCACCACAATGGCCAGTGCACTGCTGGCAGCCGCAGCTCCCG
ATTCGGCACCATTCATGGCCGACGAGTGCCTGATGGCCATACCAGAGATC
GAGGGCATCGATTACACCACCAAGGAGTACCTCAACTTCGTCAATCACAT
TCAGGCCACCGTGGAGCGCCTCAATGCGGAGGTGGGCGGGGATACGCCGC
ACTGGTCGCCTCATCGCGTGGAGCTGGCCCTCTGGTCACACTATGTGGCC
AATGATCTCAGTCCCGAGATGCTCGACGATATGCCGCCGCCTGGATCCGG
CGCCTCCACTGGCACCGGTTCACTCAGCACAAACGGCAACAGCAGCAAGG
TGCTCGATGGCGACGATACCAACGATGGTGTGGGTGTTGATTTGGACGAC
GAAAGCCAAGGAGCAGGCGGTCGCAACACTGCTACAGAATCGGAGACAGA
GAATGAGAACACCAACCCGGCTGCTCTGACGCCTCTACAGTCGGGCGAGG
CCAAGAACAACGCAGCTGCCGTTGGCGCCGCCCTGCAGGACGGTGACTCC
AACTTTGTTTCGAACGATTCCACCTCCCAGGAGCCGATCATCGATGACAA
CGATGGCACCACACAGACAACGGCCACCACTTCCACAGAGGACGGTGAGC
CCATCGCCCTAGACATTGGCATTGGCATCGGTTCGAGTGGAACACCGCTC
GCCTCGGACTCTGAAAGCAATCAGGAGGCGCCGCCCAAGACCAACAGCCT
GCCCATCCTGACTCCCACACAGCACTCGAGCCAGAATCAGAATCAAAAGC
AGTCGCCGAGCCAGCCCCACAAAACTAACAATTCGATCACCAACAACGGT
CAGCCTGCTCCTTTGGCAGAAGAGGAAGCGGTTACAGCAGCACCACAGCC
AGCCAGCAAAGCGACTGCAGCACCAGCCAATGGAAATGGTAACGGGAACG
GCGTCCTGGGCGACGAGGATGAGGATGAGGCGGAGGACGAGGAGGAAGAT
GAGCTGGACGAGGAGGAGGATAATGAGGCGGAGCTAGAGGCTGACGAGAG
CAATAGCAGCAACGGCATTGTGAGGGACAGTAAACTGCAGCAGCTGGCGG
CGAACAAGGCGGTGGATGCGGTTTCACCGGTAGCAGCGGGTGCAGACTCG
GCACCAGCCATTGGACAGAAGCGTACTGCCCTGCACTGCGATATGGAGCT
GAAGAACGCCGGCGGAGTGGGTGTGGGCGTGGGGGAGAAGTCACCGGATC
TAAAGAAACTGCGCAGCGAATGA

(SEQ ID NO:108)

MSNGKATVSFFETGSTKQFEYCYQLYPQVLKLKAEKRCKKPQELIRLDQW
YQNELPKLIKARGKDAHMVYDELVQSMKWKQSRGKFYPQLSYLVKVNTPR
AVIQETKKAFRKLPNLEQAITALSNLKGVGTTMASALLAAAAPDSAPFMA
DECLMAIPEIEGIDYTTKEYLNFVNHIQATVERLNAEVGGDTPHWSPHRV
ELALWSHYVANDLSPEMLDDMPPPGSGASTGTGSLSTNGNSSKVLDGDDT
NDGVGVDLDDESQGAGGRNTATESETENENTNPAALTPLQSGEAKNNAAA
VGAALQDGDSNFVSNDSTSQEPIIDDNDGTTQTTATTSTEDGEPIALDIG
IGIGSSGTPLASDSESNQEAPPKTNSLPILTPTQHSSQNQNQKQSPSQPH
KTNNSITNNGQPAPLAEEEAVTAAPQPASKATAAPANGNGNGNGVLGDED
EDEAEDEEEDELDEEEDNEAELEADESNSSNGIVRDSKLQQLAANKAVDA
VSPVAAGADSAPAIGQKRTALHCDMELKNAGGVGVGVGEKSPDLKKLRSE

Human homologue of Complete Genome candidate
CG2446—none

Putative function
glycosylation/membrane protein

### Example 2C

### Category 1

Line ID—fs(1)06

Phenotype—Female sterile (semi-sterile), 2-3 fully matured eggs seen in each of the ovarioles

Annotated Drosophila genome genomic segment containing P element insertion site (and map position)—AE003449 (9B6-7)

P element insertion site sequence

| (SEQ ID NO:109) | |
|---|---|
| | CTNCATGNTGNAGGAGACAAGGCGTTCTATATTATATAGNNGATTTTNNTGTATATA |
| | AAGGAAGANCTGNGCTAANGNAANAGGCATCTCGATGANTTTNATAATNAGGGCAA |
| | NTGGTANNAANGGTTTATGCCAAAGTATTACACACCAGGGNTGGGCACAACAGATC |
| | TTAACTNANNATAGGNNATTGGNATAANCTTAAATTTGTAAGATTNTGNAATAATAT |
| | AGTAGAGANNNTCAATACGCATTANTAATNGTGACGATCCCNAGCATAAACTCAAA |
| | AAAANCTTATANTTTTATAAAGGCNANNCCNNACTAANNAATTAAANGAANNNCNG |
| | NCGCCNCNAAANGATGATTGNGCTATATAANNANANNATTGATNGAGGCACTTATA |
| | TTATTATAATTAAAACACTTAATTATTNTGTGTGAAATGATTGCACTNNNNATTGGG |
| | CNAGAGCCTNNNNCGTATTGANANNNNNNNATTTNGGCTNNANCTGTAAATATCNT |
| | ACAAACTCGTNATTGCTAAATAACTTTTGTATNCCCCNCTGGTCACTCTGACTTAAA |
| | CGTNNTTCGNNAAAACAGCGGCTGATCACTGANGTTTTCTCCCGNNTTTCGCTNTCA |
| | ANCCGAANTANAAACAGGNGAANNTCCCNGATAATTTGNGGNNTANCCCACTGATC |
| | ACAGNGCCCNNGGATNNNCAAGGAANNGCGATCGAAACCCGNCCTGGNGNAACAC |
| | NNTTTCCC |

Annotated Drosophila genome Complete Genome candidate

CG2968—hydrogen transporting ATP synthase

(SEQ ID NO:110)

CAAAAACAGCGGCTGATCACTGAAGTTTTCTCGTGTTTTTCGCTATCAAA
CCGAAATAAAAACAGCCCAAAATGTCCTTCGTTAAGAACGCCCGTTTGCT
GGCCGCCCGCGGCGCTCGCTTGGCCCAGAACCGCAGCTACTCGGATGAGA
TGAAGCTGACCTTCGCCGCCGCCAACAAAACCTTCTACGATGCCGCTGTG
GTGCGCCAAATCGATGTGCCTTCCTTCTCGGGATCCTTCGGCATCCTGGC
CAAGCACGTGCCCACTCTGGCTGTCCTGAAGCCCGGCGTTGTCCAGGTGG
TGGAAAACGATGGCAAGACCCTCAAGTTCTTCGTCTCCAGCGGTTCCGTC
ACCGTCAACGAGGATTCCTCCGTTCAGGTTCTGGCCGAGGAGGCCCACAA
CATCGAGGACATCGATGCCAATGAGGCGCGCCAGCTGCTCGCGAAATACC
AGTCACAGCTTAGCTCCGCTGGCGACGACAAGGCCAAGGCCCAGGCTGCC
ATTGCCGTGGAGGTCGCCGAAGCGTTAGTCAAGGCTGCCGAATAGACGTA
ATCACCACACAACCGCCACCAATAAACCACAATCGATGCTTTGTGTCTGA
AATAAATAAAAAACATAACGATCACCTTAAAAAGCCAGAGAGTTATGAAA
CAATAAAAAAGCGA

(SEQ ID NO:111)

MSFVKNARLLAARGARLAQNRSYSDEMKLTFAAANKTFYDAAVVRQIDVP
SFSGSFGILAKHVPTLAVLKPGVVQVVENDGKTLKFFVSSGSVTVNEDSS
VQVLAEEAHNIEDIDANEARQLLAKYQSQLSSAGDDKAKAQAAIAVEVAE
ALVKAAE

Human homologue of Complete Genome candidate

CAA45016—H(+)-transporting ATP synthase, delta-subunit of the human mitochondrial ATP synthase complex

| | | |
|---|---|---|
| 1 | gtcctcctcg ccctccaggc cgcccgcgcc gcgccggagt ccgctgtccg ccagctaccc | (SEQ ID NO:112) |
| 61 | gcttcctgcc gcccgccgct gccatgctgc ccgccgcgct gctccgccgc ccgggacttg | |
| 121 | gccgcctcgt ccgccacgcc cgtgcctatg ccgaggccgc cgccgccccg gctgccgcct | |
| 181 | ctggccccaa ccagatgtcc ttcaccttcg cctctcccac gcaggtgttc ttcaacggtg | |
| 241 | ccaacgtccg gcaggtggac gtgcccacgc tgaccggagc cttcggcatc ctggcggccc | |
| 301 | acgtgcccac gctgcaggtc ctgcggccgg ggctggtcgt ggtgcatgca gaggacggca | |
| 361 | ccacctccaa atactttgtg agcagcggtt ccatcgcagt gaacgccgac tcttcggtgc | |
| 421 | agttgttggc cgaagaggcc gtgacgctgg acatgttgga cctgggggca gccaaggcaa | |
| 481 | acttggagaa ggcccaggcg gagctggtgg ggacagctga cgaggccacg cgggcagaga | |
| 541 | tccagatccg aatcgaggcc aacgaggccc tggtgaaggc cctggagtag gcggtgcgta | |
| 601 | cccggtgtcc cgaggcccgg ccaggggctg ggcagggatg ccaggtgggc ccagccagct | |
| 661 | cctggggtcc cggccacctg gggaagccgc gcctgccaag gaggccacca gagggcagtg | |
| 721 | caggcttctg cctgggcccc aggccctgcc tgtgttgaaa gctctgggga ctgggccagg | |
| 781 | gaagctcctc ctcagctttg agctgtggct gccacccatg gggctctcct tccgcctctc | |
| 841 | aagatccccc cagcctgacg ggccgcttac catcccctct gccctgcaga gccagccgcc | |
| 901 | aaggttgacc tcagcttcgg agccacctct ggatgaactg cccccagccc ccgccccatt | |
| 961 | aaagacccgg aagcctgaaa aaaaaaaaaa aaaa | |
| 1 | mlpaallrrp glgrlvrhar ayaeaaaapa aasgpnqmsf tfasptqvff nganvrqvdv | (SEQ ID NO:113) |
| 61 | ptltgafgil aahvptlqvl rpglvvvhae dgttskyfvs sgsiavnads svqllaeeav | |
| 121 | tldmldlgaa kanlekaqae lvgtadeatr aeiqiriean ealvkale | |

Putative function
hydrogen transporting ATP synthase

### Category 2—Male Steriles

### Example 3

### Category 2

Line ID—167

Phenotype—lethal phase pharate adult, cytokinesis defect.
Some onion stage cysts with large nebenkerns

Annotated Drosophila genome genomic segment containing P element insertion site (and map position)—AE003428 (3F4-5)

P element insertion site—293,654

Annotated Drosophila genome Complete Genome candidate

CG2829—BcDNA:GH07910 tousled kinase (2 splice variants)

(SEQ ID NO:114)

AGTTTCATTCGGGGATGCTTGGCCTATCGCAAGGAGGATCGCATGGATGT
GTTCGCACTGGCCAGGCACGAGTACATTCAGCCACCGATACCGAAACATG
GGCGCGGTTCGCTCAATCAGCAACAGCAGGCGCAACAACAGCAGCAGCAA
CAACAGCAACAGCAGCAGCAACAGTCGTCGACGTCACAGGCCAATTCTAC
AGGCCAGACATCTTTCTCTGCCCACATGTTTGGCAATATGAATCAGTCGA
GTTCGTCCTAGATGAGAGCGACTGCAAAAAAATCGGAATAAACACGGTTA
TAATATATAAGTACAAATAAACCATATATATGTGTTTATGTTATGTATAT
ATACATAAAGGAAAATAACAAGGCAAATGTGAAAATTAGTGCAAACTGAA
CGAAAAGACAAAAATAAAACAAAAGGAAACCCAAATGTGATAATATTGTA
ATATAATGTGAAAAGCAAAACACACACAAATACACAACTCACGCACTTAG
CCACGTATGTGTGTGCAGAAAAATATGCGGCGCTTAAAAAAGATGTCCCC
CGGCGCCCATTTGCAGATGTCCCCGCAGAACACTTCGTCCCTAAGTCAAC
ACCATCCACATCAACAGCAACAGTTACAACCCCCACAGCAGCAACAACAG
CATTTCCCTAACCATCACAGCGCCCAGCAACAGTCGCAGCAGCAGCAGCA
ACAGGAGCAACAGAATCCCCAGCAGCAGGCGCAACAGCAGCAGCAGATAC
TCCCACATCAACATTTGCAGCACCTGCACAAGCATCCGCATCAGCTGCAA
CTGCATCAGCAGCAGCAACAACAACTCCACCAGCAACAGCAGCAACACTT
CCACCAGCAGTCGCTGCAAGGGCTGCATCAGGGTAGCAGCAATCCGGATT
CGAATATGAGCACTGGCTCCTCGCATAGCGAGAAGGATGTCAATGATATG
CTGAGTGGCGGTGCAGCAACGCCAGGAGCTGCAGCAGCAGCGATTCAACA
GCAACATCCCGCCTTTGCGCCCACACTGGGAATGCAGCAACCACCGCCGC
CCCCACCTCAACACTCCAATAATGGAGGCGAGATGGGCTACTTGTCGGCA
GGCACGACCACGACGACGTCGGTGTTAACGGTAGGCAAGCCTCGGACGCC
AGCGGAGCGGAAACGGAAGCGAAAAATGCCTCCATGTGCCACTAGTGCGG
ATGAGGCGGGGAGTGGCGGTGGCTCTGGCGGAGCAGGAGCAACCGTTGTT
AACAACAGCAGCCTGAAGGGCAAATCATTGGCCTTTCGTGATATGCCCAA
GGTAAACATGAGCCTGAATCTGGGCGATCGTCTGGGAGGATCTGCAGGAA
GCGGAGTAGGAGCCGGTGGCGCCGGAAGCGGGGGAGGTGGCGCTGGTTCC
GGTTCTGGAAGCGGTGGCGGCAAAAGCGCCCGCCTGATGCTGCCAGTCAG
CGACAACAAGAAGATCAACGACTATTTCAATAAGCAGCAAACGGGCGTGG
GCGTCGGTGTGCCAGGTGGTGCGGGAGGCAATACCGCTGGCCTTCGAGGA
TCACATACGGGAGGTGGCAGCAAGTCACCCTCATCCGCCCAGCAGCAGCA
AACGGCGGCACAGCAGCAGGGAAGCGGTGTTGCGACGGGAGGCAGTGCAG
GCGGTTCCGCTGGCAACCAGGTGCAAGTGCAAACGAGCAGCGCTTACGCC
CTTTACCCACCAGCTAGTCCCCAAACCCAGACGTCACAGCAACAGCAGCA
GCAGCAACCGGGATCAGACTTTCACTATGTCAACTCCAGCAAGGCGCAGC
AACAACAGCAGCGTCAACAGCAACAGACTTCCAATCAAATGGTTCCTCCA
CACGTGGTCGTTGGCCTTGGTGGTCATCCACTGAGCCTCGCGTCCATTCA
GCAGCAGACGCCCTTATCCCAGCAGCAACAGCAGCAACAACAGCAGCAGC
AACAGCAGCAACTGGGACCACCGACCACATCGACGGCCTCCGTCGTGCCA
ACGCATCCGCATCAACTCGGATCCCTGGGAGTTGTTGGGATGGTCGGTGT
GGGTGTTGGCGTGGGCGTTGGAGTAAATGTGGGTGTGGGACCACCACTGC
CACCACCACCGCCGATGGCCATGCCAGCGGCCATTATCACTTATAGTAAG
GCCACTCAAACGGAGGTGTCGCTGCATGAATTGCAGGAGCGCGAAGCGGA
GCACGAATCGGGCAAGGTGAAGCTAGACGAGATGACACGGCTGTCCGATG
AACAAAAGTCCCAAATTGTTGGCAACCAGAAGACGATTGACCAGCACAAG
TGCCACATAGCCAAGTGTATTGATGTGGTCAAGAAGCTGTTGAAGGAGAA
GAGCAGCATCGAGAAGAAGGAGGCGCGACAGAAGTGCATGCAGAATCGCC
TCAGGCTCGGACAGTTTGTTACCCAACGAGTGGGCGCCACATTCCAGGAG
AACTGGACGGACGGCTATGCGTTCCAGGAGCTGAGTCGGCGGCAAGAAGA
AATAACCGCTGAGCGTGAAGAGATAGATCGGCAGAAAAAGCAGCTGATGA
AAAAGCGTCCGGCGGAGTCCGGACGCAAGCGCAACAACAACAGTAACCAG
AACAACCAGCAGCAGCAGCAACAGCAACACCAGCAACAGCAGCAGCAACA
AAATTCCAACTCGAACGATTCCACGCAGCTGACGAGCGGAGTTGTTACCG
GTCCAGGCAGTGATCGTGTGAGCGTAAGCGTCGACAGCGGATTGGGTGGC
AATAATGCGGGCGCGATCGGTGGCGGAACCGTTGGTGGTGGCGTTGGAGG
TGGTGGTGTTGGAGGCGGTGGTGTCGGAGGCGGCGGTGGACGTGGACTTT
CTCGCAGCAATTCGACGCAGGCCAATCAGGCTCAATTGCTGCACAACGGC
GGTGGTGGTTCGGGCGGCAATGTCGGCAACTCGGGCGGCGTTGGCGACCG
CTTGTCAGATCGAGGAGGAGGAGGTGGCGGCATCGGCGGAAACGATAGCG
GCAGCTGCTCGGACTCGGGCACTTTCCTGAAGCCAGACCCCGTATCGGGT
GCCTACACAGCGCAGGAGTATTACGAGTACGATGAGATCCTCAAGTTGCG
ACAAAATGCCCTCAAAAAGGAGGACGCCGACCTGCAGCTGGAGATGGAGA
AGCTGGAGCGGGAGCGCAATCTGCACATCCGAGAGCTCAAGCGGATTCTT
AACGAGGATCAGTCCCGCTTTAACAATCATCCCGTGCTGAATGATCGCTA
TCTTCTGTTGATGCTCCTGGGCAAGGGCGGCTTCTCAGAGGTCCACAAGG
CCTTCGACCTGAAGGAGCAACGCTATGTCGCATGTAAGGTGCACCAATTA
AACAAGGATTGGAAGGAGGATAAGAAAGCTAATTATATCAAACACGCTTT
GCGGGAATACAACATTCACAAGGCACTGGATCATCCGCGGGTCGTCAAGC
TATACGATGTCTTCGAGATCGATGCGAATTCCTTTTGCACAGTGCTCGAA
TACTGTGATGGCCACGATCTGGACTTCTATTTGAAGCAACATAAGACTAT
ACCCGAGCGTGAAGCGCGCTCGATAATAATGCAGGTTGTATCTGCACTCA
AGTATCTAAATGAGATTAAGCCTCCAGTTATCCACTACGATCTGAAGCCC
GGCAACATTCTGCTTACCGAGGGCAACGTCTGCGGCGAGATTAAGATCAC
CGACTTCGGTCTGTCAAAGGTGATGGACGACGAGAATTACAATCCCGATC
ACGGCATGGATCTGACCTCTCAGGGGGCGGGAACCTACTGGTATCTGCCA
CCCGAGTGCTTTGTCGTGGGCAAAAATCCGCCGAAAATCTCCTCCAAAGT
GGACGTATGGAGTGTGGGTGTTATCTTCTACCAGTGTCTGTACGGCAAAA
AGCCCTTCGGTCACAATCAGTCGCAGGCCACGATTCTCGAGGAGAATACG
ATCCTGAAGGCCACCGAAGTGCAGTTCTCCAACAAGCCAACCGTTTCTAA
CGAGGCCAAG

(SEQ ID NO:115)

MCVQKNMRRLKKMSPGAHLQMSPQNTSSLSQHHPHQQQQLQPPQQQQQHF
PNHHSAQQQSQQQQQQEQQNPQQQAQQQQQILPHQHLQHLHKHPHQLQLH
QQQQQQLHQQQQQHFHQQSLQGLHQGSSNPDSNMSTGSSHSEKDVNDMLS
GGAATPGAAAAAIQQQHPAFAPTLGMQQPPPPPPQHSNNGGEMGYLSAGT
TTTTSVLTVGKPRTPAERKRKRKMPPCATSADEAGSGGGSGGAGATVVNN
SSLKGKSLAFRDMPKVNMSLNLGDRLGGSAGSGVGAGGAGSGGGGAGSGS
GSGGGKSARLMLPVSDNKKINDYFNKQQTGVGVGVPGGAGGNTAGLRGSH
TGGGSKSPSSAQQQQTAAQQQGSGVATGGSAGGSAGNQVQVQTSSAYALY
PPASPQTQTSQQQQQQQPGSDFHYVNSSKAQQQQQRQQQQTSNQMVPPHV
VVGLGGHPLSLASIQQQTPLSQQQQQQQQQQQQQQLGPPTTSTASVVPTH
PHQLGSLGVVGMVGVGVGVGVGVNVGVGPPLPPPPPMAMPAAIITYSKAT
QTEVSLHELQEREAEHESGKVKLDEMTRLSDEQKSQIVGNQKTIDQHKCH
IAKCIDVVKKLLKEKSSIEKKEARQKCMQNRLRLGQFVTQRVGATFQENW
TDGYAFQELSRRQEEITAEREEIDRQKKQLMKKRPAESGRKRNNNSNQNN
QQQQQQQHQQQQQQQNSNSNDSTQLTSGVVTGPGSDRVSVSVDSGLGGNN
AGAIGGGTVGGGVGGGGVGGGGVGGGGGRGLSRSNSTQANQAQLLHNGGG
GSGGNVGNSGGVGDRLSDRGGGGGGIGGNDSGSCSDSGTFLKPDPVSGAY
TAQEYYEYDEILKLRQNALKKEDADLQLEMEKLERERNLHIRELKRILNE
DQSRFNNHPVLNDRYLLLMLLGKGGFSEVHKAFDLKEQRYVACKVHQLNK
DWKEDKKANYIKHALREYNIHKALDHPRVVKLYDVFEIDANSFCTVLEYC
DGHDLDFYLKQHKTIPEREARSIIMQVVSALKYLNEIKPPVIHYDLKPGN
ILLTEGNVCGEIKITDFGLSKVMDDENYNPDHGMDLTSQGAGTYWYLPPE
CFVVGKNPPKISSKVDVWSVGVIFYQCLYGKKPFGHNQSQATILEENTIL
KATEVQFSNKPTVSNEAK

(SEQ ID NO:116)

AGTTTCATTCGGGGATGCTTGGCCTATCGCAAGGAGGATCGCATGGATGT
GTTCGCACTGGCCAGGCACGAGTACATTCAGCCACCGATACCGAAACATG
GGCGCGGTTCGCTCAATCAGCAACAGCAGGCGCAACAACAGCAGCAGCAA
CAACAGCAACAGCAGCAGCAACAGTCGTCGACGTCACAGGCCAATTCTAC
AGGCCAGACATCTTTCTCTGCCCACATGTTTGGCAATATGAATCAGTCGA
GTTCGTCCTAGTGGTGTCGGTGTCGTTTTGGTTTTGTCGGCGGTTGCTAA
ACACAATTTAAGTTCACTCGGTTAGCAGACATTACACACTGCCTGCTCTC
ATACATATTTACGCACTTGTATATACATGCAATGTGCCTGTGTGTGCGCA
AGAAACCAGAAAAAACGAAAAGTACAACATTCGTTGAGTCGCGTTCGGCT
TAATTTTTTTTTGTGTTACCGTGTGTGTGTTTGTGCTTTGGATTTGCCAA
TTTTAGCCGACTGGCTCTCAGTGTCGAACTTAAACTTAAAGAGCGAGCAA
CGTGACGTGTCGCCCAGTGTCGCTTAAAATTCGCGCACACAACTTCCTAC
TACAAAAAAACGAAAGAAAGAAGGAGAAAAAACGTTAAAGATGTCCCCCG
GCGCCCATTTGCAGATGTCCCCGCAGAACACTTCGTCCCTAAGTCAACAC
CATCCACATCAACAGCAACAGTTACAACCCCCACAGCAGCAACAACAGCA
TTTCCCTAACCATCACAGCGCCCAGCAACAGTCGCAGCAGCAGCAGCAAC
AGGAGCAACAGAATCCCCAGCAGCAGGCGCAACAGCAGCAGCAGATACTC
CCACATCAACATTTGCAGCACCTGCACAAGCATCCGCATCAGCTGCAACT
GCATCAGCAGCAGCAACAACAACTCCACCAGCAACAGCAGCAACACTTCC
ACCAGCAGTCGCTGCAAGGGCTGCATCAGGGTAGCAGCAATCCGGATTCG
AATATGAGCACTGGCTCCTCGCATAGCGAGAAGGATGTCAATGATATGCT
GAGTGGCGGTGCAGCAACGCCAGGAGCTGCAGCAGCAGCGATTCAACAGC
AACATCCCGCCTTTGCGCCCACACTGGGAATGCAGCAACCACCGCCGCCC
CCACCTCAACACTCCAATAATGGAGGCGAGATGGGCTACTTGTCGGCAGG
CACGACCACGACGACGTCGGTGTTAACGGTAGGCAAGCCTCGGACGCCAG
CGGAGCGGAAACGGAAGCGAAAAATGCCTCCATGTGCCACTAGTGCGGAT
GAGGCGGGGAGTGGCGGTGGCTCTGGCGGAGCAGGAGCAACCGTTGTTAA
CAACAGCAGCCTGAAGGGCAAATCATTGGCCTTTCGTGATATGCCCAAGG
TAAACATGAGCCTGAATCTGGGCGATCGTCTGGGAGGATCTGCAGGAAGC
GGAGTAGGAGCCGGTGGCGCCGGAAGCGGGGGAGGTGGCGCTGGTTCCGG
TTCTGGAAGCGGTGGCGGCAAAAGCGCCCGCCTGATGCTGCCAGTCAGCG
ACAACAAGAAGATCAACGACTATTTCAATAAGCAGCAAACGGGCGTGGGC
GTCGGTGTGCCAGGTGGTGCGGGAGGCAATACCGCTGGCCTTCGAGGATC
ACATACGGGAGGTGGCAGCAAGTCACCCTCATCCGCCCAGCAGCAGCAAA
CGGCGGCACAGCAGCAGGGAAGCGGTGTTGCGACGGGAGGCAGTGCAGGC
GGTTCCGCTGGCAACCAGGTGCAAGTGCAAACGAGCAGCGCTTACGCCCT
TTACCCACCAGCTAGTCCCCAAACCCAGACGTCACAGCAACAGCAGCAGC
AGCAACCGGGATCAGACTTTCACTATGTCAACTCCAGCAAGGCGCAGCAA
CAACAGCAGCGTCAACAGCAACAGACTTCCAATCAAATGGTTCCTCCACA
CGTGGTCGTTGGCCTTGGTGGTCATCCACTGAGCCTCGCGTCCATTCAGC
AGCAGACGCCCTTATCCCAGCAGCAACAGCAGCAACAACAGCAGCAGCAA
CAGCAGCAACTGGGACCACCGACCACATCGACGGCCTCCGTCGTGCCAAC
GCATCCGCATCAACTCGGATCCCTGGGAGTTGTTGGGATGGTCGGTGTGG
GTGTTGGCGTGGGCGTTGGAGTAAATGTGGGTGTGGGACCACCACTGCCA
CCACCACCGCCGATGGCCATGCCAGCGGCCATTATCACTTATAGTAAGGC
CACTCAAACGGAGGTGTCGCTGCATGAATTGCAGGAGCGCGAAGCGGAGC
ACGAATCGGGCAAGGTGAAGCTAGACGAGATGACACGGCTGTCCGATGAA
CAAAAGTCCCAAATTGTTGGCAACCAGAAGACGATTGACCAGCACAAGTG
CCACATAGCCAAGTGTATTGATGTGGTCAAGAAGCTGTTGAAGGAGAAGA
GCAGCATCGAGAAGAAGGAGGCGCGACAGAAGTGCATGCAGAATCGCCTC
AGGCTCGGACAGTTTGTTACCCAACGAGTGGGCGCCACATTCCAGGAGAA
CTGGACGGACGGCTATGCGTTCCAGGAGCTGAGTCGGCGGCAAGAAGAAA
TAACCGCTGAGCGTGAAGAGATAGATCGGCAGAAAAAGCAGCTGATGAAA
AAGCGTCCGGCGGAGTCCGGACGCAAGCGCAACAACAACAGTAACCAGAA
CAACCAGCAGCAGCAGCAACAGCAACACCAGCAACAGCAGCAGCAACAAA
ATTCCAACTCGAACGATTCCACGCAGCTGACGAGCGGAGTTGTTACCGGT
CCAGGCAGTGATCGTGTGAGCGTAAGCGTCGACAGCGGATTGGGTGGCAA
TAATGCGGGCGCGATCGGTGGCGGAACCGTTGGTGGTGGCGTTGGAGGTG
GTGGTGTTGGAGGCGGTGGTGTCGGAGGCGGCGGTGGACGTGGACTTTCT
CGCAGCAATTCGACGCAGGCCAATCAGGCTCAATTGCTGCACAACGGCGG
TGGTGGTTCGGGCGGCAATGTCGGCAACTCGGGCGGCGTTGGCGACCGCT
TGTCAGATCGAGGAGGAGGAGGTGGCGGCATCGGCGGAAACGATAGCGGC
AGCTGCTCGGACTCGGGCACTTTCCTGAAGCCAGACCCCGTATCGGGTGC
CTACACAGCGCAGGAGTATTACGAGTACGATGAGATCCTCAAGTTGCGAC
AAAATGCCCTCAAAAAGGAGGACGCCGACCTGCAGCTGGAGATGGAGAAG
CTGGAGCGGGAGCGCAATCTGCACATCCGAGAGCTCAAGCGGATTCTTAA
CGAGGATCAGTCCCGCTTTAACAATCATCCCGTGCTGAATGATCGCTATC
TTCTGTTGATGCTCCTGGGCAAGGGCGGCTTCTCAGAGGTCCACAAGGCC
TTCGACCTGAAGGAGCAACGCTATGTCGCATGTAAGGTGCACCAATTAAA
CAAGGATTGGAAGGAGGATAAGAAAGCTAATTATATCAAACACGCTTTGC
GGGAATACAACATTCACAAGGCACTGGATCATCCGCGGGTCGTCAAGCTA
TACGATGTCTTCGAGATCGATGCGAATTCCTTTTGCACAGTGCTCGAATA
CTGTGATGGCCACGATCTGGACTTCTATTTGAAGCAACATAAGACTATAC
CCGAGCGTGAAGCGCGCTCGATAATAATGCAGGTTGTATCTGCACTCAAG
TATCTAAATGAGATTAAGCCTCCAGTTATCCACTACGATCTGAAGCCCGG
CAACATTCTGCTTACCGAGGGCAACGTCTGCGGCGAGATTAAGATCACCG
ACTTCGGTCTGTCAAAGGTGATGGACGACGAGAATTACAATCCCGATCAC
GGCATGGATCTGACCTCTCAGGGGGCGGGAACCTACTGGTATCTGCCACC
CGAGTGCTTTGTCGTGGGCAAAAATCCGCCGAAAATCTCCTCCAAAGTGG
ACGTATGGAGTGTGGGTGTTATCTTCTACCAGTGTCTGTACGGCAAAAAG
CCCTTCGGTCACAATCAGTCGCAGGCCACGATTCTCGAGGAGAATACGAT
CCTGAAGGCCACCGAAGTGCAGTTCTCCAACAAGCCAACCGTTTCTAACG
AGGCCAAG

(SEQ ID NO:117)

MSPGAHLQMSPQNTSSLSQHHPHQQQQLQPPQQQQQHFPNHHSAQQQSQQ
QQQQEQQNPQQQAQQQQQILPHQHLQHLHKHPHQLQLHQQQQQQLHQQQQ
QHFHQQSLQGLHQGSSNPDSNMSTGSSHSEKDVNDMLSGGAATPGAAAAA
IQQQHPAFAPTLGMQQPPPPPPQHSNNGGEMGYLSAGTTTTTSVLTVGKP
RTPAERKRKRKMPPCATSADEAGSGGGSGGAGATVVNNSSLKGKSLAFRD
MPKVNMSLNLGDRLGGSAGSGVGAGGAGSGGGGAGSGSGSGGGKSARLML
PVSDNKKINDYFNKQQTGVGVGVPGGAGGNTAGLRGSHTGGGSKSPSSAQ
QQQTAAQQQGSGVATGGSAGGSAGNQVQVQTSSAYALYPPASPQTQTSQQ
QQQQQPGSDFHYVNSSKAQQQQQRQQQQTSNQMVPPHVVVGLGGHPLSLA
SIQQQTPLSQQQQQQQQQQQQQQLGPPTTSTASVVPTHPHQLGSLGVVGM
VGVGVGVGVGVNVGVGPPLPPPPPMAMPAAIITYSKATQTEVSLHELQER
EAEHESGKVKLDEMTRLSDEQKSQIVGNQKTIDQHKCHIAKCIDVVKKLL
KEKSSIEKKEARQKCMQNRLRLGQFVTQRVGATFQENWTDGYAFQELSRR
QEEITAEREEIDRQKKQLMKKRPAESGRKRNNNSNQNNQQQQQQQHQQQQ
QQQNSNSNDSTQLTSGVVTGPGSDRVSVSVDSGLGGNNAGAIGGGTVGGG
VGGGGVGGGGVGGGGGRGLSRSNSTQANQAQLLHNGGGGSGGNVGNSGGV
GDRLSDRGGGGGGIGGNDSGSCSDSGTFLKPDPVSGAYTAQEYYEYDEIL
KLRQNALKKEDADLQLEMEKLERERNLHIRELKRILNEDQSRFNNHPVLN
DRYLLLMLLGKGGFSEVHKAFDLKEQRYVACKVHQLNKDWKEDKKANYIK
HALREYNIHKALDHPRVVKLYDVFEIDANSFCTVLEYCDGHDLDFYLKQH
KTIPEREARSIIMQVVSALKYLNEIKPPVHIYDLKPGNILLTEGNVCGEI
KITDFGLSKVMDDENYNPDHGMDLTSQGAGTYWYLPPECFVVGKNPPKIS
SKVDVWSVGVIFYQCLYGKKPFGHNQSQATILEENTILKATEVQFSNKPT
VSNEAK

Human homologue of Complete Genome candidate

AAF03095—tousled-like kinase2

| | | |
|---|---|---|
| 1 | ccgggcgggg ggttgcggcg ctcaggagag gccccggctc cgccccgggc ctgcccaggg | (SEQ ID NO:118) |
| 61 | ggagagcgga gctccgcagc cgggtcgggt cggggcccct cccgggagga gcgtggagcg | |
| 121 | cggcggcggc ggcggcagca gaaatgatgg aagaattgca tagcctggac ccacgacggc | |
| 181 | aggaattatt ggaggccagg tttactggag taggtgttag taagggacca cttaatagtg | |
| 241 | agtcttccaa ccagagcttg tgcagcgtcg gatccttgag tgataaagaa gtagagactc | |
| 301 | ccgagaaaaa gcagaatgac cagcgaaatc ggaaaagaaa agctgaacca tatgaaacta | |
| 361 | gccaagggaa aggcactcct aggggacata aaattagtga ttactttgag tttgctgggg | |
| 421 | gaagcgcgcc aggaaccagc cctggcagaa gtgttccacc agttgcacga tcctcaccgc | |
| 481 | aacattcctt atccaatccc ttaccgcgac gagtagaaca gcccctctat ggtttagatg | |
| 541 | gcagtgctgc aaaggaggca acggaggagc agtctgctct gccaaccctc atgtcagtga | |
| 601 | tgctagcaaa acctcggctt gacacagagc agctggcgca aaggggagct ggcctctgct | |
| 661 | tcacttttgt ttcagctcag caaaacagtc cctcatctac gggatctggc aacacagagc | |
| 721 | attcctgcag ctcccaaaaa cagatctcca tccagcacag acggacccag tccgacctca | |
| 781 | caatagaaaa aatatctgca ctagaaaaca gtaagaattc tgacttagag aagaaggagg | |
| 841 | gaagaataga tgatttatta agagccaact gtgatttgag acggcagatt gatgaacagc | |
| 901 | aaaagatgct agagaaatac aaggaacgat taaatagatg tgtgacaatg agcaagaaac | |
| 961 | tccttataga aaagtcaaaa caagagaaga tggcgtgtag agataagagc atgcaagacc | |
| 1021 | gcttgagact gggccacttt actactgtcc gacacggagc ctcatttact gaacagtgga | |
| 1081 | cagatggtta tgcttttcag aatcttatca agcaacagga aaggataaat tcacagaggg | |
| 1141 | aagagataga aagacaacgg aaaatgttag caaagcggaa acctcctgcc atgggtcagg | |
| 1201 | cccctcctgc aaccaatgag cagaaacagc ggaaaagcaa gaccaatgga gctgaaaatg | |
| 1261 | aaacgttaac gttagcagaa taccatgaac aagaagaaat cttcaaactc agattaggtc | |
| 1321 | atcttaaaaa ggaggaagca gagatccagg cagagctgga gagactagaa agggttagaa | |
| 1381 | atctacatat cagggaacta aaaaggatac ataatgaaga taattcacaa tttaaagatc | |
| 1441 | atccaacgct aaatgacaga tatttgttgt tacatctttt gggtagagga ggtttcagtg | |
| 1501 | aagtttacaa ggcatttgat ctaacagagc aaagatacgt agctgtgaaa attcaccagt | |
| 1561 | taaataaaaa ctggagagat gagaaaaagg agaattacca caagcatgca tgtagggaat | |
| 1621 | accggattca taaagagctg gatcatccca gaatagttaa gctgtatgat tacttttcac | |
| 1681 | tggatactga ctcgttttgt acagtattag aatactgtga gggaaatgat ctggacttct | |
| 1741 | acctgaaaca gcacaaatta atgtcggaga aagaggcccg gtccattatc atgcagattg | |
| 1801 | tgaatgcttt aaagtactta aatgaaataa aacctcccat catacactat gacctcaaac | |
| 1861 | caggtaatat tcttttagta aatggtacag cgtgtggaga gataaaaatt acagattttg | |
| 1921 | gtctttcgaa gatcatggat gatgatagct acaattcagt ggatggcatg gagctaacat | |
| 1981 | cacaaggtgc tggtacttat tggtatttac caccagagtg ttttgtggtt gggaaagaac | |
| 2041 | caccaaagat ctcaaataaa gttgatgtgt ggtcggtggg tgtgatcttc tatcagtgtc | |
| 2101 | tttatggaag gaagcctttt ggccataacc agtctcagca agacatccta caagagaata | |
| 2161 | cgattcttaa agctactgaa gtgcagttcc cgccaaagcc agtagtaaca cctgaagcaa | |
| 2221 | aggcgtttat tcgacgatgc ttggcctacc gaaagaggga ccgcattgat gtccagcagc | |
| 2281 | tggcctgtga tccctacttg ttgcctcaca tccgaaagtc agtctctaca agtagccctg | |
| 2341 | ctggagctgc tattgcatca acctctgggg cgtccaataa cagttcttct aattgagact | |
| 2401 | gactccaagg ccacaaactg ttcaacacac acaaagtgga caaatggcgt tcagcagcgg | |
| 2461 | gtttggaaca tagcgaatcc gaatggatct gatgaaacct gtaccaggtg cttttatttt | |
| 2521 | cttgcttttt tcccatccat agagcatgac agcatcgatt ctcattgagg agaaaccttg | |
| 2581 | ggcagctccg gccaggcctt gtaggaaaag gccccgcccg aggttccagc gtcaacggcc | |
| 2641 | actgtgtgtg gctgctctga gtgaggaaaa aattaaaaag aaaaactggt tccatgtact | |
| 2701 | gtgaacttga aaacttgcag actcaggggg gtccctgatg cagtgcttca gatgaagaat | |
| 2761 | gtggacttga aaatacagac tgggctagtc cagtgtctat atttaaactt gttcttttct | |
| 2821 | tttaataaag tttaggtaac atctcctgaa aagcttgtag cacaaaggct cagctgggga | |
| 2881 | tggtgtttga cttcggagga aaaaagttgc tattgcccgt taaaggcact agagttagtg | |
| 2941 | ttttatccct aaataatttc aatttttaaa aacatgcagc ttccctctcc ccttttttat | |
| 3001 | ttttgaaaga atacatttgg tcataaagtg aaacccgtat tagcaagtac gaggcaatgt | |
| 3061 | tcattccaat cagatgcagc tttctcctcc gtctggtctc ctgtttgcaa ttgcttccct | |
| 3121 | catctcagta gggaaaaaat tgagtgggag tactgagatg tgtgggtttt tgccattgga | |
| 3181 | caaagaatga ggttagaaga ctgcagcttg gagtctctct aggttttcaa ctatttcttc | |
| 3241 | acaatttgaa cacttgacgg ttgtcccttt taatttattt gaagtgctat ttttttaaat | |
| 3301 | aaaggttcat ctgtccatgc aaaaaaa | |
| 1 | meelhsldpr rqellearft gvgvskgpln sessnqslcs vgslsdkeve tpekkqndqr | (SEQ ID NO:119) |
| 61 | nrkrkaepye tsqgkgtprg hkisdyfefa ggsapgtspg rsvppvarss pqhslsnplp | |
| 121 | rrveqplygl dgsaakeate eqsalptlms vmlakprldt eqlaqrgagl cftfvsaqqn | |
| 181 | spsstgsgnt ehscssqkqi siqhrrtqsd ltiekisale nsknsdlekk egriddllra | |
| 241 | ncdlrrqide qqkmlekyke rlnrcvtmsk klliekskqe kmacrdksmq drlrlghftt | |
| 301 | vrhgasfteq wtdgyafqnl ikqqerinsq reeierqrkm lakrkppamg qappatneqk | |
| 361 | qrksktngae netltlaeyh eqeeifklrl ghlkkeeaei qaelerlerv mlhirelkr | |
| 421 | ihnednsqfk dhptlndryl llhllgrggf sevykafdlt eqryvavkih qlnknwrdek | |
| 481 | kenyhkhacr eyrihkeldh privklydyf sldtdsfctv leycegndld fylkqhklms | |
| 541 | ekearsiimq ivnalkylne ikppiihydl kpgnillvng tacgeikitd fglskimddd | |
| 601 | synsvdgmel tsqgagtywy lppecfvvgk eppkisnkvd vwsvgvifyq clygrkpfgh | |
| 661 | nqsqqdilqe ntilkatevq fppkpvvtpe akafirrcla yrkrdridvq qlacdpyllp | |
| 721 | hirksvstss pagaaiasts gasnnsssn | |

Putative function
Serine threonine kinase involved in replication and cell cycle

### Example 4

### Category 2

Line ID—224

Phenotype—Semi-lethal male and female, cytokinesis defect. Onion stage cysts have variable sized Nebenkerns. Also has a mitotic phenotype: Tangled unevenly condensed chromosomes, anaphases with lagging chromosomes and bridges

Annotated Drosophila genome genomic segment containing P element insertion site (and map position)—AE003450 (9C)

P element insertion site—139,674

Annotated Drosophila genome Complete Genome candidate

CG2096—flapwing, phosphatase type 1

(SEQ ID NO:120)

ATCTGTAAGTGAAGTCCACTAACAACCGGTTTACTTGCAGTGCGCAGCTG
CCGAACGGGCAAACAGGTCCAGATGACGGAGGCGGAGGTGCGTGGCCTCT
GTCTCAAGTCGCGCGAGATCTTCTTGCAACAGCCCATCCTGCTGGAACTG
GAGGCACCGCTGATCATCTGCGGCGACATCCACGGCCAGTACACAGACCT
GTTGCGCCTGTTCGAGTACGGCGGATTCCCTCCGGCTGCCAACTACTTGT
TCCTCGGCGACTACGTCGATCGGGGCAAGCAGTCCCTGGAGACCATCTGT
CTGCTGCTGGCCTACAAGATCAAATATCCGGAGAACTTCTTCTTGTTGCG
CGGCAACCACGAGTGCGCCAGTATTAATAGGATTTACGGCTTCTACGATG
AGTGCAAGCGCCGATACAATGTCAAACTGTGGAAGACTTTCACAGATTGC
TTCAACTGTCTGCCGGTAGCCGCCATTATTGACGAAAAGATCTTCTGCTG
CCACGGCGGCCTCAGTCCCGATCTTCAGGGCATGGAGCAGATCCGTCGCC
TAATGCGACCCACAGATGTGCCGGATACCGGGTTACTGTGCGATCTTCTG
TGGAGTGATCCCGACAAGGATGTTCAGGGTTGGGGCGAGAATGATCGCGG
TGTGAGCTTCACCTTCGGTGTGGATGTGGTCTCCAAGTTTTTGAACCGCC
ACGAGCTGGACTTGATCTGCCGTGCACATCAGGTTGTGGAGGATGGCTAT
GAGTTCTTTGCGCGTCGGCAACTGGTCACGTTGTTCTCGGCGCCCAATTA
CTGTGGAGAGTTCGACAATGCCGGCGGAATGATGACCGTGGACGACACGC
TGATGTGCTCATTCCAGATCCTGAAACCATCCGAGAAGAAGGCCAAGTAT
CTGTACAGCGGAATGAACTCGTCGCGACCCACAACACCGCAGCGCAGCGC
CCCAATGCTTGCGACCAACAAGAAGAAATAATATATCCATCCGCTTCCAT
TTCCTTAAAGGTTCAACAAACAACAGAAATAAACTTTTACATAGATACAC
ACATATATACATATAAATATAACGAAACGATAGAAAAGGAGAGCGTTAGG
CGATAGTAGAGAAAGGGCAAATGATAAATTAAATGTGTGAGCTATTAAAG
CAAGCAAAATCGAAGTGCATGAATATCAACATCTATGTGAATCCGTCATT
ATCTGTTATCTGATGTGTCATCTGTATCCAACTTGATTACCTTATCCGTG
TACCTGCTAGTTGCAGCAGCAACATCAGGAGCAACAACACCAGCAGCAGC
AGCAGCAGAAACATCAGTGAAACACTCAGAGGCCCATAGTTAAGTCGATT
CCTGCATTTGATGATTATCTGTTGAATGGAAATTGTGACAACGTCCCCGT
AACAGCAGCTCCCAGATCCAAAACTCCCGAAACATGCAGATAAATAAATA
CATTAAAAGTACAGCGATGTTAAGCAATGAATTTATATATAGGCTTATTA
ATGTAAACT

(SEQ ID NO:121)

MTEAEVRGLCLKSREIFLQQPILLELEAPLIICGDIHGQYTDLLRLFEYG
GFPPAANYLFLGDYVDRGKQSLETICLLLAYKIKYPENFFLLRGNHECAS
INRIYGFYDECKRRYNVKLWKTFTDCFNCLPVAAIIDEKIFCCHGGLSPD
LQGMEQIRRLMRPTDVPDTGLLCDLLWSDPDKDVQGWGENDRGVSFTFGV
DVVSKFLNRHELDLICRAHQVVEDGYEFFARRQLVTLFSAPNYCGEFDNA
GGMMTVDDTLMCSFQILKPSEKKAKYLYSGMNSSRPTTPQRSAPMLATNK
KK

Human homologue of Complete Genome candidate

NP_{—}002700 protein phosphatase 1, catalytic subunit, beta isoform

| | | |
|---|---|---|
| 1 | cctgggtctg acgcggccct gttcgagggg gcctctcttg tttatttatt tattttccgt | (SEQ ID NO:122) |
| 61 | gggtgcctcc gagtgtgcgc gcgctctcgc tacccggcgg ggagggggtg gggggagggc | |
| 121 | ccgggaaaag ggggagttgg agccggggtc gaaacgccgc gtgacttgta ggtgagagaa | |
| 181 | cgccgagccg tcgccgcagc ctccgccgcc gagaagccct tgttcccgct gctgggaagg | |
| 241 | agagtctgtg ccgacaagat ggcggacggg gagctgaacg tggacagcct catcacccgg | |
| 301 | ctgctggagg tacgaggatg tcgtccagga aagattgtgc agatgactga agcagaagtt | |
| 361 | cgaggcttat gtatcaagtc tcgggagatc tttctcagcc agcctattct tttggaattg | |
| 421 | gaagcaccgc tgaaaatttg tggagatatt catggacaat atacagattt actgagatta | |
| 481 | tttgaatatg gaggtttccc accagaagcc aactatcttt tcttaggaga ttatgtggac | |
| 541 | agaggaaagc agtctttgga aaccatttgt ttgctattgg cttataaaat caaatatcca | |
| 601 | gagaacttct ttctcttaag aggaaaccat gagtgtgcta gcatcaatcg catttatgga | |
| 661 | ttctatgatg aatgcaaacg aagatttaat attaaattgt ggaagacctt cactgattgt | |
| 721 | tttaactgtc tgcctatagc agccattgtg gatgagaaga tcttctgttg tcatggagga | |
| 781 | ttgtcaccag acctgcaatc tatggagcag attcggagaa ttatgagacc tactgatgtc | |
| 841 | cctgatacag gtttgctctg tgatttgcta tggtctgatc cagataagga tgtgcaaggc | |
| 901 | tggggagaaa atgatcgtgg tgtttccttt acttttggag ctgatgtagt cagtaaattt | |
| 961 | ctgaatcgtc atgatttaga tttgatttgt cgagctcatc aggtggtgga agatggatat | |
| 1021 | gaattttttg ctaaacgaca gttggtaacc ttattttcag ccccaaatta ctgtggcgag | |
| 1081 | tttgataatg ctggtggaat gatgagtgtg gatgaaactt tgatgtgttc atttcagata | |
| 1141 | ttgaaaccat ctgaaaagaa agctaaatac cagtatggtg gactgaattc tggacgtcct | |
| 1201 | gtcactccac ctcgaacagc taatccgccg aagaaaaggt gaagaaagga attctgtaaa | |
| 1261 | gaaaccatca gatttgttaa ggacatactt cataatatat aagtgtgcac tgtaaaacca | |
| 1321 | tccagccatt tgacaccctt tatgatgtca cacctttaac ttaaggagac gggtaaagga | |
| 1381 | tcttaaattt ttttctaata gaaagatgtg ctacactgta ttgtaataag tatactctgt | |
| 1441 | tatagtcaac aaagttaaat ccaaattcaa aattatccat taaagttaca tcttcatgta | |
| 1501 | tcacaatttt taaagttgaa aagcatccca gttaaactag atgtgatagt taaaccagat | |
| 1561 | gaaagcatga tgatccatct gtgtaatgtg gttttagtgt tgcttggttg tttaattatt | |
| 1621 | ttgagcttgt tttgtttttg tttgttttca ctagaataat ggcaaatact tctaattttt | |
| 1681 | ttccctaaac atttttaaaa gtgaaatatg ggaagagctt tacagacatt caccaactat | |
| 1741 | tattttccct tgtttatcta cttagatatc tgtttaatct tactaagaaa actttcgcct | |
| 1801 | cattacatta aaaaggaatt ttagagattg attgttttaa aaaaaaatac gcacattgtc | |
| 1861 | caatccagtg attttaatca tacagtttga ctgggcaaac tttacagctg atagtgaata | |
| 1921 | ttttgcttta tacaggaatt gacactgatt tggatttgtg cactctaatt tttaacttat | |
| 1981 | tgatgctcta ttgtgcagta gcatttcatt taagataagg ctcatatagt attacccaac | |
| 2041 | tagttggtaa tgtgattatg tggtaccttg gctttaggtt ttcattcgca cggaacacct | |
| 2101 | tttggcatgc ttaacttcct ggtaacacct tcacctgcat tggttttctt tttctttttt | |
| 2161 | ctttcttttt tttttttttt ttttttttga gttgttgttt gtttttagat ccacagtaca | |
| 2221 | tgagaatcct tttttgacaa gccttggaaa gctgacactg tctctttttc ctccctctat | |
| 2281 | acgaaggatg tatttaaatg aatgctggtc agtgggacat tttgtcaact atgggtattg | |
| 2341 | ggtgcttaac tgtctaatat tgccatgtga atgttgtata cgattgtaag gcttatgtca | |
| 2401 | ctaaagattt ttattctgat tttttcataa tcaaaggtca tatgatactg tatagacaag | |
| 2461 | ctttgtagtg aagtatagta gcaataattt ctgtacctga tcaagtttat tgcagccttt | |
| 2521 | cttttcctat ttcttttttt taagggttag tattaacaaa tggcaatgag tagaaaagtt | |
| 2581 | aacatgaaga ttttagaagg agagaactta caggacacag atttgtgatt ctttgactgt | |
| 2641 | gacactattg gatgtgattc taaaagcttt tattgagcat tgtcaaattt gtaagcttca | |
| 2701 | tagggatgga catcatatct ataatgccct tctatatgtg ctaccataga tgtgacattt | |
| 2761 | ttgaccttaa tatcgtcttt gaaaatgtta aattgagaaa cctgttaact tacattttat | |
| 2821 | gaattggcac attgtattac ttactgcaag agatatttca ttttcagcac agtgcaaaag | |
| 2881 | ttctttaaaa tgcatatgtc tttttttcta attccgtttt gttttaaagc acattttaaa | |
| 2941 | tgtagttttc tcatttagta aaagttgtct aattgatatg aagcctgact gatttttttt | |
| 3001 | ttccttacag tgagacattt aagcacacat tttattcaca tagatactat gtccttgaca | |
| 3061 | tattgaaatg attcttttct gaaagtattc atgatctgca tatgatgtat taggttaggt | |
| 3121 | cacaaaggtt ttatctgagg tgatttaaat aacttcctga ttggagtgtg taagctgagc | |
| 3181 | gatttctaat aaaattttag ttgtacactt ttagtagtca tagtgaagca ggtctagaaa | |
| 3241 | ataagccttt ggcagggaaa aagggcaatg ttgattaatc tcagtattaa accacattaa | |
| 3301 | tctgtatccc attgtctggc ttttgtaaat tcatccaggt caagactaag tatgttggtt | |
| 3361 | aataggaatc cttttttttt tttaaagact aaatgtgaaa aaataatcac tacttaagct | |
| 3421 | aattaatatt ggtcattaaa tttaaaggat ggaaatttat catgtttaaa aattattcaa | |
| 3481 | gcactcttaa aaccacttaa acagcctcca gtcataaaaa tgtgttcttt acaaatattt | |
| 3541 | gcttggcaac acgacttgaa ataaataaaa ctttgtttct taggagaaaa | |
| 1 | madgelnvds litrllevrg crpgkivqmt eaevrglcik sreiflsqpi lleleaplki | (SEQ ID NO:123) |
| 61 | cgdihgqytd llrlfeyggf ppeanylflg dyvdrgkqsl eticlllayk ikypenffll | |
| 121 | rgnhecasin riygfydeck rrfniklwkt ftdcfnclpi aaivdekifc chgglspdlq | |
| 181 | smeqirrimr ptdvpdtgll cdllwsdpdk dvqgwgendr gvsftfgadv vskflnrhdl | |
| 241 | dlicrahqvv edgyeffakr qlvtlfsapn ycgefdnagg mmsvdetlmc sfqilkpsek | |
| 301 | kakyqyggln sgrpvtpprt anppkkr | |

Putative function
Protein phosphatase

### Example 5

### Category 2

Line ID—231

Phenotype—Semi-lethal male and female, cytokinesis defect. In some cysts, variable sized Nebenkerns

Annotated Drosophila genome genomic segment containing P element insertion site (and map position)—AE003429 (3F)

P element insertion site—153,730

Annotated Drosophila genome Complete Genome candidate

CG5014—vap-33-1 vesicle associated membrane protein

(SEQ ID NO:124)

CACATCACTAGCTGACAGAATATATGGCTTTTTTACATTTTGCGTTTTCA
ACTGAAGTTTGCGAAGAAACCGAAGCGTGGTAAACCACTGAAATCGAAAA
TATCGACAGAAAAGCGACCTAAAGTCGGTGAAGAAGTCGCACGTTGATCG
TTGTGTTTTTTTCCCGAAATTTTCTGCAAAAAGCCCGTGCGTGCGTGAGT
TTCTCTGGCTCTTGCTTTTTTTTTGTCCATGCGTGTGTGTGTGGTCGCAT
AAATTTACCGATATTTCGCCTGTGAGAGCGAAACGAACGAAAAACGAAAG
AAAAAAAGAGAGACGAGTAAAGTAAAACGAAACAGGCATAAAAACAGCAG
CAGTTTTCTTGATATATTTGGCTAAAAAACGCAAACCAAACAGCCAGCAA
GAACAACAAATAGCTGGGCAAAAACAGGACGCACAAAAAATAAAATTAAA
ACGATAAGAGGCGAAAAGCGGAGAGAGTGAAATTCTCGGCAGCAACAACG
ACAAGAACAACACCAGGAGCAGCAGCAACAACAACAACAAAAGCCAGCCG
CCACAATGAGCAAATCACTCTTTGATCTTCCGTTGACCATTGAACCAGAA
CATGAGTTGCGTTTTGTGGGTCCCTTCACCCGACCCGTTGTCACAATCAT
GACTCTGCGCAACAACTCGGCTCTGCCTCTGGTCTTCAAGATCAAGACAA
CCGCCCCGAAACGCTACTGCGTACGTCCAAACATCGGCAAGATAATTCCC
TTTCGATCAACCCAGGTGGAGATCTGCCTTCAGCCATTCGTCTACGATCA
GCAGGAGAAGAACAAGCACAAGTTCATGGTGCAGAGCGTCCTGGCACCCA
TGGATGCTGATCTAAGCGATTTAAATAAATTGTGGAAGGATCTGGAGCCC
GAGCAGCTGATGGACGCCAAACTGAAGTGCGTTTTCGAGATGCCCACCGC
TGAGGCAAATGCTGAGAACACCAGCGGTGGTGGTGCCGTTGGCGGCGGAA
CCGGAGCTGCCGGAGGCGGAAGCGCGGGTGCCAATACTAGCTCAGCCAGC
GCTGAGGCGCTCGAGAGCAAGCCGAAGCTCTCCAGCGAGGATAAGTTTAA
GCCATCCAATTTGCTCGAAACGTCTGAGAGTCTGGACTTGCTGTCCGGAG
AGATCAAAGCGCTGCGTGAATGCAACATTGAATTGCGAAGAGAGAATCTT
CACTTGAAGGATCAAATCACACGTTTCCGGAGCTCGCCGGCCGTCAAACA
GGTGAATGAGCCCTATGCCCCAGTCCTGGCTGAGAAGCAGATTCCGGTCT
TTTACATTGCAGTTGCCATTGCTGCGGCCATCGTTAGCCTCCTGCTGGGC
AAATTCTTTCTCTGA

(SEQ ID NO:125)

MSKSLFDLPLTIEPEHELRFVGPFTRPVVTIMTLRNNSALPLVFKIKTTA
PKRYCVRPNIGKIIPFRSTQVEICLQPFVYDQQEKNKHKFMVQSVLAPMD
ADLSDLNKLWKDLEPEQLMDAKLKCVFEMPTAEANAENTSGGGAVGGGTG
AAGGGSAGANTSSASAEALESKPKLSSEDKFKPSNLLETSESLDLLSGEI
KALRECNIELRRENLHLKDQITRFRSSPAVKQVNEPYAPVLAEKQIPVFY
IAVAIAAAIVSLLLGKFFL

Human homologue of Complete Genome candidate

AAD13577 VAMP-associated protein B

| | | |
|---|---|---|
| 1 | gcgcgcccac ccggtagagg acccccgccc gtgccccgac cggtccccgc ctttttgtaa | (SEQ ID NO:126) |
| 61 | aacttaaagc gggcgcagca ttaacgcttc ccgccccggt gacctctcag gggtctcccc | |
| 121 | gccaaaggtg ctccgccgct aaggaacatg gcgaaggtgg agcaggtcct gagcctcgag | |
| 181 | ccgcagcacg agctcaaatt ccgaggtccc ttcaccgatg ttgtcaccac caacctaaag | |
| 241 | cttggcaacc cgacagaccg aaatgtgtgt tttaaggtga agactacagc accacgtagg | |
| 301 | tactgtgtga ggcccaacag cggaatcatc gatgcagggg cctcaattaa tgtatctgtg | |
| 361 | atgttacagc ctttcgatta tgatcccaat gagaaaagta aacacaagtt tatggttcag | |
| 421 | tctatgtttg ctccaactga cacttcagat atggaagcag tatggaagga ggcaaaaccg | |
| 481 | gaagacctta tggattcaaa acttagatgt gtgtttgaat tgccagcaga gaatgataaa | |
| 541 | ccacatgatg tagaaataaa taaaattata tccacaactg catcaaagac agaaacacca | |
| 601 | atagtgtcta agtctctgag ttcttctttg gatgacaccg aagttaagaa ggttatggaa | |
| 661 | gaatgtaaga ggctgcaagg tgaagttcag aggctacggg aggagaacaa gcagttcaag | |
| 721 | gaagaagatg gactgcggat gaggaagaca gtgcagagca acagccccat ttcagcatta | |
| 781 | gccccaactg ggaaggaaga aggccttagc acccggctct tggctctggt ggttttgttc | |
| 841 | tttatcgttg gtgtaattat tgggaagatt gccttgtaga ggtagcatgc acaggatggt | |
| 901 | aaattggatt ggtggatcca ccatatcatg ggatttaaat ttatcataac catgtgtaaa | |
| 961 | aagaaattaa tgtatgatga catctcacag gtcttgcctt taaattaccc ctccctgcac | |
| 1021 | acacatacac agatacacac acacaaatat aatgtaacga tcttttagaa agttaaaaat | |
| 1081 | gtatagtaac tgattgaggg ggaaaagaat gatctttatt aatgacaagg gaaaccatga | |
| 1141 | gtaatgccac aatggcatat tgtaaatgtc attttaaaca ttggtaggcc ttggtacatg | |
| 1201 | atgctggatt acctctctta aaatgacacc cttcctcgcc tgttggtgct ggcccttggg | |
| 1261 | gagctggagc ccagcatgct ggggagtgcg gtcagctcca cacagtagtc cccacgtggc | |
| 1321 | ccactcccgg cccaggctgc tttccgtgtc ttcagttctg tccaagccat cagctccttg | |
| 1381 | ggactgatga acagagtcag aagcccaaag gaattgcact gtggcagcat cagacgtact | |
| 1441 | cgtcataagt gagaggcgtg tgttgactga ttgacccagc gctttggaaa taaatggcag | |
| 1501 | tgctttgttc acttaaaggg accaagctaa atttgtattg gttcatgtag tgaagtcaaa | |
| 1561 | ctgttattca gagatgttta atgcatattt aacttattta atgtatttca tctcatgttt | |
| 1621 | tcttattgtc acaagagtac agttaatgct gcgtgctgct gaactctgtt gggtgaactg | |
| 1681 | gtattgctgc tggagggctg tgggctcctc tgtctctgga gagtctggtc atgtggaggt | |
| 1741 | ggggtttatt gggatgctgg agaagagctg ccaggaagtg ttttttctgg gtcagtaaat | |
| 1801 | aacaactgtc ataggcaggg aaattctcag tagtgacagt caactctagg ttaccttttt | |
| 1861 | taatgaagag tagtcagtct tctagattgt tcttatacca cctctcaacc attactcaca | |
| 1921 | cttccagcgc ccaggtccaa gtttgagcct gacctcccct tggggaccta gcctggagtc | |
| 1981 | aggacaaatg gatcgggctg caaagggtta gaagcgaggg caccagcagt tgtgggtggg | |
| 2041 | gagcaaggga agagagaaac tcttcagcga atccttctag tactagttga gagtttgact | |
| 2101 | gtgaattaat tttatgccat aaaagaccaa cccagttctg tttgactatg tagcatcttg | |
| 2161 | aaaagaaaaa ttataataaa gccccaaaat taaga | |
| 1 | makveqvlsl epqhelkfrg pftdvvttnl klgnptdrnv cfkvkttapr rycvrpnsgi | (SEQ ID NO:127) |
| 61 | idagasinvs vmlqpfdydp nekskhkfmv qsmfaptdts dmeavwkeak pedlmdsklr | |
| 121 | cvfelpaend kphdveinki isttasktet pivskslsss lddtevkkvm eeckrlqgev | |
| 181 | qrlreenkqf keedglrmrk tvqsnspisa laptgkeegl strllalvvl ffivgviigk | |
| 241 | ial | |

Putative function
Membrane associated protein which may be involved in priming synaptic vesicles

### Example 6

### Category 2

Line ID—248

Phenotype—Male sterile, cytokinesis defect. Cytokinesis defect, different meiotic stages within one cyst, variable sized nuclei, 2-4 nuclei. Also has a mitotic phenotype: semi-lethal, rod-like overcondensed chromosomes, high mitotic index, lagging chromosomes and bridges.

Annotated Drosophila genome genomic segment containing P element insertion site (and map position)—AE003431 (4D 1)

P element insertion site—299,078

Annotated Drosophila genome Complete Genome candidate

CG6998—cutup (dynein light chain)

(SEQ ID NO:128)

CAAAACGTTCAGTTGTGTTTCAGTTGTCGAGAAGTCAGGGTGTTTCTACC
TTCCATTTACCGTTCCAGTGTAAAATTCAGGCGACACGCTTAGCGTTACC
AAGGAGAACCGCTAAAAAGGGCCACTTTTCAAACGGTTAGATTCCAGTGA
AGTTGTAAGCACACAGGGAACCTAAAAAAAAAAAAAACAGCCAAAATGTC
TGATCGCAAGGCCGTGATTAAAAATGCCGACATGAGCGAGGAGATGCAGC
AGGATGCCGTCGATTGTGCGACACAGGCCCTCGAGAAGTACAACATTGAA
AAGGACATTGCGGCCTACATCAAGAAGGAGTTCGACAAAAAATACAATCC
CACATGGCATTGCATTGTCGGTCGCAACTTTGGATCGTATGTCACACACG
AGACGCGCCACTTTATTTACTTCTATTTGGGCCAGGTGGCTATTTTACTG
TTTAAGAGCGGTTAAAGTATTGTCGAGTCGGATGAAGTGGTGGTGAGGAG
GCTGATGGAGATGCAGCAGCTGCCCCGCCAGCAGCAACAACAGCAGGGGC
AGCAGTCGCATTTCGGAGCATCAGAGGATGAGGATCTAGAGCAGAAACAG
CAACAACCA

(SEQ ID NO:129)

MSDRKAVIKNADMSEEMQQDAVDCATQALEKYNIEKDIAAYIKKEFDKKY
NPTWHCIVGRNFGSYVTHETRHFIYFYLGQVAILLFKSG

Human homologue of Complete Genome candidate

AAH10744 Similar to RIKE cDNA 6720463E02 gene

| | | |
|---|---|---|
| 1 | gctgtgaggc gccagtgcgg agcgggcggg cgggcgggcg ggcgggcggc gcgaggcgga | (SEQ ID NO:130) |
| 61 | gcgcgggcgg ccggcgaaac tccaagggcg gaccgcggca gggagcgatc ggcctcgggc | |
| 121 | tgcgggagcc ggagaccgcg gcggcggcgg ctgctgcagc tgcaggagga gcccagggaa | |
| 181 | caccgcccct gcctgtgctc tgcctcgggc catcgctcct ccccagggcc cagtgcggac | |
| 241 | tcgcctccgt gaagtgtcac accatgtctg accggaaggc agtgatcaag aacgcagaca | |
| 301 | tgtctgagga catgcaacag gatgccgttg actgcgccac gcaggccatg gagaagtaca | |
| 361 | atatagagaa ggacattgct gcctatatca agaaggaatt tgacaagaaa tataacccta | |
| 421 | cctggcattg tatcgtgggc cgaaattttg gcagctacgt cacacacgag acaaagcact | |
| 481 | tcatctattt ttacttgggt caagttgcaa tcctcctctt caagtcaggc taggtggcca | |
| 541 | tggtgaaggt gtcagtggcg gcggcagcga tggcaagcag gcggcgttgc tgggactgtt | |
| 601 | ttgcactgga gccagcatca ggatgtcctc tccaatggct gtgctactgc atggactgta | |
| 661 | tactcgattt catgtgtatg tcgcagtaaa caaaaccaaa cctcaaaaaa aaaaaaaaaa | |
| 721 | aaaaaaaaaa aaaaa | |
| 1 | msdrkavikn admsedmqqd avdcatqame kyniekdiaa yikkefdkky nptwhcivgr | (SEQ ID NO:131) |
| 61 | nfgsyvthet khfiyfylgq vaillfksg | |

Putative function
Dynein light chain, a microtubule motor protein

### Example 7

### Category 2

Line ID—bb1-E1

Phenotype—Male sterile. Asynchronous meiotic divisions, cysts with large Nebenkern and 1-2 larger nuclei, testis from 2-3 old males become smaller. High mitotic index, colchicine type overcondensaton, many anaphases and telophases, no decondensation in telophase. Also has a mitotic phenotype: High mitotic index, coichicines-type overcondensed chromosomes, many ana- and relophases, no decondensation in telophase

Annotated Drosophila genome genomic segment containing P element insertion site (and map position)—AE003431 (4E)

P element insertion site—not determined

Annotated Drosophila genome Complete Genome candidate

CG2984—Pp2C 1 protein phosphatase

(SEQ ID NO:132)

TGTTCGCAAGTCGAGAGCAGAATCGAACGGCAAAAAATGCTGGCGAACAA
CAAATCATCAAGGTAAAACTGCGCGCCTTGGTCATTAAGTCTTTCATCGA
GGATAAAAGACCGATGTCTTTTAACGTTATTGCTGTAAGCAAAAGCAGAA
ATCACAATCTACTCATAAATCCTCGATTTGGTGCAAATTAAAGGAAATTC
ATCGGTTTTTGGCGGCCAGTTGCAAACACAAAATACTAAATACGCTAGAT
GGAGCACGCATACACGCAAGCTCGTTGGCGAACGTAAATTACATACATCA
TATAGATAGTCGTCCCGCTTGCACTGCCCGTCACAGCGAGGGCTGCGAGA
GCGAGAGCGGGAGAGAGAAAGGCCTGAGTCGCTTTTTCTTCTTGTACTTT
ATATATTTTTTATTGTTTTTTTGTGTTGTGTTGCGTTGTACGTGTGTGTG
AGAGTGCCAAATGTCAACGGAAATTACAACACTGCGAGACGGAGAAGTCT
AAAAGGCAGAAGAAGAAGAAGCAGCAGCAGGCAGCATAAACAAAACTCGG
GGGAAAAATGTTGCCCGCCAATAACAGGAGTAGCACCAGCACCCATACCA
ACACAAATGCCAACACAATCAACGCCACTACCAATACCACCAACAGATGC
CTCATCAATACGGCCATCGAAAAAACGGTAGTCCGTTTGCGAGAGACGGC
AGCGAATAGCGCACCAGCTCCAGCCACAGCCTCCGTTACTCGCCACGGCG
GCAGCAGCAGCGGCAATAACAACAATAACAGTGCATGCCATCCAGCACTG
GATGCCAGCAGTGATGTTGTTGTTGTTGAACCGGCAGCGGTAGGAGTCGC
ACAGGAGGAAGAGGAAGAGCCGGAGCAAAGGCCAGAGAGGATCAGCATAC
CCATTCCCGACCTGGCGTTCACCGAGATGGAAGCATATGCCGAGGATATA
GTCGTCGATATGGAGGGGGGATCACCAGCCAAGCCTTTAAATCCAAAGAA
ACAACGTTTAAACTCAGCAACAACCACAACAATAAATCGCTCGAGGGGCG
GCGGAGCGGCACAGAGTCGATTACGCCGGTCGGCGGCCATCGTTCCACCG
CGATCGATTCCAGAGAGCTGTGCCAGCAGCAGCAATTCCAATTCGAGCAG
CAGTTCCAACAGTAATTCCAGTTCCAGCTCCGCTACAGGAAGTAGCGCAT
CCACCGGCAATCCGTCGCCGTGCTCCTCCCTGGGCGTCAATATGCGCGTA
ACTGGACAATGCTGCCAGGGAGGCCGGAAATACATGGAGGATCAGTTCTC
GGTGGCCTACCAGGAATCACCGATCACCCACGAACTGGAATACGCATTTT
TTGGCATCTACGACGGACACGGCGGTCCCGAGGCCGCGCTCTTCGCCAAG
GAGCACCTTATGCTCGAGATCGTCAAGCAGAAGCAGTTCTGGTCTGATCA
GGATGAGGATGTCCTGCGGGCAATACGCGAGGGATACATCGCCACACATT
TCGCCATGTGGCGGGAACAAGAGAAATGGCCACGCACTGCCAATGGGCAT
CTGAGCACCGCCGGCACCACCGCCACAGTGGCCTTTATGCGTCGCGAGAA
GATCTACATTGGTCATGTGGGTGATTCTGGGATCGTTTTGGGTTACCAGA
ACAAGGGCGAACGCAACTGGCGTGCTCGTCCACTGACCACGGACCACAAG
CCGGAGTCACTGGCAGAGAAGACGAGAATCCAGCGTTCCGGCGGCAATGT
TGCCATCAAATCGGGAGTTCCGCGAGTGGTATGGAACCGACCCAGGGACC
CAATGCATCGCGGTCCCATTCGCCGCAGAACTCTGGTAGATGAAATACCC
TTTTTGGCGGTGGCTCGTTCCCTGGGCGATCTCTGGAGCTACAATTCCCG
CTTCAAGGAATTCGTTGTGAGTCCCGATCCGGATGTCAAAGTGGTTAAAA
TAAATCCCAGTACCTTTAGATGCTTAATTTTCGGCACCGATGGCCTGTGG
AATGTGGTGACCGCCCAGGAGGCGGTGGACAGTGTGCGCAAGGAGCATCT
AATCGGCGAGATACTCAACGAGCAGGACGTTATGAATCCCAGCAAGGCGC
TGGTGGATCAGGCCCTCAAAACCTGGGCCGCCAAGAAGATGCGTGCGGAC
AACACGTCCGTTGTGACTGTGATACTAACACCAGCGGCCCGCAATAATTC
GCCCACAACGCCAACACGTTCCCCATCCGCGATGGCACGCGACAATGATC
TGGAGGTGGAGCTACTGCTGGAGGAGGACGACGAGGAGCTGCCGACACTG
GATGTGGAGAACAACTACCCTGACTTTCTCATCGAGGAGCATGAGTATGT
GCTGGACCAGCCGTACAGTGCATTGGCCAAGCGACATTCGCCTCCGGAAG
CCTTCCGCAACTTCGACTACTTCGATGTGGACGAGGACGAGTTGGATGAA
GATGAGGAAACAGTGGAAGAAGACGAGGAGGAGGAGGAGGAAGAGGAGGA
AACCAAATCGGTGGGAATTCTACAGCAAAGTTTGTTCAACCCCAGAAAAA
CGTGGCGCAAGTCAACCATCAACAATTCCTGGAGTGGCGTCACCGAACCG
GAACCGGAACCCGATCCCGAACCAGATCGAATAGATGTCTTAACACTGGA
CATGTACTCCCACACCAGCATTGACAAGGGCACCAATTATGGCGGCAGCA
TAGCCCAGTCCTCAATAGATCCTGCGGAGACGGCTGAAAATCGTGAGCTG
AGTGAGTTGGAGCAGCATCTGGAGAGTAGCTACAGTTTCGCCGAGTCGTA
CAACTCCCTGTTAAACGAGCAGGAGGAGCAGGAGGCACGCTCACGTTCAG
CAGCAGCAGCAGCCGCCGCCGCAGAAGCAGCAGCAGTAGAAGCACAACAA
ACCACTGCCCATTCCGCATCCGTTGTGCTGGACCGCAGCATGTTGGAGAT
CATCCAGGAGCAGCAGCACTATCAGCAGCAAGAGGGCTATTCGCTAACGC
AACTAGAGACCAGACGTGAAAGGGAGCGGCTGACCGAATCGTGGCCACAG
CAGCCGGCTGAGCTGCTCGAGCTGGATGCTCTACTGCAGCAGGAGCGTGC
CGAGGAGGAGCAGGTAGCCCTGGAGCAGCAGCAGCAGCGCGAACAGCAAA
TGGAGCAAATGGAGGTGGAGGCCATTAGTAGTTCGGGACAGCACGAATTT
GCTTACCCAGTGACCACCGCCACAGCCAGCGAGTGGTGTGCTACATTACA
AGAAGACGAGGAGGAGTTGGACTCCACAGTAATAGACATAGTAATTCAAC
CCGAACAAGAGTTGCAGGACAATGAAGTGAGCTCCACGTTGCCCGCCACA
CCCACTCATGTGGAGCCTGAGCAGATTGTGGACAAGATGGAGCCCCTGAA
GGTTCAGGAGATGCTAACCGCGGTCGAAAAACCTCCATCCAAGCAGGAAA
AGAAGCTGCCGAAGAAGCAAGAGACCAAACAGGTTGCTGTGCTAGATACA
GTGGCCGAGATGCCCAAAGAGGATGCCCATGCCGTGCACTATATATTCCA
GCGCATTCAAAAGGTTCAGGACTCTGAGGCAACACCAGTGGCCGTGACGA
ATTCCACAATGGCTGACGCCCTGCCCACCGAATCTAGTGGACTGGGAGGA
TCTATGACCGCGCCCCGAATCCGACGCTATCGCAACGTGCCCAACGAGAA
CCATCAGCACATGCAGACGCGTCGTCGTCAGATCTTCAAGCATGTCAAGC
CAAAGTCCTTCATACAGTCCAGTGCTGCGGCGATTGTGGCCTATGGAGAC
AGCACCGAAACGGTCGGAGGAACAGCCGGAGCATCTGGCACACCTGCAGC
TGGGCGTGTAGGCGGGGGCGGTGGCGGCGGCGGCGGCAGAGGATCGGCCA
GTGGTGGGAGCAGTCCAGCGGTGGCAGCCAATAGTCGGCGGAGCGTCAAT
GTGGTGGCCAATGCGAGTGGAAACAGCGCTAGCAAAGTTGTGCCCAGCAG
CAGTTCCATGATGATGACCCGCCGCAGTCACACCTTGACGGCCAGCGGTG
GTGTGAACAAAAGGCAGCTGCGCAGCAGTCTCTGCACCTTGGGCCTGGGT
GTGGGTGTCGGTGTCGGTCTGGGCATGGACCTGGACATGACCAAGCGCAC
GCTAAGGACAAGGAATGTACCCGCTTTGTCGGGCGGTTCAGCCACGCCAT
CTAGCAATTCGTCGCCAGCCAGCGGAGGCAGCAGTCCAGCCGGTTTCACA
AGCCCAGCCAGTCCGGTCATCACGTCCAGGGGAAGCGGATCGCGTACTAC
CGCCTCGCCAGCCAGGCGCCTAAAACGCAGTCATGAGGATCGGGAGCAAA
GAATGAGCTTGCGACGGAGCACTCTGAGTGGCAGTGCCAGCGGCAGTGGG
CTGGTGGGCACTGGTGGGTCGCCCTCGAATGTGAAATCAAATCGCCTGCA
GGCCTGCAATGGAGCCATCTCTGCGCGTCCGCCGCCCTCGCCGAAGAAAC
TGAATGCAGCCGTGCCCACATTGGCAATTGGAACGCGTGCATATACGGCG
GCGTTGGCGGCGGCGGCGGATCACCTGAACAAGCGGTGGTCGTTGCGCAG
CAGCAGTGGCAACTCTGGCAATCTGATAACCGCCATCAGTTGCTACAGTG
ACAGGAGCAGGGCGGCGACTGCGGCGGGATCACCGGGATCTGGAGGCGGG
GCAGCGGGACCACCAGGAGCATCTTTGGCCGCATCCACAGTCGGCACGCG
AAGGCGCTAGGCTAGATTGTAACGAAACATGCGAGCAACTTGCAAGTACA
AATCCTAAGCAACGGAAAATTTTAGATCCTAGTATACTACTTTACTGAAA
ACGCAAAATTGCATAATTTAACCAATTTTTTTATGTGCACAACACACACA
C

(SEQ ID NO:133)

MLPANNRSSTSTHTNTNANTINATTNTTNRCLINTAIEKTVVRLRETAAN
SAPAPATASVTRHGGSSSGNNNNNSACHPALDASSDVVVVEPAAVGVAQE
EEEEPEQRPERISIPIPDLAFTEMEAYAEDIVVDMEGGSPAKPLNPKKQR
LNSATTTTINRSRGGGAAQSRLRRSAAIVPPRSIPESCASSSNSNSSSSS
NSNSSSSSATGSSASTGNPSPCSSLGVNMRVTGQCCQGGRKYMEDQFSVA
YQESPITHELEYAFFGIYDGHGGPEAALFAKEHLMLEIVKQKQFWSDQDE
DVLRAIREGYIATHFAMWREQEKWPRTANGHLSTAGTTATVAFMRREKIY
IGHVGDSGIVLGYQNKGERNWRARPLTTDHKPESLAEKTRIQRSGGNVAI
KSGVPRVVWNRPRDPMHRGPIRRRTLVDEIPFLAVARSLGDLWSYNSRFK
EFVVSPDPDVKVVKINPSTFRCLIFGTDGLWNVVTAQEAVDSVRKEHLIG
EILNEQDVMNPSKALVDQALKTWAAKKMRADNTSVVTVILTPAARNNSPT
TPTRSPSAMARDNDLEVELLLEEDDEELPTLDVENNYPDFLIEEHEYVLD
QPYSALAKRHSPPEAFRNFDYFDVDEDELDEDEETVEEDEEEEEEEEETK
SVGILQQSLFNPRKTWRKSTINNSWSGVTEPEPEPDPEPDRIDVLTLDMY
SHTSIDKGTNYGGSIAQSSIDPAETAENRELSELEQHLESSYSFAESYNS
LLNEQEEQEARSRSAAAAAAAAEAAAVEAQQTTAHSASVVLDRSMLEIIQ
EQQHYQQQEGYSLTQLETRRERERLTESWPQQPAELLELDALLQQERAEE
EQVALEQQQQREQQMEQMEVEAISSSGQHEFAYPVTTATASEWCATLQED
EEELDSTVDIVIQPEQELQDNEVSSTLPATPTHVEPEQIVDKMEPLKVQ
EMLTAVEKPPSKQEKKLPKKQETKQVAVLDTVAEMPKEDAHAVHYIFQRI
QKVQDSEATPVAVTNSTMADALPTESSGLGGSMTAPRIRRYRNVPNENHQ
HMQTRRRQIFKHVKPKSFIQSSAAAIVAYGDSTETVGGTAGASGTPAAGR
VGGGGGGGGGRGSASGGSSPAVAANSRRSVNVVANASGNSASKVVPSSSS
MMMTRRSHTLTASGGVNKRQLRSSLCTLGLGVGVGVGLGMDLDMTKRTLR
TRNVPALSGGSATPSSNSSPASGGSSPAGFTSPASPVITSRGSGSRTTAS
PARRLKRSHEDREQRMSLRRSTLSGSASGSGLVGTGGSPSNVKSNRLQAC
NGAISARPPPSPKKLNAAVPTLAIGTRAYTAALAAAADHLNKRWSLRSSS
GNSGNLITAISCYSDRSRAATAAGSPGSGGGAAGPPGASLAASTVGTRRR

Human homologue of Complete Genome candidate

AAB61637 Wip1

| | | |
|---|---|---|
| 1 | ctggctctgc tcgctccggc gctccggccc agctctcgcg gacaagtcca gacatcgcgc | (SEQ ID NO:134) |
| 61 | gccccccctt ctccgggtcc gccccctccc ccttctcggc gtcgtcgaag ataaacaata | |
| 121 | gttggccggc gagcgcctag tgtgtctccc gccgccggat tcggcgggct gcgtgggacc | |
| 181 | ggcgggatcc cggccagccg gccatggcgg ggctgtactc gctgggagtg agcgtcttct | |
| 241 | ccgaccaggg cgggaggaag tacatggagg acgttactca aatcgttgtg gagcccgaac | |
| 301 | cgacggctga agaaaagccc tcgccgcggc ggtcgctgtc tcagccgttg cctccgcggc | |
| 361 | cgtcgccggc cgcccttccc ggcggcgaag tctcggggaa aggcccagcg gtggcagccc | |
| 421 | gagaggctcg cgaccctctc ccggacgccg gggcctcgcc ggcacctagc cgctgctgcc | |
| 481 | gccgccgttc ctccgtggcc tttttcgccg tgtgcgacgg gcacggcggg cgggaggcgg | |
| 541 | cacagtttgc ccgggagcac ttgtggggtt tcatcaagaa gcagaagggt ttcacctcgt | |
| 601 | ccgagccggc taaggtttgc gctgccatcc gcaaaggctt tctcgcttgt caccttgcca | |
| 661 | tgtggaagaa actggcggaa tggccaaaga ctatgacggg tcttcctagc acatcaggga | |
| 721 | caactgccag tgtggtcatc attcggggca tgaagatgta tgtagctcac gtaggtgact | |
| 781 | caggggtggt tcttggaatt caggatgacc cgaaggatga ctttgtcaga gctgtggagg | |
| 841 | tgacacagga ccataagcca gaacttccca aggaaagaga acgaatcgaa ggacttggtg | |
| 901 | ggagtgtaat gaacaagtct ggggtgaatc gtgtagtttg gaaacgacct cgactcactc | |
| 961 | acaatggacc tgttagaagg agcacagtta ttgaccagat tccttttctg gcagtagcaa | |
| 1021 | gagcacttgg tgatttgtgg agctatgatt tcttcagtgg tgaatttgtg gtgtcacctg | |
| 1081 | aaccagacac aagtgtccac actcttgacc ctcagaagca caagtatatt atattgggga | |
| 1141 | gtgatggact ttggaatatg attccaccac aagatgccat ctcaatgtgc caggaccaag | |
| 1201 | aggagaaaaa atacctgatg ggtgagcatg gacaatcttg tgccaaaatg cttgtgaatc | |
| 1261 | gagcattggg ccgctggagg cagcgtatgc tccgagcaga taacactagt gccatagtaa | |
| 1321 | tctgcatctc tccagaagtg gacaatcagg gaaactttac caatgaagat gagttatacc | |
| 1381 | tgaacctgac tgacagccct tcctataata gtcaagaaac ctgtgtgatg actccttccc | |
| 1441 | catgttctac accaccagtc aagtcactgg aggaggatcc atggccaagg gtgaattcta | |
| 1501 | aggaccatat acctgccctg gttcgtagca atgccttctc agagaatttt ttagaggttt | |
| 1561 | cagctgagat agctcgagag aatgtccaag gtgtagtcat accctcaaaa gatccagaac | |
| 1621 | cacttgaaga aaattgcgct aaagccctga ctttaaggat acatgattct ttgaataata | |
| 1681 | gccttccaat tggccttgtg cctactaatt caacaaacac tgtcatggac caaaaaaatt | |
| 1741 | tgaagatgtc aactcctggc caaatgaaag cccaagaaat tgaaagaacc cctccaacaa | |
| 1801 | actttaaaag gacattagaa gagtccaatt ctggccccct gatgaagaag catagacgaa | |
| 1861 | atggcttaag tcgaagtagt ggtgctcagc ctgcaagtct ccccacaacc tcacagcgaa | |
| 1921 | agaactctgt taaactcacc atgcgacgca gacttagggg ccagaagaaa attggaaatc | |
| 1981 | ctttacttca tcaacacagg aaaactgttt gtgtttgctg aaatgcatct gggaaatgag | |
| 2041 | gtttttccaa acttaggata taagagggct ttttaaattt ggtgccgatg ttgaactttt | |
| 2101 | tttaagggga gaaaattaaa agaaatatac agtttgactt tttggaattc agcagtttta | |
| 2161 | tcctggcctt gtacttgctt gtattgtaaa tgtggatttt gtagatgtta gggtataagt | |
| 2221 | tgctgtaaaa tttgtgtaaa tttgtatcca cacaaattca gtctctgaat acacagtatt | |
| 2281 | cagagtctct gatacacagt aattgtgaca atagggctaa atgtttaaag aaatcaaaag | |
| 2341 | aatctattag attttagaaa aacatttaaa ctttttaaaa tacttattaa aaaatttgta | |
| 2401 | taagccactt gtcttgaaaa ctgtgcaact ttttaaagta aattattaag cagactggaa | |
| 2461 | aagtgatgta ttttcatagt gacctgtgtt tcacttaatg tttcttagag ccaagtgtct | |
| 2521 | tttaaacatt attttttatt tctgatttca taattcagaa ctaaattttt catagaagtg | |
| 2581 | ttgagccatg ctacagttag tcttgtccca attaaaatac tatgcagtat ctcttacatc | |
| 2641 | agtagcattt ttctaaaacc ttagtcatca gatatgctta ctaaatcttc agcatagaag | |
| 2701 | gaagtgtgtt tgcctaaaac aatctaaaac aattcccttc tttttcatcc cagaccaatg | |
| 2761 | gcattattag gtcttaaagt agttactccc ttctcgtgtt tgcttaaaat atgtgaagtt | |
| 2821 | ttccttgcta tttcaataac agatggtgct gctaattccc aacatttctt aaattatttt | |
| 2881 | atatcataca gttttcattg attatatggg tatatattca tctaataaat cagtgaactg | |
| 2941 | ttcctcatgt tgctgaaaaa aaaaaaaaaa aaa | |
| 1 | maglyslgvs vfsdqggrky medvtqivve peptaeekps prrslsqplp prpspaalpg | (SEQ ID NO:135) |
| 61 | gevsgkgpav aareardplp dagaspapsr ccrrrssvaf favcdghggr eaaqfarehl | |
| 121 | wgfikkqkgf tssepakvca airkgflach lamwkklaew pktmtglpst sgttasvvii | |
| 181 | rgmkmyvahv gdsgvvlgiq ddpkddfvra vevtqdhkpe lpkererieg lggsvmnksg | |
| 241 | vnrvvwkrpr lthngpvrrs tvidqipfla varalgdlws ydffsgefvv spepdtsvht | |
| 301 | ldpqkhkyii lgsdglwnmi ppqdaismcq dqeekkylmg ehgqscakml vnralgrwrq | |
| 361 | rmlradntsa ivicispevd nqgnftnede lylnltdsps ynsqetcvmt pspcstppvk | |
| 421 | sleedpwprv nskdhipalv rsnafsenfl evsaeiaren vqgvvipskd pepleencak | |
| 481 | altlrihdsl nnslpiglvp tnstntvmdq knlkmstpgq mkaqeiertp ptntkrtlee | |
| 541 | snsgplmkkh rrnglsrssg aqpaslptts qrknsvkltm rrrlrgqkki gnpllhqhrk | |
| 601 | tvcvc | |

Putative function
Protein phosphatase, with p53 dependent expression, so may be inhibitory to division

### Example 8

### Category 2

Line ID—ms(1)04

Phenotype—Cytokinesis defect, small testis, no meiosis observed, variable sized Nebenkerns with 2-4N nuclei

Annotated Drosophila genome genomic segment containing P element insertion site (and map position)—AE003442 (7C-D)

P element insertion site—not determined

Annotated Drosophila genome Complete Genome candidate

CG1524—RpS14A ribosomal protein (2 splice variants)

(SEQ ID NO:136)

GATATCCGGTTAACGCAAGTGTTGCTGATCGACAAACAAACCCAGAATGG
CACCCAGGAAGGCTAAAGTTCAGAAGGAGGAGGTTCAGGTCCAGCTGGGA
CCCCAAGTTCGCGACGGCGAGATCGTGTTCGGAGTGGCTCACATCTACGC
CAGCTTCAACGACACCTTCGTCCATGTCACTGATCTGTCCGGCCGTGAGA
CCATCGCTCGTGTCACCGGAGGCATGAAGGTGAAGGCCGATCGTGATGAG
GCTTCGCCCTACGCCGCTATGTTGGCCGCTCAGGATGTGGCTGAGAAGTG
CAAGACACTGGGCATTACTGCCCTGCATATTAAGCTGCGTGCCACCGGCG
GCAACAAGACCAAGACCCCCGGACCCGGCGCCCAGTCCGCTCTGCGTGCT
TTGGCCCGTTCGTCCATGAAGATTGGCCGCATCGAGGATGTGACGCCCAT
CCCATCGGACTCCACCCGCAGGAAGGGCGGTCGCCGTGGTCGTCGTCTGT
AGATGGCAGTATCTGGAAAGCAGTAGTCTATGTTTGCGGTCGAAATACAA
TACTGC

(SEQ ID NO:137)

MAPRKAKVQKEEVQVQLGPQVRDGEIVFGVAHIYASFNDTFVHVTDLSGR
ETIARVTGGMKVKADRDEASPYAAMLAAQDVAEKCKTLGITALHIKLRAT
GGNKTKTPGPGAQSALRALARSSMKIGRIEDVTPIPSDSTRRKGGRRGRR
L

(SEQ ID NO:138)

CAAGTGGTTCGTCTTTAATTTTTCCCTCTTAATTTTTGCGAAAAAAAACC
CGACTTTGAGCCCCTAAACTTAAAAAATGTGCCTTCCTCCAGAGTGTTCA
GAGCGTCGACTGAAAATGACAAACAAGCTGCCCGGCAGCTAATTTTTTTT
TACATTTTTTGTTTTGTTTGTTCGCACGCATTTGTTTTTATTTGTGAAAC
ACGTGGTATAAATGTGGAAATTCCCTTGCTATTCCCGCAGTTGCTGATCG
ACAAACAAACCCAGAATGGCACCCAGGAAGGCTAAAGTTCAGAAGGAGGA
GGTTCAGGTCCAGCTGGGACCCCAAGTTCGCGACGGCGAGATCGTGTTCG
GAGTGGCTCACATCTACGCCAGCTTCAACGACACCTTCGTCCATGTCACT
GATCTGTCCGGCCGTGAGACCATCGCTCGTGTCACCGGAGGCATGAAGGT
GAAGGCCGATCGTGATGAGGCTTCGCCCTACGCCGCTATGTTGGCCGCTC
AGGATGTGGCTGAGAAGTGCAAGACACTGGGCATTACTGCCCTGCATATT
AAGCTGCGTGCCACCGGCGGCAACAAGACCAAGACCCCCGGACCCGGCGC
CCAGTCCGCTCTGCGTGCTTTGGCCCGTTCGTCCATGAAGATTGGCCGCA
TCGAGGATGTGACGCCCATCCCATCGGACTCCACCCGCAGGAAGGGCGGT
CGCCGTGGTCGTCGTCTGTAGATGGCAGTATCTGGAAAGCAGTAGTCTAT
GTTTGCGGTCGAAATACAATACTGC

(SEQ ID NO:139)

MAPRKAKVQKEEVQVQLGPQVRDGEIVFGVAHIYASFNDTFVHVTDLSGR
ETIARVTGGMKVKADRDEASPYAAMLAAQDVAEKCKTLGITALHIKLRAT
GGNKTKTPGPGAQSALRALARSSMKIGRIEDVTPIPSDSTRRKGGRRGRR
L

Human homologue of Complete Genome candidate

A25220 ribosomal protein S14, cytosolic

```
(SEQ ID NO:140)
    1 ctccgccctc tcccactctc tctttccggt gtggagtctg gagacgacgt gcagaaatgg
  61 cacctcgaaa ggggaaggaa aagaaggaag aacaggtcat cagcctcgga cctcaggtgg
121 ctgaaggaga gaatgtattt ggtgtctgcc atatctttgc atccttcaat gacacttttg
181 tccatgtcac tgatctttct ggcaaggaaa ccatctgccg tgtgactggt gggatgaagg
241 taaaggcaga ccgagatgaa tcctcaccat atgctgctat gttggctgcc caggatgtgg
301 cccagaggtg caaggagctg ggtatcaccg ecetacacat caaactccgg gccacaggag
361 gaaataggac caagacccct ggacctgggg cccagtcggc cctcagagcc cttgcccgct
421 cgggtatgaa gatcgggcgg attgaggatg tcacccccat cccctctgac agcactcgca
481 ggaagggggg tcgccgtggt cgccgtctgt gaacaagatt cctcaaaata ttttctgtta
541 ataaattgcc ttcatgtaaa aaaaaaaaaa aaaaaaaaaa aaaaaaaaa
(SEQ ID NO:141)
    1 maprkgkekk eeqvislgpq vaegenvfgv chifasfndt fvhvtdlsgk eticrvtggm
  61 kvkadrdess pyaamlaaqd vaqrckelgi taihikirat ggnrtktpgp gaqsalrala
121 rsgmkigrie dvtpipsdst rrkggrrgrr l
```

Putative function
Ribosomal protein

### Example 9

### Category 2

Line ID—thb-a

Phenotype—Male sterile. Cytokinesis defect, larger Nebenkerns with 2-4N nuclei

Annotated Drosophila genome genomic segment containing P element insertion site (and map position)—(10B1-2)

P element insertion site—not determined

Annotated Drosophila genome Complete Genome candidate
2 candidates:

CG1453—kinesin-like protein KIF2 homolog

```
(SEQ ID NO:142)
AAACTAAAAAATTGTGTTGCTGACATCTGGTCGCTTGCAAAACTATTTCT
AGCAGATTTTGTGATATTTCGTTGTGATCGGTCGATAAATCCGCCAGTTT
TTTTTTTAATGGAAAGTGCTAACACATTGTAGCGGTTGGGAAGATAGCAG
GAAAGAGCCAGCGGGCTGCCGTTTTTCCTTTTTGTTATCCGTTGCCAGAC
GCAACGAAAACGACAGTTGGCATTTGAATTCAGCACAAACACACATACTA
ACGCCGACCCGCAAGCAGCACACACACACACACTGGGACACTCGAAAAAA
AAAAAACAGACGCTGTCGGCGACCTCGACAAGCAGTTGGGTTCGATTTAG
TTGTCAATGCCTTGAATTCGGTTCGGGGCTTAGTTTCCACAAGTTTATCG
CTCGTCAAGAAACAACGAAATAAAATTATTTTCGACCTAAAAAATCTGAC
TAAATTGTGTTTTTTGTTTATGTATTTATTTAGGCACATTTTGCACACCA
CAACGTAGTTACTACATCTACGACTAACGGAACTCCTCCTGCAAGCAGTG
GAAGTTGCTGTCCATCAAGCAGTACTCGGAGTTAACGCAGGATAAGCCGG
GAGAAAGAGAAAGAGATCGGTGGAGAATAGAGATATACAGGTGGAGTCAA
AGAGGAAGGATCATGGACATGATTACGGTGGGGCAGAGCGTCAAGATCAA
GCGGACGGATGGCCGCGTCCACATGGCCGTGGTGGCGGTGATCAACCAGT
CGGGCAAGTGCATCACAGTCGAATGGTACGAGCGCGGCGAAACGAAGGGC
AAGGAGGTAGAACTGGACGCCATACTCACGCTCAATCCGGAGCTAATGCA
AGATACTGTCGAACAGCACGCCGCCCCGGAGCCCAAGAAACAAGCCACCG
CGCCGATGAACCTCTCGCGTAATCCCACACAATCGGCTATCGGTGGCAAT
CTCACCAGCCGTATGACCATGGCCGGAAACATGCTGAACAAGATCCAGGA
AAGCCAGTCGATTCCCAATCCGATTGTCAGCAGCAATAGCGTGAATACAA
ACAGCAACTCCAACACTACGGCCGGCGGAGGTGGTGGCACCACAACGTCG
ACGACCACTGGATTACAGCGTCCACGGTACTCGCAAGCTGCTACCGGCCA
GCAGCAGACAAGGATCGCCTCGGCGGTGCCTAATAACACATTGCCCAATC
CCAGCGCGGCAGCCAGTGCTGGTCCGGCGGCACAAGGAGTCGCCACTGCG
GCCACAACCCAGGGAGCTGGCGGCGCTAGTACCCGGCGATCGCACGCATT
GAAAGAGGTGGAGCGACTGAAGGAGAATCGCGAGAAGCGACGCGCCCGAC
AGGCCGAGATGAAGGAGGAGAAGGTGGCGCTGATGAACCAGGATCCGGGC
AATCCAAACTGGGAGACGGCGCAAATGATACGCGAATATCAGAGCACGCT
GGAATTTGTGCCGCTGCTCGATGGCCAGGCCGTCGATGACCATCAGATCA
CAGTGTGCGTGCGCAAGCGTCCCATTAGCCGCAAGGAGGTCAATCGCAAG
GAGATCGATGTCATTTCGGTGCCGCGCAAGGACATGCTCATCGTGCACGA
GCCGCGCAGCAAGGTCGACCTCACCAAGTTCCTGGAGAACCACAAGTTTC
GCTTCGACTACGCCTTCAACGACACGTGCGACAATGCCATGGTATACAAA
TACACAGCCAAGCCGTTGGTGAAAACCATTTTCGAGGGCGGAATGGCGAC
GTGCTTCGCCTACGGCCAGACGGGATCGGGCAAAACGCACACCATGGGCG
GTGAGTTTAATGGAAAGGTGCAGGACTGCAAGAACGGCATCTACGCCATG
GCGGCCAAGGATGTCTTTGTGACCCTGAATATGCCGCGTTACCGCGCCAT
GAATCTAGTCGTCTCGGCCAGTTTCTTTGAGATTTACAGTGGCAAGGTCT
TCGATCTTCTGTCCGACAAGCAGAAACTGCGCGTCCTGGAGGATGGTAAA
CAGCAAGTGCAGGTGGTGGGACTCACCGAGAAGGTGGTCGATGGCGTCGA
GGAGGTACTGAAGCTCATCCAGCACGGCAATGCTGCCCGAACATCCGGCC
AGACGTCGGCCAACTCCAATTCGTCGCGTTCGCACGCCGTTTTCCAGATT
GTGCTGCGGCCGCAGGGCTCGACGAAGATCCATGGCAAGTTCTCGTTCAT
CGATCTGGCGGGCAATGAGCGGGGCGTGGACACTTCCTCGGCCGATCGGC
AGACGCGTATGGAGGGTGCCGAGATTAACAAATCGCTGCTGGCCCTCAAG
GAGTGCATTCGTGCGTTGGGCAAACAGTCGGCCCACTTGCCCTTCCGTGT
CTCCAAACTCACCCAGGTGCTGCGCGACTCGTTCATTGGCGAGAAGAGCA
AGACGTGCATGATAGCCATGATCTCGCCGGGACTTAGCTCCTGCGAGCAC
ACGCTCAACACGCTGCGCTATGCGGATCGTGTCAAGGAGCTGGTGGTCAA
GGATATCGTCGAAGTTTGCCCTGGCGGCGACACCGAGCCCATCGAGATCA
CGGACGACGAGGAGGAGGAGGAGCTCAACATGGTGCATCCGCACTCGCAT
CAGCTGCATCCCAATTCGCATGCACCGGCCAGCCAGTCGAATAATCAGCG
TGCTCCGGCCTCTCATCACTCGGGGGCGGTCATTCACAACAATAATAATA
ACAACAACAAGAACGGAAACGCCGGCAACATGGACCTGGCCATGCTGAGT
TCGCTGAGCGAACACGAGATGTCCGACGAGCTGATTGTGCAGCACCAGGC
CATCGACGACCTGCAGCAGACGGAGGAGATGGTGGTGGAGTATCATCGCA
CCGTTAATGCCACACTGGAGACCTTCCTCGCCGAGTCGAAGGCGCTGTAC
AATCTGACCAACTATGTGGACTACGACCAGGACTCGTACTGCAAACGGGG
CGAGTCGATGTTCTCGCAGCTGCTGGACATCGCCATCCAGTGCCGCGACA
TGATGGCCGAATATCGCGCCAAGTTGGCCAAGGAGGAGATGCTGTCGTGC
AGCTTCAATTCGCCGAATGGCAAGCGTTAGT
(SEQ ID NO:143)
    1 mitvgqsvki krtdgrvhma vvavinqsgk citvewyerg etkgkeveld ailtlnpelm
  61 qdtveqhaap epkkqatapm nlsrnptqsa iggnltsrmt magnmlnkiq esqsipnpiv
121 ssnsvntnsn snttaggggg tttstttglq rprysqaatg qqqtriasav pnntlpnpsa
181 aasagpaaqg vataattqga ggastrrsha lkeverlken rekrrarqae mkeekvalmn
241 qdpgnpnwet aqmireyqst lefvplldgq avddhqitvc vrkrpisrke vnrkeidvis
301 vprkdmlivh eprskvdltk flenhkfrfd yafndtcdna mvykytakpl vktifeggma
361 tcfaygqtgs gkthtmggef ngkvqdckng iyamaakdvf vtlnmpryra mnlvvsasff
421 eiysgkvfdl lsdkqklrvl edgkqqvqvv gltekvvdgv eevlkliqhg naartsgqts
481 ansnssrsha vfqivlrpqg stkihgkfsf idlagnergv dtssadrqtr megaeinksl
541 lalkeciral gkqsahlpfr vskltqvlrd sfigeksktc miamispgls scehtlntlr
601 yadrvkelvv kdivevcpgg dtepieitdd eeeeelnmvh phshqlhpns hapasqsnnq
661 rapashhsga vihnnnnnnn kngnagnmdl amlsslsehe msdelivqhq aiddlqqtee
721 mvveyhrtvn atletflaes kalynltnyv dydqdsyckr gesmfsqlld iaiqcrdmma
781 eyraklakee mlscsfnspn gkr
CG18292-novel
(SEQ ID NO:144)
CGTAATAACGCCTCCTGATATCGATATCGATATCATATCACAAAAAACAA
TAAACCAAAAAAGAAACGCTAAAAACTAGTAGTTTTGTGTGCCAGGAAAA
CGGAAAGGTGGACATAGTTAAGTTACCACAACAACCGACGGATATCGACT
CCAGACACCACATCGCCCAGCGCCACCATGGACATCATGGATATCCAGGC
CGTAGAGTCCAAGCTGAGTGACGTCACGGTGACACCGATACCGCGCAGCC
AAGTGCAGAATTTCTACAATTACCAGCAGCAGCGGGAGCAGCGCGAGCAG
CAGCCCCAAATCCAGATATCGGCCATCCACCACTCGCGTGGATCCGTTGG
CGGAGGAGGCGGATCCAACTCATCCAACGCTGCCACCGACTACTCCACGA
GCAGCGGTGGCAAGCGGGAGCGGGACCGCTCCTCCGCCAGCGACTACAGC
AGCTCGTCCAGCAAGCAGAGCTCCGCTGCAGCGGCCAATGCAGCAGCAGC
TGCCGCCGCCGTCGCTGCCCTCCAATACTCCCCGCAGTTCCTCCAGGCCC
AGCTGGCGCTACTCCAGCAGCAGTCGAACACGACGGCCACGCCGGCAGCC
GTCGCCGCTGCGGCCCTCTCGCTGGCCAACATGTGCTCCAGCAATGGTGG
TCAGCGGAATTCCGGTGCCGGCGTTTCCTCCACCTCCTCTGGCAGCAATG
GCCAGAGCATGGGCCTGAATCTGAGCTCATCGCAGCTAAAGTACCCGCCA
CCCTCCACCTCGCCCGTGGTGGTGACCACCCAAACTTCGGCCAATATCAC
CACGCCGCTGACCTCCACGGCCAGCCTGCCCTCAGTGGGCCCGGGCAATG
GGCTGACCAAGTACGCCCAGCTGCTGGCCGTCATTGAGGAGATGGGCCGC
GATATCCGGCCCACGTACACGGGCTCGCGCAGCTCCACGGAGCGTCTCAA
GCGGGGCATTGTCCATGCCCGCATCCTGGTGCGCGAATGCCTCATGGAAA
CGGAGCGTGCGGCGCGCCAATGA
(SEQ ID NO:145)
    1 mdiqaveskl sdvtvtpipr sqvqnfynyq qqreqreqqp qiqisaihhs rgsvgggggs
  61 nssnaatdys tssggkrerd rssasdysss sskqssaaaa naaaaaaava alqyspqflq
121 aqlallqqqs nttatpaava aaalslanmc ssnggqrnsg agvsstssgs ngqsmglnls
181 ssqlkyppps tspvvvttqt sanittplts taslpsvgpg ngltkyaqll avieemgrdi
241 rptytgsrss terlkrgivh arilvreclm eteraarq
```

Human homologue of Complete Genome candidate

(CG1453)—CAA69621—kinesin-2

```
(SEQ ID NO:146)
      1 ggccgaatac atcaagcaat ggtaacatct ttaaatgaag ataatgaaag tgtaactgtt
    61 gaatggatag aaaatggaga tacaaaaggc aaagagattg acctggagag catcttttca
  121 cttaaccctg accttgttcc tgatgaagaa attgaaccca gtccagaaac acctccacct
  181 ccagcatcct cagccaaagt aaacaaaatt gtaaagaatc gacggactgt agcttctatt
  241 aagaatgacc ctccttcaag agataataga gtggttggtt cagcacgtgc acggcccagt
  301 caatttcctg aacagtcttc ctctgcacaa cagaatggta gtgtttcaga tatatctcca
  361 gttcaagctg caaaaaagga atttggaccc ccttcacgta gaaaatctaa ttgtgtgaaa
  421 gaagtagaaa aactgcaaga aaaacgagag aaaaggagat tgcaacagca agaacttaga
  481 gaaaaaagag cccaggacgt tgatgctaca aacccaaatt atgaaattat gtgtatgatc
  541 agagacttta gaggaagttt ggattataga ccattaacaa cagcagatcc tattgatgaa
  601 cataggatat gtgtgtgtgt aagaaaacga ccactcaata aaaaagaaac tcaaatgaaa
  661 gatcttgatg taatcacaat tcctagtaaa gatgttgtga tggtacatga accaaaacaa
  721 aaagtagatt taacaaggta cctagaaaac caaacatttc gttttgatta tgcctttgat
  781 gactcagctc ctaatgaaat ggtttacagg tttactgcta aaccactagt ggaaactata
  841 tttgaaaggg gaatggctac atgctttgct tatgggcaga ctggaagtgg aaaaactcat
  901 actatgggtg gtgacttttc aggaaagaac caagattgtt ctaaaggaat ttatgcatta
  961 gcagctcgag atgtcttttt aatgctaaag aagccaaact ataagaagct agaacttcaa
1021 gtatatgcaa ccttctttga aatttatagt ggaaaggtgt ttgacttgct aaacaggaaa
1081 acaaaattaa gagttctaga agatggaaaa cagcaggttc aagtggtggg attacaggaa
1141 cgggaggtca aatgtgttga agatgtactg aaactcattg acataggcaa cagttgcaga
1201 acatccggtc aaacatctgc aaatgcacat tcatctcgga gccatgcagt gtttcagatt
1261 attcttagaa ggaaaggaaa actacatggc aaattttctc tcattgattt ggctggaaat
1321 gaaagaggag ctgatacttc cagtgcggac aggcaaacta ggcttgaagg tgctgaaatt
1381 aataaaagcc ttttagcact caaggagtgc atcagagcct taggtagaaa taaacctcat
1441 actcctttcc gtgcaagtaa actcactcag gtgttaagag attctttcat aggtgaaaac
1501 tctcgtacct gcatgattgc cacaatctct ccaggaatgg catcctgtga aaatactctt
1561 aatacattaa gatatgcaaa tagggtcaaa gaattgactg tagatccaac tgctgctggt
1621 gatgttcgtc caataatgca ccatccacca aaccagattg atgacttaga gacacagtgg
1681 ggtgtgggga gttcccctca gagagatgat ctaaaacttc tttgtgaaca aaatgaagaa
1741 gaagtctctc cacagttgtt tactttccac gaagctgttt cacaaatggt agaaatggaa
1801 gaacaagttg tagaagatca cagggcagtg ttccaggaat ctattcggtg gttagaagat
1861 gaaaaggccc tcttagagat gactgaagaa gtagattatg atgtcgattc atatgctaca
1921 caacttgaag ctattcttga gcaaaaaata gacattttaa ctgaactgcg ggataaagtg
1981 aaatctttcc gtgcagctct acaagaggag gaacaagcca gcaagcaaat caacccgaag
2041 agaccccgtg ccctttaaac cggcatttgc tgctaaagga tacccagaac cctcactact
2101 gtaacataca acggttcagc tgtaagggcc atttgaaagt ttggaatttt aagtgtctgt
2161 ggaaaatgtt ttgtccttca cctgaattac atttcaattt tgtgaaacac tcttttgtct
2221 acaaaatgct tctagtccag gaggcacaac caagaactgg gattaatgaa gcattttgtt
2281 tcatttacac aaatagtgat ttacttttgg agatccttgt cagttttatt ttctatttga
2341 tgaagtaaga ctgtggactc aatccagagc cagatagtag gggaagccac agcatttcct
2401 tttaactcag ttcaattttt gtagtgagac tgagcagttt taaatccttt gcgtgcatgc
2461 atacctcatc agtgattgta cataccttgc ccactcctag agacagctgt gctcactttt
2521 cctgctttgt gccttgatta aggctactga ccctaaattt ctgaagcaca gccaagaaaa
2581 attacattcc ttgtcattgt aaattacctt tgtgtgtaca tttttactgt atttgagaca
2641 ttttttgtgt gtgactagtt aattttgcag gatgtgccat atcattgaac ggaactaaag
2701 tctgtgacag tggatatagc tgctggacca ttccatctta tatgtaaaga aatctggaat
2761 tattatttta aaaccatata acatgtgatt ataatttttc ttagcatttt ctttgtaaag
2821 aactacaata taaactagtt ggtgtataat aaaaagtaat gaaattctga agaaaaaaaa
2881 aaaaaaaaaa aaaaaaaaaa aaaaa
(SEQ ID NO:147)
      1 mvtslnedne svtvewieng dtkgkeidle sifslnpdlv pdeeiepspe tppppassak
    61 vnkivknrrt vasikndpps rdnrvvgsar arpsqfpeqs ssaqqngsvs dispvqaakk
  121 efgppsrrks ncvkeveklq ekrekrrlqq qelrekraqd vdatnpnyei mcmirdfrgs
  181 ldyrpittad pidehricvc vrkrplnkke tqmkdldvit ipskdvvmvh epkqkvdltr
  241 ylenqtfrfd yafddsapne mvyrftakpl vetifergma tcfaygqtgs gkthtmggdf
  301 sgknqdcskg iyalaardvf lmlkkpnykk lelqvyatff eiysgkvfdl lnrktklrvl
  361 edgkqqvqvv glqerevkcv edvlklidig nscrtsgqts anahssrsha vfqiilrrkg
  421 klhgkfslid lagnergadt ssadrqtrle gaeinkslla lkeciralgr nkphtpfras
  481 kltqvlrdsf igensrtcmi atispgmasc entlntlrya nrvkeltvdp taagdvrpim
  541 hhppnqiddl etqwgvgssp qrddlkllce qneeevspql ftfheavsqm vemeeqvved
  601 hravfqesir wledekalle mteevdydvd syatqleail eqkidiltel rdkvksfraa
  661 lqeeeqaskq inpkrpral
```

(CG18292)—BAA22937—cdk2-associated protein 1; cdk2ap1, deleted in oral cancer 1 (doc-1, alias DORC1)

```
(SEQ ID NO:148)
      1 accgcccggc ctcgccgccg ccgccgccgc cctcgcggcc tggccccgcc gcgcccggcg
    61 cgcccgccgc ccggggggat gtcttacaaa ccgaacttgg ccgcgcacat gcccgccgcc
  121 gccctcaacg ccgctgggag tgtccactcg ccttccacca gcatggcaac gtcttcacag
  181 taccgccagc tgctcagtga ctacgggcca ccgtccctag gctacaccca gggaactggg
  241 aacagccagg tgccccaaag caaatacgcg gagctgctgg ccatcattga agagctgggg
  301 aaggagatca gacccacgta cgcagggagc aagagtgcca tggagaggct gaagcgcggc
  361 atcattcacg ctagaggact ggttcgggag tgcttggcag aaacggaacg gaatgccaga
  421 tcctagctgc cttgttggtt ttgaaggatt tccatctttt tacaagatga gaagttacag
  481 ttcatctccc ctgttcagat gaaacccttg ttttcaaaat ggttacagtt tcgtttttcc
  541 tcccatggtt cacttggctc tgaacctaca gtctcaaaga ttgagaaaag attttgcagt
  601 taattaggat ttgcatttta agtagttagg aactgcccag gttttttttg ttttttaagc
  661 attgatttaa aagatgcacg gaaagttatc ttacagcaaa ctgtagtttg cctccaagac
  721 accattgtct ccctttaatc ttctcttttg tatacatttg ttacccatgg tgttctttgt
  781 tccttttcat aagctaatac cactgtaggg attttgtttt gaacgcatat tgacagcacg
  841 ctttacttag tagccggttc ccatttgcca tacaatgtag gttctgctta atgtaacttc
  901 ttttttgctt aagcatttgc atgactatta gtgcttcaaa gtcaattttt aaaaatgcac
  961 aagttataaa tacagaagaa agagcaaccc accaaaccta acaaggaccc ccgaacactt
1021 tcatactaag actgtaagta gatctcagtt ctgcgtttat tgtaagttga taaaaacatc
1081 tgggaggaaa tgactaaaac tgtttgcatc tttgtatgta tttattactt gatgtaataa
1141 agcttatttt cattaacc
(SEQ ID NO:149)
      1 msykpnlaah mpaaalnaag svhspstsma tssqyrqlls dygppslgyt qgtgnsqvpq
    61 skyaellaii eelgkeirpt yagsksamer lkrgiiharg lvreclaete mars
```

Putative function
(CG1453)—Motor protein
(CG18292)—Cdk2 associated, candidate tumor supressor

### Example 9A

### Category 2

Line ID—ms(l)13

Phenotype—Male sterile, Cytokinesis defect: variable sized Nebenkerns with 4N nuclei, some nuclei detached from Nebenkern

Annotated Drosophila genome genomic segment containing P element insertion site (and map position)—AE003436 (5D1)

P element insertion site sequence

(SEQ ID NO:150)

CATCATGTATCATACATTGAAGACGGATTAGCACCGTCGACCACGAAAAA
AGAACGCAAGGAAATCGTGCAAAATGTTCAAAAAGTACGTATGGCATGAG
TTAGATGGGGACATCAGACTAACCATAGCAATTCGATCTGTGCAGATTCG
AAGAGAAGGACAGCATTTCCAGCATTCAGCAGCTGAAGTCGTCTGTGCAG
AAGGGCATACGTGCCAAGTTGCTGGAGGCCTATCCCAAGTTGGAGAGTCA
CATCGACCTGATCCTGCCCAAGAAGGACTCGTACCGCATCGCCAAGTGGT
AGGATGGCTCAGTTCTTGCCACAGCACATAACTCCATTCATATTCCCGAT
CCCTACTCCTCCACCAGCCATGACCACATCGAACTGCTGCTAAACGGAGC
CGGCGACCAGGTGTTCTTTCGCCACCGCGATGGCCCCTGGATGCCTACCC
TGCGCAACTGTTGGGAAGGGCGATCGGTGCGGGCCTCTTCGCTATTACGC
CAGCTGGCGAAAGGGGGGATGTGCTGCAAGGCGATTAAGTTGGGTAACGC
CAGGGTTTTCCCAGNCACGACGTTGNAAAACGACGGNCANNGCCAAGCTC
TGCTGCT

Annotated Drosophila genome Complete Genome candidate

CG594 1—novel protein with a PUA domain

(SEQ ID NO:151)

CGGATTAGCACCGTCGACCACGAAAAAAGAACGCAAGGAAATCGTGCAAA
ATGTTCAAAAAATTCGAAGAGAAGGACAGCATTTCCAGCATTCAGCAGCT
GAAGTCGTCTGTGCAGAAGGGCATACGTGCCAAGTTGCTGGAGGCCTATC
CCAAGTTGGAGAGTCACATCGACCTGATCCTGCCCAAGAAGGACTCGTAC
CGCATCGCCAAGTGCCATGACCACATCGAACTGCTGCTAAACGGAGCCGG
CGACCAGGTGTTCTTTCGCCACCGCGATGGCCCCTGGATGCCTACCCTGC
GCCTCCTGCACAAGTTCCCCTACTTCGTGACCATGCAGCAAGTGGACAAA
GGCGCCATCCGCTTCGTCCTGAGCGGAGCGAACGTCATGTGTCCCGGCCT
CACATCGCCAGGCGCCTGTATGACGCCGGCCGACAAGGACACCGTGGTGG
CCATCATGGCTGAGGGCAAGGAGCACGCCCTGGCCGTTGGACTCCTCACG
TTATCCACACAGGAAATTCTGGCGAAGAACAAAGGCATCGGTATCGAGAC
GTACCACTTCCTCAACGACGGCCTGTGGAAGTCGAAGCCCGTGAAGTAGG
CGAAATAGGAATCTGCACTTGCACTTTTTA

(SEQ ID NO:152)

MFKKFEEKDSISSIQQLKSSVQKGIRAKLLEAYPKLESHIDLILPKKDSY
RIAKCHDHIELLLNGAGDQVFFRHRDGPWMPTLRLLHKFPYFVTMQQVDK
GAIRFVLSGANVMCPGLTSPGACMTPADKDTVVAIMAEGKEHALAVGLLT
LSTQEILAKNKGIGIETYHFLNDGLWKSKPVK

Human homologue of Complete Genome candidate

MCT-1(multiple copies in a T-cell malignancies) (BAA86055), a novel candidate oncogene involved in cell cycle which has a domain similar to cyclin H

```
(SEQ ID NO:153)
    1 gctacctcca actgctgagg aaccggttgc ctaaaaggag ccggcaaaag cgcctacgtg
  61 gagtccagag gagcggaagt agtcagattt gactgagagc cgtaaagcgc ggctggctct
121 cgttttccgg ataacgacta cagctccgac tgtcagtgcc ggccttcctc gtgtgagggg
181 atctgccgga cccctgcaaa ttcaatttct ttcccattcc gggcccttcc ctatcgtcgc
241 ccccttcacc ttggatcatg ttcaagaaat ttgatgaaaa agaaaatgtg tccaactgca
301 tccagttgaa aacttcagtt attaagggta ttaagaatca attgatagag caatttccag
361 gtattgaacc atggcttaat caaatcatgc ctaagaaaga tcctgtcaaa atagtccgat
421 gccatgaaca tatagaaatc cttacagtaa atggagaatt actctttttt agacaaagag
481 aagggccttt ttatccaacc ctaagattac ttcacaaata tccttttatc ctgccacacc
541 agcaggttga taaaggagcc atcaaatttg tactcagtgg agcaaatatc atgtgtccag
601 gcttaacttc tcctggagct aagctttacc ctgctgcagt agataccatt gttgctatca
661 tggcagaagg aaaacagcat gctctatgtg ttggagtcat gaagatgtct gcagaagaca
721 ttgagaaagt caacaaagga attggcattg aaaatatcca ttatttaaat gatgggctgt
781 ggcatatgaa gacatataaa tgagcctcag aaggaatgca cttgggctaa atatggatat
841 tgtgctgtat ctgtgtttgt gtctgtgtgt gacagcatga agataatgcc tgtggttatg
901 ctgaataaat tcaccagatg ctaaaaaaaa aaaaaaaaaa aaa
(SEQ ID NO:154)
    1 mfkkfdeken vsnciqlkts vikgiknqli eqfpgiepwl nqimpkkdpv kivrchehie
  61 iltvngellf frqregpfyp tlrllhkypf ilphqqvdkg aikfvlsgan imcpgltspg
121 aklypaavdt ivaimaegkq halcvgvmkm saediekvnk gigienihyl ndglwhmkty
181 k
```

Putative function
Role in cell cycle progression

### Category 3—Mitotic (Neuroblast) Phenotypes

### Example 10

### Category 3

Line ID—187

Phenotype—lethal phase between pupil and pharate adult (P-pA). High mitotic index, rod-like overcondensed chromosomes, a few circular metaphases, many overcondensed anaphases and telophases, a few tetraploid cells

Annotated Drosophila genome genomic segment containing P element insertion site (and map position)—AE003445 (8B3-7)

P element insertion site—174,362

Annotated Drosophila genome Complete Genome candidate

CG10701 moesin, cytoskeletal binding protein (4 splice variants)

(SEQ ID NO:155)

ACGCCGCATGCACTTTTTTATCTATGATATTATGTTTATTATTTCATTAT
TGAATCGGGAAAACCAAACGTTTTTTTTTTTTTCGTATACAAATCCATTT
GCAGTTTGTAAACTTTAGCGTGCATTCGCATCTAATAGTGATATGTTTTC
GCTTTTCACAGGTGATGAACCAGGACGTGAAGAAGGAGAATCCCTTGCAG
TTTAGGTTCCGTGCCAAATTCTATCCCGAGGATGTGGCCGAGGAGCTGAT
CCAGGACATTACACTGCGTCTGTTCTACCTGCAGGTGAAGAATGCCATAC
TGACCGACGAGATCTATTGTCCGCCAGAGACATCCGTGCTGCTCGCCTCG
TACGCCGTCCAGGCGCGTCATGGTGACCACAATAAGACCACCCACACAGC
CGGCTTTCTGGCCAACGATCGCCTGCTGCCGCAGCGCGTCATCGACCAGC
ACAAGATGTCCAAGGACGAGTGGGAGCAGTCGATTATGACCTGGTGGCAG
GAGCATCGCAGCATGCTGCGCGAGGATGCCATGATGGAGTATCTGAAGAT
CGCCCAAGACCTGGAGATGTACGGCGTTAACTACTTTGAGATCCGCAACA
AGAAGGGCACGGATCTTTGGCTGGGCGTAGACGCACTGGGTCTGAACATT
TACGAGCAGGACGATAGGTTGACGCCGAAAATTGGTTTCCCATGGTCCGA
GATTCGCAACATTTCGTTCTCGGAGAAGAAGTTCATCATCAAGCCGATCG
ACAAGAAGGCTCCGGACTTTATGTTCTTTGCGCCACGTGTCCGCATCAAC
AAGCGCATTCTGGCCCTCTGCATGGGCAACCACGAGCTGTACATGCGTCG
CCGCAAGCCGGACACCATCGATGTGCAGCAGATGAAGGCGCAGGCGCGCG
AGGAGAAGAATGCCAAACAGCAGGAACGTGAGAAGCTGCAGCTGGCGCTG
GCCGCACGCGAACGCGCTGAAAAGAAGCAGCAGGAGTACGAGGATCGGCT
AAAGCAGATGCAGGAGGACATGGAGCGTTCGCAGCGCGATCTGCTTGAGG
CGCAGGACATGATCCGCCGGCTGGAGGAGCAGCTGAAGCAGCTGCAGGCC
GCCAAGGATGAGCTGGAGCTGCGCCAGAAGGAGCTGCAGGCGATGCTGCA
GCGCCTCGAGGAGGCCAAGAATATGGAGGCCGTCGAGAAGCTCAAGCTCG
AGGAGGAGATCATGGCCAAGCAGATGGAGGTGCAGCGCATTCAGGACGAG
GTCAACGCCAAGGATGAGGAGACAAAGCGTCTGCAGGACGAAGTGGAAGA
CGCCCGACGCAAGCAGGTCATTGCGGCTGAAGCCGCTGCCGCTCTGCTGG
CCGCGTCGACAACGCCGCAGCATCACCACGTGGCCGAGGATGAGAACGAG
AACGAGGAGGAGCTGACGAACGGCGATGCCGGTGGCGATGTGTCGCGCGA
CCTGGACACCGACGAGCATATCAAGGACCCCATCGAGGACAGACGCACGC
TGGCCGAGCGCAACGAACGCTTGCACGATCAGCTCAAGGCTCTGAAACAA
GATTTGGCGCAGTCTCGCGACGAGACGAAAGAGACGGCAAACGATAAGAT
TCATCGCGAGAACGTTCGCCAGGGACGTGACAAGTACAAGACGCTCCGCG
AGATTCGTAAGGGCAACACAAAGCGTCGCGTCGATCAGTTTGAGAACATG
TAAAAGCTATCAAAGATCAGAGATCGATAGTGCGCGGGAAAGAGAGAGGG
AGCGGTGAGACTCCAGAAAGA

(SEQ ID NO:156)

MNQDVKKENPLQFRFRAKFYPEDVAEELIQDITLRLFYLQVKNAILTDEI
YCPPETSVLLASYAVQARHGDHNKTTHTAGFLANDRLLPQRVIDQHKMSK
DEWEQSIMTWWQEHRSMLREDAMMEYLKIAQDLEMYGVNYFEIRNKKGTD
LWLGVDALGLNIYEQDDRLTPKIGFPWSEIRNISFSEKKFIIKPIDKKAP
DFMFFAPRVRINKRILALCMGNHELYMRRRKPDTIDVQQMKAQAREEKNA
KQQEREKLQLALAARERAEKKQQEYEDRLKQMQEDMERSQRDLLEAQDMI
RRLEEQLKQLQAAKDELELRQKELQAMLQRLEEAKNMEAVEKLKLEEEIM
AKQMEVQRIQDEVNAKDEETKRLQDEVEDARRKQVIAAEAAAALLAASTT
PQHHHVAEDENENEEELTNGDAGGDVSRDLDTDEHIKDPIEDRRTLAERN
ERLHDQLKALKQDLAQSRDETKETANDKIHRENVRQGRDKYKTLREIRKG
NTKRRVDQFENM

(SEQ ID NO:157)

GACAACAGAATCGAATCGTCGCTTTTCCGCTTTTAACCATCGTGTCGCGT
TGGTCGGTTGGTTTTCCCGCGTAGCTTGTGGCTGCTCAAGAATATATATA
TATTTCCCAGACGGAGATTTGCATTGAAAAGGCGTAATAATTCAAAAGCT
ACTGCGCAATCCGTTTTCGGTGCCCAAAATGGTCGTCGTCTCCGACAGCC
GCGTCCGTTTGCCGCGTTACGGCGGAGTCAGCGTCAAACGGAAAACGCTA
AATGTGCGCGTCACGACAATGGACGCGGAACTGGAGTTCGCCATTCAGTC
GACGACGACGGGCAAGCAATTGTTTGACCAGGTGGTGAAGACGATCGGCC
TGCGAGAGGTTTGGTTCTTTGGACTCCAGTACACCGACTCCAAGGGCGAC
TCCACATGGATCAAGCTGTACAAAAAGCCCGAATCGCCGGCCATAAAGAC
AATAAAATATTTAAAGCGTGTAAAGAAGTATGTGGACAAAAAGACAGCCG
ACAGCAATGGAGTAAATCATTTAGAGACGAGCGAAGAGGATGACGACGCC
GATGATATGACTGGATCAATGCCGTTTTCGACATGGGTGATGAACCAGGA
CGTGAAGAAGGAGAATCCCTTGCAGTTTAGGTTCCGTGCCAAATTCTATC
CCGAGGATGTGGCCGAGGAGCTGATCCAGGACATTACACTGCGTCTGTTC
TACCTGCAGGTGAAGAATGCCATACTGACCGACGAGATCTATTGTCCGCC
AGAGACATCCGTGCTGCTCGCCTCGTACGCCGTCCAGGCGCGTCATGGTG
ACCACAATAAGACCACCCACACAGCCGGCTTTCTGGCCAACGATCGCCTG
CTGCCGCAGCGCGTCATCGACCAGCACAAGATGTCCAAGGACGAGTGGGA
GCAGTCGATTATGACCTGGTGGCAGGAGCATCGCAGCATGCTGCGCGAGG
ATGCCATGATGGAGTATCTGAAGATCGCCCAAGACCTGGAGATGTACGGC
GTTAACTACTTTGAGATCCGCAACAAGAAGGGCACGGATCTTTGGCTGGG
CGTAGACGCACTGGGTCTGAACATTTACGAGCAGGACGATAGGTTGACGC
CGAAAATTGGTTTCCCATGGTCCGAGATTCGCAACATTTCGTTCTCGGAG
AAGAAGTTCATCATCAAGCCGATCGACAAGAAGGCTCCGGACTTTATGTT
CTTTGCGCCACGTGTCCGCATCAACAAGCGCATTCTGGCCCTCTGCATGG
GCAACCACGAGCTGTACATGCGTCGCCGCAAGCCGGACACCATCGATGTG
CAGCAGATGAAGGCGCAGGCGCGCGAGGAGAAGAATGCCAAACAGCAGGA
ACGTGAGAAGCTGCAGCTGGCGCTGGCCGCACGCGAACGCGCTGAAAAGA
AGCAGCAGGAGTACGAGGATCGGCTAAAGCAGATGCAGGAGGACATGGAG
CGTTCGCAGCGCGATCTGCTTGAGGCGCAGGACATGATCCGCCGGCTGGA
GGAGCAGCTGAAGCAGCTGCAGGCCGCCAAGGATGAGCTGGAGCTGCGCC
AGAAGGAGCTGCAGGCGATGCTGCAGCGCCTCGAGGAGGCCAAGAATATG
GAGGCCGTCGAGAAGCTCAAGCTCGAGGAGGAGATCATGGCCAAGCAGAT
GGAGGTGCAGCGCATTCAGGACGAGGTCAACGCCAAGGATGAGGAGACAA
AGCGTCTGCAGGACGAAGTGGAAGACGCCCGACGCAAGCAGGTCATTGCG
GCTGAAGCCGCTGCCGCTCTGCTGGCCGCGTCGACAACGCCGCAGCATCA
CCACGTGGCCGAGGATGAGAACGAGAACGAGGAGGAGCTGACGAACGGCG
ATGCCGGTGGCGATGTGTCGCGCGACCTGGACACCGACGAGCATATCAAG
GACCCCATCGAGGACAGACGCACGCTGGCCGAGCGCAACGAACGCTTGCA
CGATCAGCTCAAGGCTCTGAAACAAGATTTGGCGCAGTCTCGCGACGAGA
CGAAAGAGACGGCAAACGATAAGATTCATCGCGAGAACGTTCGCCAGGGA
CGTGACAAGTACAAGACGCTCCGCGAGATTCGTAAGGGCAACACAAAGCG
TCGCGTCGATCAGTTTGAGAACATGTAAAAGCTATCAAAGATCAGAGATC
GATAGTGCGCGGGAAAGAGAGAGGGAGCGGTGAGACTCCAGAAAGA

(SEQ ID NO:158)

MVVVSDSRVRLPRYGGVSVKRKTLNVRVTTMDAELEFAIQSTTTGKQLFD
QVVKTIGLREVWFFGLQYTDSKGDSTWIKLYKKPESPAIKTIKYLKRVKK
YVDKKTADSNGVNHLETSEEDDDADDMTGSMPFSTWVMNQDVKKENPLQF
RFRAKFYPEDVAEELIQDITLRLFYLQVKNAILTDEIYCPPETSVLLASY
AVQARHGDHNKTTHTAGFLANDRLLPQRVIDQHKMSKDEWEQSIMTWWQE
HRSMLREDAMMEYLKIAQDLEMYGVNYFEIRNKKGTDLWLGVDALGLNIY
EQDDRLTPKIGFPWSEIRNISFSEKKFIIKPIDKKAPDFMFFAPRVRINK
RILALCMGNHELYMRRRKPDTIDVQQMKAQAREEKNAKQQEREKLQLALA
ARERAEKKQQEYEDRLKQMQEDMERSQRDLLEAQDMIRRLEEQLKQLQAA
KDELELRQKELQAMLQRLEEAKNMEAVEKLKLEEEIMAKQMEVQRIQDEV
NAKDEETKRLQDEVEDARRKQVIAAEAAAALLAASTTPQHHHVAEDENEN
EEELTNGDAGGDVSRDLDTDEHIKDPIEDRRTLAERNERLHDQLKALKQD
LAQSRDETKETANDKIHRENVRQGRDKYKTLREIRKGNTKRRVDQFENM

(SEQ ID NO:159)

CCAAAGCGAAACGGGAGCTCTTGGCACGTGCCCTGCTCACATCCCGTTAA
TCCATCGACCCCTAAACAAATCGTGGGGGATTCTCCTCTGCACGCCACCT
TCATCGATGGGTGTCAATTTTTTACTCTTTTTTTTTTCTATTTGGCTTCT
AAATGTGCGCGTCACGACAATGGACGCGGAACTGGAGTTCGCCATTCAGT
CGACGACGACGGGCAAGCAATTGTTTGACCAGGTGGTGAAGACGATCGGC
CTGCGAGAGGTTTGGTTCTTTGGACTCCAGTACACCGACTCCAAGGGCGA
CTCCACATGGATCAAGCTGTACAAAAAGCCCGAATCGCCGGCCATAAAGA
CAATAAAATATTTAAAGCGTGTAAAGAAGTATGTGGACAAAAAGACAGCC
GACAGCAATGGAGTAAATCATTTAGAGACGAGCGAAGAGGATGACGACGC
CGATGATATGACTGGATCAATGCCGTTTTCGACATGGGTGATGAACCAGG
ACGTGAAGAAGGAGAATCCCTTGCAGTTTAGGTTCCGTGCCAAATTCTAT
CCCGAGGATGTGGCCGAGGAGCTGATCCAGGACATTACACTGCGTCTGTT
CTACCTGCAGGTGAAGAATGCCATACTGACCGACGAGATCTATTGTCCGC
CAGAGACATCCGTGCTGCTCGCCTCGTACGCCGTCCAGGCGCGTCATGGT
GACCACAATAAGACCACCCACACAGCCGGCTTTCTGGCCAACGATCGCCT
GCTGCCGCAGCGCGTCATCGACCAGCACAAGATGTCCAAGGACGAGTGGG
AGCAGTCGATTATGACCTGGTGGCAGGAGCATCGCAGCATGCTGCGCGAG
GATGCCATGATGGAGTATCTGAAGATCGCCCAAGACCTGGAGATGTACGG
CGTTAACTACTTTGAGATCCGCAACAAGAAGGGCACGGATCTTTGGCTGG
GCGTAGACGCACTGGGTCTGAACATTTACGAGCAGGACGATAGGTTGACG
CCGAAAATTGGTTTCCCATGGTCCGAGATTCGCAACATTTCGTTCTCGGA
GAAGAAGTTCATCATCAAGCCGATCGACAAGAAGGCTCCGGACTTTATGT
TCTTTGCGCCACGTGTCCGCATCAACAAGCGCATTCTGGCCCTCTGCATG
GGCAACCACGAGCTGTACATGCGTCGCCGCAAGCCGGACACCATCGATGT
GCAGCAGATGAAGGCGCAGGCGCGCGAGGAGAAGAATGCCAAACAGCAGG
AACGTGAGAAGCTGCAGCTGGCGCTGGCCGCACGCGAACGCGCTGAAAAG
AAGCAGCAGGAGTACGAGGATCGGCTAAAGCAGATGCAGGAGGACATGGA
GCGTTCGCAGCGCGATCTGCTTGAGGCGCAGGACATGATCCGCCGGCTGG
AGGAGCAGCTGAAGCAGCTGCAGGCCGCCAAGGATGAGCTGGAGCTGCGC
CAGAAGGAGCTGCAGGCGATGCTGCAGCGCCTCGAGGAGGCCAAGAATAT
GGAGGCCGTCGAGAAGCTCAAGCTCGAGGAGGAGATCATGGCCAAGCAGA
TGGAGGTGCAGCGCATTCAGGACGAGGTCAACGCCAAGGATGAGGAGACA
AAGCGTCTGCAGGACGAAGTGGAAGACGCCCGACGCAAGCAGGTCATTGC
GGCTGAAGCCGCTGCCGCTCTGCTGGCCGCGTCGACAACGCCGCAGCATC
ACCACGTGGCCGAGGATGAGAACGAGAACGAGGAGGAGCTGACGAACGGC
GATGCCGGTGGCGATGTGTCGCGCGACCTGGACACCGACGAGCATATCAA
GGACCCCATCGAGGACAGACGCACGCTGGCCGAGCGCAACGAACGCTTGC
ACGATCAGCTCAAGGCTCTGAAACAAGATTTGGCGCAGTCTCGCGACGAG
ACGAAAGAGACGGCAAACGATAAGATTCATCGCGAGAACGTTCGCCAGGG
ACGTGACAAGTACAAGACGCTCCGCGAGATTCGTAAGGGCAACACAAAGC
GTCGCGTCGATCAGTTTGAGAACATGTAAAAGCTATCAAAGATCAGAGAT
CGATAGTGCGCGGGAAAGAGAGAGGGAGCGGTGAGACTCCAGAAAGA

(SEQ ID NO:160)

MGVNFLLFFFSIWLLNVRVTTMDAELEFAIQSTTTGKQLFDQVVKTIGLR
EVWFFGLQYTDSKGDSTWIKLYKKPESPAIKTIKYLKRVKKYVDKKTADS
NGVNHLETSEEDDDADDMTGSMPFSTWVMNQDVKKENPLQFRFRAKFYPE
DVAEELIQDITLRLFYLQVKNAILTDEIYCPPETSVLLASYAVQARHGDH
NKTTHTAGFLANDRLLPQRVIDQHKMSKDEWEQSIMTWWQEHRSMLREDA
MMEYLKIAQDLEMYGVNYFEIRNKKGTDLWLGVDALGLNIYEQDDRLTPK
IGFPWSELRNISFSEKKFIIKPIDKKAPDFMFFAPRVRINKRILALCMGN
HELYMRRRKPDTIDVQQMKAQAREEKNAKQQEREKLQLALAARERAEKKQ
QEYEDRLKQMQEDMERSQRDLLEAQDMIRRLEEQLKQLQAAKDELELRQK
ELQAMLQRLEEAKNMEAVEKLKLEEEIMAKQMEVQRJQDEVNAKDEETKR
LQDEVEDARRKQVIAAEAAAALLAASTTPQHHHVAEDENENEEELTNGDA
GGDVSRDLDTDEHIKDPIEDRRTLAERNERLHDQLKALKQDLAQSRDETK
ETANDKIHRENVRQGRDKYKTLREIRKGNTKRRVDQFENM

(SEQ ID NO:161)

AAAGCTCACGAAAAACACGCGGCAATTGGATAAGAAACGAAATTGTTGAT
CCAACGCGAGGAAGAAGAAGAATTGTGAAGCAAGAAGAAGCGAAAACAAA
CTGCGATTGCAGCACAAAAACAATAAAGAGTTCAGACGATAATATCCTGG
AAAGAAAACATTTCGTTTCGATAAGTACGACAAGACACGAAACAACAAAA
TGTCTCCAAAAGCGCTAAATGTGCGCGTCACGACAATGGACGCGGAACTG
GAGTTCGCCATTCAGTCGACGACGACGGGCAAGCAATTGTTTGACCAGGT
GGTGAAGACGATCGGCCTGCGAGAGGTTTGGTTCTTTGGACTCCAGTACA
CCGACTCCAAGGGCGACTCCACATGGATCAAGCTGTACAAAAAGGTGATG
AACCAGGACGTGAAGAAGGAGAATCCCTTGCAGTTTAGGTTCCGTGCCAA
ATTCTATCCCGAGGATGTGGCCGAGGAGCTGATCCAGGACATTACACTGC
GTCTGTTCTACCTGCAGGTGAAGAATGCCATACTGACCGACGAGATCTAT
TGTCCGCCAGAGACATCCGTGCTGCTCGCCTCGTACGCCGTCCAGGCGCG
TCATGGTGACCACAATAAGACCACCCACACAGCCGGCTTTCTGGCCAACG
ATCGCCTGCTGCCGCAGCGCGTCATCGACCAGCACAAGATGTCCAAGGAC
GAGTGGGAGCAGTCGATTATGACCTGGTGGCAGGAGCATCGCAGCATGCT
GCGCGAGGATGCCATGATGGAGTATCTGAAGATCGCCCAAGACCTGGAGA
TGTACGGCGTTAACTACTTTGAGATCCGCAACAAGAAGGGCACGGATCTT
TGGCTGGGCGTAGACGCACTGGGTCTGAACATTTACGAGCAGGACGATAG
GTTGACGCCGAAAATTGGTTTCCCATGGTCCGAGATTCGCAACATTTCGT
TCTCGGAGAAGAAGTTCATCATCAAGCCGATCGACAAGAAGGCTCCGGAC
TTTATGTTCTTTGCGCCACGTGTCCGCATCAACAAGCGCATTCTGGCCCT
CTGCATGGGCAACCACGAGCTGTACATGCGTCGCCGCAAGCCGGACACCA
TCGATGTGCAGCAGATGAAGGCGCAGGCGCGCGAGGAGAAGAATGCCAAA
CAGCAGGAACGTGAGAAGCTGCAGCTGGCGCTGGCCGCACGCGAACGCGC
TGAAAAGAAGCAGCAGGAGTACGAGGATCGGCTAAAGCAGATGCAGGAGG
ACATGGAGCGTTCGCAGCGCGATCTGCTTGAGGCGCAGGACATGATCCGC
CGGCTGGAGGAGCAGCTGAAGCAGCTGCAGGCCGCCAAGGATGAGCTGGA
GCTGCGCCAGAAGGAGCTGCAGGCGATGCTGCAGCGCCTCGAGGAGGCCA
AGAATATGGAGGCCGTCGAGAAGCTCAAGCTCGAGGAGGAGATCATGGCC
AAGCAGATGGAGGTGCAGCGCATTCAGGACGAGGTCAACGCCAAGGATGA
GGAGACAAAGCGTCTGCAGGACGAAGTGGAAGACGCCCGACGCAAGCAGG
TCATTGCGGCTGAAGCCGCTGCCGCTCTGCTGGCCGCGTCGACAACGCCG
CAGCATCACCACGTGGCCGAGGATGAGAACGAGAACGAGGAGGAGCTGAC
GAACGGCGATGCCGGTGGCGATGTGTCGCGCGACCTGGACACCGACGAGC
ATATCAAGGACCCCATCGAGGACAGACGCACGCTGGCCGAGCGCAACGAA
CGCTTGCACGATCAGCTCAAGGCTCTGAAACAAGATTTGGCGCAGTCTCG
CGACGAGACGAAAGAGACGGCAAACGATAAGATTCATCGCGAGAACGTTC
GCCAGGGACGTGACAAGTACAAGACGCTCCGCGAGATTCGTAAGGGCAAC
ACAAAGCGTCGCGTCGATCAGTTTGAGAACATGTAAAAGCTATCAAAGAT
CAGAGATCGATAGTGCGCGGGAAAGAGAGAGGGAGCGGTGAGACTCCAGA
AAGA

(SEQ ID NO:162)

MSPKALNVRVTTMDAELEFAIQSTTTGKQLFDQVVKTIGLREVWFFGLQY
TDSKGDSTWIKLYKKVMNQDVKKENPLQFRFRAKFYPEDVAEELIQDITL
RLFYLQVKNAILTDEIYCPPETSVLLASYAVQARHGDHNKTTHTAGFLAN
DRLLPQRVIDQHKMSKDEWEQSIMTWWQEHRSMLREDAMMEYLKIAQDLE
MYGVNYFEIRNKKGTDLWLGVDALGLNIYEQDDRLTPKIGFPWSEIRNIS
FSEKKFIIKPIDKKAPDFMFFAPRVRINKRILALCMGNHELYMRRRKPDT
IDVQQMKAQAREEKNAKQQEREKLQLALAARERAEKKQQEYEDRLKQMQE
DMERSQRDLLEAQDMIRRLEEQLKQLQAAKDELELRQKELQAMLQRLEEA
KNMEAVEKLKLEEEIMAKQMEVQRIQDEVNAKDEETKRLQDEVEDARRKQ
VIAAEAAAALLAASTTPQHHHVAEDENENEEELTNGDAGGDVSRDLDTDE
HIKDPIEDRRTLAERNERLHDQLKALKQDLAQSRDETKETANDKIHRENV
RQGRDKYKTLREIRKGNTKRRVDQFENM

Human homologue of Complete Genome candidate

A41289 human moesin

```
(SEQ ID NO:163)
      1 ggcacgaggc cagccgaatc caagccgtgt gtactgcgtg ctcagcactg cccgacagtc
    61 ctagctaaac ttcgccaact ccgctgcctt tgccgccacc atgcccaaaa cgatcagtgt
  121 gcgtgtgacc accatggatg cagagctgga gtttgccatc cagcccaaca ccaccgggaa
  181 gcagctattt gaccaggtgg tgaaaactat tggcttgagg gaagtttggt tctttggtct
  241 gcagtaccag gacactaaag gtttctccac ctggctgaaa ctcaataaga aggtgactgc
  301 ccaggatgtg cggaaggaaa gccccctgct ctttaagttc cgtgccaagt tctaccctga
  361 ggatgtgtcc gaggaattga ttcaggacat cactcagcgc ctgttctttc tgcaagtgaa
  421 agagggcatt ctcaatgatg atatttactg cccgcctgag accgctgtgc tgctggcctc
  481 gtatgctgtc cagtctaagt atggcgactt caataaggaa gtgcataagt ctggctacct
  541 ggccggagac aagttgctcc cgcagagagt cctggaacag cacaaactca acaaggacca
  601 gtgggaggag cggatccagg tgtggcatga ggaacaccgt ggcatgctca gggaggatgc
  661 tgtcctggaa tatctgaaga ttgctcaaga tctggagatg tatggtgtga actacttcag
  721 catcaagaac aagaaaggct cagagctgtg gctgggggtg gatgccctgg gtctcaacat
  781 ctatgagcag aatgacagac taactcccaa gataggcttc ccctggagtg aaatcaggaa
  841 catctctttc aatgataaga aatttgtcat caagcccatt gacaaaaaag ccccggactt
  901 cgtcttctat gctccccggc tgcggattaa caagcggatc ttggccttgt gcatggggaa
  961 ccatgaacta tacatgcgcc gtcgcaagcc tgataccatt gaggtgcagc agatgaaggc
1021 acaggcccgg gaggagaagc accagaagca gatggagcgt gctatgctgg aaaatgagaa
1081 gaagaagcgt gaaatggcag agaaggagaa agagaagatt gaacgggaga aggaggagct
1141 gatggagagg ctgaagcaga tcgaggaaca gactaagaag gctcagcaag aactggaaga
1201 acagacccgt agggctctgg aacttgagca ggaacggaag cgtgcccaga gcgaggctga
1261 aaagctggcc aaggagcgtc aagaagctga agaggccaag gaggccttgc tgcaggcctc
1321 ccgggaccag aaaaagactc aggaacagct ggccttggaa atggcagagc tgacagctcg
1381 aatctcccag ctggagatgg cccgacagaa gaaggagagt gaggctgtgg agtggcagca
1441 gaaggcccag atggtacagg aagacttgga gaagacccgt gctgagctga agactgccat
1501 gagtacacct catgtggcag agcctgctga gaatgagcag gatgagcagg atgagaatgg
1561 ggcagaggct agtgctgacc tacgggctga tgctatggcc aaggaccgca gtgaggagga
1621 acgtaccact gaggcagaga agaatgagcg tgtgcagaag cacctgaagg ccctcacttc
1681 ggagctggcc aatgccagag atgagtccaa gaagactgcc aatgacatga tccatgctga
1741 gaacatgcga ctgggccgag acaaatacaa gaccctgcgc cagatccggc agggcaacac
1801 caagcagcgc attgacgaat ttgagtctat gtaatgggca cccagcctct agggacccct
1861 cctccctttt tccttgtccc cacactccta cacctaactc acctaactca tactgtgctg
1921 gagccactaa ctagagcagc cctggagtca tgccaagcat ttaatgtagc catgggacca
1981 aacctagccc cttagccccc acccacttcc ctgggcaaat gaatggctca ctatggtgcc
2041 aatggaacct cctttctctt ctctgttcca ttgaatctgt atggctagaa tatcctactt
2101 ctccagccta gaggtacttt ccacttgatt ttgcaaatgc ccttacactt actgttgtcc
2161 tatgggagtc aagtgtggag taggttggaa gctagctccc ctcctctccc ctccactgtc
2221 ttcttcaggt cctgagatta cacggtggag tgtatgcggt ctaggaatga gacaggacct
2281 agatatcttc tccagggatg tcaactgacc taaaatttgc ecteccatec cgtttagagt
2341 tatttaggct ttgtaacgat tgggggaata aaaagatgtt cagtcatttt tgtttctacc
2401 tcccagatcg gatctgttgc aaactcagcc tcaataagcc ttgtcgttga ctttagggac
2461 tcaatttctc cccagggtgg atgggggaaa tggtgccttc aagaccttca ccaaacatac
2521 tagaagggca ttggccattc tattgtggca aggctgagta gaagatccta ccccaattcc
2581 ttgtaggagt ataggccggt ctaaagtgag ctctatgggc agatctaccc cttacttatt
2641 attccagatc tgcagtcact tcgtgggatc tgcccctccc tgcttcaata cccaaatcct
2701 ctccagctat aacagtaggg atgagtaccc aaaagctcag ccagccccat caggactctt
2761 gtgaaaagag aggatatgtt cacacctagc gtcagtattt tccctgctag gggttttagg
2821 tctcttcccc tctcagagct acttgggcca tagctcctgc tccacagcca tcccagcctt
2881 ggcatctaga gcttgatgcc agtaggctca actagggagt gagtgcaaaa agctgagtat
2941 ggtgagagaa gcctgtgccc tgatccaagt ttactcaacc ctctcaggtg accaaaatcc
3001 ccttctcatc actcccctca aagaggtgac tgggccctgc ctctgtttga caaacctcta
3061 acccaggtct tgacaccagc tgttctgtcc cttggagctg taaaccagag agctgctggg
3121 ggattctggc ctagtccctt ccacaccccc accccttgct ctcaacccag gagcatccac
3181 ctccttctct gtctcatgtg tgctcttctt ctttctacag tattatgtac tctactgata
3241 tctaaatatt gatttctgcc ttccttgcta atgcaccatt agaagatatt agtcttgggg
3301 caggatgatt ttggcctcat tactttacca cccccacacc tggaaagcat atactatatt
3361 acaaaatgac attttgccaa aattattaat ataagaagct ttcagtatta gtgatgtcat
3421 ctgtcactat aggtcataca atccattctt aaagtacttg ttatttgttt ttattattac
3481 tgtttgtctt ctccccaggg ttcagtccct caaggggcca tcctgtccca ccatgcagtg
3541 ccccctagct tagagcctcc ctcaattccc cctggccacc accccccact ctgtgcctga
3601 ccttgaggag tcttgtgtgc attgctgtga attagctcac ttggtgatat gtcctatatt
3661 ggctaaattg aaacctggaa ttgtggggca atctattaat agctgcctta aagtcagtaa
3721 cttaccctta gggaggctgg gggaaaaggt tagattttgt attcaggggt tttttgtgta
3781 ctttttgggt ttttaaaaaa ttgtttttgg aggggtttat gctcaatcca tgttctattt
3841 cagtgccaat aaaatttagg tgacttcaaa aaaaaaaaa
(SEQ ID NO:164)
      1 mpktisvrvt tmdaelefai qpnttgkqlf dqvvktiglr evwffglqyq dtkgfstwlk
    61 lnkkvtaqdv rkespllfkf rakfypedvs eeliqditqr lfflqvkegi lnddiycppe
  121 tavllasyav qskygdfnke vhksgylagd kllpqrvleq hklnkdqwee riqvwheehr
  181 gmlredavle ylkiaqdlem ygvnyfsikn kkgselwlgv dalglniyeq ndrltpkigf
  241 pwseirnisf ndkkfvikpi dkkapdfvfy aprlrinkri lalcmgnhel ymrrrkpdti
  301 evqqmkaqar eekhqkqmer amlenekkkr emaekekeki erekeelmer lkqieeqtkk
  361 aqqeleeqtr raleleqerk raqseaekla kerqeaeeak eallqasrdq kktqeqlale
  421 maeltarisq lemarqkkes eavewqqkaq mvqedlektr aelktamstp hvaepaeneq
  481 deqdengaea sadiradama kdrseeertt eaeknervqk hlkaltsela nardeskkta
  541 ndmihaenmr lgrdkyktlr qirqgntkqr idefesm
```

Putative function
Cytoskeletal binding protein linking to plama membrane, involved in cytokinesis and cell shape

### Example 11

### Category 3

Line ID—226

Phenotype—Lethal phase pharate adult. High mitotic index, rod-like overcondensed chromosomes, lagging chromosomes and bridges in anaphase, highly condensed

Annotated Drosophila genome genomic segment containing P element insertion site (and map position)—AE003423 (2F1-2)

P element insertion site—226,527

Annotated Drosophila genome Complete Genome candidate

CG2865—EG:25E8.4

(SEQ ID NO:165)

AGAAAACCACATAAACAAGCCAGCAAACAAGGCACACACTTGCTTGAAAA
ACGCACAATGACCTTGCCCACAAACACACACGCATCTGCAAACGACGGCG
GCAGCGGCAACAACAACCACAGCAATATCAGCAGTAACAACAGCAGCAGC
AGCGACGAAGACTCAGACATGTTTGGACCACCCCGCTGCTCCCCGCCCAT
CGGCTATCACCATCACCGTTCCCGTGTGCCCATGATCTCGCCAAAGCTGC
GGCAGCGCGAGGAGCGCAAGCGGATCCTCCAGCTCTGCGCCCACAAGATG
GAGAGGATCAAGGACTCGGAGGCGAACCTGCGGCGCAGCGTCTGCATCAA
CAACACCTACTGCCGCCTGAATGACGAACTGCGGCGCGAGAAGCAGATGC
GCTACCTCCAGAATCTGCCCAGAACCAGCGACAGCGGCGCAAGCACCGAA
CTGGCGCGTGAGAATCTCTTCCAGCCGAACATGGACGACGCCAAGCCGGC
CGGCAATAGCACTAGCAATAATATCAACGCCAACGGCAAGCCTTCATCCT
CTTTTGGCGATGCCTTTGGCTCCTCAAACGGATCATCGTCGGGTCGCGGC
GGAATTTGCTCCCTGGAGAATCAACCGCCCGAGCGTCAGCAGTTGGGGAC
GCCCGCTGGTGCCTCCGCTCCCGAGGCGGCCAATTCGGCGCCCCTTTCCG
TTTCGGGCTCGGCATCGGAACGCGTGAATAACCGAAAACGCCACCTGTCC
AGCTGCAACTTGGTCAACGATCTGGAAATACTGGACAGGGAGCTGAGCGC
CATCAATGCACCCATGCTGCTAATCGATCCAGAGATTACCCAAGGAGCCG
AACAGCTGGAGAAGGCCGCCTTGTCCGCCAGCAGGAAGAGATTGAGGAGC
AATAGCGGCAGCGAGGACGAAAGTGATCGCCTGGTGCGCGAGGCTCTGTC
CCAGTTCTACATACCGCCACAGCGCCTCATCTCCGCCATTGAGGAGTGTC
CCCTGGATGTGGTTGGCTTGGGTATGGGAATGAATGTGAATGTGAATGTG
GGAGGAATTAGTGGAATCGGTGGCATCGGAGGAGCTGCAGGCGCTGGCGT
CGAAATGCCCGGAGGCAAACGGATGAAGCTGAATGACCATCACCATCTCA
ATCACCATCACCATTTGCACCATCATCTGGAGCTGGTCGATTTCGACATG
AACCAAAACCAAAAGGATTTCGAGGTGATCATGGACGCCTTGAGGCTGGG
AACGGCGACACCGCCGAGCGGCGCCAGCAGCGATTCTTGCGGACAGGCGG
CGATGATGAGCGAGTCGGCCAGCGTGTTCCACAATCTGGTGGTCACCTCG
TTGGAGACATGA

(SEQ ID NO:166)

MTLPTNTHASANDGGSGNNNHSNISSNNSSSSDEDSDMFGPPRCSPPIGY
HHHRSRVPMISPKLRQREERKRILQLCAHKMERIKDSEANLRRSVCINNT
YCRLNDELRREKQMRYLQNLPRTSDSGASTELARENLFQPNMDDAKPAGN
STSNNINANGKPSSSFGDAFGSSNGSSSGRGGICSLENQPPERQQLGTPA
GASAPEAANSAPLSVSGSASERVNNRKRHLSSCNLVNDLEILDRELSAIN
APMLLIDPEITQGAEQLEKAALSASRKRLRSNSGSEDESDRLVREALSQF
YLPPQRLISAIEECPLDVVGLGMGMNVNVNVGGISGIGGIGGAAGAGVEM
PGGKRMKLNDHHHLNHHHHLHHHLELVDFDMNQNQKDFEVIMDALRLGTA
TPPSGASSDSCGQAAMMSESASVFHNLVVTSLET

Human homologue of Complete Genome candidate
CG2865—none

Putative function
Putative phosphatidylinositol 3-kinase

### Example 12

### Category 3

Line ID—269

Phenotype—Lethal phase pupal—pharate adult. High mitotic index, colchicines-type overcondensation, high frequency of polyploids

Annotated Drosophila genome genomic segment containing P element insertion site (and map position)—AE003568 (19F)

P element insertion site—197,805

Annotated Drosophila genome Complete Genome candidate

CG1696—novel protein

(SEQ ID NO:167)

AAAACTCATCGATGCTGCGAAAGTGCGATAGTATCGAATAAACATGAGTG
TGTGCATGAGTGTGGGAATTTATTAAACAAAAACGAAACGCGGACAAACT
ATATTTATGTAATAAACACTAAGCCGCAGCGCCAACGAGTAATGAACAGT
CCACGGCCAGGTCGTACTATTCAGGCGAACGCACCTCGCAATCGACTGCA
ATCAAAGTGCAATAGCTCAATCAATTGATTCGTTTTGCTCAACCAAAAAC
AAAATCTATTCCCAAATCGGTGCGATAGTTGCCAAAATATAAAAACTACA
CTACGCTAAAAAAAAAACAATACACTCACACACTGGCGTACAAGACAACA
AAAGAGAAGAAGAAGAGCAGACGCCAGATATAAAAAGCCCCCAAAAGAAT
TGGAAATAAGACCATACCCCTCCTTCTCCCTTGAAAAGGGACCTTAAAAC
TAGGCGACACCGAATAATTGAACTCAAGTAAAAAACCGGGAAAAGAGAAA
AACACTTTCAACAAAATATCTAGAAGCCTTGTTATCGATTTTGTTCCGGG
TTTTTTTTGTGTGAGTGTGTGTTGTGTGAAGCGCGCCCGCGGGTGTGTGG
GTGAGTGTGCGTGTGGCTCTCGGCGCGTTATCAAAAACAACAACAATTCG
TTGCAAAAGAAAAAATAAAGTAGAGGAGGCGGAAGAAGAAGAGGAATCTG
CTCGCACCGCGGTCAATCGCGGATCGTGGTCGATTTATCGAATTAATCGC
CCCGAACAAAAAAAACACCGTACAAGGACTTGCACTATTTCCAATGATTT
CGCTGCTGCAAATGAAATTCCGTGCGCTTTTGTTGTTGCTATCAAAAGTA
TGGACATGCATTTGTTTCATGTTCAATCGCCAAGTGCGAGCTTTTATCCA
GTATCAACCGGTTAAATACGAACTCTTCCCGTTGTCACCCGTCTCGCGGC
ACCGCCTGAGCCTGGTGCAGCGCAAGACCCTCGTTCTGGACCTGGACGAA
ACGCTAATCCACTCCCATCACAATGCGATGCCCCGGAATACGGTGAAGCC
GGGCACGCCGCACGATTTCACTGTCAAAGTGACCATCGATCGGAATCCAG
TGCGCTTTTTCGTGCACAAGCGACCGCATGTGGACTACTTCCTGGACGTG
GTCTCGCAGTGGTACGATCTGGTGGTCTTCACGGCCAGCATGGAGATTTA
CGGAGCGGCGGTGGCAGACAAGCTGGACAACGGACGAAACATCCTCCGGA
GGCGATACTACAGACAGCACTGCACGCCCGACTACGGATCCTACACCAAA
GACCTGTCGGCCATCTGCAGTGACCTAAATAGGATATTTATCATCGACAA
TTCGCCCGGCGCCTATCGCTGTTTTCCCAACAACGCCATACCCATCAAGA
GTTGGTTCTCGGACCCGATGGACACGGCGCTGCTGTCGCTGCTGCCCATG
CTGGATGCGCTGAGGTTCACGAACGACGTGAGATCGGTGCTGTCGAGGAA
CTTGCACCTGCACCGCCTCTGGTAGCAGGTGGGCCGCCTGTCGCTAGTTT
AGTTTA

(SEQ ID NO:168)

MISLLQMKFRALLLLLSKVWTCICFMFNRQVRAFIQYQPVKYELFPLSPV
SRHRLSLVQRKTLVLDLDETLIHSHHNAMPRNTVKPGTPHDFTVKVTIDR
NPVRFFVHKRPHVDYFLDVVSQWYDLVVFTASMEIYGAAVADKLDNGRNI
LRRRYYRQHCTPDYGSYTKDLSAICSDLNRIFIIDNSPGAYRCFPNAIP
IKSWFSDPMDTALLSLLPMLDALRFTNDVRSVLSRNLHLHRLW

Human homologue of Complete Genome candidate

NP_{—}056158 hypothetical protein

| | | |
|---|---|---|
| 1 | gccggggccg gcggtgccgg ggtcatcggg atgatgcgga cgcagtgtct gctggggctg | (SEQ ID NO:169) |
| 61 | cgcgcgttcg tggccttcgc cgccaagctc tggagcttct tcatttacct tttgcggagg | |
| 121 | cagatccgca cggtaattca gtaccaaact gttcgatatg atatcctccc cttatctcct | |
| 181 | gtgtcccgga atcggctagc ccaggtgaag aggaagatcc tggtgctgga tctggatgag | |
| 241 | acacttattc actcccacca tgatggggtc ctgaggccca cagtccggcc tggtacgcct | |
| 301 | cctgacttca tcctcaaggt ggtaatagac aaacatcctg tccggttttt tgtacataag | |
| 361 | aggccccatg tggatttctt cctggaagtg gtgagccagt ggtacgagct ggtggtgttt | |
| 421 | acagcaagca tggagatcta tggctctgct gtggcagata aactggacaa tagcagaagc | |
| 481 | attcttaaga ggagatatta cagacagcac tgcactttgg agttgggcag ctacatcaag | |
| 541 | gacctctctg tggtccacag tgacctctcc agcattgtga tcctggataa ctccccaggg | |
| 601 | gcttacagga gccatccaga caatgccatc cccatcaaat cctggttcag tgaccccagc | |
| 661 | gacacagccc ttctcaacct gctcccaatg ctggatgccc tcaggttcac cgctgatgtt | |
| 721 | cgttccgtgc tgagccgaaa ccttcaccaa catcggctct ggtgacagct gctccccctc | |
| 781 | cacctgagtt ggggtggggg ggaaagggag ggcgagccct tgggatgccg tctgatgccc | |
| 841 | tgtccaatgt gaggactgcc tgggcagggt ctgcccctcc cacccctctc tgccctggga | |
| 901 | gccctacact ccacttggag tctggatgga cacatgggcc aggggctctg aagcagcctc | |
| 961 | actcttaact tcgtgttcac actccatgga aaccccagac tgggacacag gcggaagcct | |
| 1021 | aggagagccg aatcagtgtt tgtgaagagg caggactggc cagagtgaca gacatacggt | |
| 1081 | gatccaggag gctcaaagag aagccaagtc agctttgttg tgatttgatt ttttttaaaa | |
| 1141 | aactcttgta caaaactgat ctaattcttc actcctgctc caagggctgg gctgtgggtg | |
| 1201 | ggatactggg attttgggcc actggatttt ccctaaattt gtcccccctt tactctccct | |
| 1261 | ctatttttct ctccttagac tccctcagac ctgtaaccag ctttgtgtct tttttccttt | |
| 1321 | tctctctttt aaaccatgca ttataacttt gaaacc | |
| 1 | mmrtqcllgl rafvafaakl wsffiyllrr qirtviqyqt vrydilplsp vsrnrlaqvk | (SEQ ID NO:170) |
| 61 | rkilvldlde tlihshhdgv lrptvrpgtp pdfilkvvid khpvrffvhk rphvdfflev | |
| 121 | vsqwyelvvf tasmeiygsa vadkldnsrs ilkrryyrqh ctlelgsyik dlsvvhsdls | |
| 181 | sivildnspg ayrshpdnai pikswfsdps dtallnllpm ldalrftadv rsvlsrnlhq | |
| 241 | hrlw | |

Putative function
unknown

### Example 13

### Category 3

Line ID—291

Phenotype—Lethal phase pupal—pharate adult. High mitotic index, colchicines-type overcondensed chromosomes, many strongly stained nuclei

Annotated Drosophila genome genomic segment containing P element insertion site (and map position)—AE003427 (3D5)

P element insertion site—131,166

Annotated Drosophila genome Complete Genome candidate

CG10798—dm diminutive, dMyc1

(SEQ ID NO:171)

GTCGCGTGTTCAGTTCACCGCGGGTAATTCAGAGAATCGCTTTGTGGATT
GGATTTTTGCCTGTTTTCCGCCCGATACAAAAAAAAAAAACCAAACGCTA
TATAAATAGTTCTGTAGTAAAACCTGAAGCAACACGTTTTAAAATATACA
ACTACTACTAACAACTGTCACAGCCAAGTTACAAAAGTGCTAAATCCCAG
AAATAACCTAAGAGCCGACTTAAAACCGCGCAAATACATAAAAAAAAATC
TTCTCCAAAGCAGAAACAAAAACTTGTGAAAAACTAGAATTAAAAAAAGA
TTTTTTAAAAAAAATCAGCTAGTGCAAAATAAACGGGAAGAATTTTTTTT
TGTGTCCCTTTTTTTGGTGTTTTTTCTCCGTCTTTCCCCTTCTTTGACGC
AAAAAAAAAAGTGCCCAACTTGCTGGCGGCACGGGAACGGGATAGAAATA
GATATAGCCGAAAGCGACTGGAAAGCAAAGGAAGCTAACTAAATTGGATT
ACAATCAATTAAATAGAGACGGATACGGAAACTATGTTCAGCGAGACAGG
CATATAACTCAGGAACTTAAGATATATAGAAAGAAAAAAAAACCCAGACA
ACATAATCGCAATGGCCCTTTACCGCTCTGATCCGTATTCCATAATGGAC
GACCAACTTTTTTCAAATATTTCAATATTCGATATGGATAATGATCTGTA
CGATATGGACAAACTCCTTTCGTCGTCCACCATTCAGAGTGATCTCGAGA
AGATCGAGGACATGGAAAGTGTATTTCAAGACTATGACTTAGAGGAGGAT
ATGAAGCCAGAGATCCGCAACATCGACTGCATGTGGCCGGCGATGTCCAG
CTGTTTGACCAGCGGTAACGGTAATGGAATAGAGAGCGGAAACAGTGCAG
CCTCGTCGTACAGCGAAACCGGTGCCGTATCCCTGGCGATGGTTTCCGGC
TCTACGAATCTCTACAGCGCGTATCAACGATCGCAGACGACAGATAACAC
CCAGTCAAATCAACAGCATGTCGTCAACAGTGCCGAGAACATGCCGGTGA
TCATCAAGAAGGAGCTCGCAGATCTGGACTACACGGTCTGTCAGAAGCGC
CTCCGTTTGAGCGGCGGTGACAAGAAGTCACAGATCCAGGACGAGGTCCA
TTTAATACCGCCCGGCGGAAGTTTGCTCCGCAAGCGGAACAACCAGGACA
TTATCCGCAAATCGGGCGAATTGAGCGGCAGCGATAGCATAAAATACCAG
AGACCAGACACACCTCACAGTCTTACCGACGAGGTGGCCGCCTCAGAGTT
TAGACATAACGTCGACTTGCGTGCCTGCGTGATGGGCAGCAATAATATCT
CGCTGACCGGCAATGATAGCGATGTCAACTACATTAAGCAAATCAGCAGG
GAGCTTCAGAATACCGGCAAGGATCCGTTGCCGGTGCGTTACATCCCGCC
GATCAACGATGTCCTCGATGTGCTCAACCAGCATTCCAATTCGACGGGTG
GCCAACAGCAGTTGAACCAACAGCAACTGGACGAGCAACAACAGGCCATC
GATATAGCCACTGGACGCAACACAGTGGATTCTCCGCCGACGACCGGCTC
TGATAGTGACTCCGATGACGGTGAACCCCTCAACTTTGACCTGCGCCATC
ATCGCACTAGCAAAAGCGGCAGCAATGCCAGCATCACCACCAACAACAAC
AACAGCAACAACAAAAACAACAAATTGAAGAACAACAGCAACGGCATGCT
GCACATGATGCACATCACCGATCACAGCTACACGCGCTGCAACGATATGG
TGGACGATGGTCCCAATTTGGAGACCCCCTCAGATTCCGATGAGGAAATC
GATGTCGTTTCATATACGGACAAGAAGCTACCCACAAATCCCTCGTGCCA
CTTGATGGGCGCCCTACAGTTCCAGATGGCCCATAAGATCTCGATTGATC
ACATGAAGCAAAAACCGCGCTACAATAACTTCAATCTGCCGTACACACCG
GCCAGCAGCAGTCCAGTGAAATCGGTGGCCAACTCGCGTTATCCATCACC
GTCGAGCACACCGTATCAGAACTGCTCCTCCGCTTCGCCGTCCTACTCGC
CGCTATCCGTGGACTCTTCAAATGTCAGCTCGAGCAGCTCCAGTTCCAGT
TCGCAGTCAAGCTTCACCACCTCCAGTTCGAACAAGGGACGCAAACGATC
CAGTCTGAAGGATCCAGGCTTGTTGATCTCCTCCAGCAGCGTTTATCTGC
CGGGAGTCAATAACAAAGTGACGCATAGCTCCATGATGAGCAAAAAGAGT
CGTGGCAAGAAGGTGGTTGGCACCTCGTCTGGCAATACATCTCCGATATC
GTCTGGCCAGGATGTGGATGCCATGGATCGTAATTGGCAGCGGCGCAGTG
GTGGAATTGCCACTAGCACAAGCTCCAACAGCAGTGTCCATCGGAAGGAC
TTTGTTTTGGGCTTTGATGAGGCCGATACGATCGAGAAGCGCAATCAGCA
CAATGATATGGAGCGTCAGCGACGCATTGGACTCAAGAACCTCTTTGAGG
CTCTAAAGAAACAGATTCCCACAATTAGGGACAAGGAGCGGGCTCCCAAG
GTAAATATCCTGCGAGAGGCGGCCAAGCTATGCATCCAGCTGACCCAGGA
GGAGAAGGAGCTTAGTATGCAGCGCCAGCTTTTGTCGCTGCAGCTGAAGC
AACGTCAGGACACTCTGGCCAGTTACCAAATGGAGTTGAACGAATCGCGC
TCGGTTAGTGGATAGTGTTGTCTCATACTATCGGCTTAAAGCGGCGGCGT
AGGGCTAGGATAACCCCCAATGTATATGCAAGATTTGTATATCCTCCTAC
TTTTTTTTTTTTGCAATTTACTTTGATTTAGCTTCGATCCTTTCTTGACA
TTAAGCCCTAAATATGATTTTTTTCTGGAGAACTTCAATATCAGTTAGTA
GGTTATGTTTAACGATTTGCTTGCGCTTTTTCCGCTTTTTTTTTTGTTTT
TTTACCATACCATACCATAC

(SEQ ID NO:172)

MDDQLFSNISIFDMDNDLYDMDKLLSSSTIQSDLEKIEDMESVFQDYDLE
EDMKPEIRNIDCMWPAMSSCLTSGNGNGIESGNSAASSYSETGAVSLAMV
SGSTNLYSAYQRSQTTDNTQSNQQHVVNSAENMPVIIKKELADLDYTVCQ
KRLRLSGGDKKSQIQDEVHLIPPGGSLLRKRNNQDIIRKSGELSGSDSIK
YQRPDTPHSLTDEVAASEFRHNVDLRACVMGSNNISLTGNDSDVNYIKQI
SRELQNTGKDPLPVRYIPPINDVLDVLNQHSNSTGGQQQLNQQQLDEQQQ
AIDIATGRNTVDSPPTTGSDSDSDDGEPLNFDLRHHRTSKSGSNASITTN
NNNSNNKNNKLKNNSNGMLHMMHITDHSYTRCNDMVDDGPNLETPSDSDE
EIDVVSYTDKKLPTNPSCHLMGALQFQMAHKISIDHMKQKPRYNNFNLPY
TPASSSPVKSVANSRYPSPSSTPYQNCSSASPSYSPLSVDSSNVSSSSSS
SSSQSSFTTSSSNKGRKRSSLKDPGLLISSSSVYLPGVNNKVTHSSMMSK
KSRGKKVVGTSSGNTSPISSGQDVDAMDRNWQRRSGGIATSTSSNSSVHR
KDFVLGFDEADTIEKRNQHNDMERQRRIGLKNLFEALKKQIPTIRDKERA
PKVNILREAAKLCIQLTQEEKELSMQRQLLSLQLKQRQDTLASYQMELNE
SRSVSG

Human homologue of Complete Genome candidate

CAA23831 c-myc oncogene

| | | |
|---|---|---|
| 1 | ctgctcgcgg ccgccaccgc cgggccccgg ccgtccctgg ctcccctcct gcctcgagaa | (SEQ ID NO:173) |
| 61 | gggcagggct tctcagaggc ttggcgggaa aaaagaacgg agggagggat cgcgctgagt | |
| 121 | ataaaagccg gttttcgggg ctttatctaa ctcgctgtag taattccagc gagaggcaga | |
| 181 | gggagcgagc gggcggccgg ctagggtgga agagccgggc gagcagagct gcgctgcggg | |
| 241 | cgtcctggga agggagatcc ggagcgaata gggggcttcg cctctggccc agccctcccg | |
| 301 | cttgatcccc caggccagcg gtccgcaacc cttgccgcat ccacgaaact ttgcccatag | |
| 361 | cagcgggcgg gcactttgca ctggaactta caacacccga gcaaggacgc gactctcccg | |
| 421 | acgcggggag gctattctgc ccatttgggg acacttcccc gccgctgcca ggacccgctt | |
| 481 | ctctgaaagg ctctccttgc agctgcttag acgctggatt tttttcgggt agtggaaaac | |
| 541 | cagcagcctc ccgcgacgat gcccctcaac gttagcttca ccaacaggaa ctatgacctc | |
| 601 | gactacgact cggtgcagcc gtatttctac tgcgacgagg aggagaactt ctaccagcag | |
| 661 | cagcagcaga gcgagctgca gcccccggcg cccagcgagg atatctggaa gaaattcgag | |
| 721 | ctgctgccca ccccgcccct gtcccctagc cgccgctccg ggctctgctc gccctcctac | |
| 781 | gttgcggtca cacccttctc ccttcgggga gacaacgacg gcggtggcgg gagcttctcc | |
| 841 | acggccgacc agctggagat ggtgaccgag ctgctgggag gagacatggt gaaccagagt | |
| 901 | ttcatctgcg acccggacga cgagaccttc atcaaaaaca tcatcatcca ggactgtatg | |
| 961 | tggagcggct tctcggccgc cgccaagctc gtctcagaga agctggcctc ctaccaggct | |
| 1021 | gcgcgcaaag acagcggcag cccgaacccc gcccgcggcc acagcgtctg ctccacctcc | |
| 1081 | agcttgtacc tgcaggatct gagcgccgcc gcctcagagt gcatcgaccc ctcggtggtc | |
| 1141 | ttcccctacc ctctcaacga cagcagctcg cccaagtcct gcgcctcgca agactccagc | |
| 1201 | gccttctctc cgtcctcgga ttctctgctc tcctcgacgg agtcctcccc gcagggcagc | |
| 1261 | cccgagcccc tggtgctcca tgaggagaca ccgcccacca ccagcagcga ctctgaggag | |
| 1321 | gaacaagaag atgaggaaga aatcgatgtt gtttctgtgg aaaagaggca ggctcctggc | |
| 1381 | aaaaggtcag agtctggatc accttctgct ggaggccaca gcaaacctcc tcacagccca | |
| 1441 | ctggtcctca agaggtgcca cgtctccaca catcagcaca actacgcagc gcctccctcc | |
| 1501 | actcggaagg actatcctgc tgccaagagg gtcaagttgg acagtgtcag agtcctgaga | |
| 1561 | cagatcagca acaaccgaaa atgcaccagc cccaggtcct cggacaccga ggagaatgtc | |
| 1621 | aagaggcgaa cacacaacgt cttggagcgc cagaggagga acgagctaaa acggagcttt | |
| 1681 | tttgccctgc gtgaccagat cccggagttg gaaaacaatg aaaaggcccc caaggtagtt | |
| 1741 | atccttaaaa aagccacagc atacatcctg tccgtccaag cagaggagca aaagctcatt | |
| 1801 | tctgaagagg acttgttgcg gaaacgacga gaacagttga aacacaaact tgaacagcta | |
| 1861 | cggaactctt gtgcgtaagg aaaagtaagg aaaacgattc cttctaacag aaatgtcctg | |
| 1921 | agcaatcacc tatgaacttg tttcaaatgc atgatcaaat gcaacctcac aaccttggct | |
| 1981 | gagtcttgag actgaaagat ttagccataa tgtaaactgc ctcaaattgg actttgggca | |
| 2041 | taaaagaact tttttatgct taccatcttt tttttttctt taacagattt gtatttaaga | |
| 2101 | attgttttta aaaaatttta a | |
| 1 | mplnvsftnr nydldydsvq pyfycdeeen fyqqqqqsel qppapsediw kkfellptpp | (SEQ ID NO:174) |
| 61 | lspsrrsglc spsyvavtpf slrgdndggg gsfstadqle mvtellggdm vnqsficdpd | |
| 121 | detfikniii qdcmwsgfsa aaklvsekla syqaarkdsg spnparghsv cstsslylqd | |
| 181 | lsaaasecid psvvfpypln dssspkscas qdssafspss dsllsstess pqgspeplvl | |
| 241 | heetppttss dseeeqedee eidvvsvekr qapgkrsesg spsagghskp phsplvlkrc | |
| 301 | hvsthqhnya appstrkdyp aakrvkldsv rvlrqisnnr kctsprssdt eenvkrrthn | |
| 361 | vlerqrrnel krsffalrdq ipelenneka pkvvilkkat ayilsvqaee qkliseedll | |
| 421 | rkrreqlkhk leqlrnsca | |

Putative function
C-myc oncogene, transcription factor

### Example 14

### Category 3

Line ID—316

Phenotype—Lethal phase larval stage 3
Pre-pupal-pupal. Small optic lobes, missing or small imaginal discs, badly defined chromosomes.

Annotated Drosophila genome genomic segment containing P element insertion site (and map position)—AE003506 (16B-C)

P element insertion site—27,868

Annotated Drosophila genome Complete Genome candidate

CG8465—novel protein (3 splice variants)

(SEQ ID NO:175)

TGACAGTCCGCCTCTAATTTAATTTCGTTTGTGCACATTTTGTTTGAAAG
ACGCTTAAGATTATTGGGTTTTGTTTCATGTATTGTGCCCTTTGTGCTAA
AAGTGCATCCGCCATTTTACGCAGAGATGTCGACCTATTTCGGGGTCTAT
ATCCCGACCTCCAAAGCGGGCTGTTTTGAGGGATCGGTGTCGCAGTGCAT
CGGCTCCATAGCCGCGGTGAACATAAAGCCATCCAATCCGGCGTCTGGAT
CGGCATCAGTAGCATCGGGATCGCCATCCGGCTCGGCGGCATCCGTGCAA
ACGGGCAACGCAGACGATGGCAGTGCTGCCACCAAGTACGAGGATCCCGA
CTATCCACCGGACTCGCCACTGTGGCTGATCTTCACGGAGAAATCCAAGG
CGCTGGACATCCTGCGACACTACAAGGAGGCGCGCCTCCGCGAGTTTCCC
AATCTGGAGCAGGCGGAGAGTTACGTTCAGTTTGGGTTCGAGAGCATCGA
GGCGCTCAAGAGATTTTGCAAGGCAAAGCCCGAAAGCAAGCCCATTCCGA
TAATCAGCGGTAGCGGTTACAAGAGCTCACCGACCTCGACGGACAATTCG
TGCTCCTCCTCGCCGACGGGTAACGGCAGTGGCTTCATCATTCCCCTGGG
AAGCAATTCCTCAATGTCGAATTTACTGCTCAGTGACTCACCGACTTCCT
CGCCGAGCAGCTCCAGCAACGTCATTGCCAATGGGCGACAGCAGCAGATG
CAGCAGCAACAGCAGCAGCAGCCGCAGCAGCCGGATGTGTCCGGAGAAGG
CCCTCCTTTCCGGGCGCCCACCAAACAGGAACTGGTAGAGTTTCGCAAGC
AAATCGAAGGTGGTCACATAGACCGGGTGAAGAGGATTATATGGGAGAAT
CCACGATTTTTGATCAGCAGCGGTGATACGCCCACCAGTTTGAAGGAGGG
CTGTCGCTATAATGCCATGCACATCTGCGCCCAGGTCAATAAGGCCAGGA
TCGCTCAGTTGCTGTTAAAGACCATTTCGGATCGGGAGTTCACTCAGCTT
TACGTTGGCAAGAAGGGCAGTGGCAAGATGTGTGCTGCCCTCAACATCAG
TCTCCTGGACTATTACCTGAACATGCCGGACAAGGGGCGCGGCGAAACAC
CGCTCCACTTTGCCGCAAAGAACGGTCATGTGGCCATGGTCGAGGTTCTC
GTTTCCTATCCGGAGTGCAAATCGCTGCGGAATCATGAGGGCAAGGAGCC
CAAGGAAATCATCTGCCTGCGTAATGCTAATGCTACACATGTGACCATCA
AGAAGCTGGAGCTGCTCTTGTACGATCCGCATTTTGTGCCCGTACTAAGA
TCCCAGTCAAATACACTGCCGCCAAAAGTGGGTCAACCGTTCTCGCCCAA
AGATCCACCGAACCTGCAACACAAAGCGGACGATTACGAGGGCCTCAGCG
TGGACCTGGCAATCAGTGCGCTGGCGGGACCCATGTCCCGCGAAAAGGCC
ATGAACTTCTATCGCCGTTGGAAGACACCACCGCGGGTCAGCAACAATGT
GATGTCGCCGCTGGCTGGTTCACCATTTAGCTCGCCGGTGAAAGTAACCC
CAAGCAAGTCGATCTTTGACCGAAGTGCTGGAAACTCGAGTCCAGTCCAC
TCAGGACGCAGAGTGCTCTTTAGTCCATTGGCGGAGGCGACCAGCTCACC
AAAACCGACGAAAAACGTGCCCAATGGCACCAATGAGTGCGAGCACAACA
ATAATAATGTGAAGCCAGTGTATCCGTTGGAGTTCCCGGCGACACCCATT
CGAAAAATGAAACCGGATTTATTCATGGCCTATCGCAATAACAATAGCTT
TGATTCGCCATCTTTGGCCGATGACTCCCAAATCCTGGACATGAGCCTAA
GCCGCAGCCTGAATGCGTCGCTAAATGACAGCTTCCGTGAGCGGCACATC
AAGAACACTGATATCGAGAAGGGTCTGGAGGTGGTCGGCCGCCAACTGGC
ACGACAGGAGCAGTTAGAGTGGCGCGAGTACTGGGATTTTCTCGATTCAT
TTTTGGACATTGGTACGACCGAAGGCCTGGCCCGTCTTGAAGCGTATTTC
CTGGAAAAGACCGAACAGCAGGCGGATAAATCAGAAACGGTCTGGAACTT
TGCCCATCTGCATCAGTATTTCGATTCGATGGCCGGCGAGCAACAGCAGC
AACTCCGAAAGGATAAAAATGAGGCTGCGGGAGCAACTTCGCCATCCGCC
GGAGTCATGACTCCGTACACATGCGTAGAGAAGTCGCTGCAAGTGTTCGC
CAAGCGCATCACTAAAACGTTGATCAACAAAATCGGCAACATGGTGTCCA
TCAACGACACGCTGCTCTGTGAGCTCAAAAGACTGAAATCGCTGATTGTC
AGCTTCAAGGATGATGCCCGCTTCATTAGCGTGGACTTTAGCAAGGTGCA
TTCACGTATCGCCCACCTGGTGGCCAGCTATGTGACCCACTCGCAGGAGG
TCAGCGTAGCCATGCGTCTACAATTGTTGCAGATGCTCCGAAGTTTGCGG
CAACTGCTGGCCGACGAGCGTGGTCGAGAACAGCATTTGGGCTGCGTGTG
CGCTAGTCTATTGCTGATGCTGGAACAGGCGCCGACATCCGCCGTGCATC
TACCAGACACTCTGAAGACCGAGGAGCTATGTTGCGCCGCCTGGGAGACG
GAGCAGTGTTGCGCCTGTCTGTGGGACGCAAATCTCAGCCGTAAGACCAG
TCGTCGAAAGCGCACTAAGTCGCTGCGGGCAGCTGCTGTTGTTCAGTCTC
AGGGTCAGCTTCAGGATACTTCGGGATCGACAGGGTCGTCCGCCTTGCAC
GCTTCGCTTGGTGTGGGATCGACCAGTTTGGGAGCATCGAGGGTCGTGGC
GTCCGCTTCGAAAGATGCTTGGCGCCGTCAACAAAGCGACGACGAGGACT
ACGACAGCGATGAGCAAGTAATCTTTTTCGACTGCACTAATGTTACGCTG
CCTTATGGAAGCAGCAGCGAGGACGAGGAAAACTTCCGTACGCCGCCGCA
AAGCTTGTCGCCAGGTATTTCCATGGATTTGGAGCCGCGTTACGAGTTGT
TTATTTTTGGAAACGAGCCAACCAAGCGAGATTTGGATGTGCTGAATGCC
CTTTCCAATGTCGACATTGATAAGGAAACACTGCCGCATGTCTACGCCTG
GAAGACTGCCATGGAGAGCTACTCCTGTGCTGAAATGAATCTGAACGTCA
AGGTTCAAAAGCCGGAGCCTTGGTATTCTGGAACCAGTTCTAGCCACAAC
AGCCAACCATTGTTGCATCCCAAGCGTCTGCTTGCCACGCCAAAGCTGAA
TGCCGTGGTCAGCGGCAGACGCGGATCCGGACCATTGACGGCGCCAGTTA
CACCGCGTCTGGCGCGAACTCCGTCCGCCGCCAGTATTCAAGTTGCATCC
GAGACGAATGGCGAGTCGGTCGGAACTGCTGTGACTCCGGCATCGCCGAT
TTTGAGTTTTGCCGCCTTGACGGCAGCGACGCAGTCATTCCAAACACCAT
TGAACAAGGTGCGCGGCTTGTTCAGCCAATATCGGGATCAACGGTCCTAT
AACGAGGGGGACACGCCGCTGGGCAATCGGAACTGAAACGGAATCGGCCC
GGAAACAGAAACAGAAACAGCGACTGATTGATGAAAGGCCGACTGCATAC
TTACCCCCCTGAATAGCCGGTGTCGTCCATTGTCCCTTTTAATGTTAATC
GCATGTATATTA

(SEQ ID NO:176)

MSTYFGVYIPTSKAGCFEGSVSQCIGSIAAVNIKPSNPASGSASVASGSP
SGSAASVQTGNADDGSAATKYEDPDYPPDSPLWLIFTEKSKALDILRHYK
EARLREFPNLEQAESYVQFGFESIEALKRFCKAKPESKPIPIISGSGYKS
SPTSTDNSCSSSPTGNGSGFIIPLGSNSSMSNLLLSDSPTSSPSSSSNVI
ANGRQQQMQQQQQQQPQQPDVSGEGPPFRAPTKQELVEFRKQIEGGHIDR
VKRIIWENPRFLISSGDTPTSLKEGCRYNAMHICAQVNKARIAQLLLKTI
SDREFTQLYVGKKGSGKMCAALNISLLDYYLNMPDKGRGETPLHFAAKNG
HVAMVEVLVSYPECKSLRNHEGKEPKEIICLRNANATHVTIKKLELLLYD
PHFVPVLRSQSNTLPPKVGQPFSPKDPPNLQHKADDYEGLSVDLAISALA
GPMSREKAMNFYRRWKTPPRVSNNVMSPLAGSPFSSPVKVTPSKSIFDRS
AGNSSPVHSGRRVLFSPLAEATSSPKPTKNVPNGTNECEHNNNNVKPVYP
LEFPATPIRKMKPDLFMAYRNNNSFDSPSLADDSQILDMSLSRSLNASLN
DSFRERHIKNTDIEKGLEVVGRQLARQEQLEWREYWDFLDSFLDIGTTEG
LARLEAYFLEKTEQQADKSETVWNFAHLHQYFDSMAGEQQQQLRKDKNEA
AGATSPSAGVMTPYTCVEKSLQVFAKRITKTLINKIGNMVSINDTLLCEL
KRLKSLIVSFKDDARFISVDFSKVHSRIAHLVASYVTHSQEVSVAMRLQL
LQMLRSLRQLLADERGREQHLGCVCASLLLMLEQAPTSAVHLPDTLKTEE
LCCAAWETEQCCACLWDANLSRKTSRRKRTKSLRAAAVVQSQGQLQDTSG
STGSSALHASLGVGSTSLGASRVVASASKDAWRRQQSDDEDYDSDEQVIF
FDCTNVTLPYGSSSEDEENFRTPPQSLSPGISMDLEPRYELFIFGNEPTK
RDLDVLNALSNVDIDKETLPHVYAWKTAMESYSCAEMNLNVKVQKPEPWY
SGTSSSHNSQPLLHPKRLLATPKLNAVVSGRRGSGPLTAPVTPRLARTPS
AASIQVASETNGESVGTAVTPASPILSFAALTAATQSFQTPLNKVRGLFS
QYRDQRSYNEGDTPLGNRN

(SEQ ID NO:177)

TTGATGTTACCCTATTTTTACCGTTGCCTTCGCTTGCCATCAGCGGAACT
TTACATTTTTTCACGGAGTTGTGAAGAAGTTGCCTGTTATTTGGTGTTGA
TGTCAAACCATTTTAACCGCTTACCTTGCAGTGCATCCGCCATTTTACGC
AGAGATGTCGACCTATTTCGGGGTCTATATCCCGACCTCCAAAGCGGGCT
GTTTTGAGGGATCGGTGTCGCAGTGCATCGGCTCCATAGCCGCGGTGAAC
ATAAAGCCATCCAATCCGGCGTCTGGATCGGCATCAGTAGCATCGGGATC
GCCATCCGGCTCGGCGGCATCCGTGCAAACGGGCAACGCAGACGATGGCA
GTGCTGCCACCAAGTACGAGGATCCCGACTATCCACCGGACTCGCCACTG
TGGCTGATCTTCACGGAGAAATCCAAGGCGCTGGACATCCTGCGACACTA
CAAGGAGGCGCGCCTCCGCGAGTTTCCCAATCTGGAGCAGGCGGAGAGTT
ACGTTCAGTTTGGGTTCGAGAGCATCGAGGCGCTCAAGAGATTTTGCAAG
GCAAAGCCCGAAAGCAAGCCCATTCCGATAATCAGCGGTAGCGGTTACAA
GAGCTCACCGACCTCGACGGACAATTCGTGCTCCTCCTCGCCGACGGGTA
ACGGCAGTGGCTTCATCATTCCCCTGGGAAGCAATTCCTCAATGTCGAAT
TTACTGCTCAGTGACTCACCGACTTCCTCGCCGAGCAGCTCCAGCAACGT
CATTGCCAATGGGCGACAGCAGCAGATGCAGCAGCAACAGCAGCAGCAGC
CGCAGCAGCCGGATGTGTCCGGAGAAGGCCCTCCTTTCCGGGCGCCCACC
AAACAGGAACTGGTAGAGTTTCGCAAGCAAATCGAAGGTGGTCACATAGA
CCGGGTGAAGAGGATTATATGGGAGAATCCACGATTTTTGATCAGCAGCG
GTGATACGCCCACCAGTTTGAAGGAGGGCTGTCGCTATAATGCCATGCAC
ATCTGCGCCCAGGTCAATAAGGCCAGGATCGCTCAGTTGCTGTTAAAGAC
CATTTCGGATCGGGAGTTCACTCAGCTTTACGTTGGCAAGAAGGGCAGTG
GCAAGATGTGTGCTGCCCTCAACATCAGTCTCCTGGACTATTACCTGAAC
ATGCCGGACAAGGGGCGCGGCGAAACACCGCTCCACTTTGCCGCAAAGAA
CGGTCATGTGGCCATGGTCGAGGTTCTCGTTTCCTATCCGGAGTGCAAAT
CGCTGCGGAATCATGAGGGCAAGGAGCCCAAGGAAATCATCTGCCTGCGT
AATGCTAATGCTACACATGTGACCATCAAGAAGCTGGAGCTGCTCTTGTA
CGATCCGCATTTTGTGCCCGTACTAAGATCCCAGTCAAATACACTGCCGC
CAAAAGTGGGTCAACCGTTCTCGCCCAAAGATCCACCGAACCTGCAACAC
AAAGCGGACGATTACGAGGGCCTCAGCGTGGACCTGGCAATCAGTGCGCT
GGCGGGACCCATGTCCCGCGAAAAGGCCATGAACTTCTATCGCCGTTGGA
AGACACCACCGCGGGTCAGCAACAATGTGATGTCGCCGCTGGCTGGTTCA
CCATTTAGCTCGCCGGTGAAAGTAACCCCAAGCAAGTCGATCTTTGACCG
AAGTGCTGGAAACTCGAGTCCAGTCCACTCAGGACGCAGAGTGCTCTTTA
GTCCATTGGCGGAGGCGACCAGCTCACCAAAACCGACGAAAAACGTGCCC
AATGGCACCAATGAGTGCGAGCACAACAATAATAATGTGAAGCCAGTGTA
TCCGTTGGAGTTCCCGGCGACACCCATTCGAAAAATGAAACCGGATTTAT
TCATGGCCTATCGCAATAACAATAGCTTTGATTCGCCATCTTTGGCCGAT
GACTCCCAAATCCTGGACATGAGCCTAAGCCGCAGCCTGAATGCGTCGCT
AAATGACAGCTTCCGTGAGCGGCACATCAAGAACACTGATATCGAGAAGG
GTCTGGAGGTGGTCGGCCGCCAACTGGCACGACAGGAGCAGTTAGAGTGG
CGCGAGTACTGGGATTTTCTCGATTCATTTTTGGACATTGGTACGACCGA
AGGCCTGGCCCGTCTTGAAGCGTATTTCCTGGAAAAGACCGAACAGCAGG
CGGATAAATCAGAAACGGTCTGGAACTTTGCCCATCTGCATCAGTATTTC
GATTCGATGGCCGGCGAGCAACAGCAGCAACTCCGAAAGGATAAAAATGA
GGCTGCGGGAGCAACTTCGCCATCCGCCGGAGTCATGACTCCGTACACAT
GCGTAGAGAAGTCGCTGCAAGTGTTCGCCAAGCGCATCACTAAAACGTTG
ATCAACAAAATCGGCAACATGGTGTCCATCAACGACACGCTGCTCTGTGA
GCTCAAAAGACTGAAATCGCTGATTGTCAGCTTCAAGGATGATGCCCGCT
TCATTAGCGTGGACTTTAGCAAGGTGCATTCACGTATCGCCCACCTGGTG
GCCAGCTATGTGACCCACTCGCAGGAGGTCAGCGTAGCCATGCGTCTACA
ATTGTTGCAGATGCTCCGAAGTTTGCGGCAACTGCTGGCCGACGAGCGTG
GTCGAGAACAGCATTTGGGCTGCGTGTGCGCTAGTCTATTGCTGATGCTG
GAACAGGCGCCGACATCCGCCGTGCATCTACCAGACACTCTGAAGACCGA
GGAGCTATGTTGCGCCGCCTGGGAGACGGAGCAGTGTTGCGCCTGTCTGT
GGGACGCAAATCTCAGCCGTAAGACCAGTCGTCGAAAGCGCACTAAGTCG
CTGCGGGCAGCTGCTGTTGTTCAGTCTCAGGGTCAGCTTCAGGATACTTC
GGGATCGACAGGGTCGTCCGCCTTGCACGCTTCGCTTGGTGTGGGATCGA
CCAGTTTGGGAGCATCGAGGGTCGTGGCGTCCGCTTCGAAAGATGCTTGG
CGCCGTCAACAAAGCGACGACGAGGACTACGACAGCGATGAGCAAGTAAT
CTTTTTCGACTGCACTAATGTTACGCTGCCTTATGGAAGCAGCAGCGAGG
ACGAGGAAAACTTCCGTACGCCGCCGCAAAGCTTGTCGCCAGGTATTTCC
ATGGATTTGGAGCCGCGTTACGAGTTGTTTATTTTTGGAAACGAGCCAAC
CAAGCGAGATTTGGATGTGCTGAATGCCCTTTCCAATGTCGACATTGATA
AGGAAACACTGCCGCATGTCTACGCCTGGAAGACTGCCATGGAGAGCTAC
TCCTGTGCTGAAATGAATCTGAACGTCAAGGTTCAAAAGCCGGAGCCTTG
GTATTCTGGAACCAGTTCTAGCCACAACAGCCAACCATTGTTGCATCCCA
AGCGTCTGCTTGCCACGCCAAAGCTGAATGCCGTGGTCAGCGGCAGACGC
GGATCCGGACCATTGACGGCGCCAGTTACACCGCGTCTGGCGCGAACTCC
GTCCGCCGCCAGTATTCAAGTTGCATCCGAGACGAATGGCGAGTCGGTCG
GAACTGCTGTGACTCCGGCATCGCCGATTTTGAGTTTTGCCGCCTTGACG
GCAGCGACGCAGTCATTCCAAACACCATTGAACAAGGTGCGCGGCTTGTT
CAGCCAATATCGGGATCAACGGTCCTATAACGAGGGGGACACGCCGCTGG
GCAATCGGAACTGAAACGGAATCGGCCCGGAAACAGAAACAGAAACAGCG
ACTGATTGATGAAAGGCCGACTGCATACTTACCCCCCTGAATAGCCGGTG
TCGTCCATTGTCCCTTTTAATGTTAATCGCATGTATATTA

(SEQ ID NO:178)

MSTYFGVYIPTSKAGCFEGSVSQCIGSIAAVNIKPSNPASGSASVASGSP
SGSAASVQTGNADDGSAATKYEDPDYPPDSPLWLIFTEKSKALDILRHYK
EARLREFPNLEQAESYVQFGFESIEALKRFCKAKPESKPIPIISGSGYKS
SPTSTDNSCSSSPTGNGSGFIIPLGSNSSMSNLLLSDSPTSSPSSSSNVI
ANGRQQQMQQQQQQQPQQPDVSGEGPPFRAPTKQELVEFRKQIEGGHIDR
VKRIIWENPRFLISSGDTPTSLKEGCRYNAMHICAQVNKARIAQLLLKTI
SDREFTQLYVGKKGSGKMCAALNISLLDYYLNMPDKGRGETPLHFAAKNG
HVAMVEVLVSYPECKSLRNHEGKEPKEIICLRNANATHVTIKKLELLLYD
PHFVPVLRSQSNTLPPKVGQPFSPKDPPNLQHKADDYEGLSVDLAISALA
GPMSREKAMNFYRRWKTPPRVSNNVMSPLAGSPFSSPVKVTPSKSIFDRS
AGNSSPVHSGRRVLFSPLAEATSSPKPTKNVPNGTNECEHNNNNVKPVYP
LEFPATPIRKMKPDLFMAYRNNNSFDSPSLADDSQILDMSLSRSLNASLN
DSFRERHIKNTDIEKGLEVVGRQLARQEQLEWREYWDFLDSFLDIGTTEG
LARLEAYFLEKTEQQADKSETVWNFAHLHQYFDSMAGEQQQQLRKDKNEA
AGATSPSAGVMTPYTCVEKSLQVFAKRITKTLINKIGNMVSINDTLLCEL
KRLKSLIVSFKDDARFISVDFSKVHSRIAHLVASYVTHSQEVSVAMRLQL
LQMLRSLRQLLADERGREQHLGCVCASLLLMLEQAPTSAVHLPDTLKTEE
LCCAAWETEQCCACLWDANLSRKTSRRKRTKSLRAAAVVQSQGQLQDTSG
STGSSALHASLGVGSTSLGASRVVASASKDAWRRQQSDDEDYDSDEQVIF
FDCTNVTLPYGSSSEDEENFRTPPQSLSPGISMDLEPRYELFIFGNEPTK
RDLDVLNALSNVDIDKETLPHVYAWKTAMESYSCAEMNLNVKVQKPEPWY
SGTSSSHNSQPLLHPKRLLATPKLNAVVSGRRGSGPLTAPVTPRLARTPS
AASIQVASETNGESVGTAVTPASPILSFAALTAATQSFQTPLNKVRGLFS
QYRDQRSYNEGDTPLGNRN

(SEQ ID NO:179)

AAAACAGCCAGCTCATTTATTAATGGTTTATCCCTCTCGATGCCCACACA
TCAACATTGCCATCGCCACGACGGAGCAGCGGACTCGCCACTGTGGCTGA
TCTTCACGGAGAAATCCAAGGCGCTGGACATCCTGCGACACTACAAGGAG
GCGCGCCTCCGCGAGTTTCCCAATCTGGAGCAGGCGGAGAGTTACGTTCA
GTTTGGGTTCGAGAGCATCGAGGCGCTCAAGAGATTTTGCAAGGCAAAGC
CCGAAAGCAAGCCCATTCCGATAATCAGCGGTAGCGGTTACAAGAGCTCA
CCGACCTCGACGGACAATTCGTGCTCCTCCTCGCCGACGGGTAACGGCAG
TGGCTTCATCATTCCCCTGGGAAGCAATTCCTCAATGTCGAATTTACTGC
TCAGTGACTCACCGACTTCCTCGCCGAGCAGCTCCAGCAACGTCATTGCC
AATGGGCGACAGCAGCAGATGCAGCAGCAACAGCAGCAGCAGCCGCAGCA
GCCGGATGTGTCCGGAGAAGGCCCTCCTTTCCGGGCGCCCACCAAACAGG
AACTGGTAGAGTTTCGCAAGCAAATCGAAGGTGGTCACATAGACCGGGTG
AAGAGGATTATATGGGAGAATCCACGATTTTTGATCAGCAGCGGTGATAC
GCCCACCAGTTTGAAGGAGGGCTGTCGCTATAATGCCATGCACATCTGCG
CCCAGGTCAATAAGGCCAGGATCGCTCAGTTGCTGTTAAAGACCATTTCG
GATCGGGAGTTCACTCAGCTTTACGTTGGCAAGAAGGGCAGTGGCAAGAT
GTGTGCTGCCCTCAACATCAGTCTCCTGGACTATTACCTGAACATGCCGG
ACAAGGGGCGCGGCGAAACACCGCTCCACTTTGCCGCAAAGAACGGTCAT
GTGGCCATGGTCGAGGTTCTCGTTTCCTATCCGGAGTGCAAATCGCTGCG
GAATCATGAGGGCAAGGAGCCCAAGGAAATCATCTGCCTGCGTAATGCTA
ATGCTACACATGTGACCATCAAGAAGCTGGAGCTGCTCTTGTACGATCCG
CATTTTGTGCCCGTACTAAGATCCCAGTCAAATACACTGCCGCCAAAAGT
GGGTCAACCGTTCTCGCCCAAAGATCCACCGAACCTGCAACACAAAGCGG
ACGATTACGAGGGCCTCAGCGTGGACCTGGCAATCAGTGCGCTGGCGGGA
CCCATGTCCCGCGAAAAGGCCATGAACTTCTATCGCCGTTGGAAGACACC
ACCGCGGGTCAGCAACAATGTGATGTCGCCGCTGGCTGGTTCACCATTTA
GCTCGCCGGTGAAAGTAACCCCAAGCAAGTCGATCTTTGACCGAAGTGCT
GGAAACTCGAGTCCAGTCCACTCAGGACGCAGAGTGCTCTTTAGTCCATT
GGCGGAGGCGACCAGCTCACCAAAACCGACGAAAAACGTGCCCAATGGCA
CCAATGAGTGCGAGCACAACAATAATAATGTGAAGCCAGTGTATCCGTTG
GAGTTCCCGGCGACACCCATTCGAAAAATGAAACCGGATTTATTCATGGC
CTATCGCAATAACAATAGCTTTGATTCGCCATCTTTGGCCGATGACTCCC
AAATCCTGGACATGAGCCTAAGCCGCAGCCTGAATGCGTCGCTAAATGAC
AGCTTCCGTGAGCGGCACATCAAGAACACTGATATCGAGAAGGGTCTGGA
GGTGGTCGGCCGCCAACTGGCACGACAGGAGCAGTTAGAGTGGCGCGAGT
ACTGGGATTTTCTCGATTCATTTTTGGACATTGGTACGACCGAAGGCCTG
GCCCGTCTTGAAGCGTATTTCCTGGAAAAGACCGAACAGCAGGCGGATAA
ATCAGAAACGGTCTGGAACTTTGCCCATCTGCATCAGTATTTCGATTCGA
TGGCCGGCGAGCAACAGCAGCAACTCCGAAAGGATAAAAATGAGGCTGCG
GGAGCAACTTCGCCATCCGCCGGAGTCATGACTCCGTACACATGCGTAGA
GAAGTCGCTGCAAGTGTTCGCCAAGCGCATCACTAAAACGTTGATCAACA
AAATCGGCAACATGGTGTCCATCAACGACACGCTGCTCTGTGAGCTCAAA
AGACTGAAATCGCTGATTGTCAGCTTCAAGGATGATGCCCGCTTCATTAG
CGTGGACTTTAGCAAGGTGCATTCACGTATCGCCCACCTGGTGGCCAGCT
ATGTGACCCACTCGCAGGAGGTCAGCGTAGCCATGCGTCTACAATTGTTG
CAGATGCTCCGAAGTTTGCGGCAACTGCTGGCCGACGAGCGTGGTCGAGA
ACAGCATTTGGGCTGCGTGTGCGCTAGTCTATTGCTGATGCTGGAACAGG
CGCCGACATCCGCCGTGCATCTACCAGACACTCTGAAGACCGAGGAGCTA
TGTTGCGCCGCCTGGGAGACGGAGCAGTGTTGCGCCTGTCTGTGGGACGC
AAATCTCAGCCGTAAGACCAGTCGTCGAAAGCGCACTAAGTCGCTGCGGG
CAGCTGCTGTTGTTCAGTCTCAGGGTCAGCTTCAGGATACTTCGGGATCG
ACAGGGTCGTCCGCCTTGCACGCTTCGCTTGGTGTGGGATCGACCAGTTT
GGGAGCATCGAGGGTCGTGGCGTCCGCTTCGAAAGATGCTTGGCGCCGTC
AACAAAGCGACGACGAGGACTACGACAGCGATGAGCAAGTAATCTTTTTC
GACTGCACTAATGTTACGCTGCCTTATGGAAGCAGCAGCGAGGACGAGGA
AAACTTCCGTACGCCGCCGCAAAGCTTGTCGCCAGGTATTTCCATGGATT
TGGAGCCGCGTTACGAGTTGTTTATTTTTGGAAACGAGCCAACCAAGCGA
GATTTGGATGTGCTGAATGCCCTTTCCAATGTCGACATTGATAAGGAAAC
ACTGCCGCATGTCTACGCCTGGAAGACTGCCATGGAGAGCTACTCCTGTG
CTGAAATGAATCTGAACGTCAAGGTTCAAAAGCCGGAGCCTTGGTATTCT
GGAACCAGTTCTAGCCACAACAGCCAACCATTGTTGCATCCCAAGCGTCT
GCTTGCCACGCCAAAGCTGAATGCCGTGGTCAGCGGCAGACGCGGATCCG
GACCATTGACGGCGCCAGTTACACCGCGTCTGGCGCGAACTCCGTCCGCC
GCCAGTATTCAAGTTGCATCCGAGACGAATGGCGAGTCGGTCGGAACTGC
TGTGACTCCGGCATCGCCGATTTTGAGTTTTGCCGCCTTGACGGCAGCGA
CGCAGTCATTCCAAACACCATTGAACAAGGTGCGCGGCTTGTTCAGCCAA
TATCGGGATCAACGGTCCTATAACGAGGGGGACACGCCGCTGGGCAATCG
GAACTGAAACGGAATCGGCCCGGAAACAGAAACAGAAACAGCGACTGATT
GATGAAAGGCCGACTGCATACTTACCCCCCTGAATAGCCGGTGTCGTCCA
TTGTCCCTTTTAATGTTAATCGCATGTATATTA

(SEQ ID NO:180)

MPTHQHCHRHDGAADSPLWLIFTEKSKALDILRHYKEARLREFPNLEQAE
SYVQFGFESIEALKRFCKAKPESKPIPIISGSGYKSSPTSTDNSCSSSPT
GNGSGFIIPLGSNSSMSNLLLSDSPTSSPSSSSNVIANGRQQQMQQQQQQ
QPQQPDVSGEGPPFRAPTKQELVEFRKQIEGGHIDRVKRIIWENPRFLIS
SGDTPTSLKEGCRYNAMHICAQVNKARIAQLLLKTISDREFTQLYVGKKG
SGKMCAALNISLLDYYLNMPDKGRGETPLHFAAKNGHVAMVEVLVSYPEC
KSLRNHEGKEPKEIICLRNANATHVTIKKLELLLYDPHFVPVLRSQSNTL
PPKVGQPFSPKDPPNLQHKADDYEGLSVDLAISALAGPMSREKAMNFYRR
WKTPPRVSNNVMSPLAGSPFSSPVKVTPSKSIFDRSAGNSSPVHSGRRVL
FSPLAEATSSPKPTKNVPNGTNECEHNNNNVKPVYPLEFPATPIRKMKPD
LFMAYRNNNSFDSPSLADDSQILDMSLSRSLNASLNDSFRERHIKNTDIE
KGLEVVGRQLARQEQLEWREYWDFLDSFLDIGTTEGLARLEAYFLEKTEQ
QADKSETVWNFAHLHQYFDSMAGEQQQQLRKDKNEAAGATSPSAGVMTPY
TCVEKSLQVFAKRITKTLINKIGNMVSINDTLLCELKRLKSLIVSFKDDA
RFISVDFSKVHSRIAHLVASYVTHSQEVSVAMRLQLLQMLRSLRQLLADE
RGREQHLGCVCASLLLMLEQAPTSAVHLPDTLKTEELCCAAWETEQCCAC
LWDANLSRKTSRRKRTKSLRAAAVVQSQGQLQDTSGSTGSSALHASLGVG
STSLGASRVVASASKDAWRRQQSDDEDYDSDEQVIFFDCTNVTLPYGSSS
EDEENFRTPPQSLSPGISMDLEPRYELFIFGNEPTKRDLDVLNALSNVDI
DKETLPHVYAWKTAMESYSCAEMNLNVKVQKPEPWYSGTSSSHNSQPLLH
PKRLLATPKLNAVVSGRRGSGPLTAPVTPRLARTPSAASIQVASETNGES
VGTAVTPASPILSFAALTAATQSFQTPLNKVRGLFSQYRDQRSYNEGDTP
LGNRN

Human homologue of Complete Genome candidate

BAA31667 KIAA0692 protein

| | | |
|---|---|---|
| 1 | gagattttgg ttacagtgtg ggcctgaatc ctccagagga ggaagctgtg acatccaaga | (SEQ ID NO:181) |
| 61 | cctgctcggt gccccctagt gacaccgaca cctacagagc tggagcgact gcgtctaagg | |
| 121 | agccgcccct gtactatggg gtgtgtccag tgtatgagga cgtcccagcg agaaatgaaa | |
| 181 | ggatctatgt ttatgaaaat aaaaaggaag cattgcaagc tgtcaagatg atcaaagggt | |
| 241 | cccgatttaa agctttttct accagagaag acgctgagaa atttgctaga ggaatttgtg | |
| 301 | attatttccc ttctccaagc aaaacgtcct taccactgtc tcctgtgaaa acagctccac | |
| 361 | tctttagcaa tgacaggttg aaagatggtt tgtgcttgtc ggaatcagaa acagtcaaca | |
| 421 | aagagcgagc gaacagttac aaaaatcccc gcacgcagga cctcaccgcc aagcttcgga | |
| 481 | aagctgtgga gaagggagag gaggacacct tttctgacct tatctggagc aacccccggt | |
| 541 | atctgatagg ctcaggagac aaccccacta tcgtgcagga agggtgcagg tacaacgtga | |
| 601 | tgcatgttgc tgccaaagag aaccaggctt ccatctgcca gctgactctg gacgtcctgg | |
| 661 | agaaccctga cttcatgagg ctgatgtacc ctgatgacga cgaggccatg ctgcagaagc | |
| 721 | gtatccgtta cgtggtggac ctgtacctca acacccccga caagatgggc tatgacacac | |
| 781 | cgttgcattt tgcttgtaag tttggaaatg cagatgtagt caacgtgctt tcgtcacacc | |
| 841 | atttgattgt aaaaaactca aggaataaat atgataaaac acctgaagat gtaatttgtg | |
| 901 | aaagaagcaa aaataaatct gtggaactga aggagcggat cagagagtat ttaaagggcc | |
| 961 | actactacgt gcccctcctg agagcggaag agacttcttc tccagtcatc ggggagctgt | |
| 1021 | ggtccccaga ccagacggct gaggcctctc acgtcagccg ctatggaggc agccccagag | |
| 1081 | acccggtact gaccctgaga gccttcgcag ggcccctgag tccagccaag gcagaagatt | |
| 1141 | ttcgcaagct ctggaaaact ccacctcgag agaaagcagg cttccttcac cacgtcaaga | |
| 1201 | agtcggaccc ggaaagaggc tttgagagag tgggaaggga gctagctcat gagctggggt | |
| 1261 | atccctgggt tgaatactgg gaatttctgg gctgttttgt tgatctgtct tcccaggaag | |
| 1321 | gcctgcaaag actagaagaa tatctcacac agcaggaaat aggcaaaaag gctcaacaag | |
| 1381 | aaacaggaga acgggaagcc tcctgccgag ataaagccac cacgtctggc agcaattcca | |
| 1441 | tttccgtgag ggcgtttcta gatgaagatg acatgagctt ggaagaaata aaaaatcggc | |
| 1501 | aaaatgcagc tcgaaataac agcccgccca cagtcggtgc ttttggacat acgaggtgca | |
| 1561 | gcgccttccc cttggagcag gaggcagacc tcatagaagc cgccgagccg ggaggtccac | |
| 1621 | acagcagcag aaatgggctc tgccatcctc tgaatcacag caggaccctg gcgggcaaga | |
| 1681 | gaccaaaggc cccccatggg gaggaagccc atctgccacc tgtctcggat ttgactgttg | |
| 1741 | agtttgataa actgaatttg caaaatatag gacgtagcgt ttccaagaca ccagatgaaa | |
| 1801 | gtacaaaaac taaagatcag atcctgactt caagaatcaa tgcagtagaa agagacttgt | |
| 1861 | tagagccttc tcccgcagac caactcggga atggccacag gaggacagaa agtgaaatgt | |
| 1921 | cagccaggat cgctaaaatg tccttgagtc ccagcagccc caggcacgag gatcagctcg | |
| 1981 | aggtcaccag ggaaccggcc aggcggctct tcctttttgg agaggagcca tcaaaactcg | |
| 2041 | atcaggatgt tttggccgct cttgaatgtg cagacgtcga cccccatcag ttcccggccg | |
| 2101 | tgcacagatg gaagagtgct gtcctgtgct actcaccctc ggacagacag agttggccca | |
| 2161 | gtcccgcggt gaaaggaagg ttcaagtctc agctgccaga tctcagtggc cctcacagct | |
| 2221 | acagtccggg gagaaacagc gtggctggaa gcaaccccgc aaagccaggc ctgggcagtc | |
| 2281 | ctgggcgcta cagccccgtg cacgggagcc agctccgcag gatggcgcgc ctggctgagc | |
| 2341 | ttgccgccct gtaggcttgg cgctgggctc tcggtttgtt cttcattttt aaagaaggaa | |
| 2401 | gggtcatatg tttattgcta aactgtcaaa aaggaatata ttctgattaa attattactc | |
| 2461 | ctcactttga gggtgtgaga attttagaag atttaaatgt tctatataac acttagattt | |
| 2521 | ctgatatttt ggaagaagtt agaagttaat gaaagcaaac tcagttacca attttctgga | |
| 2581 | aaatatccat gtggtaatgt agacttttta ggtggcaatt tctaggtctg aaatatagca | |
| 2641 | gaggaaaggg cgctgaggca gttgcaggca ggcagccctg tacttaccct gtactcacct | |
| 2701 | catccgacag acgctgtgga tgaggagggg cttggcggag gcgtgagcac cgatgtccct | |
| 2761 | ttgataacct gcactcacca agatgaacta tttgccgccc tgtcttttcc tgggttgggg | |
| 2821 | ggtggcatct gatggtggca gagtgcctgt tggttcgccc gtgggtctca tggttcagac | |
| 2881 | agagggaggt ggacggcagg gatcagggag coaggagcgc gcctcagact tgcagcaacc | |
| 2941 | attgtgattt gggttgttcg gaatatttaa attactgatc agaagatgaa agtagctttt | |
| 3001 | ctcttgggaa gtcttgcagc ccgtgggagt gataccagga gcaacacaga gctcagcagc | |
| 3061 | ggcgccaagg tgttccctgt ttcctcagca cgtgagcctt caccgcctgc ttcattcagg | |
| 3121 | agccagtgca gcagtaatac agtctataca ttgttctgtt ttcaaattta tcctgaggct | |
| 3181 | ttgttgagca taaatgatta tacgataaag gtatccgtta ttttggaact catttcagtt | |
| 3241 | gggatctcct gtatgcagag tgttgcattt agaggtttga gtcccatctt ggtttcttgc | |
| 3301 | cgtgctgact gtagccttca ccttgacttg aatgaaggtc tgtggttgga atgtgtgagg | |
| 3361 | agccgctgag gtgttcagga ggtgctgcct ggaggtcggt ttcttcctgg gtgttacggg | |
| 3421 | caactgctca cacagttgtt tctctgtgaa catttccagt gtttaatcca aaatgaaaac | |
| 3481 | ccaccaatgc ttttgctaac ttcagtgcct tttataaatc atttttaaat ttcctgaact | |
| 3541 | tgctttttga ggatatacag ggatattaag tagacgcagg attgtttttg tttgtaaaaa | |
| 3601 | ttctgaattg aaactttgtt ttaaaaaaag gcttctttct ttcatatgac aagagatagg | |
| 3661 | tcaggaatat tggaatcaag atttaaatgt taaaattcga ttttgttaca cagggtgtgt | |
| 3721 | tcatttgttt tgtagcagac aagatctaga tcccagacag aaacaacaca tgctattcta | |
| 3781 | aaaagccgca ttttaaaagg caccttggtt ctcaaaagaa atcagaatat ggatattcgt | |
| 3841 | agtgatgatc tgttttctct aaaatcttac catattgtct gtatatggtt gtaaattcaa | |
| 3901 | atggaaagta aaacgttttg gccctgattt tgtatgtgga ccactgctcc tgatttccca | |
| 3961 | ggtcttaggc cacctttgac tgtttctccg tttgtttgtg ggcagcgatt ccagtcccaa | |
| 4021 | cggaggcatt ctcgtgtgtc ccggggggtt atgtccttca caaaacactt aatgaaatga | |
| 4081 | attacttc | |
| 1 | dfgysvglnp peeeavtskt csvppsdtdt yragataske pplyygvcpv yedvparner | (SEQ ID NO:182) |
| 61 | iyvyenkkea lqavkmikgs rfkafstred aekfargicd yfpspsktsl plspvktapl | |
| 121 | fsndrlkdgl clsesetvnk eransyknpr tqdltaklrk avekgeedtf sdliwsnpry | |
| 181 | ligsgdnpti vqegcrynvm hvaakenqas icqltldvle npdfmrlmyp dddeamlqkr | |
| 241 | iryvvdlyln tpdkmgydtp lhfackfgna dvvnvlsshh livknsrnky dktpedvice | |
| 301 | rsknksvelk erireylkgh yyvpllraee tsspvigelw spdqtaeash vsryggsprd | |
| 361 | pvltlrafag plspakaedf rklwktppre kagflhhvkk sdpergferv grelahelgy | |
| 421 | pwveyweflg cfvdlssqeg lqrleeyltq qeigkkaqqe tgereascrd kattsgsnsi | |
| 481 | svrafldedd msleeiknrq naarnnsppt vgafghtrcs afpleqeadl ieaaepggph | |
| 541 | ssrnglchpl nhsrtlagkr pkaphgeeah lppvsdltve fdklnlqnig rsvsktpdes | |
| 601 | tktkdqilts rinaverdll epspadqlgn ghrrtesems ariakmslsp ssprhedqle | |
| 661 | vtreparrlf lfgeepskld qdvlaaleca dvdphqfpav hrwksavlcy spsdrqswps | |
| 721 | pavkgrfksq lpdlsgphsy spgrnsvags npakpglgsp gryspvhgsq lrrmarlael | |
| 781 | aal | |

Putative function
Unknown

### Example 15

### Category 3

Line ID—379

Category—Lethal phase pharate adult, Dot and rod-like overcondensed chromosomes, high mitotic index, overcondensed anaphases some with lagging chromosomes, a few tetraploid cells with overcondensed chromosomes, XYY males.

Annotated Drosophila genome genomic segment containing P element insertion site (and map position)—AE003443 (7D14-E2)

P element insertion site—130,532

Annotated Drosophila genome Complete Genome candidate
2 candidates:

CG10964—novel, similarity to dehydrogenases

(SEQ ID NO:183)

AACGAAACAGCCGGCCGTCAAAATTTTTCCTAACATTTCACTATTTTCAC
GCTTGTGTTACGGCAATAAAGTCGATTGATAAGCACGGAAAGATCTGGCT
GCGGGTCTGGTGAAATCCACAGAACACACGGAACCCGTATAGTAGTGCCG
CCCTTTATTGGTTTTATCTCAAGTACGACGCGATAAGATTTCGAGCAACT
CGATCGCGGATCTTCGGAAAAAAAAAACATGAACTCCATCCTGATAACCG
GCTGCAATCGAGGATTGGGTCTGGGCCTGGTCAAGGCGCTGCTCAATCTT
CCCCAGCCGCCGCAGCATCTATTTACCACCTGCCGGAATCGCGAGCAGGC
AAAGGAGCTGGAGGATCTAGCCAAGAACCACTCGAACATACACATACTTG
AGATTGATTTGAGAAATTTCGATGCCTATGACAAGCTAGTCGCCGACATC
GAGGGCGTGACCAAGGACCAAGGCCTCAATGTGCTCTTCAACAATGCCGG
CATAGCGCCCAAATCGGCCAGGATAACGGCCGTTCGATCGCAGGAGCTGC
TCGACACCTTGCAGACCAACACGGTTGTGCCCATCATGCTGGCCAAGGCG
TGTCTGCCGCTCCTTAAGAAGGCAGCCAAAGCGAACGAATCCCAGCCGAT
GGGCGTGGGCCGTGCCGCCATTATTAACATGTCCTCGATCCTTGGCTCCA
TCCAGGGCAACACGGACGGCGGAATGTACGCCTATCGCACCTCTAAGTCG
GCCTTGAATGCGGCCACCAAGTCGTTGAGCGTGGATCTGTATCCGCAACG
CATCATGTGCGTCAGTCTGCATCCTGGCTGGGTGAAAACCGACATGGGTG
GCTCCAGTGCCCCCTTGGACGTGCCCACCAGCACGGGACAAATTGTGCAG
ACCATCAGCAAGCTGGGCGAGAAACAGAACGGCGGTTTTGTCAACTACGA
CGGCACTCCGCTGGCCTGGTAA

(SEQ ID NO:184)

MNSILITGCNRGLGLGLVKALLNLPQPPQHLFTTCRNREQAKELEDLAKN
HSNIHILEIDLRNFDAYDKLVADIEGVTKDQGLNVLFNNAGIAPKSARIT
AVRSQELLDTLQTNTVVPIMLAKACLPLLKKAAKANESQPMGVGRAAIIN
MSSILGSIQGNTDGGMYAYRTSKSALNAATKSLSVDLYPQRIMCVSLHPG
WVKTDMGGSSAPLDVPTSTGQIVQTISKLGEKQNGGFVNYDGTPLAW

CG2151—Trxr-1thoredoxin reductase—1(2 splice variants)

(SEQ ID NO:185)

CGACAAGCCAATCGACGTCTCCCTTTCGCACGCTCGTACGAAAGTACAAA
AGCTATTGCAAAAGTTGGCTCCGCTTATTCGTTTCGTGCTTTCGCGAGTG
CCGAGAGCCGCTACAATACACGCTTAGCAGTTTTTACATTTCCGCTTCGA
CTACAACAACATTCACTACCCGCCGTTGATCCTTGTTTTCTGTCTGATTT
ACGTGGAGCACCTACCAACAAGCAACAAAATAATGGCGCCCGTGCAAGGA
TCCTACGACTACGACCTTATTGTGATTGGAGGCGGCTCAGCTGGCCTGGC
CTGCGCCAAGGAGGCAGTCCTCAATGGAGCCCGTGTGGCCTGTCTGGATT
TCGTTAAGCCCACGCCCACTCTGGGCACCAAGTGGGGCGTTGGCGGCACC
TGCGTGAACGTGGGCTGCATTCCCAAGAAGCTGATGCACCAGGCCTCCCT
TCTGGGCGAGGCTGTCCATGAGGCGGCCGCCTACGGCTGGAACGTGGACG
AAAAGATCAAGCCAGACTGGCACAAGCTGGTGCAGTCCGTACAGAACCAC
ATCAAGTCCGTCAACTGGGTGACCCGTGTGGATCTGCGCGACAAGAAAGT
GGAGTACATCAATGGACTGGGCTCCTTCGTGGACTCGCACACACTGCTGG
CCAAGCTGAAGAGCGGCGAGCGCACAATCACCGCCCAGACCTTCGTCATT
GCCGTTGGCGGCCGACCACGTTATCCGGATATTCCCGGTGCTGTCGAGTA
TGGCATCACCAGCGATGATCTGTTCAGTTTGGACCGCGAGCCCGGCAAGA
CCCTGGTGGTGGGAGCTGGCTACATTGGCTTGGAGTGCGCTGGATTCCTG
AAGGGTCTCGGCTACGAGCCCACTGTGATGGTGCGTTCTATTGTGCTGCG
TGGCTTCGACCAGCAGATGGCCGAGCTGGTGGCAGCCTCGATGGAGGAGC
GTGGCATTCCCTTCCTCCGCAAGACGGTGCCGCTGTCCGTGGAAAAGCAG
GATGATGGCAAGCTGCTCGTGAAGTACAAGAACGTGGAGACCGGCGAGGA
GGCCGAGGATGTTTACGACACCGTTCTGTGGGCCATCGGCCGCAAGGGTC
TGGTGGACGATCTGAACCTGCCCAATGCCGGCGTGACTGTGCAGAAGGAC
AAGATTCCAGTGGACTCCCAGGAGGCTACCAATGTGGCAAACATCTACGC
TGTCGGCGATATCATCTATGGCAAGCCAGAGCTGACGCCCGTCGCCGTTT
TGGCTGGCCGTTTGCTGGCCCGCCGCCTGTACGGAGGATCTACCCAGCGC
ATGGACTACAAGGATGTGGCCACCACCGTTTTCACGCCCCTGGAGTACGC
CTGCGTCGGCCTGAGCGAGGAGGATGCCGTCAAGCAGTTCGGAGCCGATG
AGATCGAGGTGTTCCACGGCTACTACAAGCCCACGGAGTTCTTCATTCCC
CAGAAGAGCGTGCGCTACTGCTACTTGAAGGCTGTGGCCGAGCGCCATGG
TGACCAGCGCGTCTATGGACTGCACTATATTGGCCCGGTGGCCGGTGAGG
TTATCCAGGGATTCGCTGCCGCTTTGAAGTCTGGCCTGACTATTAACACG
CTGATCAACACCGTGGGCATCCATCCCACTACCGCCGAAGAATTCACCCG
GCTGGCCATCACCAAGCGCTCCGGACTGGACCCCACGCCGGCCAGCTGCT
GCAGCTAAAGCGGGAACGCAGCTCAGCCGCCTGGGACGTGTCGAAGCCGC
TTGCTCCACCCGAAATCCCGTAGATGAATGGTTGTTGTCGCGGCCCAGCG
ATCGATGAGTTCAATAGTTCCGTTTCGTTTCCACAATTAACACCCAACAC
AATAGCTCTGCGCAAGGGAGGGGCACTGGGCAGCGATGGCGGGTGGAACG
ACACCAGTGGAACTACCCGCGCGACCAGCCCAACCCACGACTGCTGCGCC
GCCGACATGCACTCAAAATTTTGAATTTGTTTGAACCTATGAAATTAACT
ATGAAATCCCCTAAATGTACGGTTGAAGAATATAATTTTTCACC

(SEQ ID NO:186)

MAPVQGSYDYDLIVIGGGSAGLACAKEAVLNGARVACLDFVKPTPTLGTK
WGVGGTCVNVGCIPKKLMHQASLLGEAVHEAAAYGWNVDEKIKPDWHKLV
QSVQNHIKSVNWVTRVDLRDKKVEYINGLGSFVDSHTLLAKLKSGERTIT
AQTFVIAVGGRPRYPDIPGAVEYGITSDDLFSLDREPGKTLVVGAGYIGL
ECAGFLKGLGYEPTVMVRSIVLRGFDQQMAELVAASMEERGIPFLRKTVP
LSVEKQDDGKLLVKYKNVETGEEAEDVYDTVLWAIGRKGLVDDLNLPNAG
VTVQKDKIPVDSQEATNVANIYAVGDIIYGKPELTPVAVLAGRLLARRLY
GGSTQRMDYKDVATTVFTPLEYACVGLSEEDAVKQFGADEIEVFHGYYKP
TEFFIPQKSVRYCYLKAVAERHGDQRVYGLHYIGPVAGEVIQGFAAALKS
GLTINTLINTVGIHPTTAEEFTRLAITKRSGLDPTPASCCS

(SEQ ID NO:187)

CCCGGCCGAACCAGCGAACGTGTTTGTGTTGTGTGTTCCGCCGTCATTTT
TCTGCACCCTTTTCGCGAATAGTTTCGTTTCGCCTCCAGCTGGTAGAGTG
AAACGCCAAACGTTGAAGAAGGGGAAAGGCCAACAAGATGAACTTGTGCA
ATTCGAGATTCTCCGTTACGTTCGTGCGGCAGTGCTCGACGATTTTAACG
TCTCCTTCGGCTGGCATTATACAAAACAGAGGCTCACTGACAACAAAGGT
TCCCCATTGGATTTCCAGTAGTCTCAGCTGTGCCCATCACACGTTTCAGC
GAACTATGAACTTGACGGGACAGCGAGGATCACGCGACAGTACTGGAGCT
ACCGGTGGGAATGCTCCAGCCGGATCCGGTGCCGGCGCACCACCACCCTT
CCAGCATCCACATTGCGACAGGGCGGCCATGTACGCGCAACCGGTGCGAA
AGATGAGCACCAAAGGAGGATCCTACGACTACGACCTTATTGTGATTGGA
GGCGGCTCAGCTGGCCTGGCCTGCGCCAAGGAGGCAGTCCTCAATGGAGC
CCGTGTGGCCTGTCTGGATTTCGTTAAGCCCACGCCCACTCTGGGCACCA
AGTGGGGCGTTGGCGGCACCTGCGTGAACGTGGGCTGCATTCCCAAGAAG
CTGATGCACCAGGCCTCCCTTCTGGGCGAGGCTGTCCATGAGGCGGCCGC
CTACGGCTGGAACGTGGACGAAAAGATCAAGCCAGACTGGCACAAGCTGG
TGCAGTCCGTACAGAACCACATCAAGTCCGTCAACTGGGTGACCCGTGTG
GATCTGCGCGACAAGAAAGTGGAGTACATCAATGGACTGGGCTCCTTCGT
GGACTCGCACACACTGCTGGCCAAGCTGAAGAGCGGCGAGCGCACAATCA
CCGCCCAGACCTTCGTCATTGCCGTTGGCGGCCGACCACGTTATCCGGAT
ATTCCCGGTGCTGTCGAGTATGGCATCACCAGCGATGATCTGTTCAGTTT
GGACCGCGAGCCCGGCAAGACCCTGGTGGTGGGAGCTGGCTACATTGGCT
TGGAGTGCGCTGGATTCCTGAAGGGTCTCGGCTACGAGCCCACTGTGATG
GTGCGTTCTATTGTGCTGCGTGGCTTCGACCAGCAGATGGCCGAGCTGGT
GGCAGCCTCGATGGAGGAGCGTGGCATTCCCTTCCTCCGCAAGACGGTGC
CGCTGTCCGTGGAAAAGCAGGATGATGGCAAGCTGCTCGTGAAGTACAAG
AACGTGGAGACCGGCGAGGAGGCCGAGGATGTTTACGACACCGTTCTGTG
GGCCATCGGCCGCAAGGGTCTGGTGGACGATCTGAACCTGCCCAATGCCG
GCGTGACTGTGCAGAAGGACAAGATTCCAGTGGACTCCCAGGAGGCTACC
AATGTGGCAAACATCTACGCTGTCGGCGATATCATCTATGGCAAGCCAGA
GCTGACGCCCGTCGCCGTTTTGGCTGGCCGTTTGCTGGCCCGCCGCCTGT
ACGGAGGATCTACCCAGCGCATGGACTACAAGGATGTGGCCACCACCGTT
TTCACGCCCCTGGAGTACGCCTGCGTCGGCCTGAGCGAGGAGGATGCCGT
CAAGCAGTTCGGAGCCGATGAGATCGAGGTGTTCCACGGCTACTACAAGC
CCACGGAGTTCTTCATTCCCCAGAAGAGCGTGCGCTACTGCTACTTGAAG
GCTGTGGCCGAGCGCCATGGTGACCAGCGCGTCTATGGACTGCACTATAT
TGGCCCGGTGGCCGGTGAGGTTATCCAGGGATTCGCTGCCGCTTTGAAGT
CTGGCCTGACTATTAACACGCTGATCAACACCGTGGGCATCCATCCCACT
ACCGCCGAAGAATTCACCCGGCTGGCCATCACCAAGCGCTCCGGACTGGA
CCCCACGCCGGCCAGCTGCTGCAGCTAAAGCGGGAACGCAGCTCAGCCGC
CTGGGACGTGTCGAAGCCGCTTGCTCCACCCGAAATCCCGTAGATGAATG
GTTGTTGTCGCGGCCCAGCGATCGATGAGTTCAATAGTTCCGTTTCGTTT
CCACAATTAACACCCAACACAATAGCTCTGCGCAAGGGAGGGGCACTGGG
CAGCGATGGCGGGTGGAACGACACCAGTGGAACTACCCGCGCGACCAGCC
CAACCCACGACTGCTGCGCCGCCGACATGCACTCAAAATTTTGAATTTGT
TTGAACCTATGAAATTAACTATGAAATCCCCTAAATGTACGGTTGAAGAA
TATAATTTTTCACC

(SEQ ID NO:188)

MSTKGGSYDYDLIVIGGGSAGLACAKEAVLNGARVACLDFVKPTPTLGTK
WGVGGTCVNVGCIPKKLMHQASLLGEAVHEAAAYGWNVDEKIKPDWHKLV
QSVQNHIKSVNWVTRVDLRDKKVEYINGLGSFVDSHTLLAKLKSGERTIT
AQTFVIAVGGRPRYPDIPGAVEYGITSDDLFSLDREPGKTLVVGAGYIGL
ECAGFLKGLGYEPTVMVRSIVLRGFDQQMAELVAASMEERGIPFLRKTVP
LSVEKQDDGKLLVKYKNVETGEEAEDVYDTVLWAIGRKGLVDDLNLPNAG
VTVQKDKIPVDSQEATNVANIYAVGDIIYGKPELTPVAVLAGRLLARRLY
GGSTQRMDYKDVATTVFTPLEYACVGLSEEDAVKQFGADEIEVFHGYYKP
TEFFIPQKSVRYCYLKAVAERHGDQRVYGLHYIGPVAGEVIQGFAAALKS
GLTINTLINTVGIHPTTAEEFTRLAITKRSGLDPTPASCCS

Human homologue of Complete Genome candidate

(CG10965)—AAC50725 11-cis retinol dehydrogenase

| | | |
|---|---|---|
| 1 | taagcttcgg gcgctgtagt acctgccagc tttcgccaca ggaggctgcc acctgtaggt | (SEQ ID NO:189) |
| 61 | cacttgggct ccagctatgt ggctgcctct tctgctgggt gccttactct gggcagtgct | |
| 121 | gtggttgctc agggaccggc agagcctgcc cgccagcaat gcctttgtct tcatcaccgg | |
| 181 | ctgtgactca ggctttgggc gccttctggc actgcagctg gaccagagag gcttccgagt | |
| 241 | cctggccagc tgcctgaccc cctccggggc cgaggacctg cagcgggtgg cctcctcccg | |
| 301 | cctccacacc accctgttgg atatcactga tccccagagc gtccagcagg cagccaagtg | |
| 361 | ggtggagatg cacgttaagg aagcagggct ttttggtctg gtgaataatg ctggtgtggc | |
| 421 | tggtatcatc ggacccacac catggctgac ccgggacgat ttccagcggg tgctgaatgt | |
| 481 | gaacacaatg ggtcccatcg gggtcaccct tgccctgctg cctctgctgc agcaagcccg | |
| 541 | gggccgggtg atcaacatca ccagcgtcct gggtcgcctg gcagccaatg gtgggggcta | |
| 601 | ctgtgtctcc aaatttggcc tggaggcctt ctctgacagc ctgaggcggg atgtagctca | |
| 661 | ttttgggata cgagtctcca tcgtggagcc tggcttcttc cgaacccctg tgaccaacct | |
| 721 | ggagagtctg gagaaaaccc tgcaggcctg ctgggcacgg ctgcctcctg ccacacaggc | |
| 781 | ccactatggg ggggccttcc tcaccaagta cctgaaaatg caacagcgca tcatgaacct | |
| 841 | gatctgtgac ccggacctaa ccaaggtgag ccgatgcctg gagcatgccc tgactgctcg | |
| 901 | acacccccga acccgctaca gcccaggttg ggatgccaag ctgctctggc tgcctgcctc | |
| 961 | ctacctgcca gccagcctgg tggatgctgt gctcacctgg gtccttccca agcctgccca | |
| 1021 | agcagtctac tgaatccagc cttccagcaa gagattgttt ttcaaggaca aggactttga | |
| 1081 | tttatttctg cccccaccct ggtactgcct ggtgcctgcc acaaaata | |
| 1 | mwlplllgal lwavlwllrd rqslpasnaf vfitgcdsgf grllalqldq rgfrvlascl | (SEQ ID NO:190) |
| 61 | tpsgaedlqr vassrlhttl lditdpqsvq qaakwvemhv keaglfglvn nagvagiigp | |
| 121 | tpwltrddfq rvlnvntmgp igvtlallpl lqqargrvin itsvlgrlaa ngggycvskf | |
| 181 | gleafsdslr rdvahfgirv sivepgffrt pvtnleslek tlqacwarlp patqahygga | |
| 241 | fltkylkmqq rimnlicdpd ltkvsrcleh altarhprtr yspgwdakll wlpasylpas | |
| 301 | lvdavltwvl pkpaqavy | |

(CG2151)—XP_{—}033135 thioredoxin reductase beta

| | | |
|---|---|---|
| 1 | ccggacctca ggcccagttc agtgtacttc ccctctctac ttcctccctc cagtcccttc | (SEQ ID NO:191) |
| 61 | tccatccctc ccttttttgg ctgccccttg cctgccttcc tcgccagtag cttgcagagt | |
| 121 | agacacgatg acaccttttg caggctaaaa aggctgagag tggcactatg tgcagtgagc | |
| 181 | caccatggag gaccaagcag gtcagcggga ctatgatctc ctggtggtcg gcgggggatc | |
| 241 | tggtggcctg gcttgtgcca aggaggccgc ccagctggga aggaaggtgg ccgtggtgga | |
| 301 | ctacgtggaa ccttctcccc aaggcacccg gtggggcctc ggcggcacct gcgtcaacgt | |
| 361 | gggctgcatc cccaagaagc tgatgcacca ggcggcactg ctgggaggcc tgatccaaga | |
| 421 | tgcccccaac tatggctggg aggtggccca gcccgtgccg catgactgga ggaagatggc | |
| 481 | agaagctgtt caaaatcacg tgaaatcctt gaactggggc caccgtgtcc agcttcagga | |
| 541 | cagaaaagtc aagtacttta acatcaaagc cagctttgtt gacgagcaca cggtttgcgg | |
| 601 | cgttgccaaa ggtgggaaag agattctgct gtcagccgat cacatcatca ttgctactgg | |
| 661 | agggcggccg agatacccca cgcacatcga aggtgccttg gaatatggaa tcacaagtga | |
| 721 | tgacatcttc tggctgaagg aatcccctgg aaaaacgttg gtggtcgggg ccagctatgt | |
| 781 | ggccctggag tgtgctggct tcctcaccgg gattgggctg gacaccacca tcatgatgcg | |
| 841 | cagcatcccc ctccgcggct tcgaccagca aatgtcctcc atggtcatag agcacatggc | |
| 901 | atctcatggc acccggttcc tgaggggctg tgccccctcg cgggtcagga ggctccctga | |
| 961 | tggccagctg caggtcacct gggaggacag caccaccggc aaggaggaca cgggcacctt | |
| 1021 | tgacaccgtc ctgtgggcca taggtcgagt cccagacacc agaagtctga atttggagaa | |
| 1081 | ggctggggta gatactagcc ccgacactca gaagatcctg gtggactccc gggaagccac | |
| 1141 | ctctgtgccc cacatctacg ccattggtga cgtggtggag gggcggcctg agctgacacc | |
| 1201 | catagcgatc atggccggga ggctcctggt gcagcggctc ttcggcgggt cctcagatct | |
| 1261 | gatggactac gacaatgttc ccacgaccgt cttcaccccg ctggagtatg gctgtgtggg | |
| 1321 | gctgtccgag gaggaggcag tggctcgcca cgggcaggag catgttgagg tctatcacgc | |
| 1381 | ccattataaa ccactggagt tcacggtggc tggacgagat gcatcccagt gttatgtaaa | |
| 1441 | gatggtgtgc ctgagggagc ccccacagct ggtgctgggc ctgcatttcc ttggccccaa | |
| 1501 | cgcaggcgaa gttactcaag gatttgctct ggggatcaag tgtggggctt cctatgcgca | |
| 1561 | ggtgatgcgg accgtgggta tccatcccac atgctctgag gaggtagtca agctgcgcat | |
| 1621 | ctccaagcgc tcaggcctgg accccacggt gacaggctgc tgagggtaag cgccatccct | |
| 1681 | gcaggccagg gcacacggtg cgcccgccgc cagctcctcg gaggccagac ccaggatggc | |
| 1741 | tgcaggccag gtttgggggg cctcaaccct ctcctggagc gcctgtgaga tggtcagcgt | |
| 1801 | ggagcgcaag tgctggacag gtggcccgtg tgccccacag ggatggctca ggggactgtc | |
| 1861 | cacctcaccc ctgcacctct cagcctctgc cgccgggcac ccccccccag gctcctggtg | |
| 1921 | ccagatgatg acgacctggg tggaaaccta ccctgtgggc acccatgtcc gagccccctg | |
| 1981 | gcatttctgc aatgcaaata aagagggtac tttttctgaa gtgtg | |
| 1 | medqagqrdy dllvvgggsg glacakeaaq lgrkvavvdy vepspqgtrw glggtcvnvg | (SEQ ID NO:192) |
| 61 | cipkklmhqa allggliqda pnygwevaqp vphdwrkmae avqnhvksln wghrvqlqdr | |
| 121 | kvkyfnikas fvdehtvcgv akggkeills adhiiiatgg rprypthieg aleygitsdd | |
| 181 | ifwlkespgk tlvvgasyva lecagfltgi gldttimmrs iplrgfdqqm ssmviehmas | |
| 241 | hgtrflrgca psrvrrlpdg qlqvtwedst tgkedtgtfd tvlwaigrvp dtrslnleka | |
| 301 | gvdtspdtqk ilvdsreats vphiyaigdv vegrpeltpi aimagrllvq rlfggssdlm | |
| 361 | dydnvpttvf tpleygcvgl seeeavarhg qehvevyhah ykpleftvag rdasqcyvkm | |
| 421 | vclreppqlv lglhflgpna gevtqgfalg ikcgasyaqv mrtvgihptc seevvklris | |
| 481 | krsgldptvt gcxg | |

Putative function
(CG10964)—unknown, similarity to dehydrogenases
(CG2151)—thioredoxin reductase

### Example 16

### Category 3

Line ID—418

Phenotype—Lethal phase embryonic larval phase3-pre-pupal-pupal. High mitotic index, dot-like chromosomes, strong metaphase arrest

Annotated Drosophila genome genomic segment containing P element insertion site (and map position)—AE003431 (4C 11-16)

P element insertion site—289,752

Annotated Drosophila genome Complete Genome candidate

CG3000—rap, fizzy related

(SEQ ID NO:193)

CTTTGGCTTGTTTGCTTGAAAAAACGTAACTTTTTTTGTTGTAATGAAGG
AAGCAGCACGGGCAGTAGACCAACTCGAAATCGCGCATTGCCAACACGTA
ACGTACCAGCCCGTGTAATAACAGAAGAAACCCCGAGCCGCAACAACAAC
CCCCGAAAAGCGGTAGTTGTAAGAGTTTTCCCAAAGTGGCAGCGGCAATT
ACACGGCGAGAAACGAGTTCGCGTCGCGTCCAGCTGTTTGAAAATCAAAA
TTAACCGTTTTTAGCGCGTGAAACAAGACGTTTAGAACCGTGTTCAAAAT
CCCTCGTACATAAATTGTGTGTACATTTATATATATATATATTTTCTACG
CCACGTTAACCAGACTTTTTAAGTTTTAAATTAAAACTAAAGACGTATTA
TTTTTTTTTTTTTGAGTGTTTATATTTTTTTTTTTGCAAGTTTTGTTTGG
TTACATTTGAGTTTGTGTTGAGTTTTTGCCAGCCAAAGGCGCTTAAGATG
TTTAGTCCCGAGTACGAGAAGCGCATCCTGAAGCACTACAGTCCTGTGGC
ACGGAATCTGTTCAACAACTTCGAGTCGTCCACTACGCCCACATCTCTCG
ACCGCTTCATACCCTGCAGAGCGTACAACAACTGGCAGACGAACTTTGCG
TCAATCAACAAGTCCAATGACAACTCGCCGCAGACGAGTAAGAAGCAGCG
GGACTGCGGGGAAACGGCACGCGATAGTCTCGCCTACTCCTGCCTACTGA
AGAACGAGCTCCTCGGATCGGCAATCGACGACGTGAAGACCGCCGGCGAG
GAGCGGAATGAGAATGCCTACACGCCGGCCGCAAAGCGGAGTCTCTTCAA
GTACCAGTCACCCACCAAGCAGGACTACAATGGCGAGTGTCCGTACTCGT
TGTCACCCGTCAGCGCCAAAAGTCAGAAGCTGTTGCGATCGCCGCGCAAG
GCTACGCGCAAAATCTCTCGCATTCCCTTCAAGGTGCTAGACGCGCCCGA
GTTGCAGGACGACTTCTATCTGAACCTGGTCGACTGGTCGTCGCAGAACG
TACTGGCTGTAGGCCTGGGCAGCTGTGTCTATCTGTGGAGCGCGTGCACC
AGTCAGGTTACCCGCCTGTGTGATCTCAGTCCGGATGCGAATACGGTGAC
CTCGGTGTCGTGGAACGAGCGTGGCAACACCGTGGCCGTGGGCACACATC
ACGGCTACGTGACCGTCTGGGATGTGGCGGCCAATAAGCAGATCAACAAA
CTGAATGGCCATTCGGCGCGTGTGGGCGCCTTGGCATGGAACAGTGACAT
CCTGTCGAGCGGGTCGCGAGACCGTTGGATCATACAGCGGGATACGAGAA
CGCCGCAACTGCAATCGGAGCGCAGATTGGCCGGACATCGGCAGGAGGTG
TGCGGACTGAAATGGTCACCGGATAATCAATACTTGGCCAGTGGCGGCAA
CGATAATCGGTTGTATGTGTGGAATCAGCATTCCGTGAATCCCGTACAAT
CATACACGGAGCATATGGCGGCTGTAAAGGCGATCGCGTGGTCGCCGCAT
CACCACGGACTCCTGGCCAGCGGCGGTGGAACGGCGGATAGGTGTATCCG
TTTCTGGAATACGCTGACGGGCCAGCCCATGCAGTGCGTGGACACGGGCT
CGCAGGTTTGCAATCTGGCCTGGTCCAAGCACTCCTCGGAGCTGGTCTCC
ACGCACGGCTACTCGCAGAACCAGATACTCGTGTGGAAATATCCCTCCCT
GACGCAAGTGGCCAAGCTGACGGGCCATTCGTATCGTGTGCTCTATCTGG
CGCTGAGTCCCGATGGTGAGGCTATTGTTACGGGCGCCGGCGACGAGACG
CTGCGATTTTGGAACGTATTCAGCAAGGCGCGCAGTCAGAAGGAGAACAA
GTCCGTTCTGAATCTGTTTGCCAATATCAGATAAGGACAATAACTCCAAG
CGAGCGAAGACTGAGCGAGCGCCAAAGGCAAACACAACACAACACAAAAC
AAAACAAAACAAAGCAAAGTATAATATAAATAAAATGGATACTTGAAACC
GAAAAACAAAGCCAACCAACCAATCAGCAAAAACCAAGCTGAAGCTAACA
AACTAATCGAGCCTATATGCTATATATATACAAACGATTCTTGTTCAGCA
GTCGTTTTGTAAATTGTTGTGTGACCCCACAGCAGCAATAGATTAAATAA
ATTTAAGTTAAGCAATCTGTATAGAACGGTAATTAGCAACATTTACGTAG
GTAAACACATGCAATTTATGAAGGAATAACATCAAGAGAGATGGCTGAAA
CAAGAACTGAAAATGAAACTAAGTCTATGGAAATTGTAAGTAATTGGAAA
ATCAACAACACCACACTCACACACTATCTTTAATCGACATTTTTTGTTGC
TGCTTTTTTAAATGTATTGTTTTTTTTTTGTGGTACACCTACACTACACC
TAAGAAAATTGGATACCCCTACATATACATTTATACGTTTATATATATAT
ATTTTTTTGCTAGCCTCTAAGTAACTAACTTTATTTCAAGCAAACATTTA
TACACATATTTCGCTCACTAGAAACACTCATACCCCCGAAAACACAATGT
ATATTAAATAAACTTATACAATTTCAAAATGTGCCCCAAAAAGTA

(SEQ ID NO:194)

MFSPEYEKRILKHYSPVARNLFNNFESSTTPTSLDRFIPCRAYNNWQTNF
ASINKSNDNSPQTSKKQRDCGETARDSLAYSCLLKNELLGSAIDDVKTAG
EERNENAYTPAAKRSLFKYQSPTKQDYNGECPYSLSPVSAKSQKLLRSPR
KATRKISRIPFKVLDAPELQDDFYLNLVDWSSQNVLAVGLGSCVYLWSAC
TSQVTRLCDLSPDANTVTSVSWNERGNTVAVGTHHGYVTVWDVAANKQIN
KLNGHSARVGALAWNSDILSSGSRDRWIIQRDTRTPQLQSERRLAGHRQE
VCGLKWSPDNQYLASGGNDNRLYVWNQHSVNPVQSYTEHMAAVKAIAWSP
HHHGLLASGGGTADRCIRFWNTLTGQPMQCVDTGSQVCNLAWSKHSSELV
STHGYSQNQILVWKYPSLTQVAKLTGHSYRVLYLALSPDGEAIVTGAGDE
TLRFWNVFSKARSQKENKSVLNLFANIR

Human homologue of Complete Genome candidate

XP_{—}009259 Fzr1 protein

| | | |
|---|---|---|
| 1 | ggccgcggcc gggcctgcgg gagctgcgga ggccggaggc gggcgctgtg cggtgccagg | (SEQ ID NO:195) |
| 61 | agaggcgggg tcggcgggag ccagcgagcc acgggagcga gccaggctaa ccttgccgcg | |
| 121 | ggccgagccc tgcctcgcca tggaccagga ctatgagcgg cgcctgcttc gccagatcgt | |
| 181 | catccagaat gagaacacga tgccacgcgt cacagagatg cggcggaccc tgacgcctgc | |
| 241 | cagctcccca gtgtcctcgc ccagcaagca cggagaccgc ttcatcccct ccagagccgg | |
| 301 | agccaactgg agcgtgaact tccacaggat taacgagaat gagaagtctc ccagtcagaa | |
| 361 | ccggaaagcc aaggacgcca cctcagacaa cggcaaagac ggcctggcct actctgccct | |
| 421 | gctcaagaat gagctgctgg gtgccggcat cgagaaggtg caggacccgc agactgagga | |
| 481 | ccgcaggctg cagccctcca cgcctgagaa gaagggtctg ttcacgtatt cccttagcac | |
| 541 | caagcgctcc agccccgatg acggcaacga tgtgtctccc tactccctgt ctcccgtcag | |
| 601 | caacaagagc cagaagctgc tccggtcccc ccggaaaccc acccgcaaga tctccaagat | |
| 661 | ccccttcaag gtgctggacg cgcccgagct gcaggacgac ttctacctca atctggtgga | |
| 721 | ctggtcgtcc ctcaatgtgc tcagcgtggg gctaggcacc tgcgtgtacc tgtggagtgc | |
| 781 | ctgtaccagc caggtgacgc ggctctgtga cctctcagtg gaaggggact cagtgacctc | |
| 841 | cgtgggctgg tctgagcggg ggaacctggt ggcggtgggc acacacaagg gcttcgtgca | |
| 901 | gatctgggac gcagccgcag ggaagaagct gtccatgttg gagggccaca cggcacgcgt | |
| 961 | cggggcgctg gcctggaatg ctgagcagct gtcgtccggg agccgcgacc gcatgatcct | |
| 1021 | gcagagggac atccgcaccc cgccactgca gtcggagcgg cggctgcagg gccaccggca | |
| 1081 | ggaggtgtgc gggctcaagt ggtccacaga ccaccagctc ctcgcctcgg ggggcaacga | |
| 1141 | caacaagctg ctggtctgga atcactcgag cctgagcccc gtgcagcagt acacggagca | |
| 1201 | cctggcggcc gtgaaggcca tcgcctggtc cccacatcag cacgggctgc tggcctcggg | |
| 1261 | gggcggcaca gctgaccgct gtatccgctt ctggaacacg ctgacaggac aaccactgca | |
| 1321 | gtgtatcgac acgggctccc aagtgtgcaa tctggcctgg tccaagcacg ccaacgagct | |
| 1381 | ggtgagcacg cacggctact cacagaacca gatccttgtc tggaagtacc cctccctgac | |
| 1441 | ccaggtggcc aagctgaccg ggcactccta ccgcgtgctg tacctggcaa tgtcccctga | |
| 1501 | tggggaggcc atcgtcactg gtgctggaga cgagaccctg aggttctgga acgtctttag | |
| 1561 | caaaacccgt tcgacaaagg agtctgtgtc tgtgctcaac ctcttcacca ggatccggta | |
| 1621 | aacctgccgg gcaggaccgt gccacaccag ctgtccagag tcggaggacc ccagctcctc | |
| 1681 | agcttgcatg gactctgcct tcccagcgct tgtcccccga ggaaggcggc tgggcgggcg | |
| 1741 | gggagctggg cctggaggat cctggagtct cattaaatgc ctgattgtga accatgtcca | |
| 1801 | ccagtatctg gggtgggcac gtggtcgggg accctcagca gcaggggctc tgtctccctt | |
| 1861 | cccaaagggc gagaaccaca ttggacggtc ccggctcaga ccgtctgtac tcagagcgac | |
| 1921 | ggatgccccc tgggaccctc actgcctccg tctgttcatc acctgcccac cggagccgca | |
| 1981 | tgctcttcct ggaactgccc acgtctgcac agaacagacc accagacgcc agggctgatt | |
| 2041 | ggtgggggcc tgagaccccg gttgcccatt catggctgca ccccaccatg tcaaacccaa | |
| 2101 | gaccagcccc aaggccagac caaggcatgt aggcctgggc aggtggctcg gggccactgg | |
| 2161 | cggagccagc ctgtggatcc aagagacagt ccccacctgg gcttcacggc atccttgcag | |
| 2221 | ccacctctgc tgtcactgct cgaagcagca gtctctctgg aagcatctgt gtcatggcca | |
| 2281 | tcgcccggcg gtcagtgggc ttcagatggg cctgtgcatc ctggccaagc gtcaccctca | |
| 2341 | cactggagga ggatgtctgc tctggactta tcaccccagg agaactgaac ccggacctgc | |
| 2401 | tcactgccct ggctggagag gagcacaaca gatgccacgt cttcgtgcat tcgccaacac | |
| 2461 | gtgccctcac agggccagcg tcctccttcc ctgcgcaaga cttgcgtccc ccatgcctgc | |
| 2521 | tgggtggctg ggtcctgtgg aggccagcag cggtgtggcc cccgccccca ggctgcctgt | |
| 2581 | gtcttcacct gtcctgtcca ccagcgccaa cagccgtggg gaagccaagg agacccaagg | |
| 2641 | ggtccaggag gtgggcgccc tccatccttc gagaagcttc ccaggctcct ctgcttctct | |
| 2701 | gtctcatgct cccaggctgc acagcaggca gggagggagg caaggcaggg gagtggggcc | |
| 2761 | tgagctgagc actgccccct caccccccca ccaccccttc ccatttcatc ggtggggacg | |
| 2821 | tggagagggt ggggcgggct ggggttggag ggtcccaccc accaccctgc tgtgcttggg | |
| 2881 | aacccccact ccccactccc cacatcccaa catcctggtg tctgtcccca gtggggttgg | |
| 2941 | cgtgcatgtg tacatatgta tttgtgactt ttctttgg | |
| 1 | mdqdyerrll rqiviqnent mprvtemrrt ltpasspvss pskhgdrfip sraganwsvn | (SEQ ID NO:196) |
| 61 | fhrineneks psqnrkakda tsdngkdgla ysallknell gagiekvqdp qtedrrlqps | |
| 121 | tpekkglfty slstkrsspd dgndvspysl spvsnksqkl lrsprkptrk iskipfkvld | |
| 181 | apelqddfyl nlvdwsslnv lsvglgtcvy lwsactsqvt rlcdlsvegd svtsvgwser | |
| 241 | gnlvavgthk gfvqiwdaaa gkklsmlegh tarvgalawn aeqlssgsrd rmilqrdirt | |
| 301 | pplqserrlq ghrqevcglk wstdhqllas ggndnkllvw nhsslspvqq ytehlaavka | |
| 361 | iawsphqhgl lasgggtadr cirfwntltg qplqcidtgs qvcnlawskh anelvsthgy | |
| 421 | sqnqilvwky psltqvaklt ghsyrvlyla mspdgeaivt gagdetlrfw nvfsktrstk | |
| 481 | esvsvlnlft rir | |

Putative function
Cell cycle regulator involved in cyclin degradation

### Example 17

### Category 3

Line ID—121

Phenotype—Lethal phase larval phase 3—prepupal—pupal—pharate adult-adult. High mitotic index, dot and rod-like overcondensed chromosomes, high frequency of polyploids

Annotated Drosophila genome genomic segment containing P element insertion site (and map position)—AE003493 (12B7)

P element insertion site—not determined

Annotated Drosophila genome Complete Genome candidate

CG10988—1(1)dd4 gamma tubulin ring complex

(SEQ ID NO:197)

TAACACTGCACTAAATAATTTTAATAAATTATTTGTATGAAGTACGCGCC
AATTGGATGCGTTTTTGTCCTATCTGTCGAAGATTTCACGCATCCCGAAC
AATTGCCAGTGACTGCACGCCGTATTATAGCCAGGGAACAGCTGTGCGTT
TGCCATTGGCCAACAGTTGTTGTCCACTTCGCAATTACCAAGCCATCCAA
AATCGGCTGTTTAACGCGCGCTTGATTGGATATTTATGAACAATTCAGTG
CACCAGGATGTCGCAGGACAGGATCGCCGGCATCGATGTGGCAACCAATT
CCACTGATATATCGAATATCATTAACGAGATGATCATCTGCATCAAGGGC
AAGCAGATGCCCGAAGTTCACGAAAAAGCAATGGATCATTTAAGCAAAAT
GATTGCCGCCAATAGTCGGGTCATTCGGGACTCAAATATGTTGACTGAGC
GCGAATGTGTCCAGAAGATAATGAAACTGCTGAGCGCCCGGAATAAGAAG
GAGGAGGGCAAAACTGTGTCGGATCACTTCAATGAGCTGTACAGGAAACT
CACGTTGACCAAGTGCGATCCGCACATGAGGCACTCGCTAATGACCCATC
TACTTACGATGACCGACAATTCGGATGCCGAAAAGGCAGTTGCCAGCGAA
GATCCACGTACTCAGTGCGATAATCTCACTCAGATTCTGGTCAGTCGTCT
TAACTCAATAAGTTCCTCCATAGCCAGTCTGAATGAGATGGGAGTGGTCA
ACGGAAATGGAGTAGGAGCAGCAGCGGTAACAGGAGCAGCAGCGGTAACA
GGAGCAGCAGCGGTAACAGGAGCAGCAGCGGTAACAGGAGCAGCAGCAAG
CCACAGTTATGATGCCACACAGTCCAGCATCGGATTGAGAAAACAGTCCT
TGCCCAACTACCTGGATGCAACAAAGATGTTGCCCGAGTCTCGACATGAT
ATAGTGATGAGTGCCATTTACTCCTTCACCGGCGTTCAAGGGAAGTATTT
GAAGAAGGATGTGGTAACGGGCCGTTTCAAGCTGGATCAGCAGAACATCA
AGTTCCTGACCACCGGCCAAGCGGGCATGTTGCTGCGGCTCTCCGAACTT
GGCTACTACCACGATCGAGTGGTCAAGTTTTCGGATGTATCGACCGGTTT
CAATGCCATTGGCAGCATGGGCCAGGCCCTGATTTCCAAACTCAAGGAGG
AGCTGGCGAATTTTCACGGGCAAGTGGCAATGCTTCACGATGAAATGCAG
CGTTTTCGGCAGGCCTCGGTGAATGGAATTGCAAACAAGGGGAAAAAGGA
TAGTGGGCCCGATGCTGGCGATGAAATGACGCTATTCAAGCTGCTCGCCT
GGTATATAAAGCCACTGCACCGGATGCAGTGGTTAACCAAGATTGCCGAC
GCCTGCCAGGTAAAGAAGGGCGGTGATTTGGCATCGACCGTTTATGATTT
CCTTGACAACGGTAACGATATGGTCAATAAATTGGTGGAGGATCTCCTAA
CTGCCATTTGTGGCCCACTGGTGCGCATGATCTCCAAATGGATTCTGGAG
GGCGGCATTAGCGATATGCATAGAGAGTTCTTTGTGAAGTCCATTAAAGA
TGTGGGCGTTGATCGGCTATGGCACGATAAATTCCGCCTACGATTGCCAA
TGCTGCCCAAGTTTGTGCCCATGGATATGGCCAATAAGATACTCATGACG
GGCAAATCCATTAATTTTCTAAGAGAAATCTGCGAGGAGCAGGGTATGAT
GAAGGAGCGCGACGAACTAATGAAGGTCATGGAATCTAGTGCCTCTCAAA
TCTTTTCGTACACACCGGACACCAGTTGGCATGCGGCCGTGGAAACGTGC
TACCAGCAGACCTCCAAACATGTCCTCGACATTATGGTGGGCCCACACAA
GCTGCTGGATCATTTGCACGGAATGCGGCGCTACTTGCTGTTGGGCCAGG
GCGATTTTATTAGCATTCTGATTGAAAACATGAAGAACGAACTGGAGCGA
CCGGGCCTTGATATATATGCTAACGATCTCACCTCCATGTTGGATTCCGC
TCTGCGCTGTACGAATGCCCAGTACGATGATCCTGATATTCTAAACCATC
TCGATGTGATTGTTCAACGACCGTTCAACGGTGATATTGGCTGGAACATC
ATCTCGCTGCAGTACATTGTCCACGGACCACTGGCCGCCATGCTGGAGTC
GACCATGCCAACGTACAAGGTGCTCTTCAAGCCACTCTGGCGCATGAAGC
ACATGGAGTTTGTGCTCTCGATGAAGATCTGGAAGGAGCAGATGGGCAAC
GCAAAGGCCCTTCGTACAATGAAGTCCGAAATCGGCAAGGCGTCACACCG
CCTCAACCTTTTCACTTCCGAGATCATGCACTTTATCCACCAAATGCAGT
ACTATGTGCTATTTGAGGTCATCGAGTGCAACTGGGTGGAGCTACAGAAG
AAGATGCAGAAGGCTACTACGTTGGACGAAATCCTGGAAGCTCACGAGAA
GTTTCTGCAAACGATTTTGGTGGGCTGTTTTGTCAGCAACAAAGCGAGTG
TGGAGCATTCGCTGGAGGTGGTGTACGAGAACATTATCGAATTGGAGAAG
TGGCAGTCGAGCTTTTACAAGGACTGCTTTAAGGAGCTAAATGCCCGCAA
GGAACTGTCCAAAATTGTGGAGAAATCGGAAAAGAAGGGTGTCTACGGAC
TGACCAACAAGATGATCCTGCAGCGCGACCAGGAGGCGAAGATATTTGCC
GAAAAGATGGACATCGCCTGCCGCGGCTTAGAAGTCATAGCAACCGATTA
CGAAAAGGCTGTCAGCACTTTCCTAATGTCTCTCAACTCTAGCGACGATC
CGAATTTGCAGCTCTTTGGCACTCGGCTGGACTTCAACGAGTACTACAAG
AAGAGGGACACCAATTTGAGCAAACCCCTGACCTTCGAGCACATGCGCAT
GAGCAATGTGTTCGCCGTGAACAGTCGCTTCGTGATATGTACGCCGTCCA
CTCAGGAATAGCGACCAATGTCCATGCAATCGGTTTATCCCAGTGTCCAT
ACATCATACCAAATCCCAAATCCCATACAGCATCAGCACTCCATTCAGTT
CAATTGCTGCTAAATATTTGAGATATCTCGATATCATTGGAGCCAATCCA
ACCAAACAAACTAATCCAATTATTAACTAAGCCTTCGAATCGAAAACAAC
CTCTATACATATATATCTCAAGCTTTGCCGTCAATCGCCTGGCTGCAAGC
CATCAACTTAAGATATCTCCAATACAAAATTATTGAGTAGTTGTAACGAA
AGTATTAAGCGACAATTTGTTTGTCGAAAAACGCAACGTTCTATTTTGTT
TGCGAATCCCATAATTTTTTTTACATCGAAGCTTAGTTGAAATAGATTTT
CGTAAGTGCATTTGCCAATTGCCATGTTGTAATTAAAGAGAATAAGAGAA
TGTTACGTACTTTAAAAGAATGTTTTAAAAAAGTTAATGTTTTGAACAGT
TTTAAACCGTAATGCGAG

(SEQ ID NO:198)

MSQDRIAGIDVATNSTDISNIINEMIICIKGKQMPEVHEKAMDHLSKMIA
ANSRVIRDSNMLTERECVQKIMKLLSARNKKEEGKTVSDHFNELYRKLTL
TKCDPHMRHSLMTHLLTMTDNSDAEKAVASEDPRTQCDNLTQILVSRLNS
ISSSIASLNEMGVVNGNGVGAAAVTGAAAVTGAAAVTGAAAVTGAAASHS
YDATQSSIGLRKQSLPNYLDATKMLPESRHDIVMSAIYSFTGVQGKYLKK
DVVTGRFKLDQQNIKFLTTGQAGMLLRLSELGYYHDRVVKFSDVSTGFNA
IGSMGQALISKLKEELANFHGQVAMLHDEMQRFRQASVNGIANKGKKDSG
PDAGDEMTLFKLLAWYIKPLHRMQWLTKIADACQVKKGGDLASTVYDFLD
NGNDMVNKLVEDLLTAICGPLVRMISKWILEGGISDMHREFFVKSIKDVG
VDRLWHDKFRLRLPMLPKFVPMDMANKILMTGKSINFLREICEEQGMMKE
RDELMKVMESSASQIFSYTPDTSWHAAVETCYQQTSKHVLDIMVGPHKLL
DHLHGMRRYLLLGQGDFISILIENMKNELERPGLDIYANDLTSMLDSALR
CTNAQYDDPDILNHLDVIVQRPFNGDIGWNIISLQYIVHGPLAAMLESTM
PTYKVLFKPLWRMKHMEFVLSMKIWKEQMGNAKALRTMKSEIGKASHRLN
LFTSEIMHFIHQMQYYVLFEVIECNWVELQKKMQKATTLDEILEAHEKFL
QTILVGCFVSNKASVEHSLEVVYENIIELEKWQSSFYKDCFKELNARKEL
SKIVEKSEKKGVYGLTNKMILQRDQEAKIFAEKMDIACRGLEVIATDYEK
AVSTFLMSLNSSDDPNLQLFGTRLDFNEYYKKRDTNLSKPLTFEHMRMSN
VFAVNSRFVICTPSTQE

Human homologue of Complete Genome candidate

AAC39727—spindle pole body protein spc98 homolog GCP3

| | | |
|---|---|---|
| 1 | caggaagggc gcgggccgcg gtccctgcgc gtgcggcggc agtggcggct ctgcccggac | (SEQ ID NO:199) |
| 61 | caccgtgcac ggctccgggc gaggatggcg accccggacc agaagtcgcc gaacgttctg | |
| 121 | ctgcagaacc tgtgctgcag gatcctgggc aggagcgaag ctgatgtagc ccagcagttc | |
| 181 | cagtatgctg tgcgggtgat tggcagcaac ttcgccccaa ctgttgaaag agatgaattt | |
| 241 | ttagtagctg aaaaaatcaa gaaagagctt attcgacaac gaagagaagc agatgctgca | |
| 301 | ttattttcag aactccacag aaaacttcat tcacagggag ttttgaaaaa taaatggtca | |
| 361 | atactctacc tcttgctgag cctcagtgag gacccacgca ggcagccaag caaggtttct | |
| 421 | agctatgcta cgttatttgc tcaggcctta ccaagagatg cccactcaac cccttactac | |
| 481 | tatgccaggc ctcagaccct tcccctgagc taccaagatc ggagtgccca gtcagcccag | |
| 541 | agctccggca gcgtgggcag cagtggcatc agcagcattg gcctgtgtgc cctcagtggc | |
| 601 | cccgcgcctg cgccacaatc tctcctccca ggacagtcta atcaagctcc aggagtagga | |
| 661 | gattgccttc gacagcagtt ggggtcacga ctcgcatgga ctttaactgc aaatcagcct | |
| 721 | tcttcacaag ccactacctc aaaaggtgtc cccagtgctg tgtctcgcaa catgacaagg | |
| 781 | tccaggagag aaggggatac gggtggtact atggaaatta cagaagcagc tctggtaagg | |
| 841 | gacattttgt acgtctttca gggcatagat ggcaaaaaca tcaaaatgaa caacactgaa | |
| 901 | aattgttaca aagtagaagg aaaggcaaat ctaagtaggt ctttgagaga cacagcagtc | |
| 961 | aggctttctg agttgggatg gttgcataat aaaatcagaa gatacacgga ccagaggagc | |
| 1021 | ctggaccgct cattcggact cgtcgggcag agcttttgtg ctgccttgca ccaggaactc | |
| 1081 | agagaatact atcgattgct ctctgtttta cattctcagc tacaactaga ggatgaccag | |
| 1141 | ggtgtgaatt tgggacttga gagtagttta acacttcggc gcctcctggt ttggacctat | |
| 1201 | gatcccaaaa tacgactgaa gacccttgcg gccctagtgg accactgcca aggaaggaaa | |
| 1261 | ggaggtgagc tggcctcagc tgtccacgcc tacacaaaaa caggagaccc gtacatgcgg | |
| 1321 | tctctggtgc agcacatcct cagcctcgtg tctcatcctg ttttgagctt cctgtaccgc | |
| 1381 | tggatatatg atggggagct tgaggacact taccacgaat tttttgtagc atcagatcca | |
| 1441 | acagttaaaa cagatcgact gtggcacgac aagtatactt tgaggaaatc gatgattcct | |
| 1501 | tcgtttatga cgatggatca gtctaggaag gtccttttga taggaaaatc aataaatttc | |
| 1561 | ttgcaccaag tttgtcatga tcagactccc actacaaaga tgatagctgt gaccaagtct | |
| 1621 | gcagagtcac cccaggacgc tgcagaccta ttcacagact tggaaaatgc atttcagggg | |
| 1681 | aagattgatg ctgcttattt tgagaccagc aaatacctgt tggatgttct caataaaaag | |
| 1741 | tacagcttgc tggaccacat gcaggcaatg aggcggtacc tgcttcttgg tcaaggagac | |
| 1801 | tttataaggc acttaatgga cttgctaaaa ccagaacttg tccgtccagc tacgactttg | |
| 1861 | tatcagcata acttgactgg aattctagaa accgctgtca gagccaccaa cgcacagttt | |
| 1921 | gacagtcctg agatcctgcg aaggctggac gtgcggctgc tggaggtctc tccaggtgac | |
| 1981 | actggatggg atgtcttcag cctcgattat catgttgacg gaccaattgc aactgtgttt | |
| 2041 | actcgagaat gtatgagcca ctacctaaga gtatttaact tcctctggag ggcgaagcgg | |
| 2101 | atggaataca tcctcactga catacggaag ggacacatgt gcaatgcaaa gctcctgaga | |
| 2161 | aacatgccag agttctccgg ggtgctgcac cagtgtcaca ttttggcctc tgagatggtc | |
| 2221 | catttcattc atcagatgca gtattacatc acatttgagg tgcttgaatg ttcttgggat | |
| 2281 | gagctttgga acaaagtcca gcaggcccag gatttggatc acatcattgc tgcacacgag | |
| 2341 | gtgttcttag acaccatcat ctcccgctgc ctgctggaca gtgactccag ggcactttta | |
| 2401 | aatcaactta gagctgtgtt tgatcaaatt attgaacttc agaatgctca agatgcaata | |
| 2461 | tacagagctg ctctggaaga attgcagaga cgattacagt ttgaagagaa aaagaaacag | |
| 2521 | cgtgaaattg agggccagtg gggagtgacg gcagcagagg aagaggagga aaataagagg | |
| 2581 | attggagaat ttaaagaatc tataccaaaa atgtgctcac agttgcgaat attgacccat | |
| 2641 | ttctaccagg gtatcgtgca gcagtttttg gtgttactga cgaccagctc tgacgagagt | |
| 2701 | cttcggtttc ttagcttcag gctggacttc aacgagcatt acaaagccag ggagcccagg | |
| 2761 | ctccgtgtgt ctctgggtac cagggggcgg cgcagctccc acacgtgaag ctcgcggtcc | |
| 2821 | tcccagggag ctgcgggtga tgttcgttgc actgctagac acgaaattcc cattgacgtc | |
| 2881 | ctgcaggaac tgcatgctgc aggtgtcctg cccttccgcc cacgagtgcg ccatgtttca | |
| 2941 | gcggagcggc gtgtgggaga agccacgtcg tgtttcacat gtcggagtcg aatgcatttg | |
| 3001 | taaatcccta agtcaagtag gctggctgca ctgttcacat ttgtctctaa aagtcttcat | |
| 3061 | cgctaaaaga taccataatt tgctgaggct tcttaagctt tctatgttat aatttatatt | |
| 3121 | tgtcacttta aaaaatccat ttcttttaga aaaaattagg gtgataggat attcattagt | |
| 3181 | taagatggta acgtcattgc tattttttta acatcctctt tagaggtaat ttttgttaac | |
| 3241 | ataaccaaaa attaaattga aacaaaatgt cccaactaag aaaatatata gagcatttta | |
| 3301 | ttttttttta gtgttgtaaa atattaacct ctgtgagatc ctttgtatct taatgcatta | |
| 3361 | cctttacaca tatttattct tattttctct cctttcagag tttacatttt tatatttaat | |
| 3421 | ttactatttc agatttttaa aatagtatag aaaaaagtag gagtgataga gaacaaaaat | |
| 3481 | actcttatac agtgcaaccc aaataccgcg aatgcatcag ctaaagcagc gtgtaaatag | |
| 3541 | gagtgatgag aaagttaatg gagtatttta ttttcaaagt tcctgataag cattggaaag | |
| 3601 | aaatcgacat ggataatgaa gatttccttt ttccttgcct attttttcat tgtaaatatt | |
| 3661 | tatatactac tgaccaagat gttggggtgg gggggattgt tttttgtaaa aatgtcatta | |
| 3721 | tcaggtcaca taaatctgcc tttatgttgc ataagtgaaa atttagaaaa ttaaaagcaa | |
| 3781 | ttatctttca aaaaa | |
| 1 | matpdqkspn vllqnlccri lgrseadvaq qfqyavrvig snfaptverd eflvaekikk | (SEQ ID NO:200) |
| 61 | elirqrread aalfselhrk lhsqgvlknk wsilylllsl sedprrqpsk vssyatlfaq | |
| 121 | alprdahstp yyyarpqtlp lsyqdrsaqs aqssgsvgss gissiglcal sgpapapqsl | |
| 181 | lpgqsnqapg vgdclrqqlg srlawtltan qpssqattsk gvpsavsrnm trsrregdtg | |
| 241 | gtmeiteaal vrdilyvfqg idgknikmnn tencykvegk anlsrslrdt avrlselgwl | |
| 301 | hnkirrytdq rsldrsfglv gqsfcaalhq elreyyrlls vlhsqlqled dqgvnlgles | |
| 361 | sltlrrllvw tydpkirlkt laalvdhcqg rkggelasav haytktgdpy mrslvqhils | |
| 421 | lvshpvlsfl yrwiydgele dtyheffvas dptvktdrlw hdkytlrksm ipsfmtmdqs | |
| 481 | rkvlligksi nflhqvchdq tpttkmiavt ksaespqdaa dlftdlenaf qgkidaayfe | |
| 541 | tskylldvln kkyslldhmq amrrylllgq gdfirhlmdl lkpelvrpat tlyqhnltgi | |
| 601 | letavratna qfdspeilrr ldvrllevsp gdtgwdvfsl dyhvdgpiat vftrecmshy | |
| 661 | lrvfnflwra krmeyiltdi rkghmcnakl lrnmpefsgv lhqchilase mvhfihqmqy | |
| 721 | yitfevlecs wdelwnkvqq aqdldhiiaa hevfldtiis rclldsdsra llnqlravfd | |
| 781 | qiielqnaqd aiyraaleel qrrlqfeekk kqreiegqwg vtaaeeeeen krigefkesi | |
| 841 | pkmcsqlril thfyqgivqq flvllttssd eslrflsfrl dfnehykare prlrvslgtr | |
| 901 | grrssht | |

Putative function
Component of the centrosome

### Example 18

### Category 3

LineID—237

Phenotype—Lethal phase larval stage 3 (few pupae). High mitotic index, colchicine-type overcondensation of chromosomes, polyploid cells, ‘mininuclei’ formation

Annotated Drosophila genome genomic segment containing P element insertion site (and map position)—AE0086 (10C4-5)

P element insertion site—182,487

Annotated Drosophila genome Complete Genome candidate
2 candidates:

CG 1558—novel protein

(SEQ ID NO:201)

ATGGAGCCAGCCGAAAGTCCAGAAAAATTAATGAAATTCGTACGCCGCAG
TGACGTACTGGAATACGTGGGCAACACGAGTGCCGTCGATCTATCGAGCG
GTGATCTCTCCGACATCGATCTCAAGGACGTGCCGGCCCAACTGGAGGCC
ACTTTGAAACCGCGTCGCTATGAAGCAAGCACTTTGTTTAACATTGACCT
GGACGATATCTGGGATCCTAGCTGTCAGGAGGACGAGGTGCAGCAGTACA
AGGAGCGCGCCCAGAAGGAGCAGCAAAAGTTCTTCGACTTTGTAATGCAT
GCGGCACTGGACACGGACAATCGCAAGGTTAGCTTCAAGCCAAACAAGGA
GCAGCAGCGTTACCTAGATCAGGGACCCAATTTGCAAAACTTCGTGCGAA
GCTCGTTGGCTTTCACAAACGCGGCCATCCGATTTCAGGCGGAGCACGAG
GACATGATGGAGCTGCAGTGCAATATGGACGATCACTACCTATTCATGCG
GAACACCATGATCAACAACGCTATACACCAGAATATGGCCAACCAACGGT
GACCCTAAGCTATGCATAAATATACATATGTGAATTGTAGATATTGATAA
ATTAAATTAAGACTCAGAGATTGTAAGACGGTTTGCTTTTGGCTTATACA
GTATAATTCGCTTAGCTGCCTCGAGTACTTTGCACAATGCCTCGATGCAG
GTAACTTAAAAATGCAGCTAACTTAATTTTTTTTTTTCTATTTTCTATTT
TCTATTCACAC

(SEQ ID NO:202)

MEPAESPEKLMKFVRRSDVLEYVGNTSAVDLSSGDLSDIDLKDVPAQLEA
TLKPRRYEASTLFNIDLDDIWDPSCQEDEVQQYKERAQKEQQKFFDFVMH
AALDTDNRKVSFKPNKEQQRYLDQGPNLQNFVRSSLAFTNAAIRFQAEHE
DMMELQCNMDDHYLFMRNTMINNAIHQNMANQR

CG11697—novel protein

(SEQ ID NO:203)

ATGATTTATGCGATCGTGATACACATACTGTCCCTTCTGGTGGGCTGTTT
CTATCCAGCATTCGCGTCCTACAAGATCCTGAAAAGTCAGAATTGTAGCG
TCAATGATCTTCGCGGATGGTTAATCTACTGGATTGCCTATGGAGTTTAT
GTGGCCTTTGATTATTTCACAGCGGGTCTGCTGGCATTTATTCCATTGCT
AAGTGAGTTCAAGGTGCTTCTCCTGTTCTGGATGTTGCCCTCTGTGGGCG
GCGGCAGTGAGGTGATCTACGAGGAGTTCCTGCGATCCTTTAGCTGTAAC
GAATCCTTCGACCAGGTCCTGGGACGTATCACCTTGGAATGGGGCGAATT
GGTGTGGCAACAAGTTTGCTCCGTTCTTAGCCATTTGATGGTTTTGGCAG
ATCGCTATCTCCTGCCCAGCGGTCATCGTCCTGCCCTCCAAATAACGCCC
AGCATCGAGGATCTGGTCAACGATGCCATAGCCAAAAGGCAGTTGGAAGA
GAAGCGGAAACAGATGGGTAACTTATCTGATACCATCAACGAGGTTTTGG
GAGAAAATATCGATTTAAATATGGATCTGCTGCACGGATCCGAATCTGAT
TTATTGGTTATTAAGGAGCCTATTTCCAAGCCCAAGGAGAGACCAATACC
GCCGCCGAAGCCAATGCGTCAGCCATCATCAAGCAACCAGCAAGAAATGA
ATCTTTCGTCGCAGTTTATGTGA

(SEQ ID NO:204)

MIYAIVIHILSLLVGCFYPAFASYKILKSQNCSVNDLRGWLIYWIAYGVY
VAFDYFTAGLLAFIPLLSEFKVLLLFWMLPSVGGGSEVIYEEFLRSFSCN
ESFDQVLGRITLEWGELVWQQVCSVLSHLMVLADRYLLPSGHRPALQITP
SIEDLVNDAIAKRQLEEKRKQMGNLSDTINEVLGENIDLNMDLLHGSESD
LLVIKEPISKPKERPIPPPKPMRQPSSSNQQEMNLSSQFM

Human homologue of Complete Genome candidate
(CG1558)—none

(CG1 1697)—BAB14444 unamed protein—similar to a hypothetical protein in the region deleted in human familial adenomatous polyposis 1

| | | |
|---|---|---|
| 1 | aacgccgggc agggcggcgg gcgcgctcag tctggcggcg gctgccgtga gctgactgac | (SEQ ID NO:205) |
| 61 | gttccgggaa cgccgcagca gcccgcgccg cccgcagcct agccgagccg cgccgcccgg | |
| 121 | gcctcgcccg cccgcctgcc cgccatggtg tcatggatca tctccaggct ggtggtgctt | |
| 181 | atatttggca ccctttaccc tgcgtattat tcctacaagg ctgtgaaatc aaaggacatt | |
| 241 | aaggaatatg tcaaatggat gatgtactgg attatatttg cacttttcac cacagcagag | |
| 301 | acattcacag acatcttcct ttgttggttt ccattctatt atgaactaaa aatagcattt | |
| 361 | gtagcctggc tgctgtctcc ctacacaaaa ggctccagcc tcctgtacag gaagtttgta | |
| 421 | catcccacac tatcttcaaa agaaaaggaa atcgatgatt gtctggtcca agcaaaagac | |
| 481 | cgaagttacg atgcccttgt gcacttcggg aagcggggct tgaacgtggc cgccacagcg | |
| 541 | gctgtgatgg ctgcttccaa gggacagggt gccttatcgg agagactgcg gagcttcagc | |
| 601 | atgcaggacc tcaccaccat caggggagac ggcgcccctg ctccctcggg ccccccacca | |
| 661 | ccggggtctg ggcgggccag cggcaaacac ggccagccta agatgtccag gagtgcttct | |
| 721 | gagagcgcta gcagctcagg caccgcctag aatccttcga tctcgcttca ggaagaaaag | |
| 781 | tacctcatcc tcggccaccg aaaccacgtg agtgagatga gccaacagca ccggatccac | |
| 841 | agaatgtttc ttctctgcct taaagagcta ttcactaata acatagaaat ccgcaagctg | |
| 901 | ggtgtgcttt gagtgtgcag cctcacaaac atggcctttt ctctctcccc ttccactttt | |
| 961 | aaggatttat ttttttcccc cttttcttta ttttgctggg gagaggctaa agggaaaggt | |
| 1021 | agtaggggcg ggggtggtga cctttaagtc ttctgaggtt ggtaattttc cacaattgga | |
| 1081 | ttgtcattat agacagcagt gtgtttttta gaaagataag agaatcaccc ctatgctgct | |
| 1141 | gagatgtaca tttgtaattt atctgttgca tacttagttt ttagtcctgt aaatgcaaac | |
| 1201 | acagcatttt ttacaacttt ctttgttctt ggtacttata ctttgaacta tgatgtacat | |
| 1261 | atttatggct tttggctttt aatataatgg acttgcaagg gctgccagag gttctgatat | |
| 1321 | gtaagaaaac tgcaaaaaca aatatagaca aatattttga ttctagagaa cgtctcagat | |
| 1381 | gtgcttataa agcttccaaa tacaactcca gtaagacatc cctttccctg caggagtgtg | |
| 1441 | gtctatattc tttagatagt tgtttagtca aaagaccaga caagttacaa actaagagaa | |
| 1501 | acaatatttc acaacacagt aaagtgtgat gagaggtcag gggaacatcc cagtaaaaga | |
| 1561 | gaagagtcac aggaagctca tctcctccct ggattctgga ttaggagctt ctgaatcttt | |
| 1621 | tccagggata ggcaggtagc tcactcttgg tgcaatttct tgaggatggg aacatgtaga | |
| 1681 | gctgctggaa ggagtaattc tgtgcttgac aaaggacgat ttctccttta tcgtgaccag | |
| 1741 | tgctgccgat ttcctgacag aggagcttac actctgagca ccttgtttta gcgaactcta | |
| 1801 | gcaaaacttg tttagcttag caaaaacaaa cacacaaaaa actgagaact ctgctgtttc | |
| 1861 | agatatgcca taacatacat ctgaaacaca tgtgtaacaa tcaaaatggt gggctctaga | |
| 1921 | atggttttgg agctcgagat cttcatgggt tagacttgct ggtcagaccc aggagcacct | |
| 1981 | gtggctcaca ccttctgttc ccctcctggc ctgtgcagaa tgtaaacagc agactcatac | |
| 2041 | tcaatgggca ctacaggcct tatcagacgt tttatacaag cctggattgc ttagtagggg | |
| 2101 | aataaggcat tctctgaggg ggctttccac ttagattgag aattttattt gaaaagaatc | |
| 2161 | tggtttaaat ggcattgtgg tccgaggtag ctgctctccc cactgagagc tgagccgaaa | |
| 2221 | tataagaata atatatttgt gcttcgagtt ggtgtttctt tcagtgtaat gcatgcagtg | |
| 2281 | gtcacaaccc agttactcat aatatttgga ttgtatttgt tcgtagatat gcccagaaga | |
| 2341 | ctagagaatt agtgttatat accatataga acttactgtc agtcaactat aaacaggccc | |
| 2401 | aattaaaaac tgttccatta ctacgcaaac acatattaga ggcctttgct gatgacacat | |
| 2461 | tagctggatc ttagccaccc cagaaagggt ttgatttgaa gctgattgtt gccagatatg | |
| 2521 | catattggaa tcccatctac ccatagttcc tctgaaggtg attttgtaat ttgcaaaagg | |
| 2581 | gtataggaaa atatacctaa aagcgaattt gtggctgaga ggataaacag aagctgtttg | |
| 2641 | ctcatgttct gtgccccaca cccaccaata cctaaatctg ttaaggaaga cagaaaatgt | |
| 2701 | tttctttgtg ctcattgagt agttccagac agaagaagaa tatactcttt aaaatgtatt | |
| 2761 | tacctgttag ttggaagtac ccagaattat cagaaacgaa tgcaaaaaaa aaaaaaaaaa | |
| 2821 | aaaaaagctt acacagcttc ttagcaattt tttttttttt tgccgaaaca ataaattgcc | |
| 2881 | tttagcagca gtttaaaatc ctatcgtgaa caacctatat tttcgccatt ttacaatgga | |
| 2941 | gagttgtgac aagtacaggt tatcaagttt gcacttaact atgccaaaaa aagtttgaag | |
| 3001 | cgctctattc tcagacatgc tgtattatta cttctcattc aagattgaaa aatataaagg | |
| 3061 | tatccaaact ctgtcttaat gtaaatgtaa ctatttttcc ttcaagtgtt gactagggag | |
| 3121 | tcggtttctc tcttaaagac actcactgta caactgaaag cagctgtcat atttctggca | |
| 3181 | aaatgtgttt acgtatctga caagttgtac atttgtgtat gaactgacat aaaatgtgaa | |
| 3241 | agcctgtaag tgtacatgta gtggtgtggt gttctgtcta gaggatacaa ctgaatgttt | |
| 3301 | ttaatttgct gacttacaga cacaggctgt ttacaaaatg ctagctggaa agtctgtaat | |
| 3361 | gttcatgtca taacttttag ttaattgcca ttgagcacct gttctgagga ggtgagatgt | |
| 3421 | ggacttgtgc ttataaactg gagagtttag tcataatccc tcctggcttt gtgtgaatag | |
| 3481 | cttgctcact ttgctggcct ttgaaatgtg ttctccgtga taagctatcc atgtgtttgt | |
| 3541 | gataagagtg cttgtcaacc atgaccatct ttgagccttc ctagtcctcc acctggcaca | |
| 3601 | gtatttgaaa tggcaaagga tgtgcttcat cctctaacaa acagtgtaca ctcccagagc | |
| 3661 | tgatattctg gattgtgact gtgcacattt cctctagttc atgtctgtag tccctataga | |
| 3721 | atgatctgta ataaaatagt atactggact gtgcatcaaa gggatgtaaa attacagtat | |
| 3781 | tccaaaggtt gaagttctgc tgttttgtta taatgcctga tacacatctt gaataaagtc | |
| 3841 | ttaacatttt tctttt | |
| 1 | miyaivihil sllvgcfypa fasykilksq ncsvndlrgw liywiaygvy vafdyftagl | (SEQ ID NO:206) |
| 61 | lafipllsef kvlllfwmlp svgggseviy eeflrsfscn esfdqvlgri tlewgelvwq | |
| 121 | qvcsvlshlm vladryllps ghrpalqitp siedlvndai akrqleekrk qmgnlsdtin | |
| 181 | evlgenidln mdllhgsesd llvikepisk pkerpipppk pmrqpsssnq qemnlssqfm | |
| 241 | | |

Putative function
(CG1558)—unknown
(CG11697)—may be deleted in human cancers, possibly a receptor.

### Example 19

### Corkscrew/Shp2 (Category 3)

Corkscrew (CG3954) as a candidate gene is detected in a screen of a P-element insertion library covering the X chromosome of Drosophila melanogaster (Peter et al. 2001) as mutant phenotype in fly line 171, as described above.

Mitotic defects are observed in brain squashes: low mitotic index, few cells in mitosis and metaphases with separated chromosomes, and is placed in Category 3 as described above.

Rescue and sequencing of genomic DNA flanking the P-element insertion site indicates that the P-element is inserted into the 5′ region of two genes: CG3954 corkscrew and CG16903 cyclin/non-specific RNA polymersae II transcription factor.

Line ID—171

Phenotype—Lethal phase larval stage 1-2. Low mitotic index, few cells in mitosis, metaphase with separated chromosomes

Annotated Drosophila genome genomic segment containing P element insertion site (and map position)—AE003423 (2D1-2)

P element insertion site—42,253

Annotated Drosophila genome Complete Genome candidate
2 candidates: CG3954—corkscrew. Protein tyrosine phosphatase required for cell signaling in eye development (2 splice variants) and CG16903—cyclin/non-specific RNA polymersae II transcription factor

CG3954—Corkscrew. Protein Tyrosine Phosphatase Required for cell Signaling in Eye Splice Variant 1

(SEQ ID NO:207)

ATGCTGTTCAACAAATGTCTGGAAAAGTTGTCCAGCTCGCTGGGCAATGT
GGTCAATCACAAGCTGCAAGAGAAACAAGTCTACAACAACAACAATATCA
ACAATAACAATAACAATACGCTAAACAACAACAATGCCTACAACAATCAG
CGAAACTTTGAGTACGAAAGAGCCATACAGGCGCACTACGGAAGCAAGGG
AAGACGCTCGGAGGAGCGCGAAAGGAGCGGCAAGTTCAAGGCCAGCAAGG
GTCGGAAAGCAAAGGTCACCCCACCAACGGAGACACCCGAGGCCCAGGAG
CCGGCCTGCAAGAACTGTATGACCCACGACGAGCTGGCCCAGATCATAAA
GGGCGTGGCCAAGGGCGCTGACGCGCAACGTAATCGAGACAACCGACTGC
AGCGCAGACGTCGTCCTCTCTCCGCCCAACCCTCCGCCGCTGCCTCCGCC
TCCACATCGACGGAATCTCTGCACCGTCTTACACCCAGCCCGCAGGCTTC
CTACCCGGCCACGCCCACCTCCTGGACAGCCACACCGCCCCAGTTCCCAG
CCGCCTTCGGCGGCGCCAGCTGCTCCAACAGCACACTGTCCCTCTTGGCC
ACCATGCGCGTCCAGCTCCATGGTTACACATGGTTTCATGGCAATCTTTC
CGGAAAGGAAGCGGAAAAATTGATCCTGGAGCGGGGCAAGAATGGTTCGT
TTCTCGTCCGTGAATCTCAGAGCAAGCCTGGCGACTTCGTCCTTTCCGTG
CGCACGGACGACAAAGTAACGCATGTCATGATTCGATGGCAGGACAAGAA
GTACGACGTCGGCGGCGGGGAATCCTTTGGCACCTTGTCGGAACTGATCG
ATCACTACAAGCGTAATCCCATGGTGGAGACGTGCGGAACCGTGGTGCAT
CTGCGACAGCCATTCAACGCCACACGAATCACGGCGGCCGGCATCAATGC
CCGGGTGGAACAGCTGGTCAAGGGAGGTTTCTGGGAGGAATTCGAATCGC
TGCAACAGGACAGTCGGGACACATTCTCGCGCAACGAGGGCTACAAACAG
GAGAACCGCCTCAAGAATCGCTACCGCAACATATTGCCATACGACCACAC
GCGCGTCAAGCTGCTGGACGTGGAGCATAGCGTGGCCGGAGCCGAGTACA
TCAATGCCAACTACATACGGCTGCCCACCGACGGCGACCTGTACAACATG
AGCAGCTCGTCGGAGAGCCTGAACAGCTCGGTGCCCTCGTGCCCCGCCTG
CACGGCTGCCCAGACACAGCGGAACTGCTCCAACTGCCAGCTGCAAAACA
AGACGTGCGTGCAGTGCGCCGTGAAGAGCGCCATTCTGCCGTATAGCAAC
TGTGCCACCTGCAGCCGCAAGTCAGACTCCCTGAGCAAGCACAAGCGGAG
CGAATCCTCGGCCTCTTCATCGCCCTCCTCCGGCTCTGGGTCCGGACCAG
GATCGTCGGGCACCAGCGGAGTGAGCAGCGTCAATGGACCCGGCACACCC
ACCAATCTCACGAGCGGCACAGCCGGATGTCTGGTCGGCCTGCTGAAGAG
ACACTCGAACGACTCGTCCGGAGCTGTTTCTATATCGATGGCCGAACGGG
AACGCGAGAGGGAGCGCGAGATGTTTAAGACCTACATCGCCACCCAGGGC
TGTCTGCTCACCCAGCAAGTGAACACGGTGACGGACTTCTGGAACATGGT
CTGGCAGGAGAACACGCGGGTGATCGTCATGACCACCAAGGAGTACGAGC
GCGGCAAAGAAAAGTGCGCCCGCTACTGGCCGGACGAGGGTAGATCGGAG
CAGTTCGGCCACGCGCGGATACAGTGCGTCTCGGAGAACTCGACCAGTGA
CTATACGCTGCGCGAGTTCCTCGTCTCGTGGCGGGATCAGCCGGCGCGCC
GGATCTTTCACTACCATTTCCAGGTGTGGCCGGATCACGGAGTGCCCGCC
GATCCGGGCTGTGTGCTCAACTTCCTGCAAGATGTCAACACGCGTCAGAG
TCACCTGGCTCAAGCGGGCGAGAAGCCGGGTCCGATCTGCGTGCACTGCT
CTGCGGGCATCGGTCGCACTGGCACCTTTATTGTGATCGATATGATTCTC
GATCAGATTGTGCGCAATGGATTGGATACTGAAATCGACATCCAGCGCAC
CATTCAGATGGTCCGATCGCAGCGTTCCGGTCTTGTGCAAACCGAGGCGC
AATACAAGTTCGTCTACTATGCGGTGCAGCACTATATACAGACCCTGATC
GCCCGGAAACGAGCTGAGGAGCAGAGCCTGCAGGTTGGCCGCGAGTACAC
CAATATAAAGTACACGGGCGAAATTGGAAACGATTCACAAAGATCTCCAT
TACCACCAGCAATTTCTAGCATAAGTTTAGTTCCGAGTAAGACGCCACTG
ACGCCGACATCGGCGGATTTGGGCACTGGGATGGGCCTAAGCATGGGCGT
GGGCATGGGCGTCGGCAACAAGCACGCATCGAAGCAGCAGCCGCCGTTGC
CGGTGGTCAACTGCAACAATAATAACAACGGCATTGGCAATAGCGGCTGC
AGCAACGGCGGCGGGAGCAGCACCACCAGCAGCAGCAACGGCAGCAGCAA
CGGTAACATCAACGCCCTACTGGGCGGCATCGGCTTGGGGCTGGGCGGCA
ATATGCGCAAGTCGAACTTTTACAGCGACTCGCTGAAGCAGCAACAGCAG
CGCGAGGAGCAGGCTCCGGCGGGAGCAGGTAAGATGCAGCAGCCGGCGCC
GCCGCTGCGACCGCGTCCTGGAATACTCAAGTTGCTCACCAGTCCCGTCA
TCTTTCAGCAAAATTCAAAAACATTCCCAAAGACATGA

(SEQ ID NO:208)

MLFNKCLEKLSSSLGNVVNHKLQEKQVYNNNNINNNNNNTLNNNNAYNNQ
RNFEYERAIQAHYGSKGRRSEERERSGKFKASKGRKAKVTPPTETPEAQE
PACKNCMTHDELAQIIKGVAKGADAQRNRDNRLQRRRRPLSAQPSAAASA
STSTESLHRLTPSPQASYPATPTSWTATPPQFPAAFGGASCSNSTLSLLA
TMRVQLHGYTWFHGNLSGKEAEKLILERGKNGSFLVRESQSKPGDFVLSV
RTDDKVTHVMIRWQDKKYDVGGGESFGTLSELIDHYKRNPMVETCGTVVH
LRQPFNATRITAAGINARVEQLVKGGFWEEFESLQQDSRDTFSRNEGYKQ
ENRLKNRYRNILPYDHTRVKLLDVEHSVAGAEYINANYIRLPTDGDLYNM
SSSSESLNSSVPSCPACTAAQTQRNCSNCQLQNKTCVQCAVKSAILPYSN
CATCSRKSDSLSKHKRSESSASSSPSSGSGSGPGSSGTSGVSSVNGPGTP
TNLTSGTAGCLVGLLKRHSNDSSGAVSISMAEREREREREMFKTYIATQG
CLLTQQVNTVTDFWNMVWQENTRVIVMTTKEYERGKEKCARYWPDEGRSE
QFGHARIQCVSENSTSDYTLREFLVSWRDQPARRIFHYHFQVWPDHGVPA
DPGCVLNFLQDVNTRQSHLAQAGEKPGPICVHCSAGIGRTGTFIVIDMIL
DQIVRNGLDTEIDIQRTIQMVRSQRSGLVQTEAQYKFVYYAVQHYIQTLI
ARKRAEEQSLQVGREYTNIKYTGEIGNDSQRSPLPPAISSISLVPSKTPL
TPTSADLGTGMGLSMGVGMGVGNKHASKQQPPLPVVNCNNNNNGIGNSGC
SNGGGSSTTSSSNGSSNGNINALLGGIGLGLGGNMRKSNFYSDSLKQQQQ
REEQAPAGAGKMQQPAPPLRPRPGILKLLTSPVIFQQNSKTFPKT

CG3954—Corkscrew. Protein Tyrosine Phosphatase Required for Cell Signaling in Eye Splice Variant 2

(SEQ ID NO:209)

AGTAAAAAAATAGTTTTTTTTTTGTATCCAACCAACCAACTGTAAAAATA
AGTTTAAACAAAGCATCTACTCATAAGTTTCATTTTTTTCCGTTAAGTGT
CAACATTATTTATTTTTTAAGTGTGCATTCAATAAGAAAATGTCATCGCG
AAGATGGTTCCACCCAACGATATCTGGCATCGAAGCTGAGAAACTGCTGC
AGGAGCAGGGATTCGACGGCTCCTTCCTCGCCCGCCTCTCCTCCTCGAAT
CCGGGCGCCTTCACGCTCTCCGTGCGCCGCGGCAACGAGGTGACCCACAT
CAAAATCCAAAACAATGGCGACTTCTTTGATCTCTACGGTGGTGAAAAGT
TCGCCACACTGCCGGAACTGGTACAATACTACATGGAGAATGGCGAGCTA
AAGGAGAAGAACGGCCAGGCCATCGAACTCAAGCAGCCGCTGATCTGCGC
CGAGCCCACCACGGAAAGATGGTTTCATGGCAATCTTTCCGGAAAGGAAG
CGGAAAAATTGATCCTGGAGCGGGGCAAGAATGGTTCGTTTCTCGTCCGT
GAATCTCAGAGCAAGCCTGGCGACTTCGTCCTTTCCGTGCGCACGGACGA
CAAAGTAACGCATGTCATGATTCGATGGCAGGACAAGAAGTACGACGTCG
GCGGGGGGGAATCCTTTGGCACCTTGTCGGAACTGATCGATCACTACAAG
CGTAATCCCATGGTGGAGACGTGCGGAACCGTGGTGCATCTGCGACAGCC
ATTCAACGCCACACGAATCACGGCGGCCGGCATCAATGCCCGGGTGGAAC
AGCTGGTCAAGGGAGGTTTCTGGGAGGAATTCGAATCGCTGCAACAGGAC
AGTCGGGACACATTCTCGCGCAACGAGGGCTACAAACAGGAGAACCGCCT
CAAGAATCGCTACCGCAACATATTGCCATACGACCACACGCGCGTCAAGC
TGCTGGACGTGGAGCATAGCGTGGCCGGAGCCGAGTACATCAATGCCAAC
TACATACGGCTGCCCACCGACGGCGACCTGTACAACATGAGCAGCTCGTC
GGAGAGCCTGAACAGCTCGGTGCCCTCGTGCCCCGCCTGCACGGCTGCCC
AGACACAGCGGAACTGCTCCAACTGCCAGCTGCAAAACAAGACGTGCGTG
CAGTGCGCCGTGAAGAGCGCCATTCTGCCGTATAGCAACTGTGCCACCTG
CAGCCGCAAGTCAGACTCCCTGAGCAAGCACAAGCGGAGCGAATCCTCGG
CCTCTTCATCGCCCTCCTCCGGCTCTGGGTCCGGACCAGGATCGTCGGGC
ACCAGCGGAGTGAGCAGCGTCAATGGACCCGGCACACCCACCAATCTCAC
GAGCGGCACAGCCGGATGTCTGGTCGGCCTGCTGAAGAGACACTCGAACG
ACTCGTCCGGAGCTGTTTCTATATCGATGGCCGAACGGGAACGCGAGAGG
GAGCGCGAGATGTTTAAGACCTACATCGCCACCCAGGGCTGTCTGCTCAC
CCAGCAAGTGAACACGGTGACGGACTTCTGGAACATGGTCTGGCAGGAGA
ACACGCGGGTGATCGTCATGACCACCAAGGAGTACGAGCGCGGCAAAGAA
AAGTGCGCCCGCTACTGGCCGGACGAGGGTAGATCGGAGCAGTTCGGCCA
CGCGCGGATACAGTGCGTCTCGGAGAACTCGACCAGTGACTATACGCTGC
GCGAGTTCCTCGTCTCGTGGCGGGATCAGCCGGCGCGCCGGATCTTTCAC
TACCATTTCCAGGTGTGGCCGGATCACGGAGTGCCCGCCGATCCGGGCTG
TGTGCTCAACTTCCTGCAAGATGTCAACACGCGTCAGAGTCACCTGGCTC
AAGCGGGCGAGAAGCCGGGTCCGATCTGCGTGCACTGCTCTGCGGGCATC
GGTCGCACTGGCACCTTTATTGTGATCGATATGATTCTCGATCAGATTGT
GCGCAATGGATTGGATACTGAAATCGACATCCAGCGCACCATTCAGATGG
TCCGATCGCAGCGTTCCGGTCTTGTGCAAACCGAGGCGCAATACAAGTTC
GTCTACTATGCGGTGCAGCACTATATACAGACCCTGATCGCCCGGAAACG
AGCTGAGGAGCAGAGCCTGCAGGTTGGCCGCGAGTACACCAATATAAAGT
ACACGGGCGAAATTGGAAACGATTCACAAAGATCTCCATTACCACCAGCA
ATTTCTAGCATAAGTTTAGTTCCGAGTAAGACGCCACTGACGCCGACATC
GGCGGATTTGGGCACTGGGATGGGCCTAAGCATGGGCGTGGGCATGGGCG
TCGGCAACAAGCACGCATCGAAGCAGCAGCCGCCGTTGCCGGTGGTCAAC
TGCAACAATAATAACAACGGCATTGGCAATAGCGGCTGCAGCAACGGCGG
CGGGAGCAGCACCACCAGCAGCAGCAACGGCAGCAGCAACGGTAACATCA
ACGCCCTACTGGGCGGCATCGGCTTGGGGCTGGGCGGCAATATGCGCAAG
TCGAACTTTTACAGCGACTCGCTGAAGCAGCAACAGCAGCGCGAGGAGCA
GGCTCCGGCGGGAGCAGGTAAGATGCAGCAGCCGGCGCCGCCGCTGCGAC
CGCGTCCTGGAATACTCAAGTTGCTCACCAGTCCCGTCATCTTTCAGCAA
AATTCAAAAACATTCCCAAAGACATGA

(SEQ ID NO:210)

MSSRRWFHPTISGIEAEKLLQEQGFDGSFLARLSSSNPGAFTLSVRRGNE
VTHIKIQNNGDFFDLYGGEKFATLPELVQYYMENGELKEKNGQAIELKQP
LICAEPTTERWFHGNLSGKEAEKLILERGKNGSFLVRESQSKPGDFVLSV
RTDDKVTHVMIRWQDKKYDVGGGESFGTLSELIDHYKRNPMVETCGTVVH
LRQPFNATRITAAGINARVEQLVKGGFWEEFESLQQDSRDTFSRNEGYKQ
ENRLKNRYRNILPYDHTRVKLLDVEHSVAGAEYINANYIRLPTDGDLYNM
SSSSESLNSSVPSCPACTAAQTQRNCSNCQLQNKTCVQCAVKSAILPYSN
CATCSRKSDSLSKHKRSESSASSSPSSGSGSGPGSSGTSGVSSVNGPGTP
TNLTSGTAGCLVGLLKRHSNDSSGAVSISMAEREREREREMFKTYIATQG
CLLTQQVNTVTDFWNMVWQENTRVIVMTTKEYERGKEKCARYWPDEGRSE
QFGHARIQCVSENSTSDYTLREFLVSWRDQPARRIFHYHFQVWPDHGVPA
DPGCVLNFLQDVNTRQSHLAQAGEKPGPICVHCSAGIGRTGTFIVIDMIL
DQIVRNGLDTEIDIQRTIQMVRSQRSGLVQTEAQYKFVYYAVQHYIQTLI
ARKRAEEQSLQVGREYTNIKYTGEIGNDSQRSPLPPAISSISLVPSKTPL
TPTSADLGTGMGLSMGVGMGVGNKHASKQQPPLPVVNCNNNNNGIGNSGC
SNGGGSSTTSSSNGSSNGNINALLGGIGLGLGGNMRKSNFYSDSLKQQQQ
REEQAPAGAGKMQQPAPPLRPRPGILKLLTSPVIFQQNSKTFPKT

CG16903-Cyclin/Non-Specific RNA Polymersae II Transcription Factor

(SEQ ID NO:211)

ATTTAGTATAAAAGCACGCCTGTTATCGGCTAAATTTACAAAAAAAAAGG
GAAAATTAAAAAATTAAAACACTTAAATAAACGCTTTCCTGGGTTAACCG
CGCACGAATGGCCACCCGTGGGGCCGGCTCGACTGTGGTCCACACGACGG
TGACAGCGCTGACGGTGGAGACGATCACCAATGTCCTGACCACGGTGACT
TCGTTCCATTCGAACAGCGTCAACATTTCGAACAACAACAGCAGCAGTGG
AGCGGCCCCGGGGGCGGATGCAGCTGGCGGCGATGCAGGGGGCGTGGCAG
CGGCTCAGGCGGACGCCAACAAGCCTATCTATCCTCGGCTCTTTAACCGC
ATCGTGCTGACGCTGGAGAACAGCCTCATTCCGGAGGGCAAAATCGATGT
GACGCCATCCAGCCAGGATGGACTGGACCATGAGACGGAGAAGGACCTGC
GCATACTGGGCTGCGAGCTTATTCAGACAGCCGGAATTTTGCTGCGCTTG
CCGCAGGTTGCCATGGCCACCGGCCAGGTGCTGTTCCAGCGCTTCTTCTA
CTCGAAGAGCTTTGTGCGGCACAACATGGAGACTGTGGCCATGAGCTGCG
TGTGCCTGGCGTCCAAGATCGAGGAGGCGCCGCGCCGCATTAGAGACGTG
ATCAATGTGTTCCATCACATCAAGCAAGTGCGGGCCCAAAAGGAAATCTC
GCCCATGGTGCTAGATCCTTACTACACGAACCTCAAGATGCAGGTGATCA
AGGCCGAGCGGCGCGTCCTCAAGGAACTGGGCTTCTGTGTACACGTGAAG
CATCCGCACAAGCTGATCGTGATGTATCTGCAGGTGCTTCAGTACGAGAA
GCACGAGAAGCTGATGCAGCTCTCCTGGAACTTCATGAATGACTCGCTGA
GGACGGACGTTTTTATGCGCTACACACCAGAGGCGATTGCATGCGCCTGC
ATCTACCTGAGTGCCCGCAAGCTCAACATACCTCTGCCCAACAGCCCGCC
GTGGTTCGGCATTTTTCGGGTGCCCATGGCGGACATTACGGATATCTGCT
ACCGTGTGATGGAGCTGTACATGCGTTCCAAGCCGGTGGTGGAGAAACTG
GAGGCGGCCGTGGACGAGCTGAAAAAGCGGTACATTGATGCGCGCAACAA
AACGAAGGAGGCAAACACACCGCCGGCTGTAATCACCGTGGATCGGAACA
ATGGCTCGCACAATGCGTGGGGTGGCTTCATCCAGCGTGCTATCCCACTG
CCCTTGCCATCGGAAAAGTCGCCGCAAAAGGATTCGAGGTCACGCTCGCG
ATCCAGGACGCGCACCCATTCGCGGACACCTCGCTCCCGATCACCCAGGT
CCAGGTCGCCTAGTCGCGAGCGCACTAAGAAGACCCACCGCAGTCGATCC
TCCCGCTCGCGCTCCCGTTCGCCGCCGAAGCATAAGAAAAAGTCACGTCA
CTACTCGAGGTCGCCCACGCGCTCCAATTCGCCGCACAGCAAGCACAGGA
AGTCGAAATCCTCGCGAGAACGCTCTGAATACTACTCCAAGAAAGATCGG
TCTGGAAACCCAGGCAGTAGCAATAATCTAGGTGATGGCGACAAGTATCG
CAACTCCGTCTCCAATTCCGGCAAGCACAGTCGGTACTCCTCCTCCTCGT
CGCGTCGGAACAGCGGTGGTGGTGGAGACGGAAGAAGCGGAGGAGGAGGT
GGTGGCGGCGGTGGAGGCAACGGGAACCACGGCAGCCGAGGGGGGCACAA
GCATCGGGATGGCGATCGCTCCAGGGATCGCAAGCGCTAGTGATTGATAG
ACAAGCGAGACAAACACTCCCTTATATTTAATTGCTCTTTATTTTACAAA
TTTACAGATTATTTCTACCGATTTAGTAATGCTAATGTGTATTGAAAAAA
CGAACGCGGGTAAACAATAAATGTAACTCTTCAATC

(SEQ ID NO:212)

MATRGAGSTVVHTTVTALTVETITNVLTTVTSFHSNSVNISNNNSSSGAA
PGADAAGGDAGGVAAAQADANKPIYPRLFNRIVLTLENSLIPEGKIDVTP
SSQDGLDHETEKDLRILGCELIQTAGILLRLPQVAMATGQVLFQRFFYSK
SFVRHNMETVAMSCVCLASKIEEAPRRIRDVINVFHHIKQVRAQKEISPM
VLDPYYTNLKMQVIKAERRVLKELGFCVHVKHPHKLIVMYLQVLQYEKHE
KLMQLSWNFMNDSLRTDVFMRYTPEAIACACIYLSARKLNIPLPNSPPWF
GIFRVPMADITDICYRVMELYMRSKPVVEKLEAAVDELKKRYIDARNKTK
EANTPPAVITVDRNNGSHNAWGGFIQRAIPLPLPSEKSPQKDSRSRSRSR
TRTHSRTPRSRSPRSRSPSRERTKKTHRSRSSRSRSRSPPKHKKKSRHYS
RSPTRSNSPHSKHRKSKSSRERSEYYSKKDRSGNPGSSNNLGDGDKYRNS
VSNSGKHSRYSSSSSRRNSGGGGDGRSGGGGGGGGGGNGNHGSRGGHKHR
DGDRSRDRKLR

Human homologue of Complete Genome candidate
CG3954 homologue is Homo sapiens protein tyrosine phosphatase, non-receptor type 11 (PTPN11), also known as Shp2. Shp2 has 2 alternative transcripts having accession numbers NM_{—}002834 and NM_{—}080601.

NM 002834 Homo Sapiens Protein Tyvrosine Phosphatase, Non-Receptor Type 11 (PTPN11), Transcript Variant 1, mRNA also known as Shp2.

| | | | | | | | |
|---|---|---|---|---|---|---|---|
| 1 | cggccgcggt | ttccaggagg | aagcaaggat | gctttggaca | ctgtgcgtgg | cgcctccgcg | (SEQ ID NO:213) |
| 61 | gagcccccgc | gctgccattc | ccggccgtcg | ctcggtcctc | cgctgacggg | aagcaggaag | |
| 121 | tggcggcggg | cgtcgcgagc | ggtgacatca | cgggggcgac | ggcggcgaag | ggcgggggcg | |
| 181 | gaggaggagc | gagccgggcc | ggggggcagc | tgcacagtct | ccgggatccc | caggcctgga | |
| 241 | ggggggtctg | tgcgcggccg | gctggctctg | ccccgcgtcc | ggtcccgagc | gggcctccct | |
| 301 | cgggccagcc | cgatgtgacc | gagcccagcg | gagcctgagc | aaggagcggg | tccgtcgcgg | |
| 361 | agccggaggg | cgggaggaac | atgacatcgc | ggagatggtt | tcacccaaat | atcactggtg | |
| 421 | tggaggcaga | aaacctactg | ttgacaagag | gagttgatgg | cagttttttg | gcaaggccta | |
| 481 | gtaaaagtaa | ccctggagac | ttcacacttt | ccgttagaag | aaatggagct | gtcacccaca | |
| 541 | tcaagattca | gaacactggt | gattactatg | acctgtatgg | aggggagaaa | tttgccactt | |
| 601 | tggctgagtt | ggtccagtat | tacatggaac | atcacgggca | attaaaagag | aagaatggag | |
| 661 | atgtcattga | gcttaaatat | cctctgaact | gtgcagatcc | tacctctgaa | aggtggtttc | |
| 721 | atggacatct | ctctgggaaa | gaagcagaga | aattattaac | tgaaaaagga | aaacatggta | |
| 781 | gttttcttgt | acgagagagc | cagagccacc | ctggagattt | tgttctttct | gtgcgcactg | |
| 841 | gtgatgacaa | aggggagagc | aatgacggca | agtctaaagt | gacccatgtt | atgattcgct | |
| 901 | gtcaggaact | gaaatacgac | gttggtggag | gagaacggtt | tgattctttg | acagatcttg | |
| 961 | tggaacatta | taagaagaat | cctatggtgg | aaacattggg | tacagtacta | caactcaagc | |
| 1021 | agccccttaa | cacgactcgt | ataaatgctg | ctgaaataga | aagcagagtt | cgagaactaa | |
| 1081 | gcaaattagc | tgagaccaca | gataaagtca | aacaaggctt | ttgggaagaa | tttgagacac | |
| 1141 | tacaacaaca | ggagtgcaaa | cttctctaca | gccgaaaaga | gggtcaaagg | caagaaaaca | |
| 1201 | aaaacaaaaa | tagatataaa | aacatcctgc | cctttgatca | taccagggtt | gtcctacacg | |
| 1261 | atggtgatcc | caatgagcct | gtttcagatt | acatcaatgc | aaatatcatc | atgcctgaat | |
| 1321 | ttgaaaccaa | gtgcaacaat | tcaaagccca | aaaagagtta | cattgccaca | caaggctgcc | |
| 1381 | tgcaaaacac | ggtgaatgac | ttttggcgga | tggtgttcca | agaaaactcc | cgagtgattg | |
| 1441 | tcatgacaac | gaaagaagtg | gagagaggaa | agagtaaatg | tgtcaaatac | tggcctgatg | |
| 1501 | agtatgctct | aaaagaatat | ggcgtcatgc | gtgttaggaa | cgtcaaagaa | agcgccgctc | |
| 1561 | atgactatac | gctaagagaa | cttaaacttt | caaaggttgg | acaagggaat | acggagagaa | |
| 1621 | cggtctggca | ataccacttt | cggacctggc | cggaccacgg | cgtgcccagc | gaccctgggg | |
| 1681 | gcgtgctgga | cttcctggag | gaggtgcacc | ataagcagga | gagcatcatg | gatgcagggc | |
| 1741 | cggtcgtggt | gcactgcagt | gctggaattg | gccggacagg | gacgttcatt | gtgattgata | |
| 1801 | ttcttattga | catcatcaga | gagaaaggtg | ttgactgcga | tattgacgtt | cccaaaacca | |
| 1861 | tccagatggt | gcggtctcag | aggtcaggga | tggtccagac | agaagcacag | taccgattta | |
| 1921 | tctatatggc | ggtccagcat | tatattgaaa | cactacagcg | caggattgaa | gaagagcaga | |
| 1981 | aaagaaagag | gaaagggcac | gaatatacaa | atattaagta | ttctctagcg | gaccagacga | |
| 2041 | gtggagatca | gagccctctc | ccgccttgta | ctccaacgcc | accctgtgca | gaaatgagag | |
| 2101 | aagacagtgc | tagagtctat | gaaaacgtgg | gcctgatgca | acagcagaaa | agtttcagat | |
| 2161 | gagaaaacct | gccaaaactt | cagcacagaa | atagatgtgg | actttcaccc | tctccctaaa | |
| 2221 | aagatcaaga | acagacgcaa | gaaagtttat | gtgaagacag | aatttggatt | tggaaggctt | |
| 2281 | gcaatgtggt | tgactacctt | ttgataagca | aaatttgaaa | ccatttaaag | accactgtat | |
| 2341 | tttaactcaa | caatacctgc | ttcccaatta | ctcatttcct | cagataagaa | gaaatcatct | |
| 2401 | ctacaatgta | gacaacatta | tattttatag | aatttgtttg | aaattgagga | agcagttaaa | |
| 2461 | ttgtgcgctg | tattttgcag | attatgggga | ttcaaattct | agtaataggc | ttttttattt | |
| 2521 | ttatttttat | acccttaacc | agtttaattt | tttttttcct | cattgttggg | gatgatgaga | |
| 2581 | agaaatgatt | tgggaaaatt | aagtaacaac | gacctagaaa | agtgagaaca | atctcattta | |
| 2641 | ccatcatgta | tccagtagtg | gataattcat | tttgatggct | tctatttttg | gccaaatgag | |
| 2701 | aattaagcca | gtgcctgaga | ctgtcagaag | ttgacctttg | cactggcatt | aaagagtcat | |
| 2761 | agaaaaaa | | | | | | |

| | |
|---|---|
| MTSRRWFHPNITGVEAENLLLTRGVDGSFLARPSKSNPGDFTLS | (SEQ ID NO:214) |
| VRRNGAVTHIKIQNTGDYYDLYGGEKFATLAELVQYYMEHHGQLKEKNGDVIELKYPL | |
| NCADPTSERWFHGHLSGKEAEKLLTEKGKHGSFLVRESQSHPGDFVLSVRTGDDKGES | |
| NDGKSKVTHVMIRCQELKYDVGGGERFDSLTDLVEHYKKNPMVETLGTVLQLKQPLNT | |
| TRINAAEIESRVRELSKLAETTDKVKQGFWEEFETLQQQECKLLYSRKEGQRQENKNK | |
| NRYKNILPFDHTRVVLHDGDPNEPVSDYINANIIMPEFETKCNNSKPKKSYIATQGCL | |
| QNTVNDFWRMVFQENSRVIVMTTKEVERGKSKCVKYWPDEYALKEYGVMRVRNVKESA | |
| AHDYTLRELKLSKVGQGNTERTVWQYHFRTWPDHGVPSDPGGVLDFLEEVHHKQESIM | |
| DAGPVVVHCSAGIGRTGTFIVIDILIDIIREKGVDCDIDVPKTIQMVRSQRSGMVQTE | |
| AQYRFIYMAVQHYIETLQRRIEEEQKRKRKGHEYTNIKYSLADQTSGDQSPLPPCTPT | |
| PPCAEMREDSARVYENVGLMQQQKSFR | |

NM_{—}080601 Homo Sapiens Protein Tyrosine Phosphatase, Non-Receptor Type 11(PTPN11), Transcript Variant 2, mRNA (Version 1)

| | | |
|---|---|---|
| 1 | gcggaggagg agcgagccgg gccggggggc agctgcacag tctccgggat ccccaggcct | (SEQ ID NO:215) |
| 61 | ggaggggggt ctgtgcgcgg ccggctggct ctgccccgcg tccggtcccg agcgggcctc | |
| 121 | cctcgggcca gcccgatgtg accgagccca gcggagcctg agcaaggagc gggtccgtcg | |
| 181 | cggagccgga gggcgggagg aacatgacat cgcggagatg gtttcaccca aatatcactg | |
| 241 | gtgtggaggc agaaaaccta ctgttgacaa gaggagttga tggcagtttt ttggcaaggc | |
| 301 | ctagtaaaag taaccctgga gacttcacac tttccgttag aagaaatgga gctgtcaccc | |
| 361 | acatcaagat tcagaacact ggtgattact atgacctgta tggaggggag aaatttgcca | |
| 421 | ctttggctga gttggtccag tattacatgg aacatcacgg gcaattaaaa gagaagaatg | |
| 481 | gagatgtcat tgagcttaaa tatcctctga actgtgcaga tcctacctct gaaaggtggt | |
| 541 | ttcatggaca tctctctggg aaagaagcag agaaattatt aactgaaaaa ggaaaacatg | |
| 601 | gtagttttct tgtacgagag agccagagcc accctggaga ttttgttctt tctgtgcgca | |
| 661 | ctggtgatga caaaggggag agcaatgacg gcaagtctaa agtgacccat gttatgattc | |
| 721 | gctgtcagga actgaaatac gacgttggtg gaggagaacg gtttgattct ttgacagatc | |
| 781 | ttgtggaaca ttataagaag aatcctatgg tggaaacatt gggtacagta ctacaactca | |
| 841 | agcagcccct taacacgact cgtataaatg ctgctgaaat agaaagcaga gttcgagaac | |
| 901 | taagcaaatt agctgagacc acagataaag tcaaacaagg cttttgggaa gaatttgaga | |
| 961 | cactacaaca acaggagtgc aaacttctct acagccgaaa agagggtcaa aggcaagaaa | |
| 1021 | acaaaaacaa aaatagatat aaaaacatcc tgccctttga tcataccagg gttgtcctac | |
| 1081 | acgatggtga tcccaatgag cctgtttcag attacatcaa tgcaaatatc atcatgcctg | |
| 1141 | aatttgaaac caagtgcaac aattcaaagc ccaaaaagag ttacattgcc acacaaggct | |
| 1201 | gcctgcaaaa cacggtgaat gacttttggc ggatggtgtt ccaagaaaac tcccgagtga | |
| 1261 | ttgtcatgac aacgaaagaa gtggagagag gaaagagtaa atgtgtcaaa tactggcctg | |
| 1321 | atgagtatgc tctaaaagaa tatggcgtca tgcgtgttag gaacgtcaaa gaaagcgccg | |
| 1381 | ctcatgacta tacgctaaga gaacttaaac tttcaaaggt tggacaaggg aatacggaga | |
| 1441 | gaacggtctg gcaataccac tttcggacct ggccggacca cggcgtgccc agcgaccctg | |
| 1501 | ggggcgtgct ggacttcctg gaggaggtgc accataagca ggagagcatc atggatgcag | |
| 1561 | ggccggtcgt ggtgcactgc aggtgacagc tcctgctgcc cctctaggcc acagcctgtc | |
| 1621 | cctgtctcct agcgcccagg gcttgctttt acctacccac tcctagctct ttaactgtag | |
| 1681 | gaagaattta atatctgttt gaggcataga gcaactgcat tgagggacat tttgatccca | |
| 1741 | aggcatattt ctcctagacc ctacagcact gccattggcc atggccatgg caacatgctc | |
| 1801 | agttaaaaca gcaaagacta agtcagcatt atctctgagt ccaccagaag ttgtgcatta | |
| 1861 | aacaacttca tcctggaaaa aaaaaaaaaa aa | |
| 1 | mtsrrwfhpn itgveaenll ltrgvdgsfl arpsksnpgd ftlsvrrnga vthikiqntg | (SEQ ID NO:216) |
| 61 | dyydlyggek fatlaelvqy ymehhgqlke kngdvielky plncadptse rwfhghlsgk | |
| 121 | eaeklltekg khgsflvres qshpgdfvls vrtgddkges ndgkskvthv mircqelkyd | |
| 181 | vgggerfdsl tdlvehykkn pmvetlgtvl qlkqplnttr inaaeiesrv relsklaett | |
| 241 | dkvkqgfwee fetlqqqeck llysrkegqr qenknknryk nilpfdhtrv vlhdgdpnep | |
| 301 | vsdyinanii mpefetkcnn skpkksyiat qgclqntvnd fwrmvfqens rvivmttkev | |
| 361 | ergkskcvky wpdeyalkey gvmrvrnvke saahdytlre lklskvgqgn tertvwqyhf | |
| 421 | rtwpdhgvps dpggvldfle evhhkqesim dagpvvvhcr | |

NM_{—}080601 Homo Sapiens Protein Tyrosine Phosphatase, Non-Receptor Type 11 (PTPN11), Transcript Variant 2, mRNA (Version 2)

| | | | | | | | |
|---|---|---|---|---|---|---|---|
| 1 | cggccgcggt | ttccaggagg | aagcaaggat | gctttggaca | ctgtgcgtgg | cgcctccgcg | (SEQ ID NO:217) |
| 61 | gagcccccgc | gctgccattc | ccggccgtcg | ctcggtcctc | cgctgacggg | aagcaggaag | |
| 121 | tggcggcggg | cgtcgcgagc | ggtgacatca | cgggggcgac | ggcggcgaag | ggcgggggcg | |
| 181 | gaggaggagc | gagccgggcc | ggggggcagc | tgcacagtct | ccgggatccc | caggcctgga | |
| 241 | ggggggtctg | tgcgcggccg | gctggctctg | ccccgcgtcc | ggtcccgagc | gggcctccct | |
| 301 | cgggccagcc | cgatgtgacc | gagcccagcg | gagcctgagc | aaggagcggg | tccgtcgcgg | |
| 361 | agccggaggg | cgggaggaac | atgacatcgc | ggagatggtt | tcacccaaat | atcactggtg | |
| 421 | tggaggcaga | aaacctactg | ttgacaagag | gagttgatgg | cagttttttg | gcaaggccta | |
| 481 | gtaaaagtaa | ccctggagac | ttcacacttt | ccgttagaag | aaatggagct | gtcacccaca | |
| 541 | tcaagattca | gaacactggt | gattactatg | acctgtatgg | aggggagaaa | tttgccactt | |
| 601 | tggctgagtt | ggtccagtat | tacatggaac | atcacgggca | attaaaagag | aagaatggag | |
| 661 | atgtcattga | gcttaaatat | cctctgaact | gtgcagatcc | tacctctgaa | aggtggtttc | |
| 721 | atggacatct | ctctgggaaa | gaagcagaga | aattattaac | tgaaaaagga | aaacatggta | |
| 781 | gttttcttgt | acgagagagc | cagagccacc | ctggagattt | tgttctttct | gtgcgcactg | |
| 841 | gtgatgacaa | aggggagagc | aatgacggca | agtctaaagt | gacccatgtt | atgattcgct | |
| 901 | gtcaggaact | gaaatacgac | gttggtggag | gagaacggtt | tgattctttg | acagatcttg | |
| 961 | tggaacatta | taagaagaat | cctatggtgg | aaacattggg | tacagtacta | caactcaagc | |
| 1021 | agccccttaa | cacgactcgt | ataaatgctg | ctgaaataga | aagcagagtt | cgagaactaa | |
| 1081 | gcaaattagc | tgagaccaca | gataaagtca | aacaaggctt | ttgggaagaa | tttgagacac | |
| 1141 | tacaacaaca | ggagtgcaaa | cttctctaca | gccgaaaaga | gggtcaaagg | caagaaaaca | |
| 1201 | aaaacaaaaa | tagatataaa | aacatcctgc | cctttgatca | taccagggtt | gtcctacacg | |
| 1261 | atggtgatcc | caatgagcct | gtttcagatt | acatcaatgc | aaatatcatc | atgcctgaat | |
| 1321 | ttgaaaccaa | gtgcaacaat | tcaaagccca | aaaagagtta | cattgccaca | caaggctgcc | |
| 1381 | tgcaaaacac | ggtgaatgac | ttttggcgga | tggtgttcca | agaaaactcc | cgagtgattg | |
| 1441 | tcatgacaac | gaaagaagtg | gagagaggaa | agagtaaatg | tgtcaaatac | tggcctgatg | |
| 1501 | agtatgctct | aaaagaatat | ggcgtcatgc | gtgttaggaa | cgtcaaagaa | agcgccgctc | |
| 1561 | atgactatac | gctaagagaa | cttaaacttt | caaaggttgg | acaagggaat | acggagagaa | |
| 1621 | cggtctggca | ataccacttt | cggacctggc | cggaccacgg | cgtgcccagc | gaccctgggg | |
| 1681 | gcgtgctgga | cttcctggag | gaggtgcacc | ataagcagga | gagcatcatg | gatgcagggc | |
| 1741 | cggtcgtggt | gcactgcagg | tgacagctcc | tgctgcccct | ctaggccaca | gcctgtccct | |
| 1801 | gtctcctagc | gcccagggct | tgcttttacc | tacccactcc | tagctcttta | actgtaggaa | |
| 1861 | gaatttaata | tctgtttgag | gcatagagca | actgcattga | gggacatttt | gatcccaagg | |
| 1921 | catatttctc | ctagacccta | cagcactgcc | attggccatg | gccatggcaa | catgctcagt | |
| 1981 | taaaacagca | aagactaagt | cagcattatc | tctgagtcca | ccagaagttg | tgcattaaac | |
| 2041 | aacttcatcc | tggaaaaaaa | aaaaaaaaa | | | | |

| | |
|---|---|
| MTSRRWFHPNITGVEAENLLLTRGVDGSFLARPSKSNPGDFTLS | (SEQ ID NO:218) |
| VRRNGAVTHIKIQNTGDYYDLYGGEKFATLAELVQYYMEHHGQLKEKNGDVIELKYPL | |
| NCADPTSERWFHGHLSGKEAEKLLTEKGKHGSFLVRESQSHPGDFVLSVRTGDDKGES | |
| NDGKSKVTHVMIRCQELKYDVGGGERFDSLTDLVEHYKKNPMVETLGTVLQLKQPLNT | |
| TRINAAEIESRVRELSKLAETTDKVKQGFWEEFETLQQQECKLLYSRKEGQRQENKNK | |
| NRYKNILPFDHTRVVLHDGDPNEPVSDYINANIIMPEFETKCNNSKPKKSYIATQGCL | |
| QNTVNDFWRMVFQENSRVIVMTTKEVERGKSKCVKYWPDEYALKEYGVMRVRNVKESA | |
| AHDYTLRELKLSKVGQGNTERTVWQYHFRTWPDHGVPSDPGGVLDFLEEVHHKQESIM | |
| DAGPVVVHCR | |

Putative function
(CG3954)—protein tyrosine phosphatase
(CG16903)—cyclin, potentially involved in differentiation and neural plasticity

### Example 19B

### Validation of GENE Function by RNA Interference (RNAi) Knockdown in Drosophila Cultured Cells

To confirm the mitotic role of the target protein, knockdown of Corkscrew (CG3954) expression is performed in cultured Drosophila Dmel -2 cells using a double stranded RNA (dsRNA) from within the Corkscrew (CG3954) CDS corresponding to the following CDS sequence:

| | |
|---|---|
| GCCGAGTACATCAATGCCAACTACATACGGCTGCCCACCGACGGCGACCTGTACAA | (SEQ ID NO:219) |
| CATGAGCAGCTCGTCGGAGAGCCTGAACAGCTCGGTGCCCTCGTGCCCCGCCTGCAC | |
| GGCTGCCCAGACACAGCGGAACTGCTCCAACTGCCAGCTGCAAAACAAGACGTGCG | |
| TGCAGTGCGCCGTGAAGAGCGCCATTCTGCCGTATAGCAACTGTGCCACCTGCAGCC | |
| GCAAGTCAGACTCCCTGAGCAAGCACAAGCGGAGCGAATCCTCGGCCTCTTCATCG | |
| CCCTCCTCCGGCTCTGGGTCCGGACCAGGATCGTCGGGCACCAGCGGAGTGAGCAG | |
| CGTCAATGGACCCGGCACACCCACCAATCTCACGAGCGGCACAGCCGGATGTCTGG | |
| TCGGCCTGCTGAAGAGACACTCGAACGACTCGTCCGGAGCTGTTTCTATATCGATGG | |
| CCGAACGGGAACGCGAGAGGGAGCGCGAGATGTTTAAGACCTACATCGCCACCCA | |

dsRNA is prepared by annealing complimentary RNAs made by in vitro transcription from a PCR fragment created with the following PCR primers:

(SEQ ID NO:220)

TAATACGACTCACTATAGGGAGAGCCGAGTACATCAATGCCAACTACAT

(SEQ ID NO:221)

TAATACGACTCACTATAGGGAGATGGGTGGCGATGTAGGTCTTAAACAT

Cells are transfected with double stranded RNA in the presence of ‘Transfast’ transfection reagent. A control transfection of a non-endogenous RNA corresponding to RFP (red fluorescent protein) is carried out in parallel.

Analysis of Corkscrew CG3954 Knockdown by RNAi in D-Mel2 Cells by Cellomics Mitotic Index Assay

For the transfection, 1 μg dsRNA is added to a well of a 96-well Packard viewplate and 35 μl of logarithmically growing DMe1-2 cells diluted to 2.3×10⁵ cells/ml in fresh Drosophila-SFM/glutamine/Pen-Strep are added. Cells are incubated with the dsRNA (60 nM) in a humid chamber at 28° C. for 1 hr before addition of 100 μl Drosophila-SFM/glutamine/Pen-Strep. Cells are incubated at 28° C. for 72 hours before analysis. For the assay, cells were fixed and stained using the Cellomics Mitotic Index HitKit following manufacturers instructions. The mitotic index of cells in each well was determined using the ArrayScan HCS System, running the Application protocol Mike_{—}250502_Polgen_MitoticIndex_{—}10×_p2.0 with the 10× objective and the DualBGlp filter set. This automated screening system detects the levels of a specific antigen (phosphorylated histone H3) which is only detectable during mitosis while the chromosomes are condensed.

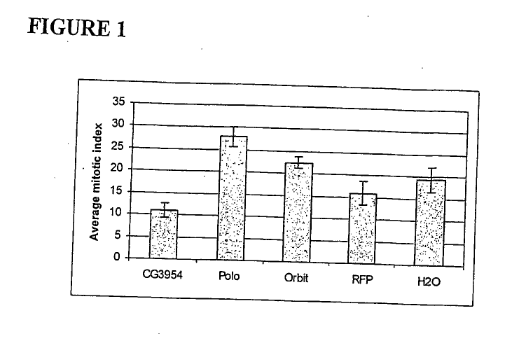

Results for Corkscrew (CG3954) are shown in FIG. 1. A reproducible and significant reduction in mitotic index is observed in this assay indicating a reduction in the number of cells able to exit S-phase and enter mitosis after RNAi

Analysis of Corkscrew CG3954 Knockdown by RNAi in D-Mel2 Cells by Microscopy

For transfection 9 μl of Transfast reagent (Promega) is added to 3μg gene specific dsRNA in 500 μl Drosophila Schneiders medium (no additives) and incubated at room temperature for 15 min. For control wells an equivalent amount of RFP dsRNA is used. This mix is added to a well of a 6-well tissue culture plate containing a glass coverslip and 500 μl of a Dme1-2 cells at 1×10⁶ cells/ml in shneiders medium. After a 1 hour incubation at 28° C., 2 mls Schneiders medium+10% FCS and pen/strep solution is added and cells are incubated at 28° C. for 48 hours. Cells on the coverslip are fixed in formaldehyde and stained with antibodies which detect α-tubulin and γ-tubulin (centrosomes), and are co-stained with DAPI to detect DNA.

An increase in the number of cells with chromosomal defects (see Table 1 below) was observed upon RNAi. The phenotypes seen were aneuploidy (65% of mitoses compared to 30% in control cells), misaligned chromosomes (80% compared to 40% in control cells), and polyploidy, however no spindle defects were observed.

| | | | % of chromosomal |
|---|---|---|---|
| | Number cells with | Number of | defects |
| | chromosomal | cells with | (no defects/total |
| dsRNA | defects | normal mitosis | cells in mitosis) |
| No RNA | 135 | 314 | 39.47 |
| RFP | 137 | 309 | 40.29 |
| CG1725 | 186 | 87 | 68.13 |

Table 1 shows mitotic defects observed by microscopy after RNAi knockdown of Corkscrew (CG3954) in Dmel2 Drosophila cultured cells.

### Example 19C

### Shp2 is a Human Homologue of Drosophila Corkscrew CG3954

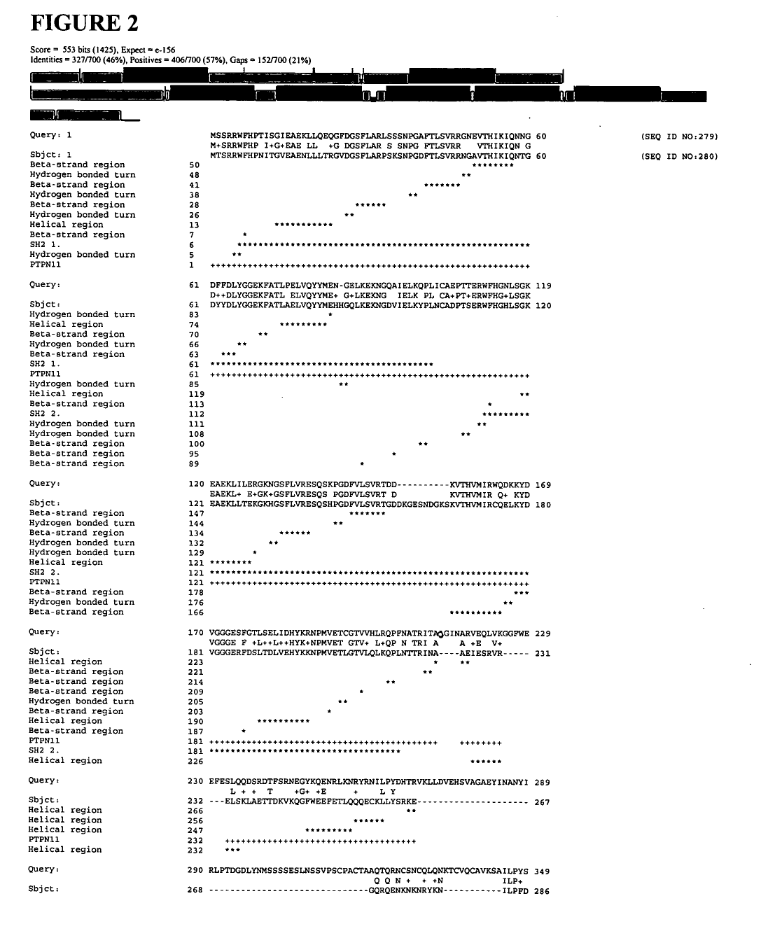

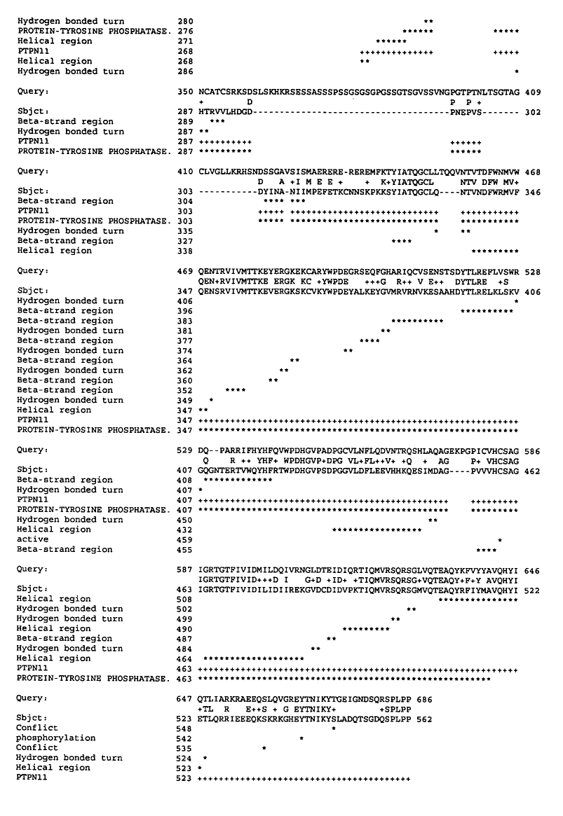

BLASTP with Drosophila Corkscrew CG3954 reveals 46% (327/700) sequence identity with the human Shp2 gene (genbank accession D13540), indicating that they are homologues. The BLASTP results are shown in FIG. 2.

The sequence of the human Shp2 gene MRNA (2 splice variants is shown in Example 19 above).

### Example 19D

### Validation of the Mitotic Role of the Human Homologue by siRNA Knockdown of Shp2 Expression in Human Cultured Cells

Generation of Shp2 siRNA Knockdowns

Knockdown of human Shp2 gene expression is achieved by siRNA (short interfering RNA, Elbashir et al, Nature 2001 May 24;411(6836):494-8). We used synthetic double stranded RNAs corresponding to two different regions of the Shp2 mRNA. siRNAs are obtained from Dhannacon (our supplier). The siRNA sequences are:

| | | | |
|---|---|---|---|
| COD1650 | shp2-1 | AACGUCAAAGAAAGCGC | Corresponds to nucleotides |
| | siRNA | CGCU | 1539-1559 in human Shp2 |
| | | (SEQ ID NO:222) | splice variants 1 and 2 see |
| | | | example 19 above) |
| COD1651 | shp2-2 | AAUUGGCCGGACAGGGA | Corresponds to nucleotides |
| | siRNA | CGUU | 1766-1786 in human Shp2 |
| | | (SEQ ID NO:223) | splice variants 1 and 2 see |
| | | | example 19 above) |

Analysis of siRNA Hu Shp2 Knockdowns in U2OS Cells by Flow Cytometry Analysis

Cells are seeded in 6-well tissue culture dishes at 1×10⁵ cells/well in 2 ml Dulbecco's Modified Eagle's Medium (DMEM) (Sigma)+10% Foetal Bovine Serum (FBS) (Perbio), and incubated overnight (37° C./5% CO₂).

For each well, 12 μl of 20 μM siRNA duplex (Dharmacon, Inc) (in RNAse-free H₂O) is mixed with 200 μl of Optimem (Invitrogen). In a separate tube 8 μl of oligofectamine reagent (Invitrogen) was mixed with 52 μl of Optimem, and incubated at room temperature for 7-10 mins. The oligofectamine/Optimem mix is then added dropwise to the siRNA/Optimem mix, and this is then mixed gently, before being incubated for 15-20 mins at room temperature. During this incubation the cells are washed once with DMEM (with no FBS or antibiotics added). 600 μl of DMEM (no FBS or antibiotics) is then added to each well.

Following the 15-20 min incubation, 128 μl of Optimem is added to the siRNA/oligofectamine/optimem mix, and this was added to the cells (in 600 μl DMEM). The transfection mix is added at the edge of each well to assist dilution before contact is made with the cells. Cells are then incubated with the transfection mix for 4 h (37° C./5% CO₂). Subsequently 1 ml DMEM+20% FBS is added to each well. Cells are then incubated at 37° C./5% CO₂ for 72 h. Cells are harvested by trypsinisation, washed in PBS, fixed in ice-cold 70% EtOH and stained with propidium iodide before Facs analysis.

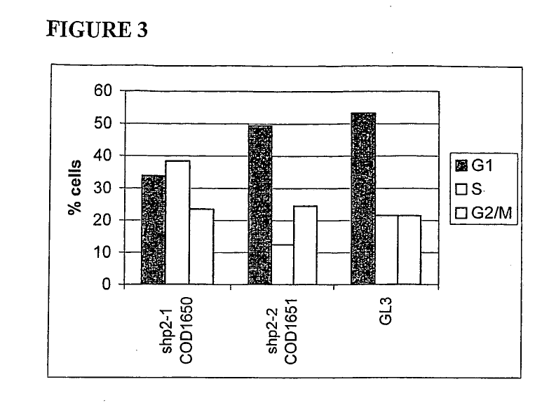

siRNA Hu Shp2 knockdowns are conducted in U2OS. As shown in FIG. 3 major changes in the distribution of cells between cell cycle compartments (G1, S, G2/M) are seen with Shp2 siRNA COD1650 which is directed to both alternative transcripts of Shp2. An accumulation of cells in the S2 compartment cell cycle, is observed with a concomitant reduction in the G1 compartment population. This indicates that a proportion of cells may unable complete S-phase and enter mitosis.

Subsequent microscopic analysis is performed in order to look at phenotypes resulting from the Shp2 siRNA induced defect and check for the presence of large multinucleate cells which may, due to their size and ploidy, be excluded from the FACS analysis.

Analysis of Hu Shp2 siRNA Knockdowns in U20S Cells by Microscopy

The transfection method for samples for microscopy is identical to that for Facs except that cells are plated in wells containing a sterile glass coverslip. Cells are incubated with siRNA for 48 hours before formaldehyde fixation and co-staining with Dapi to reveal DNA (blue) and antibodies to reveal microtubules (red) and centrosomes (green). Antibodies used are: rat anti-alpha tubulin (YL12) (supplier Serotec) with secondary antibody goat anti-rat IgG-TRITC (supplier Jackson Immunoresearch) and mouse anti-gamma-tubulin (GTU88) with secondary antibody Alexagreen488-goat anti-mouseIgG (supplier Sigrna).

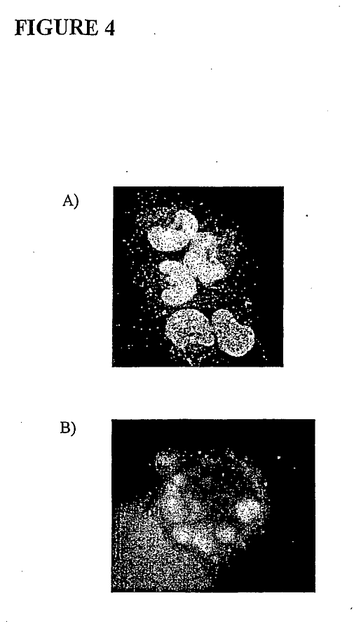

Phenotype analysis by microscopy is conducted on U2OS cells. Results from duplicate experiments in U2OS cells are shown in FIGS. 4, and Table 2 below. After siRNA no mitotic defects were seen, only a small increase in binucleate and apoptotic cells. These results are consistent with the Facs analysis, and in conjunction with the results of Corkscrew siRNA in Dme1-2 cells, they confirm that Shp2 is involved in cell cycle progression, in particular, in completing S-phase. Accordingly, modulators of Shp2 activity (as identified by the assays described above) may be used to treat any proliferative disease.

TABLE 2
Description of significant cell division defects after Shp2 siRNA
in U2OS cells.

| Gene/siRNA | Shp2/COD1650 |
|---|---|
| Cell Type | U2OS |
| Polyploidy | Normal |
| Mitotic Defects | Normal |
| Main knockout | No mitotic phenotype |
| phenotype | observed |
| Additional observations | Increased number of |
| | binuclear cells (0.6/field |
| | compared to 0.2/field in |
| | untreated) |
| | Increase in apoptotic |
| | cells |

### Example 19E

### Expression of Recombinant Hu Shp2 Protein in Insect Cells

A cDNA encoding the Human Shp2 coding region derived by RT-PCR is inserted into the baculovirus expression vector pFastbacHTc (Life Technologies). A baculovirus stock is generated and western blot of subsequent infections of Sf9 insect cells demonstrates expression of N-terminal 6-His tagged proteins of approximately 68 kD. The recombinant protein is purified by Ni—NTA resin affinity chromatography.

Similarly 6-His tagged Dlg proteins are expressed in bacteria by inserting cDNAs into bacterial expression plamids pDest17 or pET series. Protein expression in cultures of host E.coli cells transformed with recombinant plasmid is induced by addition of inducer chemical IPTG. The recombinant protein is purified by Ni—NTA resin affinity chromatography

### Example 19F

### Assay for Modulators of Shp2 Activity

Shp2 is a non-transmembrane-type protein tyrosine phosphatase that participates in the signal transduction pathways of a variety of growth factors and cytokines. Shp2 binds directly to the PDGF receptor, EGF receptor, and c-KIT in response to stimulation of cells with the corresponding receptor ligand and undergoes tyrosine phosphorylation. Shp2 is implicated in PDGF-induced RAS activation and EGF stimulation of the RAS-MAP kinase cascade that leads to DNA synthesis. Corkscrew (the putative Drosophila homolog of Shp2) is thought to be required for Ras1 activation or to function in conjunction with Ras1 during signaling by the Sevenless receptor tyrosine kinase. In addition Shp2 is implicated in insulin dependent signaling. Shp2 does not interact directly with the insulin receptor,but it binds through its SH2 domains to tyrosine-phosphorylated docking proteins such as IRS1, IRS2, and GAB1 in response to insulin. Overall Shp2 appears to play a role in growth factor-induced cell proliferation, through activation of the RAS-MAP kinase cascade. In addition to its role in receptor tyrosine kinase-mediated MAP kinase activation, Shp2 may play an important role, partly through its interaction with the membrane glycoprotein SHPS-1, in the activation of MAP kinase in response to the engagement of integrins by the extracellular matrix.

phosphotyrosyl proteins or peptides derived from SHPS-1, IRS1 or PDGF. An assay for modulators of Shp2 activity would consist of detection of dephosphorylation of ligand proteins, or phosphotyrosyl peptides derived from ligand proteins, described above e.g. phosphotyrosyl proteins or peptides derived from SHPS-1, IRS1 or PDGF (Takada et al 1998). Dephosphorylation of the substrate would be detected by quantifying the released inorganic phosphate, or by detecting loss of phosphate using an anti-phosphotyrosine antibody.

### Example 20

### Category 3

Line ID—500

Phenotype—Viable, High mitotic index, colchicines-type overcondensed chromosomes, a few polyploid cells

Annotated Drosophila genome genomic segment containing P element insertion site (and map position)—AE003422 (2C)

P element insertion site—247,403

Annotated Drosophila genome Complete Genome candidate

CG4399—EAST

(SEQ ID NO:224)

ATGTCTAGCCGGAAGGTGCCAGGAGGCTCTGGAGGAGCTGACGAATCCAC
AGCAGCAGCTGCCCCCCTGGATGATAATGCCAATGCCAGTGTGGAGATTC
CAGACAGCAGCGAGGAGCCAGCAATGGGCGTCGGCGAAGAGATGTCTATC
ATAAGCAAAACACGCACCTCAACTTTGTCAGTGGAGCCCGCTAAGGAGCC
AACAGTAACAGCAGAGCTGGAAGGCGAAAAAGAGCTGGAATCGAATCCAG
TCTCCAAAACTCCTAGGTCCACGCCTACGCCAACCCTTACGCCAGCCGTC
ACGCCTACCGCCAGTGATGGAGTGGCGGCCAAGAGCGTGAGGGTTACCCG
GCACTCGTCGCCACTGCTTCTGATCATCTCGCCCACGACAAGTAGACGTG
AGGTCGGCGACGGAGAGCTAGACACCGAGGAACCAACGGGATCGGGTGGC
CAAAGAAAGAGCTCCGTGGAGCGATCTTTGGCGCCCGTTATACGCGGACG
AAAGTCCATCAAGGATCTGAAAGAAGCCAAAGAAGTCAAGTCCGAGGAGC
CGCCTGCCGCAGCATCAGAGTCACGAGCTGCAAGTGGAGTGACGCCTGGC
CAGGTCAAGGAACAGCATGTCGCGGATGGCAACGAAATGGAATCCTTGCC
AATCACAGACAAGAAAGACCACAAAGACACAAAAGACAAGGGAGATGAGC
GGGAAACCGATCAGGAGGAAGAGAAGGAAAAATCAGCTGATACAGAAATA
ATTGCAGATACAGAAAAAACTTCGGAGAAACAAAAGTATACAGAGAAGGA
CAAAGCTGCCGATAAAGATGGAGGAAAAGAAAAAGATATTGATGCAAATA
AGGATATAGATAAGGAGAAGGAAAAGGTCAAGGAAGTACTTCCGCCAGTG
GTGCCTATAGCACCAGTGACACCCACTTGTAACCGTGTCACACGTAAATC
ACATGCCCAGGAGCAGGCGATTAACACGCGGGTCACTCGCAATCGTCGCC
AGTCCTCTACAGTTGGAGCCAACTCCACCGCGTCTTTGGTAGCTGCATCC
TCCTCAGTAACAGAGCAACCCCCTCCATCTCGCGGTCGACGGAAGAAGCC
AGTGGTGGTGGCTCCTCCCTTGGAGCCTGCGGTAAAACGGAAGCGATCGC
AAGATGTTGAAGCCGACTCAGACGCCAACAACAGCACGAAATACAGCAAG
GTGGAAGTGGTAAAGTCTGAGGAAGCTGAGGCACCAGAGGAGGACTCCAG
TGCCGTGCCCATTAAGCAGGAATCTGTTGATGGCAACGAGGTCAGTTCTA
TTTCTCCAACAGTCACGCCCACACCCACACCTGCGCCAACACCAGCTCCA
GTCCCGGGCAGTCGACGGGGTCGTGGGCGCCCGCAGAACAGGAACTCCTC
TTCGCCTGCAACCACAACGCGGGCAACGCGGCTAAGCAAGGCGGGATCAC
CGGTTATCCTGACGCCAGTAGCCCAGGAACCGGCGCCACCGAAACGGCGG
CGAGTCGGCTCCAGCACACGGAAGACTGTCTCGGCCAGCTCGCTGGCACC
CAGCTCGCAGGGCGGCGCCGGGGATGAGGACTCCAAGGACAGTATGGCCT
CGTCCATGGACGACCTGCTGATGGCCGCAGCAGATATCAAGCAGGAGAAG
CTGACGCCCGATTTCGACGATAGTTTGATGCCAGAAGGCCTGCCCTCTAC
TTCTGGTGCGTCGAGTGCCAATGGTCATTCCTGCACCGAACCGCTTACTG
TGGACACGGAAATTAATGTTAAGCCCGCTGATTCCAAAGTAAAACCAAAG
GAGTCACCGGTGGTAGCAGTCGAGGAATCTCCATCACAATCCGAAACGCA
ATCTGCAAAGGTGTCAGCGCATGCGGGGAAGGCTCCATCTCTTAGTCCAG
ATATGATAAGTGAAGGCGTGAGCGCGGTCAGTGTTCGAAAGTTTTATAAG
AAGCCTGAGTTCCTGGAAAACAATCTGGGCATTGAAAAGGATCCGGAGCT
AGGTGAAATCGTTCAGACGGTTAGTAACAATGACACGGAAACAGATGTGG
AGATGGCTGTTGATGGCGAGGTGAATCAACCGTCAACTCCCAAGTCGCAG
GATAAAAAGAAAGAGGAGCAGGAAAAGAATCAGAAATCAGGGCTAAAGGC
AGCAAAGAAGGCTCCTGCTAAGTTAGAACCTAAAGCTGAAGACATTTCTG
AAATTCTTACTGACGTTCCTGTTGATATTTCGACTGAGGCAGTAGAAATT
ATAGAAGAAGCAGAGGAAGACACTTGTTCAAATAGCTCAATCAAACCAGG
TGAGCTCCGACTGGACGAGAGCAACGATGAACCTGAACTGCTTCTTGAAG
ACGCCCTCATAGTCAATGGTGATGAGAATGAGACACCAGATCAACCGGAG
GAAAAGGAGGACCAGGTGGAGTTCTTCCATACAGGAGAATACGACGACTT
TGAGCACGAGATTATGGTGGAGCTGGCGAAGGAGGGGGTGCTAGATGCCA
GCGGCAATGCATTAAGTCAGCAAAAGGTAGAACTTGAGCATCCCGAGGAT
GTAACTCTACACGAATCAAAAAATGACATAGAAGCCGAAGAATCGGTTGA
ACGTAAGCCTCTTAAGGACCCGTCGGTTGCGGACGAAATGGAGGACATGA
ATGAGGAATCCTATATTGACATTAAGGACCAGACAAATCAACTGTTAGTT
GAACACTTGGCAGAAGAGGCCATGGAAGCGGACTGCGGTCCCGAGGATAA
CAAGGAGAACTTGTCCACGTCTGCTTCGAGCACCGCTGCCGATGGTCTAG
ATATTCAGTTGGCCATCAAGGAGGATGACGACGAGGAGAAACCGCTTGCA
GTTATCGCTGACGAACAGAAGCCTGGGCTGCTGTTGACCAATGACATGAA
AGTGGATGAGAAACCAAATGGCAAGCAGGAATCGGTCTGTGATGAGCACG
TTCAGCTGGTGCCAAACCTTCGTCAAGAACAGGAAATTCACTTACAAAAT
CTGGGCCTACTCACGCACCAGGCCGCTGAACATAGGCGCAAGTGTCTGCT
TGAGGCACAGGCCCGCCAGGCGCAAATGCAGCTCCAGCAACATCACCACC
ATCAGCACAAGCGACAAGGAGCGCGCGGAGGAGGCAGTGCCACTCATGTG
GAATCCAGCGGTACTTTGAAGACAGTCATCAAGCTGAACAGGAGCAGCAA
CGGAGGAGTAAGCGGTAGTGGCGGCCTGCCTACTGGTACAGTTATCCATG
GAGGCTGTGGCTCCTCTTCAGCTTCTTCCACGTCCTCCTCCTCGGTGGGC
AGTGCCACACGTAAGTCAAGCGGGACCTTGGGCTCAGGAGCGGGAGCAGG
AGCTGGCGTTCGCCGGCAGTCGCTTAAGATGACATTCCAGAAGGGTCGGG
CTCGTGGTCACGGTGCTGCGGATCGATCCGCCGATCAGTATGGCGCCCAC
GCCGAGGACTCCTACTACACCATTCAAAACGAGAACGAAGGTGCGAAAAA
GTTTGTTGTAACTACTGGTAATACCGGCCGCAAGACTAATAACCGTTTCA
GCTCAACTAACAACTACCACTCGACGGTAGCCTTGCACGGTAGCAACTCT
GCGCTCCAGTACTATTCGTCCCACTCGGAAAGTCAGGGACAGACGGACCA
CGGCTTCTATCAGATGGTCAAAAAGGACGAAAAGGAGAAGATCCTCATTC
CGGAAAAGGCCTCCTCGTTTAAGTTTCACCCAGGGAGACTGTGCGAAGAC
CAGTGCTACTACTGTAGCGGAAAGTTTGGCCTCTATGACACCCCCTGCCA
TGTTGGACAAATAAAGTCCGTGGAGCGCCAGCAGAAGATCCTAGCCAACG
AGGAGAAGCTCACCGTGGATAACTGCTTGTGCGACGCATGTTTTCGACAC
GTGGACCGCCGGGCAAATGTGCCATCCTATAAGAAGCGTCTTTCCGCTTC
AGGTCACTTGGAGATGGGGTCTGCAGCGGGATCTGCACTAGAGAAACAGT
TTGCTGGCGACAGCGGCGTCATTACGGAATCGGGTGGCGAAGCTGGTTCT
ACGGCAGCTGTGGCCGTGCAGCAACGATCTTGTGGCGTGAAGGACTGCGT
CGAAGCGGCACGACACTCGCTGCGGCGCAAGTGCATACGCAAGAGAGTAA
AGAAGTATCAGCTCAGCCTGGAGATTCCCGCAGGCTCGTCGAACGTGGGG
CTGTGTGAGGCACATTACAATACGGTCATCCAATTTTCCGGCTGCGTTCT
TTGCAAGCGTAGATTAGGCAAGAACCATATGTACAACATAACCACGCAGG
ACACAATTCGACTGGAAAAGGCGCTGTCCGAGATGGGCATCCCAGTTCAG
CTTGGCATGGGCACTGCAGTCTGCAAGCTGTGTCGCTATTTTGCCAACCT
TTTGATAAAGCCACCGGATAGCACCAAGGCACAAAAGGCGGAATTCGTGA
AGAACTACAGAAAGAGGCTCCTCAAGGTGCATAATCTGCAGGATGGCAGT
CATGAGCTGTCCGAAGCGGATGAAGAAGAGGCACCTAATGCAACGGAGAC
AGAAAGGCCAACCTCAGACGGACACGAAGATCCCGAGATGCCCATGGTAG
CGGACTATGATGGACCTACCGACTCCAATTCCAGTAGTTCTTCGACTGCA
GCCCTGGACACCAGCAAACAAATGTCCAAGCTTCAGGCCATCCTGCAGCA
AAATGTGGGAGCGGATGCGGCAGGAGCTGCGGGAACAGGAACTGTTGCAG
CAAGTCCCGGAGGAAGCGGATCTGGGGCAGATATCTCTAACGTATTGCGA
GGGAATCCGAACATTTCCATGCGCGAACTTTTCCACGGCGAGGAAGAGCT
GGGTGTGCAGTTCAAGGTGCCGTTCGGATGCAGCAGCAGCCAGCGTACTC
CGGAGGGCTGGACACGAGTGCAGACTTTCCTACAATACGATGAGCCGACG
CGCCGCCTCTGGGAGGAGTTGCAAAAGCCGTACGGAAATCAGAGCTCATT
TCTGCGCCACTTGATACTATTAGAGAAGTACTACCGAAACGGAGATCTCG
TCCTAGCACCGCATGCTTCCTCCAATGCCACGGTTTACACAGAGACTGTT
CGTCAGCGGCTGAATTCGTTTGATCACGGTCACTGCGGTGGATTGAACAT
CGCAGGCAGCCCTTCTTCTTCGGGTTCCGGCAAGCGCAGTGGAGTTCCTC
AACCTACGGGTGCCAGTGTGCTGGCCACCGCCCTCACAACACCCTTGACA
AGCCATTCATCCTCCTCTGCATCCATTTCCTCCGAACAGCATTCGTCGGT
TGATCCTGTCATTCCGCTGGTAGACCTCAATGATGACGATGAAGGCGAAG
ATGGGGCAGGAGGAGCGGGCGAAAGGGAGTCGACAAATAGGCAGCAGGAC
GTAATCTTGGAATGCCTTAGAACTGCCTCTGTGGACAAGCTGACTAAGCA
GCTCAGCTCGAATGCGGTGACGATTATCGCCCGGCCCAAAGACAAATCGC
AGCTCTCCTGCAACAGCGGATCCTCCACGTCCATTTCCAGCTCCTCGTCC
GCTATTTCCTCGCCGGAGGAAGTGGCCGTCACTAAGGTTACAGCAGTCGC
ACCAGTCCAGTCCAAGGATGCACCGCCACTGGCGCCAGCAAGTAGCGGTG
TTAGCAACAGTCGTAGTATCCTTAAAACCAACCTCTTGGGCATGAACAAG
GCCGTGGAACTCGTGCCCTTAACGACTGCCCCCCACGCTTACAAGCCAAC
TGGATGCCATAAGCCTGAGAAACAGCAAAAGATTCTTGACGTGGCCAATA
AGCAGCCCGGTAGCCAGGGGGAACCGGTACCATCAAGCGCCTTGCTTGGC
CTGCAGTCAAAGCTAAAGCCTCCAACGCATCAGCAGCAGGTCAGCGGATC
AGGAGCGGGAACTAGTGGTTCTCAGAAGCCATCTAATGTGGCGCAATTGC
TTAGCTCTCCACCGGAGCTAATCAGCTTGCATCGACGGCAGACCAGCGGA
GCAGCAGCGGGGTCCAGCAGCTTCCTTCAGGGCAAGAGGCTTCAACTTCC
ACGATCTGGAGCAGGGCCTTCAGGAGCGGGAACGGGAACAGGCGCTGGAG
CAGCAGGAAGCCGCAGTGCGGGTGGACCACCACCGCCCAATGTGGTCATA
CTGCCGGACGCCTTAACCCCCCAGGAGCGACACGAGAGCAAGAGCTGGAA
GCCAACGCTGATACCGCTGGAGGATCAGCACAAGGTGCCGAACAAATCAC
ATGCTCTTTATCAGACCGCCGACGGTCGAAGGTTGCCCGCCCTGGTGCAA
GTGCAGTCTGGTGGCAAGCCATACCTCATCTCTATCTTCGACTATAACCG
CATGTGCATCTTGCGAAGGGAAAAGCTGATGCGGGACCAGTTGCTCAAGA
GTAACGCCAAGCCAAAGCCGCAGAACCAGCAACAGCAGCAGGGCCAAACG
CACCAGCAGCAGCAGAATTCCGCCGCATCGGCGGCTGCCTTCTCCAACAT
GGTGAAGTTGGCCCAGCAACACACGGCGCGACAGCAGCTTCAGCAGCTGC
AACAGAAGCCACAACAGCAGCAACAATTGCCCACTTTGCAGCCAGGTGGG
GTGCGACTTGCCCGGCTGCCGCAAAAACTACTGATGCCACCACTGACTAA
TCCGCAGATTGGCAGTCAAGCACCCAACTTACAGCCGTTGCTGTCTAGTA
CGCTGGATAACAGCAACAACTGCTGGCTGTGGAAAAACTTTCCTGATCCC
AATCAGTATCTGCTAAATGGAAACGGAGGGGGTGCCGGGAGCTCCTCCAG
CAAGTTGCCACATCTCACGGCCAAACCAGCCACGGCAACTAGTAGCTCCG
GAGCGGCCAACAAATCAGCAGGAAGCCTATTTACCCTCAAGCAGCAGCAG
CACCAGCAGAAACTCATCGACAACGCTATCATGTCAAAGATACCCAAAAG
TCTGACAGTAATACCGCAGCAGATGGGTGGTAATACCGGTGGCGATATGG
GGGGCAGCAGCTCCTCCGGCAAGGACTGATGACGGCGAAGGAGGGCGCCA
TGGCCATTAGCCGTAGCGCCGGAGGTAACCCGGCGAAGTAGTAGGATCAA
CAAGCAGGCGACGTGCAGCTTAAGCGGCGATCTTCAGAACAAGAGGTGAC
CAGCGGCGGCTCCATGGATATCACAAACTCCACAATTCCATGGCTGCAGT
AGAATAAGTGATACACT

(SEQ ID NO:225)

MSSRKVPGGSGGADESTAAAAPLDDNANASVEIPDSSEEPAMGVGEEMSI
ISKTRTSTLSVEPAKEPTVTAELEGEKELESNPVSKTPRSTPTPTLTPAV
TPTASDGVAAKSVRVTRHSSPLLLIISPTTSRREVGDGELDTEEPTGSGG
QRKSSVERSLAPVIRGRKSIKDLKEAKEVKSEEPPAAASESRAASGVTPG
QVKEQHVADGNEMESLPITDKKDHKDTKDKGDERETDQEEEKEKSADTEI
IADTEKTSEKQKYTEKDKAADKDGGKEKDIDANKDIDKEKEKVKEVLPPV
VPIAPVTPTCNRVTRKSHAQEQAINTRVTRNRRQSSTVGANSTASLVAAS
SSVTEQPPPSRGRRKKPVVVAPPLEPAVKRKRSQDVEADSDANNSTKYSK
VEVVKSEEAEAPEEDSSAVPIKQESVDGNEVSSISPTVTPTPTPAPTPAP
VPGSRRGRGRPQNRNSSSPATTTRATRLSKAGSPVILTPVAQEPAPPKRR
RVGSSTRKTVSASSLAPSSQGGAGDEDSKDSMASSMDDLLMAAADIKQEK
LTPDFDDSLMPEGLPSTSGASSANGHSCTEPLTVDTEINVKPADSKVKPK
ESPVVAVEESPSQSETQSAKVSAHAGKAPSLSPDMISEGVSAVSVRKFYK
KPEFLENNLGIEKDPELGEIVQTVSNNDTETDVEMAVDGEVNQPSTPKSQ
DKKKEEQEKNQKSGLKAAKKAPAKLEPKAEDISEILTDVPVDISTEAVEI
IEEAEEDTCSNSSIKPGELRLDESNDEPELLLEDALIVNGDENETPDQPE
EKEDQVEFFHTGEYDDFEHEIMVELAKEGVLDASGNALSQQKVELEHPED
VTLHESKNDIEAEESVERKPLKDPSVADEMEDMNEESYIDIKDQTNQLLV
EHLAEEAMEADCGPEDNKENLSTSASSTAADGLDIQLAIKEDDDEEKPLA
VIADEQKPGLLLTNDMKVDEKPNGKQESVCDEHVQLVPNLRQEQEIHLQN
LGLLTHQAAEHRRKCLLEAQARQAQMQLQQHHHHQHKRQGARGGGSATHV
ESSGTLKTVIKLNRSSNGGVSGSGGLPTGTVIHGGCGSSSASSTSSSSVG
SATRKSSGTLGSGAGAGAGVRRQSLKMTFQKGRARGHGAADRSADQYGAH
AEDSYYTIQNENEGAKKFVVTTGNTGRKTNNRFSSTNNYHSTVALHGSNS
ALQYYSSHSESQGQTDHGFYQMVKKDEKEKILWEKASSFKFHPGRLCED
QCYYCSGKFGLYDTPCHVGQIKSVERQQKILANEEKLTVDNCLCDACFRH
VDRRANVPSYKKRLSASGHLEMGSAAGSALEKQFAGDSGVITESGGEAGS
TAAVAVQQRSCGVKDCVEAARHSLRRKCIRKRVKKYQLSLEIPAGSSNVG
LCEAHYNTVIQFSGCVLCKRRLGKNHMYNITTQDTIRLEKALSEMGWVQ
LGMGTAVCKICRYFANLLIKPPDSTKAQKAEFVKNYRKRLLKVHNLQDGS
HELSEADEEEAPNATETERPTSDGHEDPEMLPMVADYDGPTDSNSSSSSTA
ALDTSKQMSKLQAILQQNVGADAAGAAGTGTVAASPGGSGSGADISNVLR
GNPNISMRELFHGEEELGVQFKVPFGCSSSQRTPEGWTRVQTFLQYDEPT
RRLWEELQKPYGNQSSFLRHLILLEKYYRNGDLVLAPHASSNATVYTETV
RQRLNSFDHGHCGGLNIAGSPSSSGSGKRSGVPQPTGASVLATALTTPLT
SHSSSSASISSEQHSSVDPVIPLVDLNDDDEGEDGAGGAGERESTNRQQD
VILECLRTASVDKLTKQLSSNAVTIIARPKDKSQLSCNSGSSTSISSSSS
AISSPEEVAVTKVTAVAPVQSKDAPPLAPASSGVSNSRSTLKTNLLGMNK
AVELVPLTTAPHAYKPTGCHKIPEKQQKILDVANKQPGSQGEPVPSSALLG
LQSKLKPPTHQQQVSGSGAGTSGSQKPSNVAQLLSSPPELISLHRRQTSG
AAAGSSSFLQGKRLQLPRSGAGPSGAGTGTGAGAAGSRSAGGPPPPNVVI
LPDALTPQERHESKSWKPTLIPLEDQHKVPNKSHALYQTADGRRLPALVQ
VQSGGKPYLIS1TFDYNRMCILRREKLMRDQLLKSNAKPKPQNQQQQQGQT
HQQQQNSAASAAAFSNMVKLAQQHTARQQLQQLQQKPQQQQQLPTLQPGG
VRLARLPQKLLMPPLTNPQIGSQAPNLQPLLSSTLDNSNNCWLWKNFPDP
NQYLLNGNGGGAGSSSSKLPHLTAKPATATSSSGAANKSAGSLFTLKQQQ
HQQKKLIDNAIMSKIPKSLTVIPQQMGGNTGGDMGGSSSSGKD

Human homologue of Complete Genome candidate

AAF13722—neurofilament protein

| | | |
|---|---|---|
| 1 | atgatgagct tcggcggcgc ggacgcgctg ctgggcgccc cgttcgcgcc gctgcatggc | (SEQ ID NO:226) |
| 61 | ggcggcagcc tccactacgc gctagcccga aagggtggcg caggcgggac gcgctccgcc | |
| 121 | gctggctcct ccagcggctt ccactcgtgg acacggacgt ccgtgagctc cgtgtccgcc | |
| 181 | tcgcccagcc gcttccgtgg cgcaggcgcc gcctcaagca ccgactcgct ggacacgctg | |
| 241 | agcaacgggc cggagggctg catggtggcg gtggccacct cacgcagtga gaaggagcag | |
| 301 | ctgcaggcgc tgaacgaccg cttcgccggg tacatcgaca aggtgcggca gctggaggcg | |
| 361 | cacaaccgca gcctggaggg cgaggctgcg gcgctgcggc agcagcaggc gggccgctcc | |
| 421 | gctatgggcg agctgtacga gcgcgaggtc cgcgagatgc gcggcgcggt gctgcgcctg | |
| 481 | ggcgcggcgc gcggtcagct acgcctggag caggagcacc tgctcgagga catcgcgcac | |
| 541 | gtgcgccagc gcctagacga cgaggcccgg cagcgagagg aggccgaggc ggcggcccgc | |
| 601 | gcgctggcgc gcttcgcgca ggaggccgag gcggcgcgcg tggacctgca gaagaaggcg | |
| 661 | caggcgctgc aggaggagtg cggctacctg cggcgccacc accaggaaga ggtgggcgag | |
| 721 | ctgctcggcc agatccaggg ctccggcgcc gcgcaggcgc agatgcaggc cgagacgcgc | |
| 781 | gacgccctga agtgcgacgt gacgtcggcg ctgcgcgaga ttcgcgcgca gcttgaaggc | |
| 841 | cacgcggtgc agagcacgct gcagtccgag gagtggttcc gagtgaggct ggaccgactg | |
| 901 | tcggaggcag ccaaggtgaa cacagacgct atgcgctcag cgcaggagga gataactgag | |
| 961 | taccggcgtc agctgcaggc caggaccaca gagctggagg cactgaaaag caccaaggac | |
| 1021 | tcactggaga ggcagcgctc tgagctggag gaccgtcatc aggccgacat tgcctcctac | |
| 1081 | caggaagcca ttcagcagct ggacgctgag ctgaggaaca ccaagtggga gatggccgcc | |
| 1141 | cagctgcgag aataccagga cctgctcaat gtcaagatgg ctctggatat agagatagcc | |
| 1201 | gcttacagaa aactcctgga aggtgaagag tgtcggattg gctttggccc aattcctttc | |
| 1261 | tcgcttccag aaggactccc caaaattccc tctgtgtcca ctcacataaa ggtgaaaagc | |
| 1321 | gaagagaaga tcaaagtggt ggagaagtct gagaaagaaa ctgtgattgt ggaggaacag | |
| 1381 | acagaggaga cccaagtgac tgaagaagtg actgaagaag aggagaaaga ggccaaagag | |
| 1441 | gaggagggca aggaggaaga agggggtgaa gaagaggagg cagaaggggg agaagaagaa | |
| 1501 | acaaagtctc ccccagcaga agaggctgca tccccagaga aggaagccaa gtcaccagta | |
| 1561 | aaggaagagg caaagtcacc ggctgaggcc aagtccccag agaaggagga agcaaaatcc | |
| 1621 | ccagccgaag tcaagtcccc tgagaaggcc aagtctccag caaaggaaga ggcaaagtca | |
| 1681 | ccgcctgagg ccaagtcccc agagaaggag gaagcaaaat ctccagctga ggtcaagtcc | |
| 1741 | cccgagaagg ccaagtcccc agcaaaggaa gaggcaaagt caccggctga ggccaagtct | |
| 1801 | ccagagaagg ccaagtcccc agtgaaggaa gaagcaaagt caccggctga ggccaagtcc | |
| 1861 | ccagtgaagg aagaagcaaa atctccagct gaggtcaagt ccccggaaaa ggccaagtct | |
| 1921 | ccaacgaagg aggaagcaaa gtcccctgag aaggccaagt cccctgagaa ggccaagtcc | |
| 1981 | ccagagaagg aagaggccaa gtcccctgag aaggccaagt ccccagtgaa ggcagaagca | |
| 2041 | aagtcccctg agaaggccaa gtccccagtg aaggcagaag caaagtcccc tgagaaggcc | |
| 2101 | aagtccccag tgaaggaaga agcaaagtcc cctgagaagg ccaagtcccc agtgaaggaa | |
| 2161 | gaagcaaagt cccctgagaa ggccaagtcc ccagtgaagg aagaagcaaa gacccccgag | |
| 2221 | aaggccaagt ccccagtgaa ggaagaagcc aagtccccag agaaggccaa gtccccagag | |
| 2281 | aaggccaaga ctcttgatgt gaagtctcca gaagccaaga ctccagcgaa ggaggaagca | |
| 2341 | aggtcccctg cagacaaatt ccctgaaaag gccaaaagcc ctgtcaagga ggaggtcaag | |
| 2401 | tccccagaga aggcgaaatc tcccctgaag gaggatgcca aggcccctga gaaggagatc | |
| 2461 | ccaaaaaagg aagaggtgaa gtccccagtg aaggaggagg agaagcccca ggaggtgaaa | |
| 2521 | gtcaaagagc ccccaaagaa ggcagaggaa gagaaagccc ctgccacacc aaaaacagag | |
| 2581 | gagaagaagg acagcaagaa agaggaggca cccaagaagg aggctccaaa gcccaaggtg | |
| 2641 | gaggagaaga aggaacctgc tgtcgaaaag cccaaagaat ccaaagttga agccaagaag | |
| 2701 | gaagaggctg aagataagaa aaaagtcccc accccagaga aggaggctcc tgccaaggtg | |
| 2761 | gaggtgaagg aagacgctaa acccaaagaa aagacagagg tggccaagaa ggaaccagat | |
| 2821 | gatgccaagg ccaaggaacc cagcaaacca gcagagaaga aggaggcagc accggagaaa | |
| 2881 | aaagacacca aggaggagaa ggccaagaag cctgaggaga aacccaagac agaggccaaa | |
| 2941 | gccaaggaag atgacaagac cctctcaaaa gagcctagca agcctaaggc agaaaaggct | |
| 3001 | gaaaaatcct ccagcacaga ccaaaaagac agcaagcctc cagagaaggc cacagaagac | |
| 3061 | aaggccgcca aggggaagta aggcagggag aaaggaacat ccggaacagc caaagaaact | |
| 3121 | cagaagagtc ccggagctca aggatcagag taacacaatt ttcacttttt ctgtctttat | |
| 3181 | gtaagaagaa actgcttaga tgacggggcc tccttcttca aacaggaatt tctgttagca | |
| 3241 | atatgttagc aagagagggc actcccaggc ccctgccccc atgccctccc caggcgatgg | |
| 3301 | acaattatga tagcttatgt agctgaatgt gatacatgcc gaatgccaca cgtaaacact | |
| 3361 | tgactataaa aactgccccc ctcctttcca aataagtgca tttattgcct ctatgtgcaa | |
| 3421 | ctgacagatg accgcaataa tgaatgagca gttagaaata cattatgctt gagatgtctt | |
| 3481 | aacctattcc caaatgcctt ctgttttcca aaggagtggt caagcccttg cccagagctc | |
| 3541 | tctattctgg aagagcggtc caggtggggc cgggcactgg ccactgaatt atgccagggc | |
| 3601 | gcactttcca ctggagttca ctttcaattg cttctgtgca ataaaaccaa gtgcttataa | |
| 3661 | aatgaaaaaa aaaaaaaaaa tgctgttatt ctctttccct gggaaggctg ggggcagggc | |
| 3721 | aggggaggtc tggatgtgac accccagact gcatgggact gagcaagcat cagt | |
| 1 | mmsfggadal lgapfaplhg ggslhyalar kggaggtrsa agsssgfhsw trtsvssvsa | (SEQ ID NO:227) |
| 61 | spsrfrgaga asstdsldtl sngpegcmva vatsrsekeq lqalndrfag yidkvrqlea | |
| 121 | hnrslegeaa alrqqqagrs amgelyerev remrgavlrl gaargqlrle qehllediah | |
| 181 | vrqrlddear qreeaeaaar alarfaqeae aarvdlqkka qalqeecgyl rrhhqeevge | |
| 241 | llgqiqgsga aqaqmqaetr dalkcdvtsa lrelraqieg havqstlqse ewfrvrldrl | |
| 301 | seaakvntda mrsaqeeite yrrqlqartt elealkstkd slerqrsele drhqadiasy | |
| 361 | qeaiqqldae lrntkwemaa qlreyqdlln vkmaldieia ayrkllegee crigfgpipf | |
| 421 | slpeglpkip svsthikvks eekikvveks eketviveeq teetqvteev teeeekeake | |
| 481 | eegkeeegge eeeaeggeee tksppaeeaa spekeakspv keeakspaea kspekeeaks | |
| 541 | paevkspeka kspakeeaks ppeakspeke eakspaevks pekakspake eakspaeaks | |
| 601 | pekakspvke eakspaeaks pvkeeakspa evkspekaks ptkeeakspe kakspekaks | |
| 661 | pekeeakspe kakspvkaea kspekakspv kaeakspeka kspvkeeaks pekakspvke | |
| 721 | eakspekaks pvkeeaktpe kakspvkeea kspekakspe kaktldvksp eaktpakeea | |
| 781 | rspadkfpek akspvkeevk spekaksplk edakapekei pkkeevkspv keeekpqevk | |
| 841 | vkeppkkaee ekapatpkte ekkdskkeea pkkeapkpkv eekkepavek pkeskveakk | |
| 901 | eeaedkkkvp tpekeapakv evkedakpke ktevakkepd dakakepskp aekkeaapek | |
| 961 | kdtkeekakk peekpkteak akeddktlsk epskpkaeka ekssstdqkd skppekated | |
| 1021 | kaakgk | |

Putative function
unknown

### Example 21

### Category 3

Line ID—265

Phenotype—Lethal phase pharate adult. High mitotic index, rod like overcondensed chromosomes, few anaphases with lagging chromosomes

Annotated Drosophila genome genomic segment containing P element insertion site (and map position)—AE003509 (17B4-5)

P element insertion site—52,563

Annotated Drosophila genome Complete Genome candidate

CG6407—Wnt5

(SEQ ID NO:228)

CAGTTGTTTACAATTTGTCGTTGAGGGTGGATTACTTCGTCGCGAGTTTC
GTTCGTGCATGATGCGGTTGTGGTTGATTGTATACATACATACTATGCAC
AAATCCAGTTCTCATTTTGTTATTTTACAAATTCTCAGCGAGCGCATGAA
CTGGCAGCCTATAGCGAGCAGCTAATCACAATATTTACGGCAGATTCGTG
GACTCAAGGAAATTCAGCCAGCAGCCAATCGATTTTCTAGTGTTATCGAA
AAACATTTTTCATTCCTTCATTTCGTTCAACTAACAATACTAGTTACTAC
TAACAATACTCTGTAATAGTAATAGTAAGAGGAACAGGAATAGGAATACA
CATACTCCAAAGCGATAATGAGTTGCTACAGAAAAAGGCACTTTCTATTG
TGGCTCTTGCGTGCTGTGTGTATGTTGCACTTAACCGCGAGAGGGGCATA
TGCCACAGTTGGGTTGCAAGGAGTGCCGACATGGATATATCTCGGCCTCA
AGTCCCCCTTCATCGAGTTTGGCAACCAGGTGGAGCAGCTGGCCAATTCC
AGCATACCACTGAACATGACCAAGGACGAGCAGGCCAATATGCATCAAGA
GGGCCTACGCAAGCTCGGTACGTTCATAAAGCCAGTGGACCTGCGGGACT
CGGAGACTGGCTTCGTCAAGGCCGATCTCACCAAGAGACTGGTATTCGAT
AGACCGAACAACATTACATCTCGCCCTATTCACCCGATACAGGAGGAGAT
GGATCAGAAGCAGATAATCCTGCTCGACGAGGATACCGACGAGAATGGCC
TGCCAGCCAGTCTCACCGACGAGGATCGCAAGTTTATAGTGCCGATGGCG
CTCAAGAATATATCGCCCGATCCACGCTGGGCGGCCACTACACCGAGTCC
CTCCGCTTTGCAGCCGAACGCTAAAGCCATCTCGACCATTGTGCCCTCGC
CTCTGGCCCAGGTCGAGGGGGATCCCACGTCCAACATCGATGACCTGAAG
AAGCACATACTCTTCTTGCACAACATGACCAAGACCAATTCGAACTTCGA
GTCGAAATTCGTTAAATTCCCGAGCCTGCAAAAGGACAAGGCCAAGACAT
CGGGAGCTGGCGGTTCGCCGCCCAATCCCAAGCGGCCCCAGCGGCCGATT
CATCAGTATTCCGCGCCCATAGCCCCACCAACACCCAAGGTGCCCGCGCC
AGATGGCGGCGGCGTAGGAGGAGCAGCTTACAATCCCGGAGAGCAGCCAA
TTGGTGGCTACTATCAGAACGAGGAACTAGCGAATAATCAATCCCTTCTT
AAACCAACAGATACCGACTCCCATCCAGCGGCCGGCGGTAGCAGCCATGG
CCAGAAGAATCCCAGCGAGCCCCAGGTGATACTGCTCAACGAGACACTCT
CCACGGAGACCTCAATCGAAGCGGATCGCAGTCCATCGATAAACCAGCCC
AAGGCGGGATCGCCTGCGCGCACAACAAAGCGACCACCTTGCCTGCGCAA
TCCCGAGTCCCCGAAATGCATACGTCAGCGTCGGCGGGAGGAGCAACAGC
GGCAGCGGGAGCGGGACGAGTGGTTCCGCGGTCAGTCGCAGTACATGCAG
CCCCGGTTCGAGCCGATCATACAGACGATTAACAATACGAAGAGATTTGC
CGTATCAATCGAGATTCCAGACTCCTTTAAAGTATCCTCCGAGGGATCGG
ATGGGGAGTTGCTTTCGCGAGTCGAACGCTCGCAGCCCAGCATTAGTAGT
AGTAGTAGTAGCAGTAGTAGCAGTAGTAGGAAAATCATGCCAGACTATAT
TAAGGTATCCATGGAGAACAACACATCCGTCACGGATTATTTTAAGCACG
ACGTTGTGATGACATCGGCAGATGTCGCCAGCGATAGGGAATTCCTTATC
AAGAACATGGAGGAGCACGGAGGCGCTGGCTCCGCGAACAGTCATCACAA
TGATACGACTCCAACTGCAGACGCATATTCGGAGACAATCGATCTTAATC
CCAATAACTGCTATAGCGCAATAGGTCTAAGCAACAGCCAAAAGAAGCAA
TGTGTTAAGCACACCAGCGTGATGCCGGCCATAAGTCGTGGTGCCCGTGC
CGCCATCCAGGAGTGCCAGTTTCAGTTCAAGAATCGCCGCTGGAACTGCA
GCACAACGAACGACGAGACCGTATTTGGTCCCATGACCAGCCTGGCTGCT
CCCGAAATGGCCTTCATCCACGCCCTGGCCGCGGCCACGGTGACCAGCTT
CATAGCTCGCGCCTGCCGGGATGGCCAACTGGCCTCCTGCAGCTGCTCCC
GCGGCAGTCGACCCAAACAGCTCCACGACGACTGGAAGTGGGGCGGCTGT
GGCGACAACCTGGAGTTCGCCTACAAGTTCGCCACGGACTTCATCGATTC
GCGGGAGAAGGAAACCAATCGCGAGACGCGTGGCGTTAAGAGAAAACGCG
AGGAGATCAACAAGAATCGCATGCATTCCGATGACACGAATGCTTTTAAC
ATAGGTATTAAACGTAACAAAAACGTAGATGCTAAAAACGATACAAGTTT
GGTAGTGAGAAACGTTAGGAAAAGCACTGAGGCTGAAAACAGTCACATAC
TCAATGAGAACTTTGATCAGCACCTATTGGAACTAGAGCAGCGCATTACG
AAGGAGATACTTACATCCAAGATAGACGAGGAGGAGATGATTAAGCTGCA
GGAGAAGATCAAACAGGAGATTGTCAACACCAAGTTCTTCAAGGGTGAGC
AGCAGCCGCGCAAGAAGAAGCGAAAAAACCAGAGAGCCGCCGCCGATGCG
CCCGCCTATCCGAGGAACGGCATCAAGGAGAGCTACAAGGATGGCGGCAT
ATTGCCGCGCAGCACGGCCACTGTCAAGGCCAGGAGCCTGATGAACTTGC
ACAACAACGAGGCCGGACGTCGGGCGGTGATCAAGAAGGCCAGGATAACG
TGCAAGTGCCACGGCGTGTCCGGCTCCTGCAGCCTGATCACCTGCTGGCA
GCAATTGTCCTCCATCCGGGAGATTGGCGACTATCTGCGCGAGAAGTACG
AGGGCGCCACCAAGGTGAAGATCAACAAGCGTGGCCGCCTCCAGATCAAG
GACTTGCAATTCAAGGTGCCGACCGCTCACGATCTTATTTACCTAGACGA
AAGTCCCGACTGGTGCCGCAATAGCTATGCGCTGCATTGGCCGGGAACGC
ACGGACGTGTGTGCCACAAAAACTCGTCGGGATTGGAGAGCTGTGCCATC
CTCTGCTGCGGCCGGGGCTATAATACGAAGAACATTATAGTTAACGAACG
CTGCAATTGCAAATTTCACTGGTGTTGCCAGGTTAAATGTGAAGTTTGTA
CGAAGGTACTCGAGGAGCACACATGTAAATAGAGCGTTGATTGAATTCGA
ATGTCTTAATGTTTGTGACTAAGCCATGAAGGAAATAATCGTATTTAAAC
AGTCCTCTCCATTTTAATTGCCATTACCATACACCATCATATTGCTTCTT
CTTAAAATGCT

(SEQ ID NO:229)

MSCYRKRHFLLWLLRAVCMLHLTARGAYATVGLQGVPTWIYLGLKSPFIE
FGNQVEQLANSSIPLNMTKDEQANMHQEGLRKLGTFIKPVDLRDSETGFV
KADLTKRLVFDRPNNITSRPIHPIQEEMDQKQIILLDEDTDENGLPASLT
DEDRKFIVPMALKNISPDPRWAATTPSPSALQPNAKAISTIVPSPLAQVE
GDPTSNIDDLKKHILFLHNMTKTNSNFESKFVKFPSLQKDKAKTSGAGGS
PPNPKRPQRPIHQYSAPIAPPTPKVPAPDGGGVGGAAYNPGEQPIGGYYQ
NEELANNQSLLKPTDTDSHPAAGGSSHGQKNPSEPQVILLNETLSTETSI
EADRSPSINQPKAGSPARTTKRPPCLRNPESPKCIRQRRREEQQRQRERD
EWFRGQSQYMQPRFEPIIQTINNTKRFAVSIEIPDSFKVSSEGSDGELLS
RVERSQPSISSSSSSSSSSSRKIMPDYIKVSMENNTSVTDYFKHDVVMTS
ADVASDREFLIKNMEEHGGAGSANSHHNDTTPTADAYSETIDLNPNNCYS
AIGLSNSQKKQCVKHTSVMPAISRGARAAIQECQFQFKNRRWNCSTTNDE
TVFGPMTSLAAPEMAFIHALAAATVTSFIARACRDGQLASCSCSRGSRPK
QLHDDWKWGGCGDNLEFAYKFATDFIDSREKETNRETRGVKRKREEINIKN
RMHSDDTNAFNIGIKRNKNVDAKNDTSLVVRNVRKSTEAENSHILNENFD
QHLLELEQRITKEILTSKIDEEEMIKLQEKIKQEIVNTKFFKGEQQPRKK
KRKNQRAAADAPAYPRNGIKESYKDGGILPRSTATVKARSLMNLHNNEAG
RRAVIKKARITCKCHGVSGSCSLITCWQQLSSIREIGDYLREKYEGATKV
KINKRGRLQIKDLQFKVPTAHDLIYLDESPDWCRNSYALHWPGTHGRVCH
KNSSGLESCAILCCGRGYNTKNIIVNERCNCKFHWCCQVKCEVCTKVLEE
HTCK

Human homologue of Complete Genome candidate

AAA16842—hWNT5A

| | | |
|---|---|---|
| 1 | attaattctg gctccacttg ttgctcggcc caggttgggg agaggacgga gggtggccgc | (SEQ ID NO:230) |
| 61 | agcgggttcc tgagtgaatt acccaggagg gactgagcac agcaccaact agagaggggt | |
| 121 | cagggggtgc gggactcgag cgagcaggaa ggaggcagcg cctggcacca gggctttgac | |
| 181 | tcaacagaat tgagacacgt ttgtaatcgc tggcgtgccc cgcgcacagg atcccagcga | |
| 241 | aaatcagatt tcctggtgag gttgcgtggg tggattaatt tggaaaaaga aactgcctat | |
| 301 | atcttgccat caaaaaactc acggaggaga agcgcagtca atcaacagta aacttaagag | |
| 361 | acccccgatg ctcccctggt ttaacttgta tgcttgaaaa ttatctgaga gggaataaac | |
| 421 | atcttttcct tcttccctct ccagaagtcc attggaatat taagcccagg agttgctttg | |
| 481 | gggatggctg gaagtgcaat gtcttccaag ttcttcctag tggctttggc catatttttc | |
| 541 | tccttcgccc aggttgtaat tgaagccaat tcttggtggt cgctaggtat gaataaccct | |
| 601 | gttcagatgt cagaagtata tattatagga gcacagcctc tctgcagcca actggcagga | |
| 661 | ctttctcaag gacagaagaa actgtgccac ttgtatcagg accacatgca gtacatcgga | |
| 721 | gaaggcgcga agacaggcat caaagaatgc cagtatcaat tccgacatcg acggtggaac | |
| 781 | tgcagcactg tggataacac ctctgttttt ggcagggtga tgcagatagg cagccgcgag | |
| 841 | acggccttca catacgccgt gagcgcagca ggggtggtga acgccatgag ccgggcgtgc | |
| 901 | cgcgagggcg agctgtccac ctgcggctgc agccgcgccg cgcgccccaa ggacctgccg | |
| 961 | cgggactggc tctggggcgg ctgcggcgac aacatcgact atggctaccg ctttgccaag | |
| 1021 | gagttcgtgg acgcccgcga gcgggagcgc atccacgcca agggctccta cgagagtgct | |
| 1081 | cgcatcctca tgaacctgca caacaacgag gccggccgca ggacggtgta caacctggct | |
| 1141 | gatgtggcct gcaagtgcca tggggtgtcc ggctcatgta gcctgaagac atgctggctg | |
| 1201 | cagctggcag acttccgcaa ggtgggtgat gccctgaagg agaagtacga cagcgcggcg | |
| 1261 | gccatgcggc tcaacagccg gggcaagttg gtacaggtca acagccgctt caactcgccc | |
| 1321 | accacacaag acctggtcta catcgacccc agccctgact actgcgtgcg caatgagagc | |
| 1381 | accggctcgc tgggcacgca gggccgcctg tgcaacaaga cgtcggaggg catggatggc | |
| 1441 | tgcgagctca tgtgctgcgg ccgtgggtac gaccagttca agaccgtgca gacggagcgc | |
| 1501 | tgccactgca agttccactg gtgctgctac gtcaagtgca agaagtgcac ggagatcgtg | |
| 1561 | gaccagtttg tgtgcaagta gtgggtgcca cccagcactc agccccgctc ccaggacccg | |
| 1621 | cttatttata gaaagtacag tgattctggt ttttggtttt tagaaatatt ttttattttt | |
| 1681 | ccccaagaat tgcaaccgga accatttttt ttcctgttac catctaagaa ctctgtggtt | |
| 1741 | tattattaat attataatta ttatttggca ataatggggg tgggaaccac gaaaaatatt | |
| 1801 | tattttgtgg atctttgaaa aggtaataca agacttcttt tggatagtat agaatgaagg | |
| 1861 | gggaaataac acatacccta acttagctgt gtgggacatg gtacacatcc agaaggtaaa | |
| 1921 | gaaatacatt ttctttttct caaatatgcc atcatatggg atgggtaggt tccagttgaa | |
| 1981 | agagggtggt agaaatctat tcacaattca gcttctatga ccaaaatgag ttgtaaattc | |
| 2041 | tctggtgcaa gataaaaggt cttgggaaaa caaaacaaaa caaaacaaac ctcccttccc | |
| 2101 | cagcagggct gctagcttgc tttctgcatt ttcaaaatga taatttacaa tggaaggaca | |
| 2161 | agaatgtcat attctcaagg aaaaaaggta tatcacatgt ctcattctcc tcaaatattc | |
| 2221 | catttgcaga cagaccgtca tattctaata gctcatgaaa tttgggcagc agggaggaaa | |
| 2281 | gtccccagaa attaaaaaat ttaaaactct tatgtcaaga tgttgatttg aagctgttat | |
| 2341 | aagaattggg attccagatt tgtaaaaaga cccccaatga ttctggacac tagatttttt | |
| 2401 | gtttggggag gttggcttga acataaatga aatatcctgt attttcttag ggatacttgg | |
| 2461 | ttagtaaatt ataatagtag aaataataca tgaatcccat tcacaggttt ctcagcccaa | |
| 2521 | gcaacaaggt aattgcgtgc cattcagcac tgcaccagag cagacaacct atttgaggaa | |
| 2581 | aaacagtgaa atccaccttc ctcttcacac tgagccctct ctgattcctc cgtgttgtga | |
| 2641 | tgtgatgctg gccacgtttc caaacggcag ctccactggg tcccctttgg ttgtaggaca | |
| 2701 | ggaaatgaaa cattaggage tctgcttgga aaacagttca ctacttaggg atttttgttt | |
| 2761 | cctaaaactt ttattttgag gagcagtagt tttctatgtt ttaatgacag aacttggcta | |
| 2821 | atggaattca cagaggtgtt gcagcgtatc actgttatga tcctgtgttt agattatcca | |
| 2881 | ctcatgcttc tcctattgta ctgcaggtgt accttaaaac tgttcccagt gtacttgaac | |
| 2941 | agttgcattt ataagggggg aaatgtggtt taatggtgcc tgatatctca aagtcttttg | |
| 3001 | tacataacat atatatatat atacatatat ataaatataa atataaatat atctcattgc | |
| 3061 | agccagtgat ttagatttac agcttactct ggggttatct ctctgtctag agcattgttg | |
| 3121 | tccttcactg cagtccagtt gggattattc caaaagtttt ttgagtcttg agcttgggct | |
| 3181 | gtggccccgc tgtgatcata ccctgagcac gacgaagcaa cctcgtttct gaggaagaag | |
| 3241 | cttgagttct gactcactga aatgcgtgtt gggttgaaga tatctttttt tcttttctgc | |
| 3301 | ctcacccctt tgtctccaac ctccatttct gttcactttg tggagagggc attacttgtt | |
| 3361 | cgttatagac atggacgtta agagatattc aaaactcaga agcatcagca atgtttctct | |
| 3421 | tttcttagtt cattctgcag aatggaaacc catgcctatt agaaatgaca gtacttatta | |
| 3481 | attgagtccc taaggaatat tcagcccact acatagatag cttttttttt tttttttttt | |
| 3541 | ttttaataag gacacctctt tccaaacagg ccatcaaata tgttcttatc tcagacttac | |
| 3601 | gttgttttaa aagtttggaa agatacacat cttttcatac ccccccttag gaggttgggc | |
| 3661 | tttcatatca cctcagccaa ctgtggctct taatttattg cataatgata tccacatcag | |
| 3721 | ccaactgtgg ctctttaatt tattgcataa tgatattcac atcccctcag ttgcagtgaa | |
| 3781 | ttgtgagcaa aagatcttga aagcaaaaag cactaattag tttaaaatgt cacttttttg | |
| 3841 | gtttttatta tacaaaaacc atgaagtact ttttttattt gctaaatcag attgttcctt | |
| 3901 | tttagtgact catgtttatg aagagagttg agtttaacaa tcctagcttt taaaagaaac | |
| 3961 | tatttaatgt aaaatattct acatgtcatt cagatattat gtatatcttc tagcctttat | |
| 4021 | tctgtacttt taatgtacat atttctgtct tgcgtgattt gtatatttca ctggtttaaa | |
| 4081 | aaacaaacat cgaaaggctt attccaaatg gaag | |
| 1 | magsamsskf flvalaiffs faqvvieans wwslgmnnpv qmsevyiiga qplcsqlagl | (SEQ ID NO:231) |
| 61 | sqgqkklchl yqdhmqyige gaktgikecq yqfrhrrwnc stvdntsvfg rvmqigsret | |
| 121 | aftyavsaag vvnamsracr egelstcgcs raarpkdlpr dwlwggcgdn idygyrfake | |
| 181 | fvdarereri hakgsyesar ilmnlhnnea grrtvynlad vackchgvsg scslktcwlq | |
| 241 | ladfrkvgda lkekydsaaa mrlnsrgklv qvnsrfnspt tqdlvyidps pdycvrnest | |
| 301 | gslgtqgrlc nktsegmdgc elmccgrgyd qfktvqterc hckfhwccyv kckkcteivd | |
| 361 | qfvck | |

Putative function
Wnt oncogene

### Example 22

### Category 3

Line ID—392

Phenotype—Lethal phase larval stage 3-pharate adult, small brain and optic lobes, high mitotic index, rod-like overcondensed chromosomes, fewer ana- and telophases, overcondensed chromosomes in ana- and telophase

Annotated Drosophila genome genomic segment containing P element insertion site (and map position)—AE003495 (12D)

P element insertion site—35,688

Annotated Drosophila genome Complete Genome candidate

CG12482—novel protein

(SEQ ID NO:232)

ATGGGTTGCACCTGCTGTGACAATAAACCCAAGCCGGAGACCATTGAGAT
ATATTCGGTGAAAATCCGTGAGAATGGTACATACAAGTTGATCAAGATGC
AATTGGCGGATATTTGGAGTCACGGATGGGAGCTGCGTATCAATAACTTT
GCCGACAAGGAAAAGGTGCCGCACAACGAGAAGGATATTCGCAATCAGGT
GTCGGTGGCGCGCAAAGCCAAACAGAGTCTGTGGAACAATAATAAGCATT
TTGTGTACTGGTGCCGCTACGGAAGTCGTCAGCAGGATCTGCGAAAGCGA
CAGGTAACGACGAGTGCCAATCACGTGCTGCTGCACCTGATCAATTGA

(SEQ ID NO:233)

MGCTCCDNKPKPETIEIYSVKIRENGTYKLIKMQLADIWSHGWELRINNF
ADKEKVPHNEKDLRNQVSVARKAKQSLWNNNKHFVYWCRYGSRQQDLRKR
QVTTSANHVLLHLIN

Human homologue of Complete Genome candidate
none

Putative function
unknown

### Example 23

### Category 3

Line ID—37

Phenotype—Lethal phase larval stage 3. Small brain, few cells in mitosis, badly defined chromosomes form a broad bend, weak chromosome condensation, abnormal anaphases with broken chromosomes

Annotated Drosophila genome genomic segment containing P element insertion site (and map position)—AE003418 (1C1-2)

P element insertion site—105,970

Annotated Drosophila genome Complete Genome candidate

CG16983—skpA, SCF ubiquitin ligase subunit (3 splice variants)

(SEQ ID NO:234)

CCATTTGAAAGTATCGGTGTAATTTGTTTTCAGAGAAATTAATTTCCGTT
TACTGTGCAATTCGGTGTGAAAGTGTTCAGATTTATCAATGCGTATTCTG
CTTTCGACTTCGCCACCAATCTGTGCTGCAAGTTACCATTACCAGGTCCA
CCTGGTTCCCGCCAGTTTTCTTTCATTGTGGCTAGTTGTTGTTCGTGCCT
TCGATAAAGACGTTTAGAGGTGTTTTTAGAGTTTCGCCATCTGGTCACTA
TAGCCGTTTCGTTTTTTACATGCCCAGCATCAAGTTGCAATCTTCGGATG
AGGAGATCTTTGACACGGATATCCAGATCGCCAAGTGCTCCGGCACTATC
AAGACCATGCTGGAGGACTGCGGCATGGAGGACGATGAGAATGCCATTGT
GCCGTTGCCCAATGTGAATTCGACGATTCTTCGCAAGGTGCTTACCTGGG
CTCACTACCACAAGGACGACCCCCAGCCAACGGAGGATGATGAGAGCAAG
GAGAAGCGCACAGACGACATTATCTCATGGGATGCAGATTTCCTAAAAGT
CGACCAGGGCACACTGTTTGAGCTGATATTGGCAGCGAACTATCTGGACA
TTAAGGGCCTTCTGGAGCTCACCTGCAAGACTGTTGCAAACATGATTAAG
GGAAAGACTCCCGAGGAAATACGCAAGACCTTCAACATTAAGAAGGACTT
TTCGCCCGCCGAGGAGGAGCAGGTGCGCAAGGAGAACGAGTGGTGCGAGG
AGAAGTAAAGCGCGGCATTTCGCGGGACCAACATTAAGTTGAAACAGCTA
GGGGATTCGGGAACGAATTGGATTTGCAGCATTGCAACTTTACTTAGTTG
CTACTTTCATTTACATTTTTTTTTATTTTTAACCCCAGCAGAGACTCGAT
TTAAATTGTGTATAAATGATCTGTTGCTGATTTGATTCGCGGGGTTCATT
TTTTGTCGTAAATATATCTCATATACATACATATGCGAGATTGTAACACT
CTCTTTAACCTATTGGAGTAACACTTGATTTCACTTTAATAAATATAACT
ACCCAACAC

(SEQ ID NO:235)

MPSIKLQSSDEEIFDTDIQIAKCSGTIKTMLEDCGMEDDENAIVPLPNVN
STILRKVLTWAHYHKDDPQPTEDDESKEKRTDDIISWDADFLKVDQGTLF
ELILAANYLDIKGLLELTCKTVANMIKGKTPEEIRKTFNIKKDFSPAEEE
QVRKJENEWCEEK

(SEQ ID NO:236)

TTTCGCCATCTGGTCACTATAGCCGTTTCGTTTTTTACGTGAGTATTGTG
AATTTGGTGTGTTGATTTATATCTCAGTTGGAGCCTGCGTGGAAATAGTG
TCAGTACGTTTAAAGGCATCATCGTAAGGAAAGCCCAAAATGCCCAGCAT
CAAGTTGCAATCTTCGGATGAGGAGATCTTTGACACGGATATCCAGATCG
CCAAGTGCTCCGGCACTATCAAGACCATGCTGGAGGACTGCGGCATGGAG
GACGATGAGAATGCCATTGTGCCGTTGCCCAATGTGAATTCGACGATTCT
TCGCAAGGTGCTTACCTGGGCTCACTACCACAAGGACGACCCCCAGCCAA
CGGAGGATGATGAGAGCAAGGAGAAGCGCACAGACGACATTATCTCATGG
GATGCAGATTTCCTAAAAGTCGACCAGGGCACACTGTTTGAGCTGATATT
GGCAGCGAACTATCTGGACATTAAGGGCCTTCTGGAGCTCACCTGCAAGA
CTGTTGCAAACATGATTAAGGGAAAGACTCCCGAGGAAATACGCAAGACC
TTCAACATTAAGAAGGACTTTTCGCCCGCCGAGGAGGAGCAGGTGCGCAA
GGAGAACGAGTGGTGCGAGGAGAAGTAAAGCGCGGCATTTCGCGGGACCA
ACATTAAGTTGAAACAGCTAGGGGATTCGGGAACGAATTGGATTTGCAGC
ATTGCAACTTTACTTAGTTGCTACTTTCATTTACATTTTTTTTTATTTTT
AACCCCAGCAGAGACTCGATTTAAATTGTGTATAAATGATCTGTTGCTGA
TTTGATTCGCGGGGTTCATTTTTTGTCGTAAATATATCTCATATACATAC
ATATGCGAGATTGTAACACTCTCTTTAACCTATTGGAGTAACACTTGATT
TCACTTTAATAAATATAACTACCCAACAC

(SEQ ID NO:237)

MPSIKLQSSDEEIFDTDIQIAKCSGTIKTMLEDCGMEDDENAIVPLPNVN
STILRKVLTWAHYHKDDPQPTEDDESKEKRTDDIISWDADFLKVDQGTLF
ELILAANYLDIKGLLELTCKTVANMIKGKTPEEIRKTFNIKKDFSPAEEE
QVRKENEWCEEK

(SEQ ID NO:238)

AAACATCGAAAGTGCACAATCGTTTGTTATCTTTGTACGAAAACAACGGT
GATTTCCACACAGGCATAACCTGCAAGAGAAAGCCCAAAATGCCCAGCAT
CAAGTTGCAATCTTCGGATGAGGAGATCTTTGACACGGATATCCAGATCG
CCAAGTGCTCCGGCACTATCAAGACCATGCTGGAGGACTGCGGCATGGAG
GACGATGAGAATGCCATTGTGCCGTTGCCCAATGTGAATTCGACGATTCT
TCGCAAGGTGCTTACCTGGGCTCACTACCACAAGGACGACCCCCAGCCAA
CGGAGGATGATGAGAGCAAGGAGAAGCGCACAGACGACATTATCTCATGG
GATGCAGATTTCCTAAAAGTCGACCAGGGCACACTGTTTGAGCTGATATT
GGCAGCGAACTATCTGGACATTAAGGGCCTTCTGGAGCTCACCTGCAAGA
CTGTTGCAAACATGATTAAGGGAAAGACTCCCGAGGAAATACGCAAGACC
TTCAACATTAAGAAGGACTTTTCGCCCGCCGAGGAGGAGCAGGTGCGCAA
GGAGAACGAGTGGTGCGAGGAGAAGTAAAGCGCGGCATTTCGCGGGACCA
ACATTAAGTTGAAACAGCTAGGGGATTCGGGAACGAATTGGATTTGCAGC
ATTGCAACTTTACTTAGTTGCTACTTTCATTTACATTTTTTTTTATTTTT
AACCCCAGCAGAGACTCGATTTAAATTGTGTATAAATGATCTGTTGCTGA
TTTGATTCGCGGGGTTCATTTTTTGTCGTAAATATATCTCATATACATAC
ATATGCGAGATTGTAACACTCTCTTTAACCTATTGGAGTAACACTTGATT
TCACTTTAATAAATATAACTACCCAACAC

(SEQ ID NO:239)

MPSLKLQSSDEEIFDTDIQIAKCSGTIKTMLEDCGMEDDENAIVPLPNVN
STILRKVLTWAHYHKDDPQPTEDDESKEKRTDDIISWDADFLKVDQGTLF
ELILAANYLDIKGLLELTCKTVANMIKGKTPEEIRKTFNIKKDFSPAEEE
QVRKENEWCEEK

Human homologue of Complete Genome candidate

XP_{—}054159—hypothetical protein

```
(SEQ ID NO:240)
    1 gcctcccagc tctcgtcagc ctcctgctgg ccatctcctt aacaccaaac actatgcctt
  61 caattcagtt gcagagtttt gatggagaga tatttgcagt tgatgtggaa attgccaaac
121 aatctgtgac tatcaagacc acgttggaag atttgggaat ggatgatgaa ggagatgacc
181 cagttcctct accaaatgtg aatgcagcag tattaaaaaa ggtcattcag tggtgcaccc
241 accacaagga tgaccctcct ccccctgaag atgatgagaa caaagaaaag caaacagacg
301 atatccctgt ttgggaccaa gaattcctga aagttgctca aggaacactt tttgaactca
361 ttcgggctgc aaactactta gacatcaaag gtttgcttga tgttacatgc aagactgttg
421 ccaatatgat caaggggaaa actcctgagg agattcgcaa gacattcaat atcaaaaatg
481 actttactga agaggaggaa gcccaggtac gcaaagagaa ccagtggtgt gaagagaagt
541 gaaatgttgt gcctgacact gtaacactgt aaggat
(SEQ ID NO:241)
    1 mpsiqlqsfd geifavdvei akqsvtiktt ledlgmddeg ddpvplpnvn aavlkkviqw
  61 cthhkddppp peddenkekq tddipvwdqe flkvaqgtlf eliraanyld ikglldvtck
121 tvanmikgkt peeirktfni kndfteeeea qvrkenqwce ek
```

Putative function
Cell cycle protein, ubiquitin ligase

### Example 24

### Category 3

Line ID—186

Phenotype—Lethal phase larval stage 3. Small brain, high mitotic index, rod-like overcondensed chromosomes, fewer ana- and telophases.

Annotated Drosophila genome genomic segment containing P element insertion site (and map position)—AE003494 (12C6-7)

P element insertion site—123,540

Annotated Drosophila genome Complete Genome candidate

CG18319—bendless ubiquitin conjugating enzyme

(SEQ ID NO:242)

TTAGTCACAGCAACGCACACACACACTACCAAACGGCTACATTTTTTTTC
GAGTGTGTTCGACATTCATAATTTTTGTGGTGGAGCTGCCTGCAAAATCG
AATTTTATCAGTTTGCCAACGAAGTTATCGGCCATAACTGCAAATAAAGT
TCAGCAATAACTTGGCGCTGTTACGATCTCAACGAGAAGGTCCAGACTCA
ACCCGCGTTTCCAGTTCACCGCGTAAAAGGAACCAGCTAAACGATGTCCA
GCCTGCCACGTCGCATCATCAAGGAGACTCAACGTTTGATGCAGGAGCCA
GTGCCTGGGATCAATGCCATTCCCGATGAGAACAATGCCCGTTACTTCCA
TGTGATCGTGACCGGACCGAACGATTCGCCCTTCGAGGGCGGCGTGTTCA
AGCTGGAGCTGTTCCTACCGGAGGACTATCCAATGTCAGCGCCCAAAGTG
CGCTTCATCACGAAGATCTACCATCCGAACATCGATCGTTTGGGCCGCAT
TTGCCTCGACGTGCTGAAGGACAAGTGGAGTCCAGCCCTGCAGATCCGGA
CCATATTGCTATCCATTCAGGCACTGCTCAGTGCACCCAATCCCGACGAT
CCGCTGGCCAACGATGTGGCTGAGTTGTGGAAGGTCAACGAGGCGGAGGC
CATTCGCAATGCCCGCGAGTGGACCCAGAAATATGCCGTCGAAGACTGAA
CGCCCGAGGTCAGGAGGAAAGTCAGAAAGCGGATCCGTCAGTTGTATCGG
CGTTTTTCCAGAAAGTGGGTGCGTGACATGAACGGGCGGGTGGGTAAATT
GAATACTTTAAAAGCAACCAGAAAAACCTAAAACATACGAAAGAAAACAT
AAAATAAGAAAAAAGTAAGGAAGCAAACATAAAAAAAAACGATTTAAGAA
CACATTTTTTTTTCGAACCTTCTGGGGCGGGATATACATATAAAATATTA
ATATATATATTTTTTTCAACCAATCGATCGGGGCGATCGGCGAAATGGAG
GAGAGATAGCGAAAGCATTCTTTATGTAAGACGTATACATGTATCCGAAA
CAAACTAAAAACGAAAAAAAAAAAAAAAAAAAAAAACAGTAATTGGTTTT
AGTCGTTTCTATTGATTTGTTCGAGGGTTCTGGTGTCTATATACATATAG
CCGTATATAATTCTATGTGTAACTGAAATAACCAACCCATAACCATTAAC
ACATGTAGCATCAGATATGATAAATCAATTGGAAAGGCAAACAAGAAGGG
ATTTTGATTTCCTTTAACTCGTCATTTGAAAACTCGGCTTAAATGTCAAT
TCAAAATAGAGAATTTTGATTGTATCATTTTCAGTGTTTCAGAAAATTTA
AGATGTGATCGTCCAACTTGTAGACTTTACTTTTCTTAACTAAGAGTTCA
CCATTTCGATTGATACTTGAGCTTTGCCTGGGTTGTGTCAGAGTCCCTTT
GATAAACGATAAATAGTTTTTACTCGAAAACAATTTTTTTTAACCAAACA
ATGAAGCCTTTAAGCTATTAGTAATTTTTGAAAAAAAAAAAAATAAAAAA
TATATATATAAAAAATATACAAAAATATGATACATGATCAAAATACAATG
AATGCATACACTATATATTTATACAAAAAAAATACAAAAAGAAAAACAAA
AGTAGTGGCTTGATTGCGTGAAAATTTCAAGTGCAGTTCTCAACAAAAAT
TGTGTACAGTAATTAAATGTTTGTCACCGAAATCACTAAAGGATAATCCA
AAAAACAATAGCAACCGAAAAGCAACCATAAATCAAAGAGTAAGCGAAAA
TAAAAATTCAGTTTTCTTTAATTTTAATTAATTTTTTTCTAAGAAAAATA
AATAAAAACGAAAAATTCAAAT

(SEQ ID NO:243)

MSSLPRRIIKETQRLMQEPVPGINAIPDENNARYFHVIVTGPNDSPFEGG
VFKLELFLPEDYPMSAPKVRFITKIYHPNIDRLGRICLDVLKDKWSPALQ
IRTILLSIQALLSAPNPDDPLANDVAELWKVNEAEAIRNAREWTQKY
AVED

Human homologue of Complete Genome candidate

BAA11675—ubiquitin-conjugating enzyme E2 UbcH-ben

```
(SEQ ID NO:244)
      1 actcgtgcgt gaggegagag gagccggaga cgagaccaga ggccgaactc gggttctgac
    61 aagatggccg ggctgccccg caggatcatc aaggaaaccc agcgtttgct ggcagaacca
  121 gttcctggca tcaaagccga accagatgag agcaacgccc gttattttca tgtggtcatt
  181 gctggccctc aggattcccc ctttgaggga gggactttta aacttgaact attccttcca
  241 gaagaatacc caatggcagc ccctaaagta cgtttcatga ccaaaattta tcatcctaat
  301 gtagacaagt tgggaagaat atgtttagat attttgaaag ataagtggtc cccagcactg
  361 cagatccgca cagttctgct atcgatccag gccttgttaa gtgctcccaa tccagatgat
  421 ccattagcaa atgatgtagc ggagcagtgg aagaccaacg aagcccaagc catagaaaca
  481 gctagagcat ggactaggct atatgccatg aataatattt aaattgatac gatcatcaag
  541 tgtgcatcac ttctcctgtt ctgccaagac ttcctcctct ttgtttgcat ttaatggaca
  601 cagtcttaga aacattacag aataaaaaag cccagacatc ttcagtcctt tggtgattaa
  661 atgcacatta gcaaatctat gtcttgtcct gattcactgt cataaagcat gagcagaggc
  721 tagaagtatc atctggattg ttgtgaaacg tttaaaagca gtggcccctc cctgctttta
  781 ttcatttccc ccatcctggt ttaagtataa agcactgtga atgaaggtag ttgtcaggtt
  841 agctgcaggg gtgtgggtgt ttttatttta ttttatttta ttttattttt gaggggggag
  901 gtagtttaat tttatgggct cctttccccc ttttttggtg atctaattgc attggttaaa
  961 agcagctaac caggtcttta gaatatgctc tagccaagtc taactttatt tagacgctgt
1021 agatggacaa gcttgattgt tggaaccaaa atgggaacat taaacaaaca tcacagccct
1081 cactaataac attgctgtca agtgtagatt ccccccttca aaaaaagctt gtgaccattt
1141 tgtatggctt gtctggaaac ttctgtaaat cttatgtttt agtaaaatat tttttgttat
1201 tct
(SEQ ID NO:245)
      1 maglprriik etqrllaepv pgikaepdes naryfhvvia gpqdspfegg tfklelflpe
    61 eypmaapkvr fmtkiyhpnv dklgricldi lkdkwspalq irtvllsiqa llsapnpddp
  121 landvaeqwk tneaqaieta rawtrlyamn ni
```

Putative function
Ubiquitin conjugating enzyme

### Example 25

### Category 3

LineID—301

Phenotype—semilethal male and female, Low mitotic index, badly defined chromosomes, weak/uneven staining, fewer ana- and telophases

Annotated Drosophila genome genomic segment containing P element insertion site (and map position)—AE003422 (2B7-10)

P element insertion site—96,307

Annotated Drosophila genome Complete Genome candidate

CG14813—deltaCOP, component of cotamer involved in retrograde (golgi to ER) transport

(SEQ ID NO:246)

TCGCAGAACCGAACACGTCAGCTACGGGGATTGATTGTTAAACAACGTTT
CTATCGCCCCGCAAATCCGATCCGTAGCAGCAGTCCATCCTGCGCCGTCC
GCATCCGATCCGCGAAGTATTTTCCAGGGCAAAAACGTCAAACGCAGCAG
CAAAATGGTATTAATTGCTGCGGCTGTCTGCACGAAGAATGGCAAAGTGA
TTCTGTCACGTCAGTTCGTCGAGATGACGAAGGCACGCATCGAGGGACTG
CTGGCTGCCTTTCCCAAGCTGATGACTGCTGGCAAGCAGCACACTTACGT
GGAGACGGACTCCGTGCGCTACGTCTACCAGCCGATGGAGAAACTATATA
TGCTGCTCATCACCACTAAGGCCAGCAACATTCTGGAGGATCTGGAGACC
CTGCGCCTCTTCTCGAAAGTGATTCCCGAGTACAGCCACTCGCTCGACGA
GAAGGAGATTGTGGAGAATGCCTTCAATCTGATCTTCGCATTTGACGAGA
TCGTGGCACTCGGCTACAGGGAGAGCGTCAACTTGGCCCAGATCAAGACC
TTCGTGGAGATGGACTCACATGAGGAGAAGGTCTACCAGGCAGTGCGTCA
GACGCAGGAGCGTGATGCGCGCCAGAAGATGCGCGAGAAGGCCAAGGAAC
TGCAGCGGCAGCGCATGGAGGCCAGCAAACGGGGTGGTCCCTCCCTGGGT
GGCATTGGCAGCCGCAGCGGCGGCTTTAGCGCCGACGGAATTGGCAGTAG
CGGCGTGAGCAGCAGTTCCGGTGCCTCCAGCGCCAACACCGGCATCACCT
CCATCGATGTGGACACCAAATCCAAGGCGGCTGCCAGTAAACCAGCTTCC
CGCAATGCCCTCAAGCTAGGTGGCAAGTCCAAGGACGTCGATAGTTTCGT
GGATCAGCTGAAGAACGAGGGCGAGAAGATTGCCAATCTGGCACCGGCGG
CGCCCGCTGGAGGTTCCAGTGCTGCAGCTAGCGCCAGTGCAGCGGCCAAG
GCAGCTATCGCGTCGGACATTCACAAAGAGAGCGTACATCTGAAGATTGA
GGACAAGCTAGTAGTGCGTCTGGGACGCGATGGTGGCGTGCAGCAGTTCG
AGAACTCGGGCCTCCTGACGTTGCGCATTACGGACGAGGCCTACGGACGC
ATTTTGCTGAAGCTGTCTCCCAACCACACACAGGGCCTGCAGTTGCAGAC
CCACCCCAACGTGGACAAGGAGCTGTTCAAGTCGCGCACTACCATCGGAC
TAAAGAACTTGGGCAAGCCGTTTCCCCTTAACACCGATGTGGGTGTGCTC
AAGTGGCGCTTCGTCTCGCAGGACGAGTCGGCAGTCCCGCTGACCATTAA
CTGCTGGCCATCGGATAATGGAGAGGGTGGATGCGATGTTAACATTGAGT
ATGAACTGGAGGCGCAGCAGCTAGAGCTGCAGGACGTGGCCATTGTCATT
CCCTTGCCAATGAATGTGCAGCCTTCGGTGGCGGAGTACGACGGCACCTA
CAACTACGATTCACGCAAGCATGTGCTCCAGTGGCACATTCCAATAATCG
ATGCCGCCAACAAGTCCGGTTCTATGGAGTTCAGCTGCAGTGCCTCCATT
CCCGGTGACTTCTTCCCCTTGCAGGTGTCCTTCGTCTCGAAAACGCCGTA
TGCGGGCGTCGTGGCCCAGGATGTGGTGCAGGTGGACAGCGAGGCGGCGG
TCAAGTATTCAAGCGAGTCCATTCTGTTCGTGGAAAAGTACGAGATCGTG
TAGGCCGCGCCGCTGGCCACGCCCACCTAAGTAGTACATAAATATACATA
ATTTCCCGGGGTCATCCGATGCGATGCAATTAATTCAACTGCTGCAGCAT
GTTGAGAATTATTTTTCCATGTGCGAACTTTACATATTTATGGCGCAGAC
AGCTTCTCAGAGCGAGTAATTGATTCC

(SEQ ID NO:247)

MVLIAAAVCTKNGKVILSRQFVEMTKARIEGLLAAFPKLMTAGKQHTYVE
TDSVRYVYQPMEKLYMLLITTKASNILEDLETLRLFSKVIPEYSHSLDEK
EIVENAFNLIFAFDEIVALGYRESVNLAQIKTFVEMDSHEEKVYQAVRQT
QERDARQKMREKAKELQRQRMEASKRGGPSLGGIGSRSGGFSADGIGSSG
VSSSSGASSANTGITSIDVDTKSKAAASKPASRNALKLGGKSKDVDSFVD
QLKNEGEKIANLAPAAPAGGSSAAASASAAAKAAIASDIHKESVHLKIED
KLVVRLGRDGGVQQFENSGLLTLRITDEAYGRILLKLSPNHTQGLQLQTH
PNVDKELFKSRTTIGLKNLGKPFPLNTDVGVLKWRFVSQDESAVPLTINC
WPSDNGEGGCDVNIEYELEAQQLELQDVAIVIPLPMNVQPSVAEYDGTYN
YDSRKHVLQWHIPIIDAANKSGSMEFSCSASLPGDFFPLQVSFVSKTPYA
GVVAQDVVQVDSEAAVKYSSESILFVEKYEIV

Human homologue of Complete Genome candidate

CAA57071—archain, possible role in vesicle structure or trafficking

```
(SEQ ID NO:248)
      1 cgggcggttc ctgtcaaggg ggcagcaggt ccagagctgc tggtgctccc gttccccaga
    61 ccctacccct atccccagtg gagccggagt gcggcgcgcc ccaccaccgc cctcaccatg
  121 gtgctgttgg cagcagcggt ctgcacaaaa gcaggaaagg ctattgtttc tcgacagttt
  181 gtggaaatga cccgaactcg gattgagggc ttattagcag cttttccaaa gctcatgaac
  241 actggaaaac aacatacgtt tgttgaaaca gagagtgtaa gatatgtcta ccagcctatg
  301 gagaaactgt atatggtact gatcactacc aaaaacagca acattttaga agatttggag
  361 accctaaggc tcttctcaag agtgatccct gaatattgcc gagccttaga agagaatgaa
  421 atatctgagc actgttttga tttgattttt gcttttgatg aaattgtcgc actgggatac
  481 cgggagaatg ttaacttggc acagatcaga accttcacag aaatggattc tcatgaggag
  541 aaggtgttca gagccgtcag agagactcaa gaacgtgaag ctaaggctga gatgcgtcgt
  601 aaagcaaagg aattacaaca ggcccgaaga gatgcagaga gacagggcaa aaaagcacca
  661 ggatttggcg gatttggcag ctctgcagta tctggaggca gcacagctgc catgatcaca
  721 gagaccatca ttgaaactga taaaccaaaa gtggcacctg caccagccag gccttcaggc
  781 cccagcaagg ctttaaaact tggagccaaa ggaaaggaag tagataactt tgtggacaaa
  841 ttaaaatctg aaggtgaaac catcatgtcc tctagtatgg gcaagcgtac ttctgaagca
  901 accaaaatgc atgctccacc cattaatatg gaaagtgtac atatgaagat tgaagaaaag
  961 ataacattaa cctgtggacg agacggagga ttacagaata tggagttgca tggcatgatc
1021 atgcttagga tctcagatga caagtatggc cgaattcgtc ttcatgtgga aaatgaagat
1081 aagaaagggg tgcagctaca gacccateca aatgtggata aaaaactttt cactgcagag
1141 tctctaattg gcctgaagaa tccagagaag tcatttccag tcaacagtga cgtaggggtg
1201 ctaaagtgga gactacaaac cacagaggaa tcttttattc cactgacaat taattgctgg
1261 ccctcggaga gtggaaatgg ctgtgatgtc aacatagaat atgagctaca agaagataat
1321 ttagaactga atgatgtggt tatcaccatc ccactcccgt ctggtgtcgg cgcgcctgtt
1381 atcggtgaga tcgatgggga gtatcgacat gacagtcgac gaaataccct ggagtggtgc
1441 ctgcctgtga ttgatgccaa aaataagagt ggcagcctgg agtttagcat tgctgggcag
1501 cccaatgact tcttccctgt tcaagtttcc tttgtctcca agaaaaatta ctgtaacata
1561 caggttacca aagtgaccca ggtagatgga aacagccccg tcaggttttc cacagagace
1621 actttcctag tggataagta tgaaatcctg taataccaag aagagggagc tgaaaaggaa
1681 aattttcaga ttaataaaga agacgccaat gatggctgaa gagtttttcc cagatttaca
1741 agccactgga gacccctttt ttctgataca atgcacgatt ctctgcgcgc aaggaccctc
1801 gacteaccec catgtttcag tgtcacagag acattctttg ataaggaaat ggcacaaaca
1861 taaagggaaa ggctgctaat tttctttggc agattgtatt ggccagcagg aaagcaagct
1921 ctccagagaa tgcccccagt taaatacctc ctctaccttt acctaagttg ctcctttatt
1981 tttattttat aataataa
(SEQ ID NO:249)
      1 mvllaaavct kagkaivsrq fvemtrtrie gllaafpklm ntgkqhtfve tesvryvyqp
    61 meklymvlit tknsniledl etlrlfsrvi peycraleen eisehcfdli fafdeivalg
  121 yrenvnlaqi rtftemdshe ekvfravret qereakaemr rkakelqqar rdaerqgkka
  181 pgfggfgssa vsggstaami tetiietdkp kvapaparps gpskalklga kgkevdnfvd
  241 klksegetim sssmgkrtse atkmhappin mesvhmkiee kitltcgrdg glqnmelhgm
  301 imlrisddky grirlhvene dkkgvqlqth pnvdkklfta esliglknpe ksfpvnsdvg
  361 vlkwrlqtte esfipitinc wpsesgngcd vnieyelqed nlelndvvit iplpsgvgap
  421 vigeidgeyr hdsrrntlew clpvidaknk sgslefsiag qpndffpvqv sfvskknycn
  481 iqvtkvtqvd gnspvrfste ttflvdkyei l
```

Putative function
Role in vesicle trafficking

### Example 26

### Category 3

Line ID—148

Phenotype—Lethal phase pupal to pharate adult. Lagging chromosomes and bridges in ana- and telophase

Annotated Drosophila genome genomic segment containing P element insertion site (and map position)—AE003438 (6B-C)

P element insertion site—116,914

Annotated Drosophila genome Complete Genome candidate

CG8655—cdc7 kinase

(SEQ ID NO:250)

ATGCGTTATGACGCCTCCGCCGCTTTCGTGATGCCCTTCATGGCACATGA
CCGATTCCAGGACTTTTACACGCGCATGGATGTGCCCGAGATCCGGCAGT
ATATGCGCAATCTCCTGGTGGCACTGCGTCATGTCCACAAGTTCGATGTC
ATCCATCGCGACGTGAAGCCGAGCAACTTTCTCTACAATCGACGTCGGCG
AGAGTTTCTCCTCGTCGATTTCGGTCTGGCCCAGCATGTGAATCCTCCGG
CTGCGCGATCTTCCGGAAGTGCCGCCGCCATCGCCGCAGCCAACAACAAA
AACAACAACAATAATAACAATAATAATAGCAAACGGCCACGAGAGCGCGA
ATCAAAGGGGGATGTGCAGCAAATTGCGCTGGATGCTGGTTTGGGTGGAG
CAGTGAAGCGTATGCGTTTGCACGAGGAGTCCAACAAGATGCCCCTGAAA
CCGGTCAACGATATTGCGCCAAGCGATGCGCCGGAGCAGTCAGTAGATGG
GTCCAATCACGTCCAGCCACAGCTAGTGCAGCAAGAGCAGCAACAACTGC
AGCCGCAACAGCAGCAGCAACAACAGCAGCAGCAACAACAGTCGCAACAG
CAGCAGCAGCCGCAGCAGCAGTCGCAACAGCAGCACCCACAACGACAGCC
ACAACTGGCGCAGATGGATCAAACAGCATCGACGCCATCTGGCAGCAAGT
ACAATACGAATCGAAATGTCTCGGCAGCAGCGGCTAATAATGCCAAGTGC
GTTTGCTTTGCAAATCCCTCAGTTTGCCTCAACTGTCTGATGAAGAAGGA
GGTGCACGCCTCCAGGGCAGGAACACCTGGCTATCGGCCGCCCGAGGTTC
TGCTCAAGTACCCAGATCAGACCACTGCCGTGGACGTTTGGGCGGCGGGT
GTGATATTCCTTTCGATCATGTCAACGGTGTATCCGTTTTTCAAAGCGCC
CAACGATTTTATCGCGCTGGCCGAGATTGTAACAATATTTGGAGATCAGG
CGATACGGAAGACGGCCTTGGCTCTCGACCGTATGATCACCCTGAGCCAG
AGGTCCAGGCCACTGAATCTGCGAAAGTTGTGCCTGCGCTTTCGCTATCG
TTCCGTTTTTAGTGATGCCAAGCTCCTCAAGAGCTACGAATCTGTGGACG
GAAGCTGCGAAGTGTGCCGGAATTGTGATCAATACTTCTTCAACTGCCTA
TGCGAGGATAGCGATTACTTGACAGAGCCACTGGACGCATACGAATGTTT
TCCACCCAGCGCCTATGACCTACTGGATCGCCTGCTCGAGATTAATCCCC
ATAAACGAATTACCGCCGAAGAGGCACTAAAGCATCCATTCTTTACGGCC
GCCGAGGAGGCCGAGCAGACGGAGCAGGATCAGTTGGCCAATGGAACGCC
GCGCAAGATGCGTCGACAAAGATATCAAAGTCACAGAACGGTGGCCGCCT
CACAGGAGCAGGTCAAGCAGCAGGTTGCCCTTGATCTGCAGCAAGCGGCC
ATTAACAAGCTGTGA

(SEQ ID NO:251)

MRYDASAAFVMPFMAHDRFQDFYTRMDVPEIRQYMRNLLVALRHVHKFDV
IHRDVKPSNFLYNRRRREFLLVDFGLAQHVNPPAARSSGSAAAIAAANNK
NNNNNNNNNSKRPRERESKGDVQQIALDAGLGGAVKRMRLHEESNKMPLK
PVNDIAPSDAPEQSVDGSNHVQPQLVQQEQQQLQPQQQQQQQQQQQQSQQ
QQQPQQQSQQQHPQRQPQLAQMDQTASTPSGSKYNTNRNVSAAAANNAKC
VCFANPSVCLNCLMKKEVHASRAGTPGYRPPEVLLKYPDQTTAVDVWAAG
VIFLSIMSTVYPFFKAPNDFIALAEIVTIFGDQAIRKTALALDRMITLSQ
RSRPLNLRKLCLRFRYRSVFSDAKLLKSYESVDGSCEVCRNCDQYFFNCL
CEDSDYLTEPLDAYECFPPSAYDLLDRLLEINPHKRITAEEALKRPFFTA
AEEAEQTEQDQLANGTPRKMRRQRYQSHRTVAASQEQVKQQVALDLQQAA
INKL

Human homologue of Complete Genome candidate

AAB97512—HsCdc7

```
(SEQ lID NO:252)
      1 atggaggcgt ctttggggat tcagatggat gagccaatgg ctttttctcc ccagcgtgac
    61 cggtttcagg ctgaaggctc tttaaaaaaa aacgagcaga attttaaact tgcaggtgtt
  121 aaaaaagata ttgagaagct ttatgaagct gtaccacagc ttagtaatgt gtttaagatt
  181 gaggacaaaa ttggagaagg cactttcagc tctgtttatt tggccacagc acagttacaa
  241 gtaggacctg aagagaaaat tgctgtaaaa cacttgattc caacaagtca tcctataaga
  301 attgcagctg aacttcagtg cctaacagtg gctggggggc aagataatgt catgggagtt
  361 aaatactgct ttaggaagaa tgatcatgta gttattgcta tgccatatct ggagcatgag
  421 tcgtttttgg acattctgaa ttctctttcc tttcaagaag tacgggaata tatgcttaat
  481 ctgttcaaag ctttgaaacg cattcatcag tttggtattg ttcaccgtga tgttaagccc
  541 agcaattttt tatataatag gcgcctgaaa aagtatgcct tggtagactt tggtttggcc
  601 caaggaaccc atgatacgaa aatagagctt cttaaatttg tccagtctga agctcagcag
  661 gaaaggtgtt cacaaaacaa atcccacata atcacaggaa acaagattcc actgagtggc
  721 ccagtaccta aggagctgga tcagcagtcc accacaaaag cttctgttaa aagaccctac
  781 acaaatgcac aaattcagat taaacaagga aaagacggaa aggagggatc tgtaggcctt
  841 tctgtccagc gctctgtttt tggagaaaga aatttcaata tacacagctc catttcacat
  901 gagagccctg cagtgaaact catgaagcag tcaaagactg tggatgtact gtctagaaag
  961 ttagcaacaa aaaagaaggc tatttctacg aaagttatga atagtgctgt gatgaggaaa
1021 actgccagtt cttgcccagc tagcctgacc tgtgactgct atgcaacaga taaagtttgt
1081 agtatttgcc tttcaaggcg tcagcaggtt gcccctaggg caggtacacc aggattcaga
1141 gcaccagagg tcttgacaaa gtgccccaat caaactacag caattgacat gtggtctgca
1201 ggtgtcatat ttctttcttt gcttagtgga cgatatccat tttataaagc aagtgatgat
1261 ttaactgctt tggcccaaat tatgacaatt aggggatcca gagaaactat ccaagctgct
1321 aaaacttttg ggaaatcaat attatgtagc aaagaagttc cagcacaaga cttgagaaaa
1381 ctctgtgaga gactcagggg tatggattct agcactccca agttaacaag tgatatacag
1441 gggcatgctt ctcatcaacc agctatttca gagaagactg accataaagc ttcttgcctc
1501 gttcaaacac ctccaggaca atactcaggg aattcattta aaaaggggga tagtaatagc
1561 tgtgagcatt gttttgatga gtataatacc aatttagaag gctggaatga ggtacctgat
1621 gaagcttatg acctgcttga taaacttcta gatctaaatc cagcttcaag aataacagca
1681 gaagaagctt tgttgcatcc attttttaaa gatatgagct tgtga
(SEQ ID NO:253)
      1 measlgiqmd epmafspqrd rfqaegslkk neqnfklagv kkdieklyea vpqlsnvfki
    61 edkigegtfs svylataqlq vgpeekiavk hliptshpir iaaelqcltv aggqdnvmgv
  121 kycfrkndhv viampylehe sfldilnsls fqevreymln lfkalkrihq fgivhrdvkp
  181 snflynrrlk kyalvdfgla qgthdtkiel lkfvqseaqq ercsqnkshi itgnkiplsg
  241 pvpkeldqqs ttkasvkrpy tnaqiqikqg kdgkegsvgl svqrsvfger nfnihssish
  301 espavklmkq sktvdvlsrk latkkkaist kvmnsavmrk tasscpaslt cdcyatdkvc
  361 siclsrrqqv apragtpgfr apevltkcpn qttaidmwsa gviflsllsg rypfykasdd
  421 ltalaqimti rgsretiqaa ktfgksilcs kevpaqdlrk lcerlrgmds stpkltsdiq
  481 ghashqpais ektdhkascl vqtppgqysg nsfkkgdsns cehcfdeynt nlegwnevpd
  541 eaydlldkll dinpasrita eeallhpffk dmsl
```

Putative function
Protein kinase which regulates the G1/S phase transition and/or DNA replication in mammalian cells.

### Example 27

### Category 3

LineID—335

Phenotype—Lethal phase, pupal. Uneven chromosome condensation, lagging chromosomes in anaphase

Annotated Drosophila genome genomic segment containing P element insertion site (and map position)—AE003424 (3B1-2)

P element insertion site—286,560

Annotated Drosophila genome Complete Genome candidate

CG2621—shaggy, protein serine/threonine kinase

(SEQ ID NO:254)

ATGTTTACCTTCTACACCAATATAAATAATACACTGATCAACAACAACAA
TAATAATAATAATACTAGTAACAGTAATAATAATAATAACAACGTTATAA
GCCAGCCGATTAAAATACCGCTAACCGAGCGCTTCTCATCGCAAACATCG
ACGGGCTCGGCGGATAGCGGTGTAATTGTTTCCAGTGCATCGCAGCAGCA
ACTGCAGTTGCCACCACCACGCAGTAGCAGTGGATCGCTGAGTCTGCCAC
AAGCGCCACCTGGCGGCAAGTGGCGGCAGAAGCAGCAGCGCCAACAGTTG
CTGCTCAGCCAGGACAGCGGCATCGAAAATGGTGTCACCACTCGTCCATC
GAAAGCCAAGGACAACCAGGGTGCGGGAAAAGCCAGTCACAATGCCACAA
GCTCGAAGGAGAGCGGCGCGCAGTCGAACAGCAGCAGCGAGAGCCTGGGC
AGCAATTGCTCCGAGGCCCAGGAGCAGCAGAGAGTAAGAGCCTCCTCCGC
TCTGGAGCTCAGCAGCGTGGACACTCCCGTGATCGTCGGCGGTGTGGTCA
GTGGAGGCAACAGCATCTTGCGCAGCCGCATTAAGTACAAGAGTACGAAC
AGCACCGGAACCCAGGGATTCGATGTGGAGGATCGCATCGATGAGGTGGA
TATCTGTGATGATGATGATGTCGACTGCGATGATCGCGGATCGGAGATCG
AGGAGGAGGAGGAGGACCAAACCGAACAAGAGGAGGAGGTCGATGAGGTG
GATGCCAAGCCGAAGAACCGACTTTTGCCACCGGATCAGGCGGAACTCAC
AGTGGCGGCGGCCATGGCACGTCGACGCGATGCCAAGAGCCTGGCCACCG
ACGGTCACATATATTTCCCACTGCTCAAGATCAGCGAGGATCCGCACATT
GATTCGAAGCTGATCAATCGCAAGGATGGCCTCCAGGACACCATGTATTA
TTTGGACGAATTCGGCAGTCCAAAGTTGCGAGAGAAGTTCGCCCGCAAGC
AGAAGCAGCTGCTCGCCAAGCAGCAGAAGCAGTTGATGAAACGTGAAAGG
AGGAGCGAGGAGCAGCGCAAGAAGCGAAACACCACCGTGGCATCCAACTT
GGCGGCCAGCGGAGCGGTGGTGGACGACACCAAAGATGATTACAAACAAC
AACCACACTGTGATACTAGCTCTAGGAGCAAAAATAACTCGGTACCCAAT
CCACCCAGCAGCCATCTCCATCAGAACCACAATCATCTCGTTGTGGATGT
GCAAGAGGATGTGGATGATGTGAATGTGGTTGCCACCAGCGACGTGGACA
GTGGTGTCGTCAAGATGCGCCGCCATAGCCACGATAACCACTACGACCGA
ATTCCCCGGAGCAATGCTGCCACCATTACCACCCGCCCTCAAATCGACCA
ACAGTCGTCGCACCACCAGAACACCGAGGATGTGGAGCAAGGAGCTGAGC
CCCAAATCGATGGCGAAGCGGATCTGGATGCGGATGCGGATGCGGACAGC
GATGGGAGTGGCGAGAACGTTAAGACTGCCAAATTGGCCAGAACACAGTC
CTGCAAAAACCAAACAGGTCGCGATGGTTCTAAAATCACAACAGTTGTTG
CAACACCCGGCCAAGGCACCGATCGCGTACAAGAGGTCTCCTATACAGAC
ACAAAGGTCATCGGCAATGGCAGCTTCGGCGTCGTGTTCCAGGCAAAGCT
CTGCGATACCGGCGAACTGGTGGCAATCAAAAAAGTTTTACAAGACAGAC
GATTTAAGAATCGCGAATTGCAAATAATGCGCAAATTGGAGCATTGTAAT
ATTGTGAAGCTTTTGTACTTTTTCTATTCGAGTGGTGAAAAGCGTGATGA
AGTATTTTTGAATTTAGTCCTCGAATATATACCAGAAACCGTATACAAAG
TGGCTCGCCAATATGCCAAAACCAAGCAAACGATACCAATCAACTTTATT
CGGCTCTACATGTATCAACTGTTCAGAAGTTTGGCCTACATCCACTCGCT
GGGCATTTGCCATCGTGATATCAAGCCGCAGAATCTTCTGCTCGATCCGG
AGACGGCTGTGCTGAAGCTCTGTGACTTTGGCAGCGCCAAACAGCTGCTG
CACGGCGAGCCGAATGTATCGTATATCTGCTCCCGGTATTACCGCGCCCC
CGAGCTCATCTTTGGCGCCATCAATTATACAACAAAGATCGATGTCTGGA
GTGCCGGTTGCGTTTTGGCCGAACTGCTGCTGGGCCAGCCCATCTTCCCT
GGCGATTCCGGTGTGGATCAGCTCGTCGAGGTCATCAAGGTCCTGGGCAC
ACCGACAAGAGAACAGATACGCGAAATGAATCCAAACTACACGGAATTCA
AGTTCCCTCAGATTAAGAGTCATCCATGGCAGAAAGTTTTCCGTATACGC
ACTCCTACAGAAGCTATCAACTTGGTGTCCCTGCTGCTCGAGTATACGCC
CAGTGCCAGGATCACACCGCTCAAGGCCTGCGCACATCCGTTCTTCGATG
AGCTACGCATGGAGGGTAATCACACCTTGCCCAACGGTCGCGATATGCCG
CCGCTGTTCAACTTCACAGAGCATGAGCTCTCAATACAGCCCAGCCTAGT
GCCGCAGTTGTTGCCCAAGCATCTGCAGAACGCATCCGGACCTGGCGGCA
ATCGACCCTCGGCCGGCGGAGCAGCCTCCATTGCGGCCAGCGGCTCCACC
AGCGTCTCGTCAACGGGCAGTGGTGCCTCGGTGGAAGGATCCGCCCAGCC
ACAGTCGCAGGGTACAGCAGCAGCTGCGGGATCCGGATCGGGCGGAGCAA
CAGCAGGAACCGGCGGAGCGAGTGCCGGTGGACCCGGATCTGGTAACAAC
AGTAGCAGCGGCGGAGCATCGGGAGCGCCGTCCGCTGTGGCTGCCGGAGG
AGCCAATGCCGCCGTCGCTGGCGGTGCTGGTGGTGGTGGCGGAGCCGGTG
CGGCGACCGCAGCTGCAACAGCAACTGGCGCTATAGGCGCGACTAATGCC
GGCGGCGCCAATGTAACAGATTCATAGGGGAAATAGTAACATACATACAC
ACACTAAATATATATCCAAGCATATATATATAGTAATCATTATATATAAC
ACCTACACCCACAACAACAACAACAGCAATTATATATAATAACCATAAAC
AAGAATGGAGAAAGCCAATCCAGCAATCACAGCAAACTATATACACAACA
ACAACAATTAAATTAATTAATGCAATTGATGAAAGAACAGCAGCAGCAGC
AGCAGCAGCAGCAGCAGCAGCATCAACCGCAATTTCAAAAGAACTCTAGA
AACAGCAAAGGCATAAAATATAACAAAAGAAATATTTTACTTAGGTAAAA
CATTAAATTTATTTTAAATCTAAAATAAACTAATAAGCATTAAATAATAC
ATGATAATGGTAAATAAACACACAATAATTATAATAGTAGAGCGAGCGCT
GATCGATTGTCATTTTATTGCTGCCGC

(SEQ ID NO:255)

MFTFYTNINNTLINNNNNNNNTSNSNNNNNNVISQPIKIPLTERFSSQTS
TGSADSGVIVSSASQQQLQLPPPRSSSGSLSLPQAPPGGKWRQKQQRQQL
LLSQDSGIENGVTTRPSKAKDNQGAGKASHNATSSRESGAQSNSSSESLG
SNCSEAQEQQRVRASSALELSSVDTPVIVGGVVSGGNSILRSRIKYKSTN
STGTQGFDVEDRIDEVDICDDDDVDCDDRGSEIEEEEEDQTEQEEEVDEV
DAKPKNRLLPPDQAELTVAAAMARRRDAKSLATDGHIYFPLLKISEDPHI
DSKLINRKDGLQDTMYYLDEFGSPKLREKFARKQKQLLAKQQKQLMKRER
RSEEQRKKRNTTVASNLAASGAVVDDTKDDYKQQPHCDTSSRSKNNSVPN
PPSSHLHQNHNHLVVDVQEDVDDVNVVATSDVDSGVVKMRRHSHDNHYDR
IPRSNAATITTRPQIDQQSSHHQNTEDVEQGAEPQIDGEADLDADADADS
DGSGENVKTAKLARTQSCKNQTGRDGSKITTVVATPGQGTDRVQEVSYTD
TKVIGNGSFGVVFQAKLCDTGELVAIKKVLQDRRFKNRELQIMRKLEHCN
IVKLLYFFYSSGEKRDEVFLNLVLEYIPETVYKVARQYAKTKQTIPINFI
RLYMYQLFRSLAYIHSLGICHRDIKPQNLLLDPETAVLKLCDFGSAKQLL
HGEPNVSYICSRYYRAPELIFGAINYTTKJDVWSAGCVLAELLLGQPIFP
GDSGVDQLVEVIKVLGTPTREQIREMNPNYTEFKFPQIKSHPWQKVFRIR
TPTEAINLVSLLLEYTPSARITPLKACAHPFFDELRMEGNHTLPNGRDMP
PLFNFTEHELSLQPSLVPQLLPKHLQNASGPGGNRPSAGGAASIAASGST
SVSSTGSGASVEGSAQPQSQGTAAAAGSGSGGATAGTGGASAGGPGSGNN
SSSGGASGAPSAVAAGGANAAVAGGAGGGGGAGAATAAATATGAIGATNA
GGANVTDS

Human homologue of Complete Genome candidate

NP_{—}002084—glycogen synthase kinase 3 beta

| | | |
|---|---|---|
| 1 | ggagaaggaa ggaaaaggtg attcgcgaag agagtgatca tgtcagggcg gcccagaacc | (SEQ ID NO:256) |
| 61 | acctcctttg cggagagctg caagccggtg cagcagcctt cagcttttgg cagcatgaaa | |
| 121 | gttagcagag acaaggacgg cagcaaggtg acaacagtgg tggcaactcc tgggcagggt | |
| 181 | ccagacaggc cacaagaagt cagctataca gacactaaag tgattggaaa tggatcattt | |
| 241 | ggtgtggtat atcaagccaa actttgtgat tcaggagaac tggtcgccat caagaaagta | |
| 301 | ttgcaggaca agagatttaa gaatcgagag ctccagatca tgagaaagct agatcactgt | |
| 361 | aacatagtcc gattgcgtta tttcttctac tccagtggtg agaagaaaga tgaggtctat | |
| 421 | cttaatctgg tgctggacta tgttccggaa acagtataca gagttgccag acactatagt | |
| 481 | cgagccaaac agacgctccc tgtgatttat gtcaagttgt atatgtatca gctgttccga | |
| 541 | agtttagcct atatccattc ctttggaatc tgccatcggg atattaaacc gcagaacctc | |
| 601 | ttgttggatc ctgatactgc tgtattaaaa ctctgtgact ttggaagtgc aaagcagctg | |
| 661 | gtccgaggag aacccaatgt ttcgtatatc tgttctcggt actatagggc accagagttg | |
| 721 | atctttggag ccactgatta tacctctagt atagatgtat ggtctgctgg ctgtgtgttg | |
| 781 | gctgagctgt tactaggaca accaatattt ccaggggata gtggtgtgga tcagttggta | |
| 841 | gaaataatca aggtcctggg aactccaaca agggagcaaa tcagagaaat gaacccaaac | |
| 901 | tacacagaat ttaaattccc tcaaattaag gcacatcctt ggactaaggt cttccgaccc | |
| 961 | cgaactccac cggaggcaat tgcactgtgt agccgtctgc tggagtatac accaactgcc | |
| 1021 | cgactaacac cactggaagc ttgtgcacat tcattttttg atgaattacg ggacccaaat | |
| 1081 | gtcaaacatc caaatgggcg agacacacct gcactcttca acttcaccac tcaagaactg | |
| 1141 | tcaagtaatc cacctctggc taccatcctt attcctcctc atgctcggat tcaagcagct | |
| 1201 | gcttcaaccc ccacaaatgc cacagcagcg tcagatgcta atactggaga ccgtggacag | |
| 1261 | accaataatg ctgcttctgc atcagcttcc aactccacct gaacagtccc gacgagccag | |
| 1321 | ctgcacagga aaaaccacca gttacttgag tgtcactcag caacactggt cacgtttgga | |
| 1381 | aagaatatt | |
| 1 | msgrprttsf aesckpvqqp safgsmkvsr dkdgskvttv vatpgqgpdr pqevsytdtk | (SEQ ID NO:257) |
| 61 | vigngsfgvv yqaklcdsge lvaikkvlqd krfknrelqi mrkldhcniv rlryffyssg | |
| 121 | ekkdevylnl vldyvpetvy rvarhysrak qtlpviyvkl ymyqlfrsla yihsfgichr | |
| 181 | dikpqnllld pdtavlklcd fgsakqlvrg epnvsyicsr yyrapelifg atdytssidv | |
| 241 | wsagcvlael llgqpifpgd sgvdqlveii kvlgtptreq iremnpnyte fkfpqikahp | |
| 301 | wtkvfrprtp peaialcsrl leytptarlt pleacahsff delrdpnvkh pngrdtpalf | |
| 361 | nfttqelssn pplatilipp hariqaaast ptnataasda ntgdrgqtnn aasasasnst | |
| 421 | | |

Putative function
Serine/threonine kinase involved in winglwess signaling pathway

### Example 28

### Category 3

Dlg1 (CG1725) as a candidate gene is detected in a screen of a P-element insertion library covering the X chromosome of Drosophila melanogaster (Peter et al. 2001) as mutant phenotype in fly line 342, as described above.

Mitotic defects are observed in brain squashes: high mitotic index, overcondensed chromosomes, lagging chromosomes and a high proportion of anaphases and telophases compared to normal brains.

Rescue and sequencing of genomic DNA flanking the P-element insertion site indicates that the P-element is inserted into the 5′ region of gene Dlg1 (CG1725).

LineID—342

Phenotype—Lethal phase pupal. Higher mitotic index, colchicine-like overcondensed chromosomes, many ana- and telophases, lagging chromosomes

Annotated Drosophila genome genomic segment containing P element insertion site (and map position)—AE003486 (10B8-10)

P element insertion site—1128 and 3755

Annotated Drosophila genome Complete Genome candidate

CG1725—dig, membrane-associated guanylate kinase homologs, role in cell junctions and proliferation (version 1)

| | |
|---|---|
| CACAAACAACACGCTCGTGCGTGCGATTTAAATATATAGATGTTTCAAAA | (SEQ ID NO:258) |
| GTCAACCTCTCTGTTCGCAATTGTGTGCATTTTCGTTTGTCTAGTGCAAA | |
| AAGTTGGATAATCACAGGCGGCAAATAAAATAGTAACGAATCGAGTTCAA | |
| GAAGAAGAAGAAGAGAAGAGGAAGCAGAGGCAGCAGCGCCGGCATTTGTC | |
| CGTGTGTTGTTGTTGTTGTTTGTGCGCGGCTGTAACTTTAACCCTCGAAC | |
| GCCATAAGATTAAAAAACCAAGTATAACAATAAGTTATAAAATCAATTAA | |
| ACAAAAGCCGCTGCGATATGACAACGAGGAAAAAGAAGCGCGACGGCGGC | |
| GGCAGCGGCGGCGGATTCATCAAGAAAGTTTCGTCACTCTTCAATCTGGA | |
| TTCGGTGAATGGCGATGATAGCTGGTTATACGAGGACATTCAGCTGGAGC | |
| GCGGCAACTCCGGATTGGGCTTTTCCATTGCCGGCGGTACGGATAATCCG | |
| CACATCGGCACCGACACCTCCATCTACATCACCAAGCTCATTTCCGGTGG | |
| AGCAGCTGCCGCCGATGGACGTCTGAGCATCAACGATATCATCGTATCGG | |
| TGAACGATGTGTCCGTGGTGGATGTGCCACATGCCTCCGCCGTGGATGCC | |
| CTCAAGAAGGCGGGCAATGTTGTTAAGCTGCATGTGAAGCGAAAACGTGG | |
| AACGGCCACCACCCCGGCAGCGGGATCGGCGGCAGGAGATGCTCGGGATA | |
| GTGCGGCCAGCGGACCGAAGGTCATCGAAATCGATCTGGTCAAGGGCGGC | |
| AAGGGACTGGGCTTCTCAATTGCCGGCGGCATTGGCAACCAGCACATCCC | |
| CGGCGACAATGGCATCTATGTGACCAAGTTGATGGACGGCGGAGCAGCGC | |
| AGGTGGACGGACGTCTCTCCATCGGAGATAAGCTGATTGCAGTGCGCACC | |
| AACGGGAGCGAGAAGAACCTGGAGAACGTAACGCACGAACTGGCGGTGGC | |
| CACGTTGAAATCGATCACCGACAAGGTGACGCTGATCATTGGAAAGACAC | |
| AGCATCTGACCACCAGTGCGTCCGGCGGCGGAGGAGGAGGCCTTTCATCC | |
| GGACAACAATTGTCGCAGTCCCAATCGCAGTTGGCCACCAGCCAGAGCCA | |
| AAGTCAGGTGCATCAGCAGCAGCATGCGACGCCGATGGTCAATTCGCAGT | |
| CGACAGGTGCGCTAAATAGTATGGGACAGACGGTTGTCGATTCACCATCA | |
| ATACCACAAGCAGCCGCAGCAGTAGCAGCAGCAGCAAATGCATCTGCATC | |
| TGCATCAGTCATTGCAAGCAACAACACAATCAGCAACACCACAGTCACCA | |
| CAGTCACGGCCACGGCCACAGCCAGCAACAGTAGCAGCAAGTTGCCGCCG | |
| TCGCTTGGCGCTAACAGCAGCATTAGCATTAGCAATAGCAATAGCAATAG | |
| CAACAGCAATAATATCAACAACATTAATAGCATCAACAACAACAACAGTA | |
| GCAGCAGCAGCACGACGGCAACTGTTGCAGCAGCAACACCAACAGCAGCA | |
| TCAGCAGCAGCAGCAGCAGCATCATCTCCACCCGCCAACTCCTTCTATAA | |
| CAATGCTTCCATGCCCGCCCTGCCTGTCGAATCCAATCAAACAAACAACC | |
| GATCCCAATCACCCCAGCCGCGCCAGCCCGGGTCGCGATACGCCTCTACA | |
| AATGTCCTAGCCGCCGTTCCACCAGGAACTCCACGCGCTGTCAGCACCGA | |
| GGATATAACCAGAGAACCGCGCACCATCACCATCCAGAAGGGACCGCAGG | |
| GCCTGGGCTTCAATATCGTTGGCGGCGAGGATGGCCAGGGTATCTATGTG | |
| TCCTTCATCCTGGCCGGCGGCCCAGCGGATCTCGGGTCGGAGTTGAAGCG | |
| TGGCGACCAGCTGCTCAGCGTGAACAATGTCAATCTCACGCACGCCACCC | |
| ACGAAGAGGCAGCCCAGGCGCTCAAGACTTCTGGCGGTGTGGTGACCCTG | |
| TTGGCGCAGTACCGCCCAGAGGAGTACAATCGCTTCGAGGCACGCATTCA | |
| AGAGTTGAAACAACAGGCTGCCCTCGGTGCCGGCGGATCGGGAACGCTGC | |
| TGCGCACCACGCAAAAGCGATCGCTGTATGTGCGCGCCCTGTTTGACTAC | |
| GATCCGAATCGGGATGATGGATTGCCCTCGCGAGGATTGCCCTTTAAGCA | |
| CGGCGATATCCTGCACGTGACCAATGCCTCCGACGATGAATGGTGGCAGG | |
| CACGACGAGTTCTCGGCGACAACGAGGACGAGCAAATCGGTATTGTACCA | |
| TCGAAAAGGCGTTGGGAGCGCAAAATGCGAGCTAGGGACCGCAGCGTTAA | |
| GTTCCAGGGACATGCGGCAGCTAATAATAATCTGGATAAGCAATCGACAT | |
| TGGATCGAAAGAAAAAGAATTTCACATTCTCGCGCAAATTTCCGTTTATG | |
| AAGAGTCGCGATGAGAAGAATGAAGATGGCAGCGACCAAGAGCCCAATGG | |
| AGTTGTGAGCAGCACCAGCGAGATTGACATCAATAATGTCAACAACAACC | |
| AGTCAAATGAACCGCAACCTTCCGAGGAGAACGTGTTGTCCTACGAGGCC | |
| GTACAGCGTTTGTCCATCAACTACACGCGCCCGGTGATTATTCTGGGACC | |
| CCTGAAGGATCGCATCAACGATGACCTTATATCAGAGTATCCCGACAAGT | |
| TCGGCTCTTGTGTGCCACACACCACCCGACCCAAGCGAGAGTACGAGGTG | |
| GATGGTAGGGACTACCACTTTGTATCCTCTCGCGAGCAAATGGAACGGGA | |
| TATTCAGAATCATCTGTTCATCGAGGCGGGACAGTATAACGACAATCTGT | |
| ACGGCACATCGGTGGCCAGCGTGCGCGAAGTGGCCGAGAAGGGTAAACAC | |
| TGCATCCTGGACGTGTCCGGGAACGCCATCAAGCGACTCCAAGTTGCCCA | |
| GCTGTATCCCGTCGCCGTGTTCATCAAGCCCAAGTCGGTGGATTCAGTGA | |
| TGGAAATGAATCGTCGCATGACGGAGGAGCAGGCCAAGAAGACTTACGAG | |
| CGGGCGATTAAAATGGAGCAAGAATTCGGCGAATACTTTACGGGCGTTGT | |
| CCAAGGCGATACCATCGAGGAGATTTACAGCAAAGTGAAATCGATGATTT | |
| GGTCCCAGTCGGGACCAACCATTTGGGTACCTTCCAAGGAATCTCTATGA | |
| CCAACAGCCACCACAACTTGGACACTGCCGCCTCGAGTTCGATGTCGACC | |
| AGTCTCGAGAACAACAATAGGAGCAACAGCAGCAGCAACAAATCAGCAGC | |
| CGCAGCAGAAGACGCCGCACTGATGATGCATCACAGTAACAACAGATACT | |
| AATACAACTACAACAACAACAAGAACAACAACAACAACAGCAACCACAGC | |
| AGCAGCCACAGCGACAACAACAAAAACAACAACACTGACAACGACAGGAA | |
| ACGG | |
| MTTRKKKRDGGGSGGGFIKKVSSLFNLDSVNGDDSWLYEDIQLERGNSGLGFSIAGGTD | (SEQ ID NO:259) |
| NPHIGTDTSIYITKLISGGAAAADGRLSINDIIVSVNDVSVVDVPHASAVDALKKAGNVV | |
| KLHVKRKRGTATTPAAGSAAGDARDSAASGPKVIEIDLVKGGKGLGFSIAGGIGNQHLP | |
| GDNGIYVTKLTDGGRAQVDGRLSIGDKLIAVRTNGSEKNLENVTHELAVATLKSITDKV | |
| TLIIGKTQHLTTSASGGGGGGLSSGQQLSQSQSQLATSQSQSQVHQQQHATPMVNSQST | |
| GALNSMGQTVVDSPSIPQAAAAVAAAANASASASVIASNNTISNTTVTTVTATATASND | |
| SSKLPPSLGANSSISISNSNSNSNSNNINNINSINNNNSSSSSTTATVAAATPTAASAAAAA | |
| ASSPPANSFYNNASMPALPVESNQTNNRSQSPQPRQPGSRYASTNVLAAVPPGTPRAVS | |
| TEDITREPRTITIQKGPQGLGFNIVGGEDGQGIYVSFILAGGPADLGSELKRGDQLLSVNN | |
| VNLTHATHEEAAQALKTSGGVVTLLAQYRPEEYNRFEARIQELKQQAALGAGGSGTLL | |
| RTTQKRSLYVRALFDYDPNRDDGLPSRGLPFKHGDILHVTNASDDEWWQARRVLGDN | |
| EDEQIGIVPSKRRWERKMRARDRSVKFQGHAAANNNLDKQSTLDRKKKNFTFSRKFPF | |
| MKSRDEKNEDGSDQEPNGVVSSTSEIDINNVNNNQSNEPQPSEENVLSYEAVQRLSINYT | |
| RPVIILGPLKDRINDDLISEYPDKFGSCVPHTTRPKREYEVDGRDYHFVSSREQMERDIQN | |
| HLFIEAGQYNDNLYGTSVASVREVAEKGKHCILDVSGNAIKRLQVAQLYPVAVFIKPKS | |
| VDSVMEMNRRMTEEQAKKTYERAIKMEQEFGEYFTGVVQGDTIEEIYSKVKSMIWSQS | |
| GPTIWVPSKESL | |

CG1725—dlg, membrane-associated guanylate kinase homologs, role in cell junctions and proliferation, genbank accession number M73529 (version 2)

| | | | | | | | |
|---|---|---|---|---|---|---|---|
| 1 | cccccccccc | cccagttggg | tgtgttgttt | tcgtcgcgtt | cggttgctcg | ctttattttt | (SEQ ID NO:260) |
| 61 | ttgtttgttt | attttgtttt | gtgcaatgga | aatgtgaaca | caaatgtttc | aaaagtcaac | |
| 121 | ctctctgttc | gcaattgtgt | gcattttcgt | ttgtctagtg | caaaaagttg | gataacacag | |
| 181 | gcggcaaata | aaatagtaac | gaatcgagtt | caagaagaag | aagaagagaa | gaggaagcag | |
| 241 | aggcagcagc | gccggcattt | gtccgtgtgt | tgttgttgtt | gtttgtgcgc | ggctgtaact | |
| 301 | ttaaccctcg | aacgccataa | gattaaaaaa | ccaactataa | caataagtta | taaaatcaat | |
| 361 | taaacaaaag | ccgctgcgat | atgacaacga | ggaaaaagaa | gcgcgacggc | ggcggcagcg | |
| 421 | gcggcggatt | catcaagaaa | gtttcgtcac | tcttcaatct | ggattcggtg | aatggcgatg | |
| 481 | atagctggtt | atacgaggac | attcagctgg | agcgcggcaa | ctccggattg | ggcttttcca | |
| 541 | ttgccggcgg | tacggataat | ccgcacatcg | gcaccgacac | ctccatctac | atcaccaagc | |
| 601 | tcatttccgg | tggagcagct | gccgccgatg | gacgtctgag | catcaacgat | atcatcgtat | |
| 661 | cggtgaacga | tgtgtccgtg | gtggatgtgc | cacatgcctc | cgccgtggat | gccctcaaga | |
| 721 | aggcgggcaa | tgttgttaag | ctgcatgtga | agcgaaaacg | tggaacggcc | accaccccgg | |
| 781 | cagcgggatc | ggcggcagga | gatgctcggg | atagtgcggc | cagcggaccg | aaggtcatcg | |
| 841 | aaatcgatct | ggtcaagggc | ggcaagggac | tgggcttctc | aattgccggc | ggcattggca | |
| 901 | accagcacat | ccccggcgac | aatggcatct | atgtgaccaa | gttgacggac | ggcggacgag | |
| 961 | cgcaggtgga | cggacgtctc | tccatcggag | ataagctgat | tgcagtgcgc | accaacggga | |
| 1021 | gcgagaagaa | cctggagaac | gtaacgcacg | aactggcggt | ggccacgttg | aaatcgatca | |
| 1081 | ccgacaaggt | gacgctgatc | attggaaaga | cacagcatct | gaccaccagt | gcgtccggcg | |
| 1141 | gcggaggagg | aggcctttca | tccggacaac | aattgtcgca | gtcccaatcg | cagttggcca | |
| 1201 | ccagccagag | ccaaagtcag | gtgcatcagc | agcagcatgc | gacgccgatg | gtcaattcgc | |
| 1261 | agtcgacagg | tgcgctaaat | agtatgggac | agacggttgt | cgattcacca | tcaataccac | |
| 1321 | aagcagccgc | agcagtagca | gcagcagcaa | atgcatctgc | atctgcatca | gtcattgcaa | |
| 1381 | gcaacaacac | aatcagcaac | accacagtca | ccacagtcac | ggccacggcc | acagccagca | |
| 1442 | acgatagcag | caagttgccg | ccgtcgcttg | gcgctaacag | cagcattagc | attagcaata | |
| 1501 | gcaatagcaa | tagcaacagc | aataatatca | acaacattaa | tagcatcaac | aacaacaaca | |
| 1561 | gtagcagcag | cagcacgacg | gcaactgttg | cagcagcaac | accaacagca | gcatcagcag | |
| 1621 | cagcagcagc | agcatcatct | ccacccgcca | actccttcta | taacaatgct | tccatgcccg | |
| 1681 | ccctgcctgt | cgaatccaat | caaacaaaca | accgatccca | atcaccccag | ccgcgccagc | |
| 1741 | ccgggtcgcg | atacgcctct | acaaatgtcc | tagccgccgt | tccaccagga | actccacgcg | |
| 1801 | ctgtcagcac | cgaggatata | accagagaac | cacgcaccat | caccatccag | aagggaccgc | |
| 1861 | agggcctggg | cttcaatatc | gttggcggcg | aggatggcca | gggtatctat | gtgtccttca | |
| 1921 | tcctggccgg | cggcccagcg | gatctcgggt | cggagttgaa | gcgtggcgac | cagctgctca | |
| 1981 | gcgtgaacaa | tgtcaatctc | acgcacgcca | cccacgaaga | ggcagcccag | gcgctcaaga | |
| 2041 | cttctggcgg | tgtggtgacc | ctgttggcgc | agtaccgccc | agaggagtac | aatcgcttcg | |
| 2101 | aggcacgcat | tcaagagttg | aaacaacagg | ctgccctcgg | tgccggcgga | tcgggaacgc | |
| 2161 | tgctgcgcac | cacgcaaaag | cgatcgctgt | atgtgcgcgc | cctgtttgac | tacgatccga | |
| 2221 | atcgggatga | tggattgccc | tcgcgaggat | tgccctttaa | gcacggcgat | atcctgcacg | |
| 2281 | tgaccaatgc | ctccgacgat | gaatggtggc | aggcacgacg | agttctcggc | gacaacgagg | |
| 2341 | acgagcaaat | cggtattgta | ccatcgaaaa | ggcgttggga | gcgcaaaatg | cgagctaggg | |
| 2401 | accgcagcgt | taagttccag | ggacatgcgg | cagctaataa | taatctggat | aagcaatcga | |
| 2461 | cattggatcg | aaagaaaaag | aatttcacat | tctcgcgcaa | atttccgttt | atgaagagtc | |
| 2521 | gcgatgagaa | gaatgaagat | ggcagcgacc | aagagcccaa | tggagttgtg | agcagcacca | |
| 2581 | gcgagattga | catcaataat | gtcaacaaca | accagtcaaa | tgaaccgcaa | ccttccgagg | |
| 2641 | agaacgtgtt | gtcctacgag | gccgtacagc | gtttgtccat | caactacacg | cgcccggtga | |
| 2701 | ttattctggg | acccctgaag | gatcgcatca | acgatgacct | tatatcagag | tatcccgaca | |
| 2761 | agttcggctc | ctgtgtgcca | cacaccaccc | gacccaagcg | agagtacgag | gtggatggta | |
| 2821 | gggactacca | ctttgtatcc | tctcgcgagc | aaatggaacg | ggatattcag | aatcatctgt | |
| 2881 | tcatcgaggc | gggacagtat | aacgacaatc | tgtacggcac | atcggtggcc | agcgtgcgcg | |
| 2941 | aagtggccga | gaagggtaaa | cactgcatcc | tggacgtgtc | cgggaacgcc | atcaagcgac | |
| 3001 | tccaagttgc | ccagctgtat | cccgtcgccg | tgttcatcaa | gcccaagtcg | gtggattcag | |
| 3061 | tgatggaaat | gaatcgtcgc | atgacggagg | agcaggccaa | gaagacttac | gagcgggcga | |
| 3121 | ttaaaatgga | gcaagaattc | ggcgaatact | ttacgggcgt | tgtccagggc | gataccatcg | |
| 3181 | aggagatcta | cagcaaagtg | aaatcgatga | tttggtccca | gtcgggacca | accatttggg | |
| 3241 | taccttccaa | ggaatctcta | tga | | | | |

| | |
|---|---|
| MTTRKKKRDGGGSGGGFIKKVSSLFNLDSVNGDDSWLYEDIQLE | (SEQ ID NO:261) |
| RGNSGLGFSIAGGTDNPHIGTDTSIYITKLISGGAAAADGRLSINDIIVSVNDVSVVD | |
| VPHASAVDALKKAGNVVKLHVKRKRGTATTPAAGSAAGDARDSAASGPKVIEIDLVKG | |
| GKGLGFSIAGGIGNQHIPGDNGIYVTKLTDGGRAQVDGRLSIGDKLIAVRTNGSEKNL | |
| ENVTHELAVATLKSITDKVTLIIGKTQHLTTSASGGGGGGLSSGQQLSQSQSQLATSQ | |
| SQSQVHQQQHATPMVNSQSTGALNSMGQTVVDSPSIPQAAAAVAAAANASASASVIAS | |
| NNTISNTTVTTVTATATASNDSSKLPPSLGANSSISISNSNSNSNSNNINNINSINNN | |
| NSSSSSTTATVAAATPTAASAAAAAASSPPANSFYNNASMPALPVESNQTNNRSQSPQ | |
| PRQPGSRYASTNVLAAVPPGTPRAVSTEDITREPRTITIQKGPQGLGFNIVGGEDGQG | |
| IYVSFILAGGPADLGSELKRGDQLLSVNNVNLTHATHEEAAQALKTSGGVVTLLAQYR | |
| PEEYNRFEARIQELKQQAALGAGGSGTLLRTITQKRSLYVRALFDYDPNRDDGLPSRGL | |
| PFKHGDILHVTNASDDEWWQARRVLGDNEDEQIGIVPSKRRWERKMRARDRSVKFQGH | |
| AAANNNLDKQSTLDRKKKNFTFSRKFPFMKSRDEKNEDGSDQEPNGVVSSTSEIDINN | |
| VNNNQSNEPQPSEENVLSYEAVQRLSINYTRPVIILGPLKDRINDDLISEYPDKFGSC | |
| VPHTTRPKREYEVDGRDYHFVSSREQMERDIQNHLFIEAGQYNDNLYGTSVASVREVA | |
| EKGKHCILDVSGNAIKRLQVAQLYPVAVFIKIPKSVDSVMEMNRRMTEEQAKKTYERAI | |
| KMEQEFGEYFTGVVQGDTIEEIYSKVKSMIWSQSGPTIWVPSKESL | |

Human homologue of Complete Genome candidate

XP_{—}012060—discs, large (Drosophila) homolog 2, channel-associated protein of synapses-110′ (version 1)

| | | |
|---|---|---|
| 1 | gggaattctg gcctgggatt cagtattgct ggggggacag ataatcccca cattggagat | (SEQ ID NO:262) |
| 61 | gaccctggca tatttattac gaagattata ccaggaggtg ctgcagcaga ggatggcaga | |
| 121 | ctcagggtca atgattgtat cttgcgggtg aatgaggttg atgtgtcaga ggtttcccac | |
| 181 | agtaaagcgg tggaagccct gaaggaagca gggtctatcg ttcggctgta tgtgcgtaga | |
| 241 | agacgaccta ttttggagac cgttgtggaa atcaaactgt tcaaaggccc taaaggttta | |
| 301 | ggcttcagta ttgcaggagg tgtggggaac caacacattc ctggagacaa cagcatttat | |
| 361 | gtaactaaaa ttatagatgg aggagctgca caaaaagatg gaaggttgca agtaggagat | |
| 421 | agactactaa tggtaaacaa ctacagttta gaagaagtaa cacacgaaga ggcagtagca | |
| 481 | atattaaaga acacatcaga ggtagtttat ttaaaagttg gcaaacccac taccatttat | |
| 541 | atgactgatc cttatggtcc acctgatatt actcactctt attctccacc aatggaaaac | |
| 601 | catctactct ctggcaacaa tggcacttta gaatataaaa cctccctgcc acccatctct | |
| 661 | ccaggaaggt actcaccaat tccaaagcac atgcttgttg acgacgacta caccaggcct | |
| 721 | ccggaacctg tttacagcac tgtgaacaaa ctatgtgata agcctgcttc tcccaggcac | |
| 781 | tattcccctg ttgagtgtga caaaagcttc ctcctctcag ctccctattc ccactaccac | |
| 841 | ctaggcctgc tacctgactc tgagatgacc agtcattccc aacatagcac cgcaactcgt | |
| 901 | cagccttcaa tgactctcca acgggccgtc tccctggaag gagagcctcg caaggtagtc | |
| 961 | ctgcacaaag gctccactgg cctgggcttc aacattgtcg gtggggaaga tggagaaggt | |
| 1021 | atttttgtgt ccttcattct ggctggtgga ccagcagacc taagtgggga gctccagaga | |
| 1081 | ggagaccaga tcctatcggt gaatggcatt gacctccgtg gtgcatccca cgagcaggca | |
| 1141 | gctgctgcac taaagggggc tggacagaca gtgacgatta tagcacaata tcaacctgaa | |
| 1201 | gattacgctc gatttgaggc caaaatccat gacctacgag agcagatgat gaaccacagc | |
| 1261 | atgagctccg ggtccggatc cctgcgaacc aatcagaaac gctccctcta cgtcagagcc | |
| 1321 | atgttcgact acgacaagag caaggacagt gggctgccaa gtcaaggact tagttttaaa | |
| 1381 | tatggagata ttctccacgt tatcaatgcc tctgatgatg agtggtggca agccaggaga | |
| 1441 | gtcatgctgg agggagacag tgaggagatg ggggtcatcc ccagcaaaag gagggtggaa | |
| 1501 | agaaaggaac gtgcccgatt gaagacagtg aagtttaatg ccaaacctgg agtgattgat | |
| 1561 | tcgaaagggt cattcaatga caagcgtaaa aagagcttca tcttttcacg aaaattccca | |
| 1621 | ttctacaaga acaaggagca gagtgagcag gaaaccagtg atcctgaacg tggacaagaa | |
| 1681 | gacctcattc tttcctatga gcctgttaca aggcaggaaa taaactacac ccggccggtg | |
| 1741 | attatcctgg ggcccatgaa ggatcggatc aatgacgact tgatatctga attccctgat | |
| 1801 | aaatttggct cctgtgtgcc tcatactacg aggccaaagc gagactacga ggtggatggc | |
| 1861 | agagactatc actttgtcat ttccagagaa caaatggaga aagatatcca agagcacaag | |
| 1921 | tttatagaag ccggccagta caatgacaat ttatatggaa ccagtgtgca gtctgtgaga | |
| 1981 | tttgtagcag aaagaggcaa acactgtata cttgatgtat caggaaatgc tatcaagcgg | |
| 2041 | ttacaagttg cccagctcta tcccattgcc atcttcataa aacccaggtc tctggaacct | |
| 2101 | cttatggaga tgaataagcg tctaacagag gaacaagcca agaaaaccta tgatcgagca | |
| 2161 | attaagctag aacaagaatt tggagaatat tttacagcta ttgtccaagg agatacttta | |
| 2221 | gaagatatat ataaccaatg caagcttgtt attgaagagc aatctgggcc tttcatctgg | |
| 2281 | attccctcaa aggaaaagtt ataaattagc tactgcgcct ctgacaacga cagaagagca | |
| 2341 | tttagaagaa caaaatatat ataacatact acttggaggc ttttatgttt ttgttgcatt | |
| 2401 | tatgtttttg cagtcaatgt gaattcttac gaatgtacaa cacaaactgt atgaagccat | |
| 2461 | gaaggaaaca gaggggccaa agggtg | |
| 1 | mvnnysleev theeavailk ntsevvylkv gkpttiymtd pygppdiths ysppmenhll | (SEQ ID NO:263) |
| 61 | sgnngtleyk tslppispgr yspipkhmlv dddytrppep vystvnklcd kpasprhysp | |
| 121 | vecdksflls apyshyhlgl lpdsemtshs qhstatrqps mtlqravsle geprkvvlhk | |
| 181 | gstglgfniv ggedgegifv sfilaggpad lsgelqrgdq ilsvngidlr gasheqaaaa | |
| 241 | lkgagqtvti iaqyqpedya rfeakihdlr eqmmnhsmss gsgslrtnqk rslyvramfd | |
| 301 | ydkskdsglp sqglsfkygd ilhvinasdd ewwqarrvml egdseemgvi pskrrverke | |
| 361 | rarlktvkfn akpgvidskg sfhdkrkksf ifsrkfpfyk nkeqseqets dpergqedli | |
| 421 | lsyepvtrqe inytrpviil gpmkdrindd lisefpdkfg scvphttrpk rdyevdgrdy | |
| 481 | hfvisreqme kdiqehkfie agqyndnlyg tsvqsvrfva ergkhcildv sgnaikrlqv | |
| 541 | aqlypiaifi kprsleplme mnkrlteeqa kktydraikl eqefgeyfta ivqgdtledi | |
| 601 | ynqcklviee qsgpfiwips kekl | |

DLG2: discs, large homolog 2, chapsyn-110 channel-associated protein of synapses-110′ genbank accession number U32376 (version 2)

| | | | | | | | |
|---|---|---|---|---|---|---|---|
| 1 | aaaagcaact | gaggtcttaa | ctttcagacg | ctgaattctc | atctaattga | aattactggg | (SEQ ID NO:264) |
| 61 | cataatgcta | tatatagcca | atgaagagat | tttgagctct | cactcagtgc | cttcaagaca | |
| 121 | tgtcgttttg | tagtcagaga | aaacagagat | caatgcattt | tcaaactgac | agagggaacg | |
| 181 | gatgctcttt | agtagcacat | gcccaggatc | gtgtgtgtgg | ggcttgcgct | gtgctgagaa | |
| 241 | gctgaatacc | ggtccatatg | ctccttattt | actgcaatgt | tctttgcatg | ttactgtgca | |
| 301 | ctccggacta | acgtgaagaa | gtatcgatat | caagatgagg | acgctccaca | tgatcattcc | |
| 361 | ttacctcgac | taacccacga | agtaagaggc | ccagaactcg | tgcatgtatc | agaaaagaac | |
| 421 | ctctctcaaa | tagaaaatgt | ccatggatat | gtcctgcagt | ctcatatttc | tcctctgaag | |
| 481 | gccagtcctg | ctcctataat | tgtcaacaca | gatactttgg | acacaattcc | ttatgtcaat | |
| 541 | gggacagaaa | ttgaatatga | atttgaagaa | attacactgg | agagggggaa | ttctggcctg | |
| 601 | ggattcagta | ttgctggggg | gacagataat | ccccacattg | gagatgaccc | tggcatattt | |
| 661 | attacgaaga | ttataccagg | aggtgctgca | gcagaggatg | gcagactcag | ggtcaatgat | |
| 721 | tgtatcttgc | gggtgaatga | ggttgatgtg | tcagaggttt | cccacagtaa | agcggtggaa | |
| 781 | gccctgaagg | aagcagggtc | tatcgctcgg | ctgtatgtgc | gtagaagacg | acctattttg | |
| 841 | gagaccgttg | tggaaatcaa | actgttcaaa | ggccctaaag | gtttaggctt | cagtattgca | |
| 901 | ggaggtgtgg | ggaaccaaca | cattcctgga | gacaacagca | tttatgtaac | taaaattata | |
| 961 | gatggaggag | ctgcacaaaa | agatggaagg | ttgcaagtag | gagatagact | actaatggta | |
| 1021 | aacaactaca | gtttagaaga | agtaacacac | gaagaggcag | tagcaatatt | aaagaacaca | |
| 1081 | tcagaggtag | tttatttaaa | agttggcaac | cccactacca | tttatatgac | tgatccttat | |
| 1141 | ggtccacctg | atattactca | ctcttattct | ccaccaatgg | aaaaccatct | actctctggc | |
| 1201 | aacaatggca | ctttagaata | taaaacctcc | ctgccaccca | tctctccagg | gaggtactca | |
| 1261 | ccaattccaa | agcacatgct | tgttgacgac | gactacacca | ggcctccgga | acctgtttac | |
| 1321 | agcactgtga | acaaactatg | tgataagcct | gcttctccca | ggcactattc | ccctgttgag | |
| 1381 | tgtgacaaaa | gcttcctcct | ctcagctccc | tattcccact | accacctagg | cctgctacct | |
| 1441 | gactctgaga | tgaccagtca | ttcccaacat | agcaccgcaa | ctcgtcagcc | ttcaatgact | |
| 1501 | ctccaacggg | ccgtctccct | ggaaggagag | cctcgcaagg | tagtcctgca | caaaggctcc | |
| 1561 | actggcctgg | gcttcaacat | tgtcggtggg | gaagatggag | aaggtatttt | tgtgtccttc | |
| 1621 | attctggctg | gtggaccagc | agacctaagt | ggggagctcc | agagaggaga | ccagatccta | |
| 1681 | tcggtgaatg | gcattgacct | ccgtggtgca | tcccacgagc | aggcagctgc | tgcactaaag | |
| 1741 | ggggctggac | agacagtgac | gattatagca | caatatcaac | ctgaagatta | cgctcgattt | |
| 1801 | gaggccaaaa | tccatgacct | acgagagcag | atgatgaacc | acagcatgag | ctccgggtcc | |
| 1861 | ggatccctgc | gaaccaatca | gaaacgctcc | ctctacgtca | gagccatgtt | cgactacgac | |
| 1921 | aagagcaagg | acagtgggct | gccaagtcaa | ggacttagtt | ttaaatatgg | agatattctc | |
| 1981 | cacgttatca | atgcctctga | tgatgagtgg | tggcaagcca | ggagagtcat | gctggaggga | |
| 2041 | gacagtgagg | agatgggggt | catccccagc | aaaaggaggg | tggaaagaaa | ggaacgtgcc | |
| 2101 | cgattgaaga | cagtgaagtt | taatgccaaa | cctggagtga | ttgattcgaa | agggtcattc | |
| 2161 | aatgacaagc | gtaaaaagag | cttcatcttt | tcacgaaaat | tcccattcta | caagaacaag | |
| 2221 | gagcagagtg | agcaggaaac | cagtgatcct | gaacgtggac | aagaagacct | cattctttcc | |
| 2281 | tatgagcctg | ttacaaggca | ggaaataaac | tacacccggc | cggtgattat | cctggggccc | |
| 2341 | atgaaggatc | ggatcaatga | cgacttgata | tctgaattcc | ctgataaatt | tggctcctgt | |
| 2401 | gtgcctcata | ctacgaggcc | aaagcgagac | tacgaggtgg | atggcagaga | ctatcacttt | |
| 2461 | gtcatttcca | gagaacaaat | ggagaaagat | atccaagagc | acaagtttat | agaagccggc | |
| 2521 | cagtacaatg | acaatttata | tggaaccagt | gtgcagtctg | tgagatttgt | agcagaaaga | |
| 2581 | ggcaaacact | gtatacttga | tgtatcagga | aatgctatca | agcggttaca | agttgcccag | |
| 2641 | ctctatccca | ttgccatctt | cataaaaccc | aggtctctgg | aatctcttat | ggagatgaat | |
| 2701 | aagcgtctaa | cagaggaaca | agccaagaaa | acctatgatc | gagcaattaa | gctagaacaa | |
| 2761 | gaatttggag | aatattttac | agctattgtc | caaggagata | ctttagaaga | tatatataac | |
| 2821 | caatgcaagc | ttgttattga | agagcaatct | gggcctttca | tctggattcc | ctcaaaggaa | |
| 2881 | aagttataaa | ttagctactg | cgcctctgac | aacgacagaa | gagcatttag | aagaacaaaa | |
| 2941 | tatatataac | atactacttg | gaggctttta | tgtttttgtt | gcatttatgt | ttttgcagtc | |
| 3001 | aatgtgaatt | cttacgaatg | tacaacacaa | actgtatgaa | gccatgaagg | aaacagaggg | |
| 3061 | gccaaagggt | g | | | | | |

| | |
|---|---|
| FFACYCALRTNVKKYRYQDEDAPHDHSLPRLTHEVRGPELVHV | (SEQ ID NO:265) |
| EKNLSQIENVHGYVLQSHISPLKASPAPIIVNTDTLDTIPYVNGTEIEYEFEEITLE | |
| GNSGLGFSIAGGTDNPHIGDDPGIFITKIIPGGAAAEDGRLRVNDCILRVNEVDVSE | |
| SHSKAVEALKEAGSIARLYVRRRRPILETVVEIKLFKGPKGLGFSIAGGVGNQHIPG | |
| NSIYVTKIIDGGAAQKDGRLQVGDRLLMVNNYSLEEVTHEEAVAILKNTSEVVYLKV | |
| NPTTIYMTDPYGPPDITHSYSPPMENHLLSGNNGTLEYKTSLPPISPGRYSPIPKHM | |
| VDDDYTRPPEPVYSTVNKLCDKPASPRHYSPVECDKSFLLSAPYSHYHLGLLPDSEM | |
| SHSQHSTATRQPSMTLQRAVSLEGEPRKVVLHKGSTGLGFNIVGGEDGEGIFVSFIL | |
| GGPADLSGELQRGDQILSVNGIDLRGASHEQAAAALKGAGQTVTIIAQYQPEDYARF | |
| AKIHDLREQMMNHSMSSGSGSLRTNQKRSLYVRAMFDYDKSKDSGLPSQGLSFKYGD | |
| LHVINASDDEWWQARRVMLEGDSEEMGVIPSKRRVERKERARLKTVKFNAKPGVIDS | |
| GSFNDKRKKSFIFSRKFPFYKNKEQSEQETSDPERGQEDLILSYEPVTRQEINYTRP | |
| IILGPMKDRINDDLISEFPDKFGSCVPHTTRPKRDYEVDGRDYHFVISREQMEKDIQ | |
| HKFIEAGQYNDNLYGTSVQSVRFVAERGKHCILDVSGNAIKRLQVAQLYPIAIFIKP | |
| SLESLMEMNKRLTEEQAKKTYDRAIKLEQEFGEYFTAIVQGDTLEDIYNQCKLVIEE | |
| SGPFIWIPSKEKL | |

SLESLMEMNKRLREEQAKKTYCRAIKLEQEFGEYETAIVQGDTLEDIYNQCKLVIEE SGPFIWIPSKEKL

DLG1: discs, large (Drosophila) homolog 1, genbank accession number U13896

| | | | | | | | |
|---|---|---|---|---|---|---|---|
| 1 | gttggaaacg | gcactgctga | gtgaggttga | ggggtgtctc | ggtatgtgcg | ccttggatct | (SEQ ID NO:266) |
| 61 | ggtgtaggcg | aggtcacgcc | tctcttcaga | cagcccgagc | cttcccggcc | tggcgcgttt | |
| 121 | agttcggaac | tgcgggacgc | cggtgggcta | gggcaaggtg | tgtgccctct | tcctgattct | |
| 181 | ggagaaaaat | gccggtccgg | aagcaagata | cccagagagc | attgcacctt | ttggaggaat | |
| 241 | atcgttcaaa | actaagccaa | actgaagaca | gacagctcag | aagttccata | gaacgggtta | |
| 301 | ttaacatatt | tcagagcaac | ctctttcagg | ctttaataga | tattcaagaa | ttttatgaag | |
| 361 | tgaccttact | ggataatcca | aaatgtatag | atcgttcaaa | gccgtctgaa | ccaattcaac | |
| 421 | ctgtgaatac | ttgggagatt | tccagccttc | caagctctac | tgtgacttca | gagacactgc | |
| 481 | caagcagcct | tagccctagt | gtagagaaat | acaggtatca | ggatgaagat | acacctcctc | |
| 541 | aagagcatat | ttccccacaa | atcacaaatg | aagtgatagg | tccagaattg | gttcatgtct | |
| 601 | cagagaagaa | cttatcagag | attgagaatg | tccatggatt | tgtttctcat | tctcatattt | |
| 661 | caccaataaa | gccaacagaa | gctgttcttc | cctctcctcc | cactgtccct | gtgatccctg | |
| 721 | tcctgccagt | ccctgctgag | aatactgtca | tcctacccac | cataccacag | gcaaatcctc | |
| 781 | ccccagtact | ggtcaacaca | gatagcttgg | aaacaccaac | ttacgttaat | ggcacagatg | |
| 841 | cagattatga | atatgaagaa | atcacacttg | aaaggggaaa | ttcagggctt | ggtttcagca | |
| 901 | ttgcaggagg | tacggacaac | ccacacattg | gagatgactc | aagtattttc | attaccaaaa | |
| 961 | ttatcacagg | gggagcagcc | gcccaagatg | gaagattgcg | ggtcaatgac | tgtatattac | |
| 1021 | aagtaaatga | agtagatgtt | cgtgatgtaa | cacatagcaa | agcagttgaa | gcgttgaaag | |
| 1081 | aagcagggtc | tattgtacgc | ttgtatgtaa | aaagaaggaa | accagtgtca | gaaaaaataa | |
| 1141 | tggaaataaa | gctcattaaa | ggtcctaaag | gtcttgggtt | tagcattgct | ggaggtgttg | |
| 1201 | gaaatcagca | tattcctggg | gataatagca | tctatgtaac | caaaataatt | gaaggaggtg | |
| 1261 | cagcacataa | ggatggcaaa | cttcagattg | gagataaact | tttagcagtg | aataacgtat | |
| 1321 | gtttagaaga | agttactcat | gaagaagcag | taactgcctt | aaagaacaca | tctgattttg | |
| 1381 | tttatttgaa | agtggcaaaa | cccacaagta | tgtatatgaa | tgatggctat | gcaccacctg | |
| 1441 | atatcaccaa | ctcttcttct | cagcctgttg | ataaccatgt | tagcccatct | tccttcttgg | |
| 1501 | gccagacacc | agcatctcca | gccagatact | ccccagtttc | taaagcagta | cttggagatg | |
| 1561 | atgaaattac | aagggaacct | agaaaagttg | ttcttcatcg | tggctcaacg | ggccttggtt | |
| 1621 | tcaacattgt | aggaggagaa | gatggagaag | gaatatttat | ttcctttatc | ttagccggag | |
| 1681 | gacctgctga | tctaagtgga | gagctcagaa | aaggagatcg | tattatatcg | gtaaacagtg | |
| 1741 | ttgacctcag | agctgctagt | catgagcagg | cagcagctgc | attgaaaaat | gctggccagg | |
| 1801 | ctgtcacaat | tgttgcacaa | tatcgacctg | aagaatacag | tcgttttgaa | gctaaaatac | |
| 1861 | atgatttacg | ggagcagatg | atgaatagta | gtattagttc | agggtcaggt | tctcttcgaa | |
| 1921 | ctagccagaa | gcgatccctc | tatgtcagag | ccctttttga | ttatgacaag | actaaagaca | |
| 1981 | gtgggcttcc | cagtcaggga | ctgaacttca | aatttggaga | tatcctccat | gttattaatg | |
| 2041 | cttctgatga | tgaatggtgg | caagccaggc | aggttacacc | agatggtgag | agcgatgagg | |
| 2101 | tcggagtgat | tcccagtaaa | cgcagagttg | agaagaaaga | acgagcccga | ttaaaaacag | |
| 2161 | tgaaattcaa | ttctaaaacg | agagataaag | ggcagtcatt | caatgacaag | cgtaaaaaga | |
| 2221 | acctcttttc | ccgaaaattc | cccttctaca | agaacaagga | ccagagtgag | caggaaacaa | |
| 2281 | gtgatgctga | ccagcatgta | acttctaatg | ccagcgatag | tgaaagtagt | taccgtggtc | |
| 2341 | aagaagaata | cgtcttatct | tatgaaccag | tgaatcaaca | agaagttaat | tatactcgac | |
| 2401 | cagtgatcat | attgggacct | atgaaagaca | ggataaatga | tgacttgatc | tcagaatttc | |
| 2461 | ctgacaaatt | tggatcctgt | gttcctcata | caactagacc | aaaacgagat | tatgaggtag | |
| 2521 | atggaagaga | ttatcatttt | gtgacttcaa | gagagcagat | ggaaaaagat | atccaggaac | |
| 2581 | ataaattcat | tgaagctggc | cagtataaca | atcatctata | tggaacaagt | gttcagtctg | |
| 2641 | tacgagaagt | agcaggaaag | ggcaaacact | gtatccttga | tgtgtctgga | aatgccataa | |
| 2701 | agagattaca | gattgcacag | ctttacccta | tctccatttt | tattaaaccc | aaatccatgg | |
| 2761 | aaaatatcat | ggaaatgaat | aagcgtctaa | cagaagaaca | agccagaaaa | acatttgaga | |
| 2821 | gagccatgaa | actggaacag | gagtttactg | aacatttcac | agctattgta | cagggggata | |
| 2881 | cgctggaaga | catttacaac | caagtgaaac | agatcataga | agaacaatct | ggttcttaca | |
| 2941 | tctgggttcc | ggcaaaagaa | aagctatgaa | aactcatgtt | tctctgtttc | tcttttccac | |
| 3001 | aattccattt | tctttggcat | ctctttgccc | tttcctctgg | aaaaaa | | |

| | |
|---|---|
| MPVRKQDTQRALHLLEEYRSKLSQTEDRQLRSSIERVINIFQSN | (SEQ ID NO:267) |
| LFQALIDIQEFYEVTLLDNPKCIDRSKPSEPIQPVNTWEISSLPSSTVTSETLPSSLS | |
| PSVEKYRYQDEDTPPQEHISPQITNEVIGPELVHVSEKNLSEIENVHGFVSHSHISPI | |
| KPTEAVLPSPPTVPVIPVLPVPAENTVILPTIPQANPPPVLVNTDSLETPTYVNGTDA | |
| DYEYEEITLERGNSGLGFSIAGGTDNPHIGDDSSIFITKIITGGAAAQDGRLRVNDCI | |
| LQVNEVDVRDVTHSKAVEALKEAGSIVRLYVKRRKPVSEKIMEIKLIKGPKGLGFSIA | |
| GGVGNQHIPGDNSIYVTKIIEGGAAHKDGKLQIGDKLLAVNNVCLEEVTHEEAVTALK | |
| NTSDFVYLKVAKPTSMYMNDGYAPPDITNSSSQPVDNHVSPSSFLGQTPASPARYSPV | |
| SKAVLGDDEITREPRKVVLHRGSTGLGFNIVGGEDGEGIFISFILAGGPADLSGELRK | |
| GDRIISVNSVDLRAASHEQAAAALKNAGQAVTIVAQYRPEEYSRFEAKIHDLREQMMN | |
| SSISSGSGSLRTSQKRSLYVRALFDYDKTKDSGLPSQGLNFKFGDILHVINASDDEWW | |
| QARQVTPDGESDEVGVIPSKRRVEKKERARLKTVKFNSKTRDKGQSFNDKRKKNLFSR | |
| KFPFYKNKDQSEQETSDADQHVTSNASDSESSYRGQEEYVLSYEPVNQQEVNYTRPVI | |
| ILGPMKDRINDDLISEFPDKFGSCVPHTTRPKRDYEVDGRDYHFVTSREQMEKDIQEH | |
| KFIEAGQYNNHLYGTSVQSVREVAGKGKHCILDVSGNAIKRLQIAQLYPISIFIKPKS | |
| MENIMEMNKRLTEEQARKTFERAMKLEQEFTEHFTAIVQGDTLEDIYNQVKQIIEEQS | |
| GSYIWVPAKEKL | |

Putative function
Component of cell junctions, possible role in proliferation

### Example 28B

### Validation of GENE Function by RNA interference (RNAi) Knockdown in Drosophila Cultured Cells

To confirm the mitotic role of the target protein, knockdown of GENE expression is performed in cultured Drosophila Dme1-2 cells using a double stranded RNA (dsRNA) from within the Dlg1 (CG1725) gene corresponding to the following sequence:

| | |
|---|---|
| GGAGGCCTTTCATCCGGACAACAATTGTCGCAGTCCCAATCGCAGTTGGCCACCAGC | (SEQ ID NO:268) |
| CAGAGCCAAAGTCAGGTGCATCAGCAGCAGCATGCGACGCCGATGGTCAATTCGCA | |
| GTCGACAGGTGCGCTAAATAGTATGGGACAGACGGTTGTCGATTCACCATCAATACC | |
| ACAAGCAGCCGCAGCAGTAGCAGCAGCAGCAAATGCATCTGCATCTGCATCAGTCA | |
| TTGCAAGCAACAACACAATCAGCAACACCACAGTCACCACAGTCACGGCCACGGCC | |
| ACAGCCAGCAACAGTAGCAGCAAGTTGCCGCCGTCGCTTGGCGCTAACAGCAGCAT | |
| TAGCATTAGCCAATAGCAATAGCAATAGCAACAGCAATAATATCAACAACATTAATA | |
| GCATCAACAACAACAACAGTAGCAGCAGCAGCACGACGGCAACTGTTGCAGCAGCA | |
| ACACCAACAGCAGCATCAGCAGCAGCAGCAGCAGCATCATCTCCACCCGCCAACTC | |
| CTTCTATAA | |

dsRNA is prepared by annealing complimentary RNAs made by in vitro transcription from a PCR fragment created with the following PCR primers:

(SEQ ID NO:269)

TAATACGACTCACTATAGGGAGAGGAGGCCTTTCATCCGGACAACAAT

(SEQ ID NO:270)

TAATACGACTCACTATAGGGAGATTATAGAAGGAGTTGGCGGGTGGAG

Cells are transfected with double stranded RNA in the presence of ‘Transfast’ transfection reagent. A control transfection of a non-endogenous RNA corresponding to RFP (red fluorescent protein) is carried out in parallel.

Analysis of Dlg1 Knockdown by RNAi in D-Mel2 Cells by Cellomics Mitotic Index Assay

For the transfection, 1 μg dsRNA is added to a well of a 96-well Packard viewplate and 35 μl of logarithmically growing DMel-2 cells diluted to 2.3×10⁵ cells/ml in fresh Drosophila-SFM/glutamine/Pen-Strep are added. Cells are incubated with the dsRNA (60 nM) in a humid chamber at 28° C. for 1 hr before addition of 100 μl Drosophila-SFM/glutamine/Pen-Strep. Cells are incubated at 28° C. for 72 hours before analysis. For the assay, cells were fixed and stained using the Cellomics Mitotic Index HitKit following manufacturers instructions. The mitotic index of cells in each well was determined using the ArrayScan HCS System, running the Application protocol Mike_{—}250502_Polgen_MitoticIndex_{—}10×_p2.0 with the 10× objective and the DualBGlp filter set. This automated screening system detects the levels of a specific antigen (phosphorylated histone H3) which is only detectable during mitosis while the chromosomes are condensed.

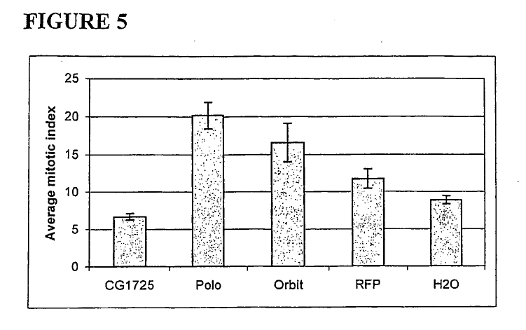

Results for Dlg1 (CG1725) are shown in FIG. 5. A reproducible and significant reduction in mitotic index is observed in this assay indicating a reduction in the number of cells entering mitosis after RNAi

Analysis of Dlg1 Knockdown by RNAi in D-Mel2 cells by Microscopy

For transfection 9 μl of Transfast reagent (Promega) is added to 3μg gene specific dsRNA in 500 μl Drosophila Schneiders medium (no additives) and incubated at room temperature for 15 min. For control wells an equivalent amount of RFP dsRNA is used. This mix is added to a well of a 6-well tissue culture plate containing a glass coverslip and 500 μl of a Dme1-2 cells at 1×10⁶ cells/ml in shneiders medium. After a 1 hour incubation at 28° C., 2 mls Schneiders medium+10% FCS and pen/strep solution is added and cells are incubated at 28° C. for 48 hours. Cells on the coverslip are fixed in formaldehyde and stained with antibodies which detect α-tubulin and γ-tubulin (centrosomes), and are co-stained with DAPI to detect DNA.

Although no pronounced increase in the frequency of chromosomal defects (see Table 3 below) was observed upon RNAi, there was a small increase (30% compared to 10% in control cells) of spindle defects, of which the majority (>90%) had multiple centrosomes (more than 2).

TABLE 3
Mitotic defects observed in Dmel-2 cells after siRNA with Dlg1
(CG1725)

| | Number cells with | Number of | % of chromosomal |
|---|---|---|---|
| | chromosomal | cells with | defects (no defects/ |
| dsRNA | defects | normal mitisis | total cells in mitosis) |
| No RNA | 135 | 314 | 39.47 |
| RFP | 137 | 309 | 40.29 |
| CG1725 | 152 | 169 | 47.35 |

### Example 28B

### Human Dlg1 and Dlg2 are Human Homologues of Drosophila Dlg1

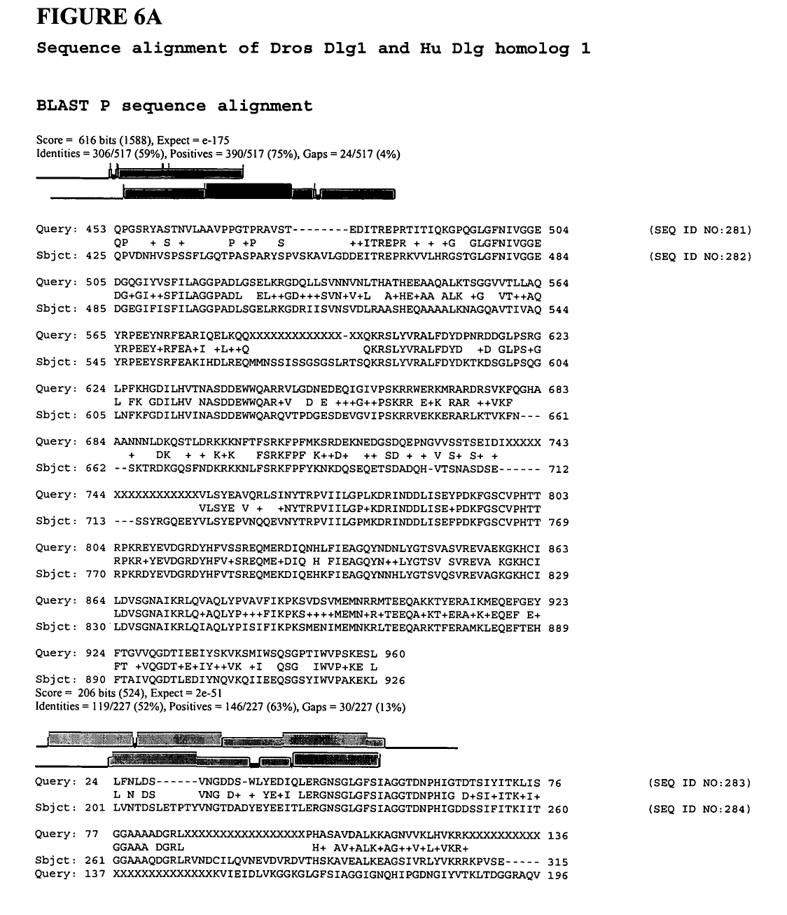

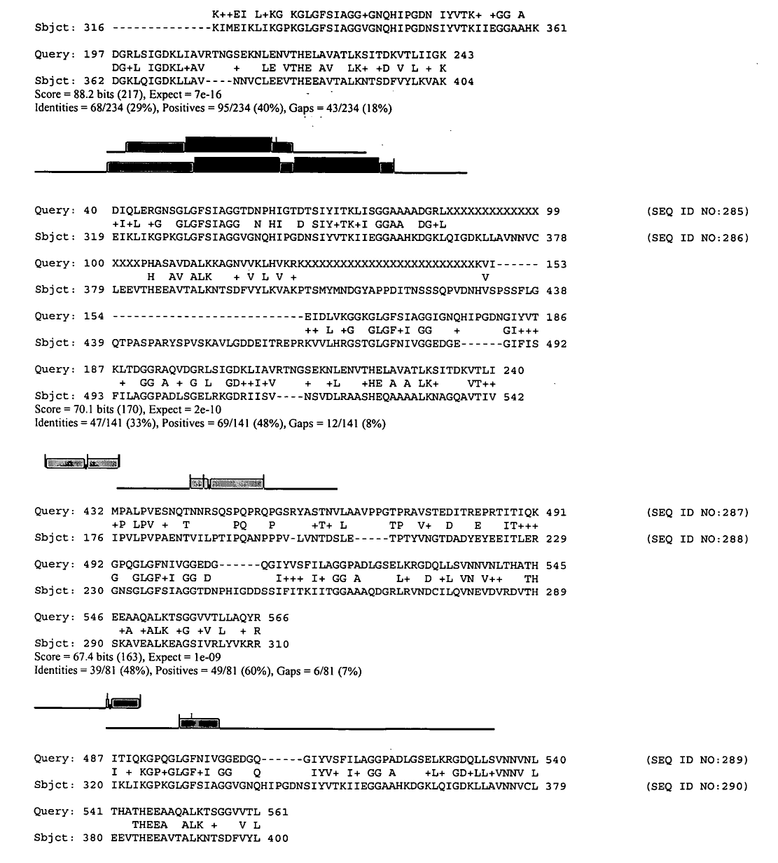

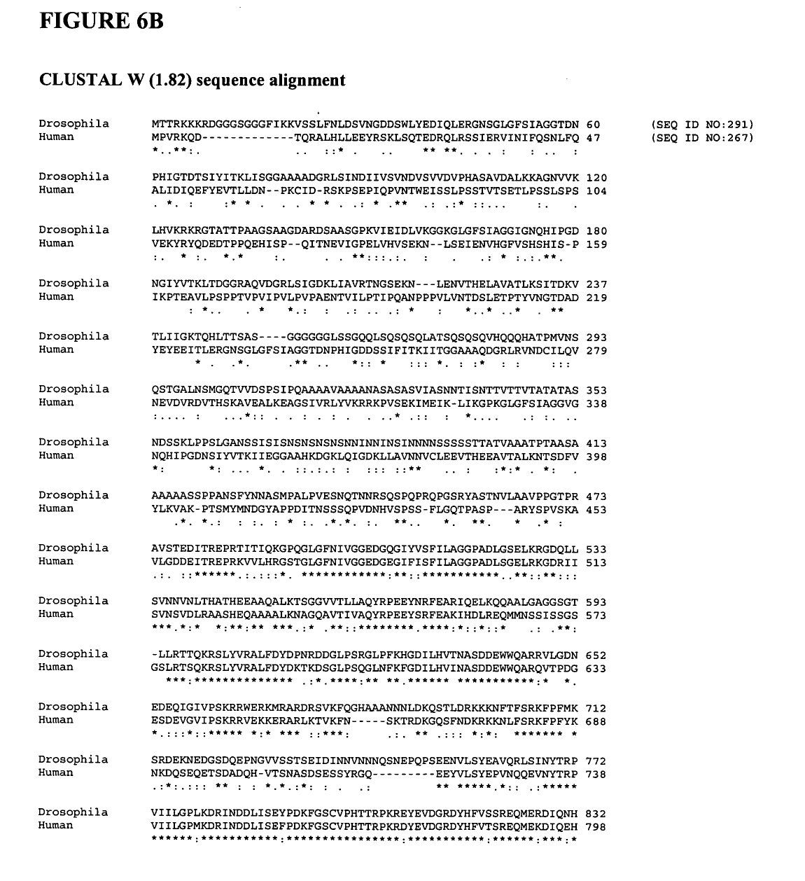

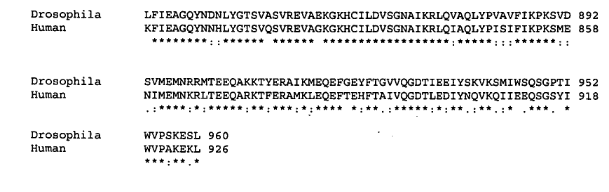

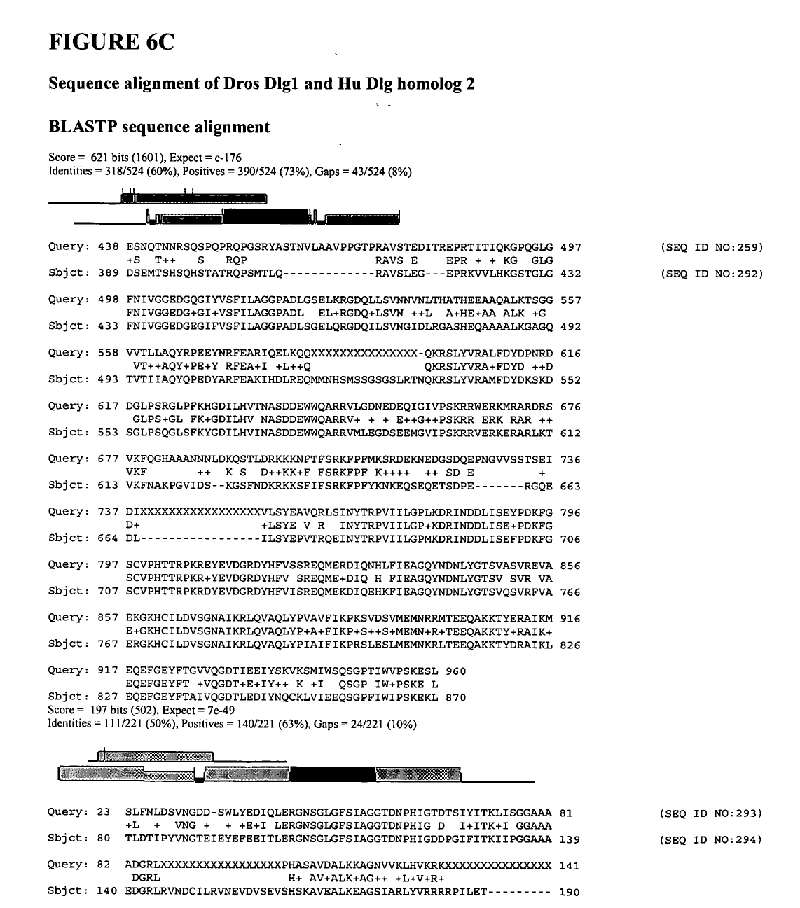

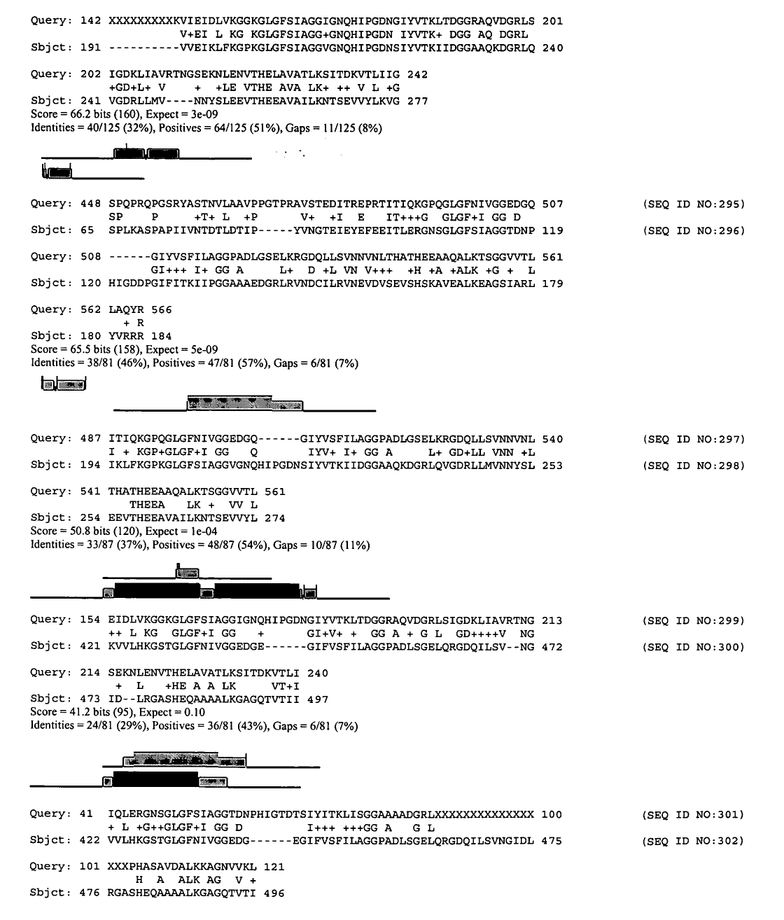

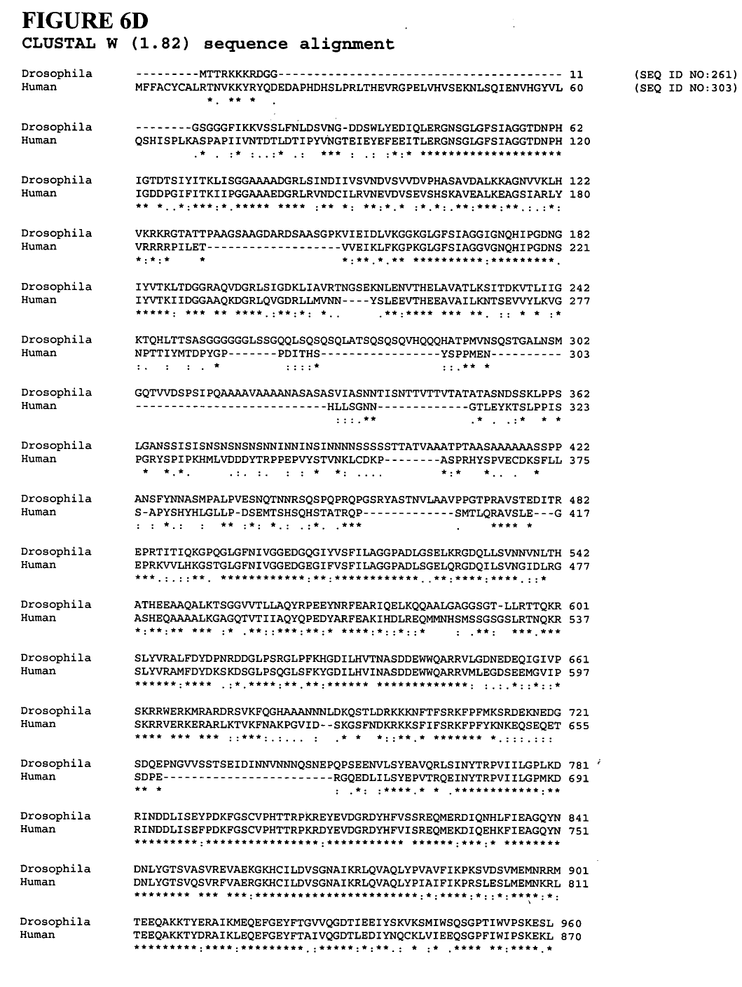

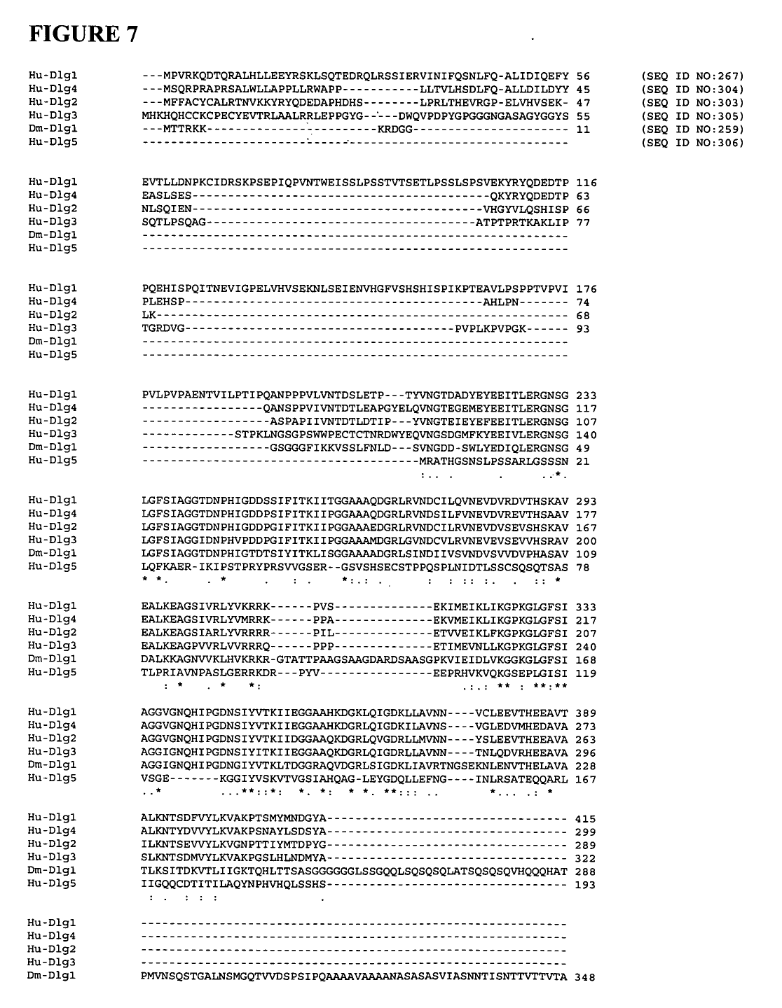

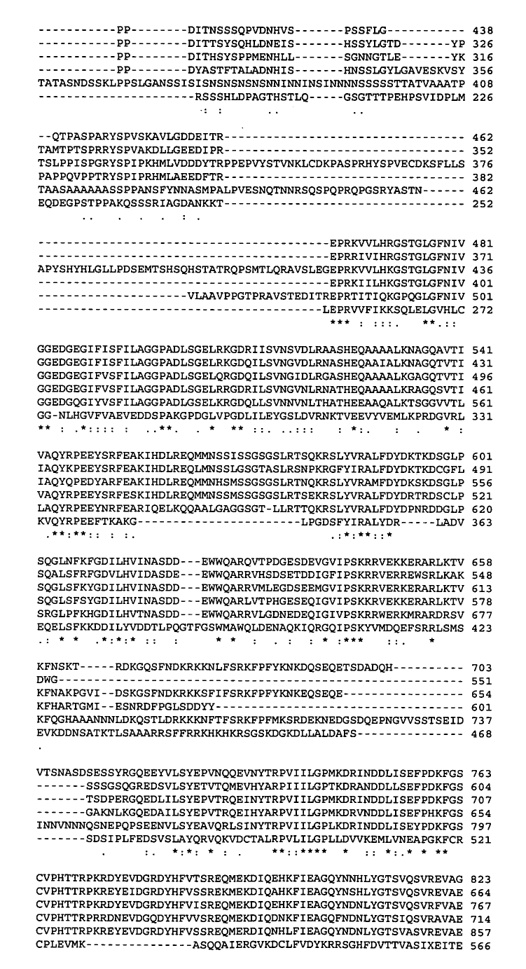

BLASTP with Drosophila Dlg1 reveals 59% (306/517) sequence identity with regions of the human discs, large (Drosophila) homolog 1 (GENBANK ACCESSION U13896), and 60% (318/524) sequence identity with regions of human discs, large (Drosophila) homolog 2 (GENBANK ACCESSION U32376) that human Dlg1 and Dlg2 are is a homologues of Drosophila Dlg1. The BLASTP results are shown in FIG. 6. FIG. 7 shows a Clustal W alignment of Drosophila Dlg1 and the five human Dlg homologues that are currently detailed in the NCBI database. Considering the homology between the human Dlg proteins, it is probable that some or all of them are functionally similar to Drosophila Dlg1.

The nucleotide sequence of the human Dlg1 and human Dlg2 genes and their deduced amino acid sequences are shown in example 28 above.

### Example 28C

### Validation of the Mitotic Role of the Human Homologue by siRNA Knockdown of GENE Expression in Human Cultured Cells

Generation of siRNA Human Dlg1 and Dlg2 Knockdowns

Knockdown of human Dlg1 and Dlg2 gene expression is achieved by siRNA (short interfering RNA, Elbashir et al, Nature May 24, 2001;411(6836):494-8). We used synthetic double stranded RNAs corresponding to two different regions of each of the human Dlg1 and Dlg2 mRNAs. Synthetic siRNAs are obtained from Dharmacon Inc (our supplier). The siRNA sequences are:

| | | | |
|---|---|---|---|
| COD1652 | dlg2-1 | AACAUUGUCGGUGGG | Corresponds to nucleotides |
| | | GAAGAU | 1576-1596 human Dlg-2 |
| | | (SEQ ID NO:271) | (see example 28 above) |
| COD1653 | dlg2-2 | AAAACCCAGGUCUCU | Corresponds to nucleotides |
| | | GGAACC | 2664-2684 in human Dlg-2 |
| | | (SEQ ID NO:272) | (see example 28 above) |
| COD1654 | dlg1-1 | AAAGGGGAAAUUCAG | Corresponds to nucleotides |
| | | GGCUUG | 871-891 in human Dlg-1 |
| | | (SEQ ID NO:273) | (see example 28 above) |
| COD1655 | dlg1-2 | AAGUAGCAGGAAAGG | Corresponds to nucleotides |
| | | GCAAAC | 2647-2667 in human Dlg-1 |
| | | (SEQ ID NO:274) | (see example 28 above) |

Analysis of siRNA Hu Dlg1 and Dlg2 Knockdowns in U2OS Cells by Flow Cytometry Analysis

Cells are seeded in 6-well tissue culture dishes at 1×10⁵ cells/well in 2 ml Dulbecco's Modified Eagle's Medium (DMEM) (Sigma)+10% Foetal Bovine Serum (FBS) (Perbio), and incubated overnight (37° C./5% CO₂).

For each well, 12 μl of 20 μM siRNA duplex (Dharmacon, Inc) (in RNAse-free H₂O) is mixed with 200 μl of Optimem (Invitrogen). In a separate tube 8 μl of oligofectamine reagent (Invitrogen) was mixed with 52 μl of Optimem, and incubated at room temperature for 7-10 mins. The oligofectamine/Optimem mix is then added dropwise to the siRNA/Optimem mix, and this is then mixed gently, before being incubated for 15-20 mins at room temperature. During this incubation the cells are washed once with DMEM (with no FBS or antibiotics added). 600 μl of DMEM (no FBS or antibiotics) is then added to each well.

Following the 15-20 min incubation, 128 μl of Optimem is added to the siRNA/oligofectamine/optimem mix, and this was added to the cells (in 600 μl DMEM). The transfection mix is added at the edge of each well to assist dilution before contact is made with the cells. Cells are then incubated with the transfection mix for 4 h (37° C./5% CO₂). Subsequently 1 ml DMEM+20% FBS is added to each well. Cells are then incubated at 37° C./5% CO₂ for 72 h. Cells are harvested by trypsinisation, washed in PBS, fixed in ice-cold 70% EtOH and stained with propidium iodide before Facs analysis.

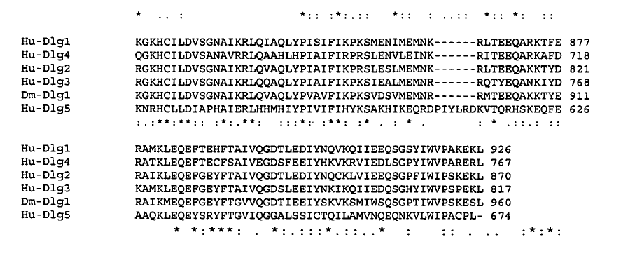

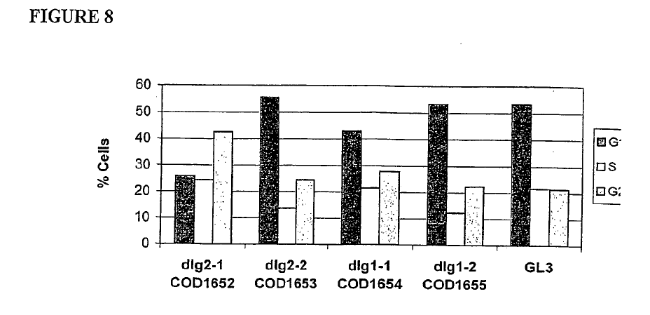

siRNA Hu Dlg1 and Dlg2 knockdowns are conducted in U2OS. As shown in FIG. 8 major changes in the distribution of cells between cell cycle compartments (G1, S, G2/M) are seen with Dlg1 siRNA COD1564 and Dlg2 siRNA COD1562. In both cases an accumulation of cells with a 2N DNA content, indicated as the G2/M compartment of the cell cycle, is observed with a concomitant reduction in the 1N DNA content G1 compartment population. This indicates that a proportion of cells may unable to exit mitosis and renter G1 and so may be unable to complete cytokinesis, or have entered the next cycle as polyploid cells.

Subsequent microscopic analysis is performed in order to phenotype the Hu Dlg1 and Dlg2 siRNA induced defect and check for the presence of large multinucleate cells which may, due to their size and ploidy, be excluded from the FACS analysis.

Analysis of Hu Dlg1 and Dlg2 siRNA Knockdowns in U2OS Cells by Microscopy

The transfection method for samples for microscopy is identical to that for Facs except that cells are plated in wells containinmg a sterile glass coverslip. Cells are incubated with siRNA for 48 hours before formaldehyde fixation and co-staining with Dapi to reveal DNA (blue) and antibodies to reveal microtubules (red) and centrosomes (green). Antibodies used are: rat anti-alpha tubulin (YL12) (supplier Serotec) with secondary antibody goat anti-rat IgG-TRITC (supplier Jackson Immunoresearch) and mouse anti-gamma-tubulin (GTU88) with secondary antibody Alexagreen488-goat anti-mouseIgG (supplier Sigma).

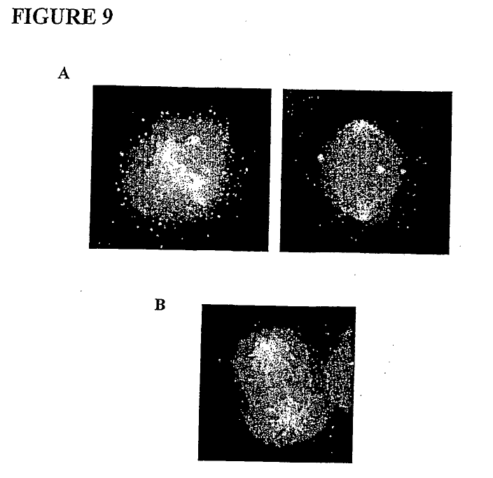

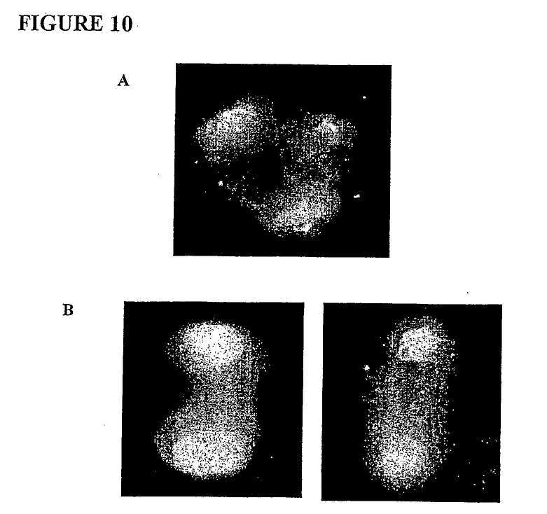

Phenotype analysis by microscopy is conducted on U2OS cells. Results from duplicate experiments in U2OS cells are shown in FIGS. 9 and 10, and Table 4 below. Generally after siRNA more of the cells in mitosis seem to be in the early stages, prometaphase rather than the later stages (metaphase, anaphase telophase) a high frequency of cells have multiple centrosomes as is also observed in RNAi with Dme1-2 cell siRNA (see above). In addition transfected cells appear to be unable to successfully carry out cytokinesis which may account for the increase in polyploid cells.

TABLE 4
Brief description of significant cell division defects after
Dlg1 and 2 siRNA in U2OS cells.

| Gene/siRNA | Dlg1/COD1564 | Dlg2/COD1562 |
|---|---|---|
| Cell Type | U2OS | U2OS |
| Polyploidy | Increased (4.8/field | Increased (4.8/field |
| | compared to 1.6/field in | compared to 1.6/field in |
| | nuntreated) | nuntreated) |
| Mitotic Defects | Increased (23% | Increased (36% compared |
| | compared to 13% in | to 13% in untreated) |
| | untreated) | |
| Main knockout | Increased number of | Increased number of |
| phenotype | multi - centrosomal cells | multi - centrosomal cells |
| | (7.3% compared to 2.6% | (6.6% compared to 2.6%) |
| | in untreated) | in untreated) |
| | Cytokinesis defects (10% | Cytokinesis defects (23% |
| | compared to 0% in | compared to 0% in |
| | untreated) | untreated) |
| | Large increase in | Large increase in |
| | apoptotic cells | apoptotic cells |
| Additional | Increase in ratio of | Increase in ratio of |
| observations | prophase to | prophase to prometaphase |
| | prometaphase (61% | (72% compared to 43% |
| | compared to 43% in | in untreated cells) |
| | untreated cells) | Decrease in ratio of |
| | Decrease in ratio of | metaphase (6% compared |
| | metaphase (5% compared | to 22% in untreated cells) |
| | to 22% in untreated cells) | Decrease in ratio of |
| | | anaphase and telophase |
| | | (19% compared to 27% |
| | | in untreated cells) |

The above results confirm that Dlg1 and Dlg2 are involved in cell cycle progression, in particular, in achieving successful cell separation during cytokinesis. The mutiplication of entrosomes in many cells after Dlg 1 or 2 RNAi may reflect failure to undergo cytokinesis so that cells prematurely enter the next cycle, or may indicate that the centrosome duplication cycle is overriding normal cell cycle checkpoints. Accordingly, modulators of Dlg1 and Dlg2 activity (as identified by the assays described above) may be used to treat any proliferative disease.

### Example 28D

### Expression of Recombinant Hu Dlg Protein in Insect Cells

A cDNA encoding the Human Dlg1 or Dlg2 coding region derived by RT-PCR is inserted into the baculovirus expression vector pFastbacHTc (Life Technologies). A baculovirus stock is generated and western blot of subsequent infections of Sf9 insect cells demonstrates expression of N-terminal 6-His tagged proteins of approximately 100 kD (Dlg1) and 97kD (Dlg2). The recombinant protein is purified by Ni—NTA resin affinity chromatography.

Similarly 6-His tagged Dlg proteins are expressed in bacteria by inserting cDNAs into bacterial expression plamids pDest17 or pET series. Protein expression in cultures of host E.coli cells transformed with recombinant plasmid is induced by addition of inducer chemical IPTG. The recombinant protein is purified by Ni—NTA resin affinity chromatography

### Example 28E

### Assay for Modulators of Dlg Activity

Digs are Membrane-associated guanylate kinase (MAGUK) homologues and contain several protein—protein interaction domains including PDZ domains, SH3 domains and a C-terminal guanylate kinase homology region that does not possess guanylate kinase activities but may act as a protein—protein interaction domain. Several proteins are known to bind huDIg1 including the adenomatous polposis coli (APC) tumor suppressor protein, the human papillomavirus E6 transforming protein, transforming adenovirus E4 protein, and the PDZ-binding kinase PBK (Gaudet et al 2000). An assay for modulators of Dlg activity would consist of an ELISA type assay where full length Dlg protein, or individual PDZ domains of Dlg protein expressed in bacteria or insect cells (as described above) are bound to a solid support, and interaction with the PDZ binding proteins described above could be measured by antibody detection of, or radioactive labelling of the PDZ binding proteins.

### Example 29

### Category 3

Line ID—419

Phenotype—Lethal phase, prepupal—pupal. High mitotic index, colchicines-like chromosome condensation, metaphase arrest

Annotated Drosophila genome genomic segment containing P element insertion site (and map position)—AE003450 (9C)

P element insertion site—292,726

Annotated Drosophila genome Complete Genome candidate

CG12638—sprint, ras associated protein

(SEQ ID NO:275)

ATGTTTGCCATATCATTGCAGCTGCTCAGCTCGCTGGCCAGCGATTTGGA
CATAATGCTAAACGATCTTCGATCGGCGCCGAGTCATGCTGCAACAGCAA
CAGCAACAGCAACAACAACGGCAACAGTTGCAACTGCAACCGCAACAACA
ACGGCCAACCGGCAGCAGCAACATCATAATCACCATAATCAGCAGCAAAT
GCAATCAAGGCAATTGCATGCACATCATTGGCAGAGCATTAACAACAATA
AGAATAACAACATTAGTAACAAAAACAACAACAACAACAACAATAATAAC
AATAACATTAATAACAATAATAATAATAATAATCATTCGGCACACCCACC
TTGCCTGATCGATATTAAGCTGAAGTCAAGCCGATCGGCAGCAACAAAAA
TAACCCATACAACAACCGCCAATCAGCTGCAGCAACAACAACGCCGCCGT
GTGGCACCCAAGCCACTGCCACGCCCACCGCGACGTACCCGCCCAACGGG
ACAAAAGGAGGTGGGGCCGTCTGAAGAGGATGGGGACACGGATGCCAGTG
ACCTGGCCAATATGACATCACCGCTGAGCGCCAGTGCAGCGGCCACTCGA
ATCAACGGCCTCTCGCCGGAAGTGAAGAAAGTCCAGCGGTTGCCACTGTG
GAATGCGCGAAACGGAAACGGAAGTACCACCACCCACTGTCACCCAACCG
GCGTCTCTGTGCAACGCCGTCTGCCCATCCAAAGTCATCAGCAGCGAATT
CTAAACCAACGATTTCATCACCAGCGAATGCATCATGGGTAA

(SEQ ID NO:276)

MFAISLQLLSSLASDLDIMLNDLRSAPSHAATATATATTTATVATATATT
TANRQQQHHNHHNQQQMQSRQLHAHHWQSI NNKNNNISNKNNNNNNNNN
NNINNNNNNNNHSAHPPCLIDIKLKSSRSAATKTHTTTANQLQQQQRRR
VAPKPLPRPPRRTRPTGQKEVGPSEEDGDTDASDLANMTSPLSASAAATR
INGLSPEVKKVQRLPLWNARNGNGSTTTHC HPTGVSVQRRLPIQSHQQRI
LNQRFHHQRM HHG

Human homologue of Complete Genome candidate

B38637—Ras inhibitor (clone JC265)—human (fragment)

```
(SEQ ID NO:277)
      1 ggccggcagc ggctgagcga catgagcatt tctacttcct cctccgactc gctggagttc
    61 gaccggagca tgcctctgtt tggctacgag gcggacacca acagcagcct ggaggactac
  121 gagggggaaa gtgaccaaga gaccatggcg ccccccatca agtccaaaaa gaaaaggagc
  181 agctccttcg tgctgcccaa gctcgtcaag tcccagctgc agaaggtgag cggggtgttc
  241 agctccttca tgaccccgga gaagcggatg gtccgcagga tcgccgagct ttcccgggac
  301 aaatgcacct acttcgggtg cttagtgcag gactacgtga gcttcctgca ggagaacaag
  361 gagtgccacg tgtccagcac cgacatgctg cagaccatcc ggcagttcat gacccaggtc
  421 aagaactatt tgtctcagag ctcggagctg gaccccccca tcgagtcgct gatccctgaa
  481 gaccaaatag atgtggtgct ggaaaaagcc atgcacaagt gcatcttgaa gcccctcaag
  541 gggcacgtgg aggccatgct gaaggacttt cacatggccg atggctcatg gaagcaactc
  601 aaggagaacc tgcagcttgt gcggcagagg aatccgcagg agctgggggt cttcgccccg
  661 acccctgatt ttgtggatgt ggagaaaatc aaagtcaagt tcatgaccat gcagaagatg
  721 tattcgccgg aaaagaaggt catgctgctg ctgcgggtct gcaagctcat ttacacggtc
  781 atggagaaca actcagggag gatgtatggc gctgatgact tcttgccagt cctgacctat
  841 gtcatagccc agtgtgacat gcttgaattg gacactgaaa tcgagtacat gatggagctc
  901 ctagacccat cgctgttaca tggagaagga ggctattact tgacaagcgc atatggagca
  961 ctttctctga taaagaattt ccaagaagaa caagcagcgc gactgctcag ctcagaaacc
1021 agagacaccc tgaggcagtg gcacaaacgg agaaccacca accggaccat cccctctgtg
1081 gacgacttcc agaattacct ccgagttgca tttcaggagg tcaacagtgg ttgcacagga
1141 aagaccctcc ttgtgagacc ttacatcacc actgaggatg tgtgtcagat ctgcgctgag
1201 aagttcaagg tgggggaccc tgaggagtac agcctctttc tcttcgttga cgagacatgg
1261 cagcagctgg cagaggacac ttaccctcaa aaaatcaagg cggagctgca cagccgacca
1321 cagccccaca tcttccactt tgtctacaaa cgcatcaaga acgatcctta tggcatcatt
1381 ttccagaacg gggaagaaga cctcaccacc tcctagaaga caggcgggac ttcccagtgg
1441 tgcatccaaa ggggagctgg aagccttgcc ttcccgcttc tacatgcttg agcttgaaaa
1501 gcagtcacct cctcggggac ccctcagtgt agtgactaag ceatecacag gccaactcgg
1561 ccaagggcaa ctttagccac gcaaggtagc tgaggtttgt gaaacagtag gattctcttt
1621 tggcaatgga gaattgcatc tgatggttca agtgtcctga gattgtttgc tacctacccc
1681 cagtcaggtt ctaggttggc ttacaggtat gtatatgtgc agaagaaaca cttaagatac
1741 aagttctttt gaattcaaca gcagatgctt gcgatgcagt gcgtcaggtg attctcactc
1801 ctgtggatgg cttcatccct g
(SEQ ID NO:278)
      1 grqrlsdmsi stsssdslef drsmplfgye adtnssledy egesdqetma ppikskkkrs
    61 ssfvlpklvk sqlqkvsgvf ssfmtpekrm vrriaelsrd kctyfgclvq dyvsflqenk
  121 echvsstdml qtirqfmtqv knylsqssel dppieslipe dqidvvleka mhkcilkplk
  181 ghveamlkdf hmadgswkql kenlqlvrqr npqelgvfap tpdfvdveki kvkfmtmqkm
  241 yspekkvmll lrvckliytv mennsgrmyg addflpvlty viaqcdmlel dteieymmel
  301 ldpsllhgeg gyyltsayga lsliknfqee qaarllsset rdtlrqwhkr rttnrtipsv
  361 ddfqnylrva fqevnsgctg ktllvrpyit tedvcqicae kfkvgdpeey slflfvdetw
  421 qqlaedtypq kikaelhsrp qphifhfvyk rikndpygii fqngeedltt s
```

Putative function
Ras associated effector protein

### REFERENCES

Altschul, S. F. and Lipman, D. J. (1990) Protein database searches for multiple alignments. Proc. Natl. Acad. Sci. USA 87: 5509-5513

Burge, C. and Karlin, S. (1997) Prediction of complete gene structures in human genomic DNA. J. Mol. Biol. 268, 78-94.

Deak, P., Omar, M. M., Saunders, R. D. C., Pal, M., Komonyi, O., Szidonya, J., Maroy, P., Zhang, Y., Ashbumer, M., Benos, P., Savakis, C., Siden-Kiamos, I., Louis, C., Bolshakov, V. N., Kafatos, F. C., Madueno, E., Modolell, J., Glover, D. M. (1997) Correlating physical and cytogenetic maps in chromosomal region 86E-87F of Drosophila melanogaster. Genetics 147:1697-1722.

Gaudet S, Branton D and Lue R A (2000) Characterisation of PDZ-binding kinase, a mitotic kinase PNAS 97, 5167-5172

Jowett, T. (1986) Preparation of nucleic acids. In “Drosophila: A Practical Approach.” Ed Roberts, D. B. IRL Press Oxford.

Lefevre, G. (1976) A photographic representation and interpretation of the polytene chromosomes of Drosophila melanogaster salivary glands. In: The Genetics and Biology of Drosophila, Eds Ashbumer, M. and Novitski, E. Academic Press.

Pirrotta, V. (1986) Cloning Drosophila genes. In: In “Drosophila: A Practical Approach.” Ed Roberts, D. B. IRL Press Oxford.

Saunders, R. D. C., Glover, D. M., Ashbumer, M., Siden-Kiamos, I., Louis, C., Monastirioti, M., Savakis, C., Kafatos, F. C.(1989) PCR amplification of DNA microdissected from a single polytene chromosome band: a comparison with conventional microcloning. Nucleic Acids Res. 17:9027-9037

Takada T, Matozaki T, Takeda H, Fukunaga K, Noguchi T, Fujioka Y, Okazaki I, Tsuda M, Yamao T, Ochi F, Kasuga M. (1998) Roles of the complex formation of SHPS-1 with SHP-2 in insulin-stimulated mitogen-activated protein kinase activation. J Biol Chem Apr. 10, 1998;273(15):9234-42

Torok, T., Tick, G., Alvarado, M., Kiss, I. (1993) P-lacW insertional mutagenesis on the second chromosome of Drosophila melanogaster: isolation of lethals with different overgrowth phenotypes. Genetics 135(1):71-80

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of treating cancer in an individual, comprising administering to said individual a composition comprising a polynucleotide selected from the group consisting of the nucleotide sequences in Table 5, or a polypeptide encoded by the polynucleotide.

2. The method of claim 1, wherein said polypeptide is a human polypeptide selected from the group consisting of the polypeptides in column 3 of Table 5.

3. A method of identifying a substance which binds to a polypeptide selected from the group consisting of the polypeptides in column 3 of Table 5, said method comprising contacting said polypeptide with a candidate substance and detecting the binding of said substance to said polypeptide.

4. A method of identifying a substance which modulates the function of a polypeptide selected from the group consisting of the polypeptides in column 3 of Table 5, said method comprising the steps of: contacting said polypeptide with a candidate substance and determining the activity of said polypeptide, wherein a change in said activity in the presence of said candidate substance is indicative of said substance modulating the function of said polypeptide.

5. A method of diagnosing a cancer in an individual, said method comprising: (a) providing a biological sample of said individual; (b) contacting said biological sample with a probe comprising a fragment of at least 15 nucleotides of a polynucleotide selected from the group consisting of the polynucleotides in Table 5; and (c) detecting the hybridisation between said probe and said biological sample, wherein the presence of hybridisation is indicative of said cancer in said individual.

6. A method of diagnosing a cancer in an individual, said method comprising: (a) providing a biological sample of said individual; (b) contacting said biological sample with an antibody which binds to a polypeptide selected from the group consisting of the polypeptides in column 3 of Table 5; and (c) detecting the binding of said antibody to said sample, wherein the presence of binding is indicative of said cancer in said individual.

7. A method of modulating the expression of a polynucleotide selected from the group consisting of the polynucleotides in Table 5 in a cell, said method comprising introducing a double stranded RNA (dsRNA) which hybridises to said polynucleotide, or an antisense RNA which hybridises to said polynucleotide, or a fragment thereof, into the cell.

8. The method of claim 7, wherein said modulating is down-regulating.

9. A polynucleotide comprising a sequence selected from the group consisting of:
(a) any one of the nucleotide sequences in Example 19 or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

10. The polynucleotide of claim 9, wherein said sequence in Example 19 is Shp2 polynucleotide sequence or its complement thereof.

11. A polynucleotide comprising a sequence selected from the group consisting of:
(a) any one of the nucleotide sequences in Example 28 or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

12. The method of claim 11, wherein said sequence in Example 28 is Dlg1 or Dlg2.

13. A polynucleotide comprising a sequence selected from the group consisting of:
(a) any one of the nucleotide sequences in Table 5 or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

14. A polynucleotide comprising a sequence selected from the group consisting of:
(a) any one of the nucleotide sequences in Examples 1 to 18, 20 to 27 and 29 or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

15. A polynucleotide comprising a sequence selected from the group consisting of:
(a) any one of the nucleotide sequences in Examples 1, 2, 2A, 2B and 2C or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

16. A polynucleotide comprising a sequence selected from the group consisting of:
(a) any one of the nucleotide sequences in Examples 3 to 9 and 9A or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

17. A polynucleotide comprising a sequence selected from the group consisting of:
(a) any one of the nucleotide sequences in Examples 10 to 29 or the complement thereof;
(b) a nucleotide sequence which hybridises to a sequence of (a) or a fragment thereof; and
(c) a polynucleotide sequence which is degenerate as a result of the genetic code to said sequence(s) in (a) or (b).

18. A polynucleotide probe comprising a fragment of at least 15 consecutive nucleotides of a polynucleotide of claim 9.

19. A polypeptide comprising an amino acid sequence selected from the group consisting of the sequences in:
(a) Example 19;
(b) Example 28;
(c) Table 5;
(d) Examples 1 to 18, 20 to 27 and 29;
(e) Examples 1 to 2, 2A, 2B and 2C;
(f) Examples 3 to 9 and 9A;
(g) Examples 10 to 29; and
(h) a homologue, variant, derivative or fragment thereof.

20. The polypeptide of claim 19, wherein said sequence in Example 19 is Shp2 polypeptide.

21. The polypeptide of claim 19, wherein said sequence in Example 28 is Dlg1 or Dlg2 polypeptide.

22. A vector comprising a polynucleotide according to claim 9.

23. An expression vector comprising a polynucleotide according to claim 9, which is operably linked to a regulatory sequence which directs the expression of said polynucleotide in a host cell.

24. An antibody which binds to a polypeptide of claim 19.

25. A method for detecting the presence or absence of a polynucleotide of claim 9 in a biological sample, said method comprising:
(a) contacting the biological sample under hybridising conditions with a probe comprising a fragment of at least 15 consecutive nucleotides of a polynucleotide having a sequence set forth in Example 19 or a complement thereof; and
(b) detecting hybridisation between said probe and said sample.

26. A method for detecting a polypeptide of claim 19 present in a biological sample which comprises:
(a) providing an antibody that binds to said polypeptide;
(b) contacting said biological sample with said antibody; and
(c) determining binding of said antibody to said biological sample.

27. A method of treating cancer in an individual comprising administering a polynucleotide of claim 9.

28. A method of treating cancer in an individual comprising administering a polypeptide of claim 19.

29. A method of treating cancer in an individual comprising administering an antibody of claim 22.

30. A method for identifying a substance which binds to a polypeptide of claim 19, said method comprising contacting said polypeptide with a candidate substance and detecting the binding of said substance to said polypeptide.

31. A method for identifying a substance which modulates the function of a polypeptide of claim 19, said method comprising the steps of: contacting the polypeptide with a candidate substance and determining the activity of said polypeptide, wherein a change in activity in the presence of said candidate substance is indicative of said substance modulating the function of said polypeptide.

32. A method of identifying a human nucleic acid sequence, by: (a) selecting a Drosophila polypeptide identified in any of Examples 11 to 39, (b) identifying a corresponding human polypeptide; and (c) identifying a nucleic acid encoding the human polypeptide of (b).

33. A method according to claim 32, in which a human homologue of the Drosophila sequence, or a human sequence similar to the Drosophila sequence, is identified in step (b).

34. A method according to claim 32, in which the human polypeptide has at least one of the biological activities, preferably substantially all the biological activities of the Drosophila polypeptide.
